(19) United States
(12) Patent Application Publication
(10) Pub. No.: US 2021/0332440 A1
(43) Pub. Date: Oct. 28, 2021

(54) DETECTION OF ADVANCED ADENOMA AND/OR EARLY STAGE COLORECTAL CANCER

(71) Applicant: Universal Diagnostics, S.L., Seville (ES)

(72) Inventors: Kristi Kruusmaa, Seville (ES); Christian Hense, Seville (ES); Marko Bitenc, Seville (ES); Juan Martinez-Barea, Seville (ES); Pol Sola de los Santos, Seville (ES); Pol Canal Noguer, Seville (ES); Marko Chersicola, Ljubljana (SI); Primo{hacek over (z)} Knap, Ljubljana (SI)

(21) Appl. No.: 17/027,221

(22) Filed: Sep. 21, 2020

Related U.S. Application Data
(60) Provisional application No. 63/011,970, filed on Apr. 17, 2020.

(51) Int. Cl. C12Q 1/6886

(52) U.S. Cl. CPC C12Q 1/6886; C12Q 2600/154

(57) ABSTRACT

The present disclosure provides, among other things, methods for adenoma and/or early stage colorectal cancer detection (e.g., screening) and compositions related thereto. In various embodiments, the present disclosure provides methods for screening that include analysis of methylation status of one or more methylation biomarkers, and compositions related thereto. In various embodiments, the present disclosure provides methods for detection (e.g., screening) that include detecting (e.g., screening) methylation status of one or more methylation biomarkers in cfDNA, e.g., in ctDNA. In various embodiments, the present disclosure provides methods for screening that include detecting (e.g., screening) methylation status of one or more methylation biomarkers in cfDNA, e.g., in ctDNA, using MSRE-qPCR and/or using massively parallel sequencing (e.g., next-generation sequencing).

### CROSS-REFERENCE

This application claims priority to and benefit of U.S. Provisional Patent Application No. 63/011,970, filed on Apr. 17, 2020, the disclosure of which is incorporated by reference in its entirety.

### SEQUENCE LISTING

The instant application contains a Sequence Listing which has been submitted electronically in ASCII format and is hereby incorporated by reference in its entirety. Said ASCII copy, created on Sep. 21, 2020, is named 2011722-0057_SL.txt and is 116,385 bytes in size.

### BACKGROUND

Screening for colorectal cancer is a critical component of cancer prevention, diagnosis, and treatment. Colorectal cancer (CRC) has been identified, according to some reports, as the third most common type of cancer and the second most frequent cause of cancer mortality in the world. According to some reports, there are over 1.8 million new cases of colorectal cancer per year and about 881,000 deaths from colorectal cancer, accounting for about 1 in 10 cancer deaths. Regular colorectal cancer screening is recommended, particular for individuals over age 45. Moreover, incidence of colorectal cancer in individuals below 50 has increased over time. Statistics suggest that current colorectal cancer screening techniques are insufficient.

Furthermore, detecting colon cancers at an early stage would result in decreased mortality rates. Detection and removal of precursors of colon cancer, including but not limited to, colonic polyps with advanced features, will reduce the incidence of CRC as these polyps are believed to represent the greatest risk of malignant progression. Removal of advanced colonic polyps would mitigate the risk of cancer initiation. Generally, about 9-16% of the asymptomatic patients aged 50 and older present with advanced adenoma findings.

Accordingly, there exists a need for methods, compositions, and systems that can provide for detection of colorectal cancer and/or advanced adenoma. In particular, there is a need for detection of colorectal cancer and/or advanced adenoma at an early stage.

### SUMMARY

The present disclosure provides, among other things, methods for detecting premalignant and malignant neoplasms such as advanced adenomas and early stage colorectal cancer with high accuracy from human biospecimens. In various embodiments, the present disclosure provides methods for colorectal cancer screening that include determination of methylation status (e.g., the number, frequency, or pattern of methylation) at one or more methylation sites found within a methylation locus, e.g., a differentially methylated region (DMR), of deoxyribonucleic acid (DNA) of a human subject, and compositions related thereto. In various embodiments, the present disclosure provides methods for advanced adenoma and/or early stage colorectal cancer (e.g., as a combined category, or advanced adenoma as a single category, or early stage colorectal cancer as a single category) that include screening methylation status for each of one or more methylation loci in cfDNA (cell free DNA), e.g., in ctDNA (circulating tumor DNA). In various embodiments, the present disclosure provides methods for colorectal cancer screening that include determining a methylation status for each of one or more methylation loci in cfDNA, e.g., in ctDNA, using, for example, quantitative polymerase chain reaction (qPCR) (e.g., methylation sensitive restriction enzyme quantitative polymerase chain reaction, MSRE-qPCR). In some embodiments, the technology uses massively parallel sequencing (e.g., next-generation sequencing) to determine methylation state, e.g., sequencing by-synthesis, real-time (e.g., single-molecule) sequencing, bead emulsion sequencing, nanopore sequencing, or the like. Various compositions and methods provided herein provide sensitivity and specificity sufficient for clinical application in screening for advanced adenoma and/or early stage colorectal cancer. Various compositions and methods provided herein are useful in advanced adenoma and/or early stage colorectal cancer screening by analysis of an accessible tissue sample of a subject, e.g., a tissue sample that is blood or a blood component (e.g., cfDNA, e.g., ctDNA), or stool.

In one aspect, the invention is directed to a method of detecting (e.g., screening for) advanced adenoma, the method comprising: determining a methylation status for each of one or both of the following, in deoxyribonucleic acid (DNA) of a human subject: (i) a methylation locus within gene NRF1; and (ii) a methylation locus within gene TMEM196; and diagnosing advanced adenoma in the human subject based at least on said determined methylation status(es).

In certain embodiments, the methylation locus of NRF1 comprises at least one (e.g., at least 2, at least 3, at least 4, or more) CpG dinucleotide.

In certain embodiments, the methylation locus of TMEM196 comprises at least one (e.g., at least 2, at least 3, at least 4, or more) CpG dinucleotide.

In certain embodiments, the methylation locus within gene NRF1 comprises at least a portion of (e.g., at least 50% of) NRF1 '565 [chr7:129720565-129720676]

(SEQ ID NO: 1)
[TAACCACCTGCACCTCTGCTGCAATGTAAACAGCAGATGTGGGCGCA
GGGTGAGAAGGGAGAGGAAGCTACGTGCAATGGCAGGTTGGGGAATAA
GGAGGCAGAGGGGCTCC].

In certain embodiments, the methylation locus within gene NRF1 comprises at least 50% of NRF1 '565 and wherein the portion of the methylation locus that overlaps with NRF1 '565 has at least 98% similarity with the overlapping portion of NRF1 '565.

In certain embodiments, the methylation locus within gene TMEM196 comprises at least a portion of (e.g., at least 50% of) TMEM196 '652 [chr7:19772652-19772800][GGAGAGCACCAAGAGGCTCCCAATAATCTGACCGCTGGTGCACATCCTTCCTCGGT CATCTTCCTTCCAGATCAGAGAGGGAAATCAACCATCTACCTTTTTTTCTTCCACTAT CCTCCTTACCCCTTCCACCCCCTACCAGATCCCAA] (SEQ ID NO: 2) [wherein the methylation locus within gene TMEM196 comprises at least 50% of TMEM196 '652 and wherein the portion of the methylation locus that overlaps with TMEM196 has at least 98% similarity with the overlapping portion of TMEM196 '652].

In certain embodiments, the method comprises determining a methylation status for a methylation locus comprising at least a portion of (e.g., at least 50% of) chr19:22709270-22709382 [GGGCCAGTTCCTCCTACCAGCTTCCTGCTGCCACCTCGGCTTCCATCAGAGGGACGC TTAGGATGGCGCAGGGGCCCGGAGACACTGTGAAGAGTCCAGGGGAATGAGGAGG G] (SEQ ID NO: 3), and wherein the diagnosing step comprises diagnosing advanced adenoma in the human subject based at least on the determined methylation status for the methylation locus comprising chr19:22709270-22709382 (SEQ ID NO: 3) [wherein the methylation locus comprises at least 50% of chr19:22709270-22709382 and wherein the portion of the methylation locus that overlaps with chr19:22709270-22709382 has at least 98% similarity with the overlapping portion of chr19:22709270-22709382].

In certain embodiments, the DNA is isolated from blood or plasma of the human subject.

In certain embodiments, the DNA is cell-free DNA of the human subject.

In certain embodiments, methylation status is determined using quantitative polymerase chain reaction (qPCR).

In certain embodiments, the methylation status is determined using methylation sensitive restriction enzyme (MSRE) qPCR.

In certain embodiments, methylation status is determined using massively parallel sequencing (e.g., next-generation sequencing) [e.g., sequencing by-synthesis, real-time (e.g., single-molecule) sequencing, bead emulsion sequencing, nanopore sequencing, or the like].

In another aspect, the invention is directed to a method of detecting (e.g., screening for) colorectal cancer, the method comprising: determining a methylation status for each of one or more (e.g., 1, 2, 3, 4, 5, 6, 7, 8, 9, 10, 11, 12, 13, 14, 15, 16, 17, 18, 19, or all 20) of the following, in deoxyribonucleic acid (DNA) of a human subject: (i) a methylation locus within gene ADSSL1; (ii) a methylation locus within gene CFAP44; (iii) a methylation locus within gene ENG; (iv) a methylation locus within gene LINC01395; (v) a methylation locus within gene NOS3; (vi) a methylation locus within gene RASA3; (vii) a methylation locus within gene SYCP1; (viii) a methylation locus within gene ZAN; (ix) a methylation locus within a genetic region comprising a portion of either or both of genes CD8B & ANAPC1P1; (x) a methylation locus within a genetic region comprising a portion of either or both of genes FLI1 & LOC101929538; (xi) a methylation locus within a genetic region comprising a portion of either or both of genes KCNQ1OT1 & KCNQ; (xii) a methylation locus within a genetic region comprising a portion of either or both of genes LOC101929234 & ZNF503-AS2; (xiii) a methylation locus within a genetic region comprising a portion of either or both of genes MAP3K6 & FCN3; (xiv) a methylation locus comprising at least a portion of (e.g., at least 50% of) ch3:75609726-75609832 [CCTGCGACGTGAATCGTCATATCCAGAGGGGGGTGATATGACTCCCCGCATCGCGG GGGCCTCACCCCATTGCGATGGGGGTCCTAAGAGCCAGGGGGAGATAGGGG] (SEQ ID NO: 21) [wherein the methylation locus comprises at least 50% of ch3:75609726-75609832 and wherein the portion of the methylation locus that overlaps with ch3:75609726-75609832 has at least 98% similarity with the overlapping portion of ch3:75609726-75609832]; (xv) a methylation locus comprising at least a portion of ch3:45036223-45036316 [TGAGCGGAGGACTGAGGAGAGGAAGGAGGGAAAGAATAGGGAGATGAAAACGCC CCGGTCTGCTGCTAAGCACAGCACAGTTACCAAAGCCAGG] (SEQ ID NO: 22) [wherein the methylation locus comprises at least 50% of ch3:45036223-45036316 and wherein the portion of the methylation locus that overlaps with ch3:45036223-45036316 has at least 98% similarity with the overlapping portion of ch3:45036223-45036316]; (xvi) a methylation locus comprising at least a portion of ch12:53694915-53695058 [CATCTCCTCCTCGCAAACCCCAAGCCAAGGCAAGCTGGATGAAGCGCTCCCTGGGC AGGCCCGGCTCTCCGTGTCCCTCCATCACCTGACCCCGCTGGCTCTCGCAGACCCCT TCCTCCACACTCACTCCTCCCGGCTCTCCTT] (SEQ ID NO: 23) [wherein the methylation locus comprises at least 50% of ch12:53694915-53695058 and wherein the portion of the methylation locus that overlaps with ch12:53694915-53695058 has at least 98% similarity with the overlapping portion of ch12:53694915-53695058]; (xvii) a methylation locus comprising at least a portion of ch12:53695032-53695180 [CCACACTCACTCCTCCCGGCTCTCCTTCTATAATCTCCTGACATCTCTTCAAATCCAA TTATTGAATTAATTGACGTACGAACCCAGAGGCAAACAGAAAGGGGCGGCAAACAC TGGGCGGCTCAGATTTATCCTTCGGCCTCCGCAGG] (SEQ ID NO: 24) [wherein the methylation locus comprises at least 50% of ch12:53695032-53695180 and wherein the portion of the methylation locus that overlaps with ch12:53695032-53695180 has at least 98% similarity with the overlapping portion of ch12:53695032-53695180]; (xviii) a methylation locus comprising at least a portion of ch12:53695146-53695232 [TGGGCGGCTCAGATTTATCCTTCGGCCTCCGCAGGGCCCGGCCGGACGAGATTTAC TGGGCCTCGAACACGGCGACAGTTCAAACCT] (SEQ ID NO: 25) [wherein the methylation locus comprises at least 50% of ch12:53695146-53695232 and wherein the portion of the methylation locus that overlaps with ch12:53695146-53695232 has at least 98% similarity with the overlapping portion of ch12:53695146-53695232]; (xix) a methylation locus comprising at least a portion of ch17:78304805-78304921 [CAGCCCTAGGGAGACAGCAGGATGGTTCCAGGAAGCCTGGGCCGCTCCCCAGATC AATGCAGGGACGGACAGCAGCCAGCAGGCTGGGCCACGGCATCAGAGCTGGGGTC AAGAGGT] (SEQ ID NO: 26) [wherein the methylation locus comprises at least 50% of ch17:78304805-78304921 and wherein the portion of the methylation locus that overlaps with ch17:78304805-78304921 has at least 98% similarity with the overlapping portion of ch17:78304805-78304921]; and (xx) a methylation locus comprising at least a portion of ch19:22709270-22709382 [GGGCCAGTTCCTCCTACCAGCTTCCTGCTGCCACCTCGGCTTCCATCAGAGGGACGC TTAGGATGGCGCAGGGGCCCGGAGACACTGTGAAGAGTCCAGGGGAATGAGGAGG G] (SEQ ID NO: 3) [wherein the methylation locus comprises at least 50% of ch19:22709270-22709382 and wherein the portion of the methylation locus that overlaps with ch19:22709270-22709382 has at least 98% similarity with the overlapping portion of ch19:22709270-22709382]; and diagnosing colorectal cancer in the human subject based at least on said determined methylation status(es).

In certain embodiments, the method comprises determining a methylation status for a methylation locus within gene ADSSL1, wherein the methylation locus within gene ADSSL1 comprises at least a portion of (e.g., at least 50% of) ch14:104736436-104736562

(SEQ ID NO: 4)
[CACAGACACCCTGAGCTTGCAACACTCCGGGCCTCTGCCGCGTGTTT
ATTTCAGGATGCCGTGGCATTTGGGTGACCTTTTGTGCTCACCATGGC
TTGCGTCGTCTCCGGGTCACTCTCGTCTGGAC].

In certain embodiments, the method comprises determining a methylation status for a methylation locus within gene CFAP44, wherein the methylation locus within gene CFAP44 comprises at least a portion of (e.g., at least 50% of) one or more of: ch3:113441434-113441539 [TGCTGAGGTCCAAACTCACCGAAGGTACTGACCGCCGCGGCTCCTCTCTTCACAGC GTCTGCCGGAGGCCTCCGTTTACTCCGGTTACCGAGACAACGCCACCCCT] (SEQ ID NO: 5); ch3:113441519-113441620 [TACCGAGACAACGCCACCCCTCTTCCAGGGAGGCGGAACCAGGGCGGGCCGTGGG GCGCATGCGCGGCCGGCGTCCAGCTCTCCGGGAACCCGGTACCTATC] (SEQ ID NO: 6); and/or ch3:113441596-113441690

(SEQ ID NO: 7)
[GCTCTCCGGGAACCCGGTACCTATCCGCCCTTTGGTCGGGCCTTCTCC
GCCTCATGACACTGGTTCAAAGCCAAACAGAAAAGCCCGACGAGTTT].

In certain embodiments, the method comprises determining a methylation status for a methylation locus within gene ENG, wherein the methylation locus within gene ENG comprises at least a portion of (e.g., at least 50% of) ch9:127828322-127828421

(SEQ ID NO: 8)
[GCCCCTGTAAAATGGGGATACAGCAGGGCACGACGTCTGTTGGTCGC
CTGGCACTGGGTCGGCCACCGAGGCCGCGCCTTGGCCTCTTTGTCCCC
TCTGG].

In certain embodiments, the method comprises determining a methylation status for a methylation locus within gene LINC01395, wherein the methylation locus within gene LINC01395 comprises at least a portion of (e.g., at least 50% of) ch11:129618087-129618193 [GGGAGCCTGGAGGGGTTGACACCGCCTGCTCCACCGCAAGCCCCTGGAGGAAGAG CCCCGCTGTGCCCGAGAGCGAGCGCGGGCAGGTGTAACTACCCGGGGCTGGG] (SEQ ID NO: 9) and/or at least a portion of (e.g., at least 50% of) ch11:129618345-129618455

(SEQ ID NO: 10)
[CCTCTGCTTCAGGTGCTTGGCTAGAGAAAGGGCGGCAAGACGGGGCA
GTGCGTGTGCGCGCGCGGGCAAGTGCATGTGAGTGCACACTTATGTGA
GCGCATGTGTGTCTGC].

In certain embodiments, the method comprises determining a methylation status for a methylation locus within gene NOS3, wherein the methylation locus within gene NOS3 comprises at least a portion of (e.g., at least 50% of) ch7:150996901-150997007

(SEQ ID NO: 11)
[CCGGATCCAGTGGGGGAAGCTGCAGGTGCGGCTGGCCAGCGACTGAG
AGACCCGGGCGCTACCAAAAGGGGAGCGGGGTGGCGGGGCAGTTCCTA
AGGCTTCCCGGG].

In certain embodiments, the method comprises determining a methylation status for a methylation locus within gene RASA3, wherein the methylation locus within gene RASA3 comprises at least a portion of (e.g., at least 50% of) ch13:114111799-114111878

(SEQ ID NO: 12)
[TGTCAAACCTCCATCTGTGGTCAGGAGTTAGGACATCCCCAGCTGCA
ATTTGAGCAAAGACGGCGCTTCCAGAGGATCAT].

In certain embodiments, the method comprises determining a methylation status for a methylation locus within gene SYCP1, wherein the methylation locus within gene SYCP1 comprises at least a portion of (e.g., at least 50% of) ch1:114855187-114855327

(SEQ ID NO: 13)
[GAAGGGAACGGGCTTTCTTTTCAGGCCAGCGTGGCAGCGGGCGGTAG
GGCGAAAGGGAGAAGGAAACGAGGGTTTATTCCGTTGCCCACTCCGCG
GTAAGCGACGTTGTAGGGCTCCACTGTAGCGAGAGCCCCGTGGATT].

In certain embodiments, the method comprises determining a methylation status for a methylation locus within gene ZAN, wherein the methylation locus within gene ZAN comprises at least a portion of (e.g., at least 50% of) ch7:100785886-100786015

(SEQ ID NO: 14)
[TGACCTCAGGTGATCCACCCGTCTCGGCCTCCCAAAGTGTTGGGATT
ACAGGCGTGAGCCGCCGCGCCCAGCCCCCTCCTCACTCTCTTTCTCTT
CCTGTAACTTCTACAGCTGGGCAAGAGCTGGGTCT].

In certain embodiments, the method comprises determining a methylation status for a methylation locus within a portion of either or both of genes GD8B & ANAPC1P1, wherein the methylation locus within the portion of either or both of genes CD8B & ANAPCIP1 comprises at least a portion of (e.g., at least 50% of) ch2:86862416-86862559

(SEQ ID NO: 15)
[GCTGGGAACTGGAGGTGCAGAGAAGGCCCCGACGCTGTTTGTAGGTT
GTGGGGGTGCAGCAAGACCTAGATCTTAAGAATTTCGAAGGACTGTGA
CGATCACCGGCTGCGCCCTGCCGGCGAGTGCCCTGGGGCTGGCTCTAT
T].

In certain embodiments, the method comprises determining a methylation status for a methylation locus within a portion of either or both of genes FLI1 & LOC101929538, wherein the methylation locus within the portion of either or both of genes FLI1 & LOC101929538 comprises at least a portion of (e.g., at least 50% of) ch11:128685299-128685448

(SEQ ID NO: 16)
[GCACCAAGAACTAACACATCCTGGAGCTGCCCGGAGTTCCGCTCCTG
CGGGCTTAGCAGGAAAGGGTGCCTAAGGTGAGTGCCCACTTGCGTCCG
ATCCTCTGGGGGCGATGCAGGGTCGGGGCGCCTCAGTGTGTCTCGCTG
CTTGTTC].

In certain embodiments, the method comprises determining a methylation status for a methylation locus within a portion of either or both of genes KCNQ1OT1 & KCNQ, wherein the methylation locus within the portion of either or both of genes KCNQ1OT1 & KCNQ comprises at least a portion of (e.g., at least 50% of) ch11:2656072-2656156

(SEQ ID NO: 17)
[TGGGCACTTGTCATCATGGGTGTTTGGAAAGCAACTCTACGTTCTAG
CCTGTGCTCCATCGTTCCTTCTACATACAAGTGATGCA].

In certain embodiments, the method comprises determining a methylation status PGP for a methylation locus within a portion of either or both of genes MAP3K6 & FCN3, wherein the methylation locus within the portion of either or both of genes MAP3K6 & FCN3 comprises at least a portion of (e.g., at least 50% of) ch1:27369167-27369316 [GCAAAGGCAAGGTGGCTGACGATCCGGAAGCTGTACAGGAGAGATAAGGGCACTG GCTGCCAGAGTGCCCTATCGAAGCATCATCCGAACCCTGCGGTAGGGGTGGCCCAC ACCACGGCCTGAGGCCCAGTCAATGCCATATTTGTGGGC] (SEQ ID NO: 19) and/or at least a portion of (e.g., at least 50% of) ch1:27369224-27369347

(SEQ ID NO: 20)
[TGCCAGAGTGCCCTATCGAAGCATCATCCGAACCCTGCGGTAGGGGT
GGCCCACACCACGGCCTGAGGCCCAGTCAATGCCATATTTGTGGGCGG
CAGCCTCAGACACTGCATAGCGACCATTG].

In certain embodiments, the method comprises determining a methylation status for a methylation locus within gene ADSSL1, wherein the methylation locus within gene ADSSL1 comprises at least one (e.g., at least 2, at least 3, at least 4, or more) CpG dinucleotide.

In certain embodiments, the method comprises determining a methylation status for a methylation locus within gene CFAP44, wherein the methylation locus within gene CFAP44 comprises at least one (e.g., at least 2, at least 3, at least 4, or more) CpG dinucleotide.

In certain embodiments, the method comprises determining a methylation status for a methylation locus within gene ENG, wherein the methylation locus within gene ENG comprises at least one (e.g., at least 2, at least 3, at least 4, or more) CpG dinucleotide.

In certain embodiments, the method comprises determining a methylation status for a methylation locus within gene LINC01395, wherein the methylation locus within gene LINC01395 comprises at least one (e.g., at least 2, at least 3, at least 4, or more) CpG dinucleotide.

In certain embodiments, the method comprises determining a methylation status for a methylation locus within gene NOS3, wherein the methylation locus within gene NOS3 comprises at least one (e.g., at least 2, at least 3, at least 4, or more) CpG dinucleotide.

In certain embodiments, the method comprises determining a methylation status for a methylation locus within gene RASA3, wherein the methylation locus within gene RASA3 comprises at least one (e.g., at least 2, at least 3, at least 4, or more) CpG dinucleotide.

In certain embodiments, the method comprises determining a methylation status for a methylation locus within gene SYCP1, wherein the methylation locus within gene SYCP1 comprises at least one (e.g., at least 2, at least 3, at least 4, or more) CpG dinucleotide.

In certain embodiments, the method comprises determining a methylation status for a methylation locus within gene ZAN, wherein the methylation locus within gene ZAN comprises at least one (e.g., at least 2, at least 3, at least 4, or more) CpG dinucleotide.

In certain embodiments, the method comprises determining a methylation status for a methylation locus within a genetic region comprising a portion of either or both of genes CD8B & ANAC1P1, wherein the methylation locus within a genetic region comprising a portion of either or both of genes CD8B & ANAPC1P1 comprises at least one (e.g., at least 2, at least 3, at least 4, or more) CpG dinucleotide.

In certain embodiments, the method comprises determining a methylation status for a methylation locus within a genetic region comprising a portion of either or both of genes FLI1 & LOC101929538, wherein the methylation locus within a genetic region comprising a portion of either or both of genes FLI1 & LOC101929538 comprises at least one (e.g., at least 2, at least 3, at least 4, or more) CpG dinucleotide.

In certain embodiments, the method comprises determining a methylation status for a methylation locus within a genetic region comprising a portion of either or both of genes KCNQ1OT1 & KCNQ, wherein the methylation locus within a genetic region comprising a portion of either or both of genes KCNQ1OT1 & KCNQ comprises at least one (e.g., at least 2, at least 3, at least 4, or more) CpG dinucleotide.

In certain embodiments, the method comprises determining a methylation status for a methylation locus within a genetic region comprising a portion of either or both of genes LOC101929234 & ZNF503-AS2, wherein the methylation locus within a genetic region comprising a portion of either or both of genes LOC101929234 & ZNF503-AS2 comprises at least one (e.g., at least 2, at least 3, at least 4, or more) CpG dinucleotide.

In certain embodiments, the method comprises determining a methylation status for a methylation locus within a genetic region comprising a portion of either or both of genes MAP3K6 & FCN3, wherein the methylation locus within a genetic region comprising a portion of either or both of genes MAP3K6 & FCN3 comprises at least one (e.g., at least 2, at least 3, at least 4, or more) CpG dinucleotide.

In certain embodiments, the DNA is isolated from blood or plasma of the human subject.

In certain embodiments, the DNA is cell-free DNA of the human subject.

In certain embodiments, the methylation status is determined using quantitative polymerase chain reaction (qPCR).

In certain embodiments, the methylation status is determined using methylation sensitive restriction enzyme (MSRE)-qPCR.

In certain embodiments, methylation status is determined using massively parallel sequencing.

In another aspect, the invention is directed to a method of screening for a colorectal neoplasm in a sample obtained from a subject (e.g., a human subject), the method comprising: determining a methylation status of each of one or more markers identified in the sample; and determining whether the subject has a colorectal neoplasm based at least in part on the determined methylation status of each of the one or more markers and a corresponding methylation status of said one or more markers representative of one or more subjects that do not have a colorectal neoplasm that is considered to be either malignant or pre-malignant (e.g., one or more patient(s) with no colonoscopy findings, with hyperplastic polyps, and/or with non-malignant gastrointestinal diseases), wherein each of the one or more markers comprises a base (e.g., a base within a CpG dinucleotide, e.g., a cytosine) in a differentially methylated region (DMR) selected from the DMRs listed in Table 1.

In certain embodiments, the colorectal neoplasm comprises colorectal cancer and/or advanced adenoma.

In certain embodiments, the sample comprises a stool sample, a colorectal tissue sample, a blood sample, or a blood product sample.

In certain embodiments, each methylation locus is equal to or less than 5000 bp (e.g., 4,000 bp or less, 3,000 bp, 2,000 bp, 1,000 bp, 950 bp, 900 bp, 850 bp, 800 bp, 750 bp, 700 bp, 650 bp, 600 bp, 550 bp, 500 bp, 450 bp, 400 bp, 350 bp, 300 bp, 250 bp, 200 bp, 150 bp, 100 bp, 50 bp, 40 bp, 30 bp, 20 bp, or 10 bp) in length.

In another aspect, the invention is directed to a kit for use in a method described herein, the kit comprising one or more oligonucleotide primer pairs (e.g., a forward and reverse primer pair, e.g., of Table 1) for amplification of one or more corresponding methylation locus/loci.

In certain embodiments, the one or more corresponding methylation loci each comprise at least one (e.g., 2, 3, 4, 5 or more) methylation sensitive restriction enzyme cleavage sites.

In another aspect, the invention is directed to a diagnostic qPCR reaction for detection (e.g., screening) of colorectal cancer (e.g., in a method described herein), the diagnostic qPCR reaction including (a) human DNA, (b) a polymerase, (c) one or more oligonucleotide primer pairs (e.g., as found in Table 1) for amplification of one or more corresponding methylation locus/loci., and, optionally, at least one methylation sensitive restriction enzyme.

In certain embodiments, each of the one or more corresponding methylation loci is equal to or less than 5000 bp (e.g., 4,000 bp or less, 3,000 bp, 2,000 bp, 1,000 bp, 950 bp, 900 bp, 850 bp, 800 bp, 750 bp, 700 bp, 650 bp, 600 bp, 550 bp, 500 bp, 450 bp, 400 bp, 350 bp, 300 bp, 250 bp, 200 bp, 150 bp, 100 bp, 50 bp, 40 bp, 30 bp, 20 bp, or 10 bp).

In certain embodiments, each of the one or more corresponding methylation locus/loci comprises at least one methylation sensitive restriction enzyme (MSRE) cleavage site (e.g., at least 2, 3, 4, 5, 6, 7, 8, 9, 10, 11, 12, 13, 14, or 15 MSRE cleavage sites).

In various aspects, methods as described herein may further comprise treatment of a cancer (e.g., colorectal cancer, advanced adenoma) based on, at least, the methylation status of one or more methylation loci.

In various aspects, methods and compositions of the present invention can be used in combination with biomarkers known in the art, e.g., as disclosed in U.S. Pat. No. 10,006,925, which is herein incorporated by reference in its entirety.

### Definitions

A or An: The articles “a” and “an” are used herein to refer to one or to more than one (i.e., to at least one) of the grammatical object of the article. By way of example, “an element” refers to one element or more than one element.

About: The term “about”, when used herein in reference to a value, refers to a value that is similar, in context, to the referenced value. In general, those skilled in the art, familiar with the context, will appreciate the relevant degree of variance encompassed by “about” in that context. For example, in some embodiments, e.g., as set forth herein, the term “about” can encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or with a fraction of a percent, of the referred value.

Administration: As used herein, the term “administration” typically refers to the administration of a composition to a subject or system, for example to achieve delivery of an agent that is, is included in, or is otherwise delivered by, the composition.

Advanced adenoma: As used herein, the term “advanced adenoma” typically refers to refer to cells that exhibit first indications of relatively abnormal, uncontrolled, and/or autonomous growth but are not yet classified as cancerous alterations. In the context of colon tissue, “advanced adenoma” refers to neoplastic growth that shows signs of high grade dysplasia, and/or size that is >=10 mm, and/or villous histological type, and/or serrated histological type with any type of dysplasia.

Agent: As used herein, the term “agent” refers to an entity (e.g., for example, a small molecule, peptide, polypeptide, nucleic acid, lipid, polysaccharide, complex, combination, mixture, system, or phenomenon such as heat, electric current, electric field, magnetic force, magnetic field, etc.).

Amelioration: As used herein, the term “amelioration” refers to the prevention, reduction, palliation, or improvement of a state of a subject. Amelioration includes, but does not require, complete recovery or complete prevention of a disease, disorder or condition.

Amplicon or amplicon molecule: As used herein, the term “amplicon” or “amplicon molecule” refers to a nucleic acid molecule generated by transcription from a template nucleic acid molecule, or a nucleic acid molecule having a sequence complementary thereto, or a double-stranded nucleic acid including any such nucleic acid molecule. Transcription can be initiated from a primer.

Amplification: As used herein, the term “amplification” refers to the use of a template nucleic acid molecule in combination with various reagents to generate further nucleic acid molecules from the template nucleic acid molecule, which further nucleic acid molecules may be identical to or similar to (e.g., at least 70% identical, e.g., at least 75%, 80%, 85%, 90%, 91%, 92%, 93%, 94%, 95%, 96%, 97%, 98%, 99%, or 100% identical to) a segment of the template nucleic acid molecule and/or a sequence complementary thereto.

Amplification reaction mixture: As used herein, the terms “amplification reaction mixture” or “amplification reaction” refer to a template nucleic acid molecule together with reagents sufficient for amplification of the template nucleic acid molecule.

Biological Sample: As used herein, the term “biological sample” typically refers to a sample obtained or derived from a biological source (e.g., a tissue or organism or cell culture) of interest, as described herein. In some embodiments, e.g., as set forth herein, a biological source is or includes an organism, such as an animal or human. In some embodiments, e.g., as set forth herein, a biological sample is or include biological tissue or fluid. In some embodiments, e.g., as set forth herein, a biological sample can be or include cells, tissue, or bodily fluid. In some embodiments, e.g., as set forth herein, a biological sample can be or include blood, blood cells, cell-free DNA, free floating nucleic acids, ascites, biopsy samples, surgical specimens, cell-containing body fluids, sputum, saliva, feces, urine, cerebrospinal fluid, peritoneal fluid, pleural fluid, lymph, gynecological fluids, secretions, excretions, skin swabs, vaginal swabs, oral swabs, nasal swabs, washings or lavages such as a ductal lavages or broncheoalveolar lavages, aspirates, scrapings, bone marrow. In some embodiments, e.g., as set forth herein, a biological sample is or includes cells obtained from a single subject or from a plurality of subjects. A sample can be a “primary sample” obtained directly from a biological source, or can be a “processed sample.” A biological sample can also be referred to as a “sample.”

Biomarker: As used herein, the term “biomarker,” consistent with its use in the art, refers to a to an entity whose presence, level, or form, correlates with a particular biological event or state of interest, so that it is considered to be a “marker” of that event or state. Those of skill in the art will appreciate, for instance, in the context of a DNA biomarker, that a biomarker can be or include a locus (such as one or more methylation loci) and/or the status of a locus (e.g., the status of one or more methylation loci). To give but a few examples of biomarkers, in some embodiments, e.g., as set forth herein, a biomarker can be or include a marker for a particular disease, disorder or condition, or can be a marker for qualitative of quantitative probability that a particular disease, disorder or condition can develop, occur, or reoccur, e.g., in a subject. In some embodiments, e.g., as set forth herein, a biomarker can be or include a marker for a particular therapeutic outcome, or qualitative of quantitative probability thereof. Thus, in various embodiments, e.g., as set forth herein, a biomarker can be predictive, prognostic, and/or diagnostic, of the relevant biological event or state of interest. A biomarker can be an entity of any chemical class. For example, in some embodiments, e.g., as set forth herein, a biomarker can be or include a nucleic acid, a polypeptide, a lipid, a carbohydrate, a small molecule, an inorganic agent (e.g., a metal or ion), or a combination thereof. In some embodiments, e.g., as set forth herein, a biomarker is a cell surface marker. In some embodiments, e.g., as set forth herein, a biomarker is intracellular. In some embodiments, e.g., as set forth herein, a biomarker is found outside of cells (e.g., is secreted or is otherwise generated or present outside of cells, e.g., in a body fluid such as blood, urine, tears, saliva, cerebrospinal fluid, and the like). In some embodiments, e.g., as set forth herein, a biomarker is methylation status of a methylation locus. In some instances, e.g., as set forth herein, a biomarker may be referred to as a “marker.”

To give but one example of a biomarker, in some embodiments e.g., as set forth herein, the term refers to expression of a product encoded by a gene, expression of which is characteristic of a particular tumor, tumor subclass, stage of tumor, etc. Alternatively or additionally, in some embodiments, e.g., as set forth herein, presence or level of a particular marker can correlate with activity (or activity level) of a particular signaling pathway, for example, of a signaling pathway the activity of which is characteristic of a particular class of tumors.

Those of skill in the art will appreciate that a biomarker may be individually determinative of a particular biological event or state of interest, or may represent or contribute to a determination of the statistical probability of a particular biological event or state of interest. Those of skill in the art will appreciate that markers may differ in their specificity and/or sensitivity as related to a particular biological event or state of interest.

Blood component: As used herein, the term “blood component” refers to any component of whole blood, including red blood cells, white blood cells, plasma, platelets, endothelial cells, mesothelial cells, epithelial cells, and cell-free DNA. Blood components also include the components of plasma, including proteins, metabolites, lipids, nucleic acids, and carbohydrates, and any other cells that can be present in blood, e.g., due to pregnancy, organ transplant, infection, injury, or disease.

Cancer: As used herein, the terms “cancer,” “malignancy,” “neoplasm,” “tumor,” and “carcinoma,” are used interchangeably to refer to a disease, disorder, or condition in which cells exhibit or exhibited relatively abnormal, uncontrolled, and/or autonomous growth, so that they display or displayed an abnormally elevated proliferation rate and/or aberrant growth phenotype. In some embodiments, e.g., as set forth herein, a cancer can include one or more tumors. In some embodiments e.g., as set forth herein, a cancer can be or include cells that are precancerous (e.g., benign), malignant, pre-metastatic, metastatic, and/or non-metastatic. In some embodiments e.g., as set forth herein, a cancer can be or include a solid tumor. In some embodiments e.g., as set forth herein, a cancer can be or include a hematologic tumor. In general, examples of different types of cancers known in the art include, for example, colorectal cancer, hematopoietic cancers including leukemias, lymphomas (Hodgkin's and non-Hodgkin's), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papillomas, and the like.

Chemotherapeutic agent: As used herein, the term “chemotherapeutic agent,” consistent with its use in the art, refers to one or more agents known, or having characteristics known to, treat or contribute to the treatment of cancer. In particular, chemotherapeutic agents include pro-apoptotic, cytostatic, and/or cytotoxic agents. In some embodiments e.g., as set forth herein, a chemotherapeutic agent can be or include alkylating agents, anthracyclines, cytoskeletal disruptors (e.g., microtubule targeting moieties such as taxanes, maytansine, and analogs thereof, of), epothilones, histone deacetylase inhibitors HDACs), topoisomerase inhibitors (e.g., inhibitors of topoisomerase I and/or topoisomerase II), kinase inhibitors, nucleotide analogs or nucleotide precursor analogs, peptide antibiotics, platinum-based agents, retinoids, vinca alkaloids, and/or analogs that share a relevant anti-proliferative activity. In some particular embodiments e.g., as set forth herein, a chemotherapeutic agent can be or include of Actinomycin, All-trans retinoic acid, an Auiristatin, Azacitidine, Azathioprine, Bleomycin, Bortezomib, Carboplatin, Capecitabine, Cisplatin, Chlorambucil, Cyclophosphamide, Curcumin, Cytarabine, Daunorubicin, Docetaxel, Doxifluridine, Doxorubicin, Epirubicin, Epothilone, Etoposide, Fluorouracil, Gemcitabine, Hydroxyurea, Idarubicin, Imatinib, Irinotecan, Maytansine and/or analogs thereof (e.g., DM1) Mechlorethamine, Mercaptopurine, Methotrexate, Mitoxantrone, a Maytansinoid, Oxaliplatin, Paclitaxel, Pemetrexed, Teniposide, Tioguanine, Topotecan, Valrubicin, Vinblastine, Vincristine, Vindesine, Vinorelbine, or a combination thereof. In some embodiments e.g., as set forth herein, a chemotherapeutic agent can be utilized in the context of an antibody-drug conjugate. In some embodiments e.g., as set forth herein, a chemotherapeutic agent is one found in an antibody-drug conjugate selected from the group consisting of: hLL1-doxorubicin, hRS7-SN-38, hMN-14-SN-38, hLL2-SN-38, hA20-SN-38, hPAM4-SN-38, hLL1-SN-38, hRS7-Pro-2-P-Dox, hMN-14-Pro-2-P-Dox, hLL2-Pro-2-P-Dox, hA20-Pro-2-P-Dox, hPAM4-Pro-2-P-Dox, hLL1-Pro-2-P-Dox, P4/D10-doxorubicin, gemtuzumab ozogamicin, brentuximab vedotin, trastuzumab emtansine, inotuzumab ozogamicin, glembatumomab vedotin, SAR3419, SAR566658, BIIB015, BT062, SGN-75, SGN-CD19A, AMG-172, AMG-595, BAY-94-9343, ASG-5ME, ASG-22ME, ASG-16M8F, MDX-1203, MLN-0264, anti-PSMA ADC, RG-7450, RG-7458, RG-7593, RG-7596, RG-7598, RG-7599, RG-7600, RG-7636, ABT-414, IMGN-853, IMGN-529, vorsetuzumab mafodotin, and lorvotuzumab mertansine. In some embodiments e.g., as set forth herein, a chemotherapeutic agent can be or comprise of farnesyl-thiosalicylic acid (FTS), 4-(4-Chloro-2-methylphenoxy)-N-hydroxybutanamide (CMH), estradiol (E2), tetramethoxystilbene (TMS), S-tocatrienol, salinomycin, or curcumin.

Combination therapy: As used herein, the term “combination therapy” refers to administration to a subject of to two or more agents or regimens such that the two or more agents or regimens together treat a disease, condition, or disorder of the subject. In some embodiments, e.g., as set forth herein, the two or more therapeutic agents or regimens can be administered simultaneously, sequentially, or in overlapping dosing regimens. Those of skill in the art will appreciate that combination therapy includes but does not require that the two agents or regimens be administered together in a single composition, nor at the same time.

Comparable: As used herein, the term “comparable” refers to members within sets of two or more conditions, circumstances, agents, entities, populations, etc., that may not be identical to one another but that are sufficiently similar to permit comparison there between, such that one of skill in the art will appreciate that conclusions can reasonably be drawn based on differences or similarities observed. In some embodiments, e.g., as sort forth herein, comparable sets of conditions, circumstances, agents, entities, populations, etc. are typically characterized by a plurality of substantially identical features and zero, one, or a plurality of differing features. Those of ordinary skill in the art will understand, in context, what degree of identity is required to render members of a set comparable. For example, those of ordinary skill in the art will appreciate that members of sets of conditions, circumstances, agents, entities, populations, etc., are comparable to one another when characterized by a sufficient number and type of substantially identical features to warrant a reasonable conclusion that differences observed can be attributed in whole or part to non-identical features thereof.

Detectable moiety: The term “detectable moiety” as used herein refers to any element, molecule, functional group, compound, fragment, or other moiety that is detectable. In some embodiments, e.g., as sort forth herein, a detectable moiety is provided or utilized alone. In some embodiments, e.g., as sort forth herein, a detectable moiety is provided and/or utilized in association with (e.g., joined to) another agent. Examples of detectable moieties include, but are not limited to, various ligands, radionuclides (e.g., ³H, ¹⁴C, ¹⁸F, ¹⁹F, ³²P, ³⁵S, ¹³⁵I, ¹²⁵I, ¹²³I, ⁶⁴Cu, ¹⁸⁷Re, ¹¹¹In, ⁹⁰Y, ^{99m}Tc, ¹⁷⁷Lu, ⁸⁹Zr etc.), fluorescent dyes, chemiluminescent agents, bioluminescent agents, spectrally resolvable inorganic fluorescent semiconductors nanocrystals (i.e., quantum dots), metal nanoparticles, nanoclusters, paramagnetic metal ions, enzymes, colorimetric labels, biotin, dioxigenin, haptens, and proteins for which antisera or monoclonal antibodies are available.

Diagnosis: As used herein, the term “Diagnosis” refers to determining whether, and/or the qualitative of quantitative probability that, a subject has or will develop a disease, disorder, condition, or state. For example, in diagnosis of cancer, diagnosis can include a determination regarding the risk, type, stage, malignancy, or other classification of a cancer. In some instances, e.g., as sort forth herein, a diagnosis can be or include a determination relating to prognosis and/or likely response to one or more general or particular therapeutic agents or regimens.

Diagnostic information: As used herein, the term “diagnostic information” refers to information useful in providing a diagnosis. Diagnostic information can include, without limitation, biomarker status information.

Differentially methylated: As used herein, the term “differentially methylated” describes a methylation site for which the methylation status differs between a first condition and a second condition. A methylation site that is differentially methylated can be referred to as a differentially methylated site. In some instances, e.g., as sort forth herein, a DMR is defined by the amplicon produced by amplification using oligonucleotide primers, e.g., a pair of oligonucleotide primers selected for amplification of the DMR or for amplification of a DNA region of interest present in the amplicon. In some instances, e.g., as sort forth herein, a DMR is defined as a DNA region amplified by a pair of oligonucleotide primers, including the region having the sequence of, or a sequence complementary to, the oligonucleotide primers. In some instances, e.g., as sort forth herein, a DMR is defined as a DNA region amplified by a pair of oligonucleotide primers, excluding the region having the sequence of, or a sequence complementary to, the oligonucleotide primers. As used herein, a specifically provided DMR can be unambiguously identified by the name of an associated gene followed by three digits of a starting position, such that, for example, a DMR starting at position 29921434 of ALK can be identified as ALK '434.

Differentially methylated region: As used herein, the term “differentially methylated region” (DMR) refers to a DNA region that includes one or more differentially methylated sites. A DMR that includes a greater number or frequency of methylated sites under a selected condition of interest, such as a cancerous state, can be referred to as a hypermethylation DMR. A DMR that includes a smaller number or frequency of methylated sites under a selected condition of interest, such as a cancerous state, can be referred to as a hypomethylation DMR. A DMR that is a methylation biomarker for colorectal cancer can be referred to as a colorectal cancer DMR. In some instances, e.g., as set forth herein, a DMR can be a single nucleotide, which single nucleotide is a methylation site. In some instances, e.g., as set forth herein, a DMR has a length of at least 10, at least 15, at least 20, at least 24, at least 50, or at least 75 base pairs. In some instances, e.g., as set forth herein, a DMR has a length of less than 1000, less than 750, less than 500, less than 350, less than 300, or less than 250 base pairs (e.g., where methylation status is determined using quantitative polymerase chain reaction (qPCR), e.g., methylation sensitive restriction enzyme quantitative polymerase chain reaction (MSRE-qPCR)). In some instances, e.g., as set forth herein, a DMR that is a methylation biomarker for advanced adenoma may also be useful in identification of colorectal cancer.

DNA region: As used herein, “DNA region” refers to any contiguous portion of a larger DNA molecule. Those of skill in the art will be familiar with techniques for determining whether a first DNA region and a second DNA region correspond, based, e.g., on sequence similarity (e.g, sequence identity or homology) of the first and second DNA regions and/or context (e.g., the sequence identity or homology of nucleic acids upstream and/or downstream of the first and second DNA regions).

Except as otherwise specified herein, sequences found in or relating to humans (e.g., that hybridize to human DNA) are found in, based on, and/or derived from the example representative human genome sequence commonly referred to, and known to those of skill in the art, as Homo sapiens (human) genome assembly GRCh38, hg38, and/or Genome Reference Consortium Human Build 38. Those of skill in the art will further appreciate that DNA regions of hg38 can be referred to by a known system including identification of particular nucleotide positions or ranges thereof in accordance with assigned numbering.

Dosing regimen: As used herein, the term “dosing regimen” can refer to a set of one or more same or different unit doses administered to a subject, typically including a plurality of unit doses administration of each of which is separated from administration of the others by a period of time. In various embodiments, e.g., as set forth herein, one or more or all unit doses of a dosing regimen may be the same or can vary (e.g., increase over time, decrease over time, or be adjusted in accordance with the subject and/or with a medical practitioner's determination). In various embodiments, e.g., as set forth herein, one or more or all of the periods of time between each dose may be the same or can vary (e.g., increase over time, decrease over time, or be adjusted in accordance with the subject and/or with a medical practitioner's determination). In some embodiments, e.g., as set forth herein, a given therapeutic agent has a recommended dosing regimen, which can involve one or more doses. Typically, at least one recommended dosing regimen of a marketed drug is known to those of skill in the art. In some embodiments, e.g., as set forth herein, a dosing regimen is correlated with a desired or beneficial outcome when administered across a relevant population (i.e., is a therapeutic dosing regimen).

Downstream: As used herein, the term“downstream” means that a first DNA region is closer, relative to a second DNA region, to the C-terminus of a nucleic acid that includes the first DNA region and the second DNA region.

Gene: As used herein, the term “gene” refers to a single DNA region, e.g., in a chromosome, that includes a coding sequence that encodes a product (e.g., an RNA product and/or a polypeptide product), together with all, some, or none of the DNA sequences that contribute to regulation of the expression of coding sequence. In some embodiments, e.g., as set forth herein, a gene includes one or more non-coding sequences. In some particular embodiments, e.g., as set forth herein, a gene includes exonic and intronic sequences. In some embodiments, e.g., as set forth herein, a gene includes one or more regulatory elements that, for example, can control or impact one or more aspects of gene expression (e.g., cell-type-specific expression, inducible expression, etc.). In some embodiments, e.g., as set forth herein, a gene includes a promoter. In some embodiments, e.g., as set forth herein, a gene includes one or both of a (i) DNA nucleotides extending a predetermined number of nucleotides upstream of the coding sequence and (ii) DNA nucleotides extending a predetermined number of nucleotides downstream of the coding sequence. In various embodiments, e.g., as set forth herein, the predetermined number of nucleotides can be 500 bp, 1 kb, 2 kb, 3 kb, 4 kb, 5 kb, 10 kb, 20 kb, 30 kb, 40 kb, 50 kb, 75 kb, or 100 kb.

Homology: As used herein, the term “homology” refers to the overall relatedness between polymeric molecules, e.g., between nucleic acid molecules (e.g., DNA molecules and/or RNA molecules) and/or between polypeptide molecules. Those of skill in the art will appreciate that homology can be defined, e.g., by a percent identity or by a percent homology (sequence similarity). In some embodiments, e.g., as set forth herein, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical. In some embodiments, e.g., as set forth herein, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar.

Hybridize: As used herein, “hybridize” refers to the association of a first nucleic acid with a second nucleic acid to form a double-stranded structure, which association occurs through complementary pairing of nucleotides. Those of skill in the art will recognize that complementary sequences, among others, can hybridize. In various embodiments, e.g., as set forth herein, hybridization can occur, for example, between nucleotide sequences having at least 70% complementarity, e.g., at least 75%, 80%, 85%, 90%, 91%, 92%, 93%, 94%, 95%, 96%, 97%, 98%, 99%, or 100% complementarity. Those of skill in the art will further appreciate that whether hybridization of a first nucleic acid and a second nucleic acid does or does not occur can dependence upon various reaction conditions. Conditions under which hybridization can occur are known in the art.

Hypomethylation: As used herein, the term “hypomethylation” refers to the state of a methylation locus having at least one fewer methylated nucleotides in a state of interest as compared to a reference state (e.g., at least one fewer methylated nucleotides in colorectal cancer than in healthy control).

Hypermethylation: As used herein, the term “hypermethylation” refers to the state of a methylation locus having at least one more methylated nucleotide in a state of interest as compared to a reference state (e.g., at least one more methylated nucleotide in colorectal cancer than in healthy control).

Identity, identical: As used herein, the terms “identity” and “identical” refers to the overall relatedness between polymeric molecules, e.g., between nucleic acid molecules (e.g., DNA molecules and/or RNA molecules) and/or between polypeptide molecules. Methods for the calculation of a percent identity as between two provided sequences are known in the art. Calculation of the percent identity of two nucleic acid or polypeptide sequences, for example, can be performed by aligning the two sequences (or the complement of one or both sequences) for optimal comparison purposes (e.g., gaps can be introduced in one or both of a first and a second sequences for optimal alignment and non-identical sequences can be disregarded for comparison purposes). The nucleotides or amino acids at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences and, optionally, taking into account the number of gaps and the length of each gap, which may need to be introduced for optimal alignment of the two sequences. The comparison of sequences and determination of percent identity between two sequences can be accomplished using a computational algorithm, such as BLAST (basic local alignment search tool).

“Improved,” “increased,” or “reduced”: As used herein, these terms, or grammatically comparable comparative terms, indicate values that are relative to a comparable reference measurement. For example, in some embodiments, e.g., as set forth herein, an assessed value achieved with an agent of interest may be “improved” relative to that obtained with a comparable reference agent or with no agent. Alternatively or additionally, in some embodiments, e.g., as set forth herein, an assessed value in a subject or system of interest may be “improved” relative to that obtained in the same subject or system under different conditions or at a different point in time (e.g., prior to or after an event such as administration of an agent of interest), or in a different, comparable subject (e.g., in a comparable subject or system that differs from the subject or system of interest in presence of one or more indicators of a particular disease, disorder or condition of interest, or in prior exposure to a condition or agent, etc.). In some embodiments, e.g., as set forth herein, comparative terms refer to statistically relevant differences (e.g., differences of a prevalence and/or magnitude sufficient to achieve statistical relevance). Those of skill in the art will be aware, or will readily be able to determine, in a given context, a degree and/or prevalence of difference that is required or sufficient to achieve such statistical significance.

Methylation: As used herein, the term “methylation” includes methylation at any of (i) C5 position of cytosine; (ii) N4 position of cytosine; and (iii) the N6 position of adenine. Methylation also includes (iv) other types of nucleotide methylation. A nucleotide that is methylated can be referred to as a “methylated nucleotide” or “methylated nucleotide base.” In certain embodiments, e.g., as set forth herein, methylation specifically refers to methylation of cytosine residues. In some instances, methylation specifically refers to methylation of cytosine residues present in CpG sites.

Methylation assay: As used herein, the term “methylation assay” refers to any technique that can be used to determine the methylation status of a methylation locus.

Methylation biomarker: As used herein, the term “methylation biomarker” refers to a biomarker that is or includes at least one methylation locus and/or the methylation status of at least one methylation locus, e.g., a hypermethylated locus. In particular, a methylation biomarker is a biomarker characterized by a change between a first state and a second state (e.g., between a cancerous state and a non-cancerous state) in methylation status of one or more nucleic acid loci.

Methylation locus: As used herein, the term “methylation locus” refers to a DNA region that includes at least one differentially methylated region. A methylation locus that includes a greater number or frequency of methylated sites under a selected condition of interest, such as a cancerous state, can be referred to as a hypermethylated locus. A methylation locus that includes a smaller number or frequency of methylated sites under a selected condition of interest, such as a cancerous state, can be referred to as a hypomethylated locus. In some instances, e.g., as set forth herein, a methylation locus has a length of at least 10, at least 15, at least 20, at least 24, at least 50, or at least 75 base pairs. In some instances, e.g., as set forth herein, a methylation locus has a length of less than 1000, less than 750, less than 500, less than 350, less than 300, or less than 250 base pairs (e.g., where methylation status is determined using quantitative polymerase chain reaction (qPCR), e.g., methylation sensitive restriction enzyme quantitative polymerase chain reaction (MSRE-qPCR)).

Methylation site: As used herein, a methylation site refers to a nucleotide or nucleotide position that is methylated in at least one condition. In its methylated state, a methylation site can be referred to as a methylated site.

Methylation status: As used herein, “methylation status,” “methylation state,” or “methylation profile” refer to the number, frequency, or pattern of methylation at methylation sites within a methylation locus. Accordingly, a change in methylation status between a first state and a second state can be or include an increase in the number, frequency, or pattern of methylated sites, or can be or include a decrease in the number, frequency, or pattern of methylated sites. In various instances, a change in methylation status in a change in methylation value.

Methylation value: As used herein, the term “methylation value” refers to a numerical representation of a methylation status, e.g., in the form of number that represents the frequency or ratio of methylation of a methylation locus. In some instances, e.g., as set forth herein, a methylation value can be generated by a method that includes quantifying the amount of intact nucleic acid present in a sample following restriction digestion of the sample with a methylation dependent restriction enzyme. In some instances, e.g., as set forth herein, a methylation value can be generated by a method that includes comparing amplification profiles after bisulfite reaction of a sample. In some instances, e.g., as set forth herein, a methylation value can be generated by comparing sequences of bisulfite-treated and untreated nucleic acids. In some instances, e.g., as set forth herein, a methylation value is, includes, or is based on a quantitative PCR result.

Nucleic acid: As used herein, in its broadest sense, the term “nucleic acid” refers to any compound and/or substance that is or can be incorporated into an oligonucleotide chain. In some embodiments e.g., as set forth herein, a nucleic acid is a compound and/or substance that is or can be incorporated into an oligonucleotide chain via a phosphodiester linkage. As will be clear from context, in some embodiments e.g., as set forth herein, the term nucleic acid refers to an individual nucleic acid residue (e.g., a nucleotide and/or nucleoside), and in some embodiments e.g., as set forth herein refers to an polynucleotide chain comprising a plurality of individual nucleic acid residues. A nucleic acid can be or include DNA, RNA, or a combinations thereof. A nucleic acid can include natural nucleic acid residues, nucleic acid analogs, and/or synthetic residues. In some embodiments e.g., as set forth herein, a nucleic acid includes natural nucleotides (e.g., adenosine, thymidine, guanosine, cytidine, uridine, deoxyadenosine, deoxythymidine, deoxy guanosine, and deoxycytidine). In some embodiments e.g., as set forth herein, a nucleic acid is or includes of one or more nucleotide analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof).

In some embodiments e.g., as set forth herein, a nucleic acid has a nucleotide sequence that encodes a functional gene product such as an RNA or protein. In some embodiments e.g., as set forth herein, a nucleic acid includes one or more introns. In some embodiments e.g., as set forth herein, a nucleic acid includes one or more genes. In some embodiments e.g., as set forth herein, nucleic acids are prepared by one or more of isolation from a natural source, enzymatic synthesis by polymerization based on a complementary template (in vivo or in vitro), reproduction in a recombinant cell or system, and chemical synthesis.

In some embodiments e.g., as set forth herein, a nucleic acid analog differs from a nucleic acid in that it does not utilize a phosphodiester backbone. For example, in some embodiments e.g., as set forth herein, a nucleic acid can include one or more peptide nucleic acids, which are known in the art and have peptide bonds instead of phosphodiester bonds in the backbone. Alternatively or additionally, in some embodiments e.g., as set forth herein, a nucleic acid has one or more phosphorothioate and/or 5′-N-phosphoramidite linkages rather than phosphodiester bonds. In some embodiments e.g., as set forth herein, a nucleic acid comprises one or more modified sugars (e.g., 2′-fluororibose, ribose, 2′-deoxyribose, arabinose, and hexose) as compared with those in natural nucleic acids.

In some embodiments, e.g., as set forth herein, a nucleic acid is or includes at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues. In some embodiments, e.g., as set forth herein, a nucleic acid is partly or wholly single stranded, or partly or wholly double stranded.

Nucleic acid detection assay: As used herein, the term “nucleic acid detection assay” refers to any method of determining the nucleotide composition of a nucleic acid of interest. Nucleic acid detection assays include but are not limited to, DNA sequencing methods, polymerase chain reaction-based methods, probe hybridization methods, ligase chain reaction, etc.

Nucleotide: As used herein, the term “nucleotide” refers to a structural component, or building block, of polynucleotides, e.g., of DNA and/or RNA polymers. A nucleotide includes of a base (e.g., adenine, thymine, uracil, guanine, or cytosine) and a molecule of sugar and at least one phosphate group. As used herein, a nucleotide can be a methylated nucleotide or an un-methylated nucleotide. Those of skill in the art will appreciate that nucleic acid terminology, such as, as examples, “locus” or “nucleotide” can refer to both a locus or nucleotide of a single nucleic acid molecule and/or to the cumulative population of loci or nucleotides within a plurality of nucleic acids (e.g., a plurality of nucleic acids in a sample and/or representative of a subject) that are representative of the locus or nucleotide (e.g., having the same identical nucleic acid sequence and/or nucleic acid sequence context, or having a substantially identical nucleic acid sequence and/or nucleic acid context).

Oligonucleotide primer: As used herein, the term oligonucleotide primer, or primer, refers to a nucleic acid molecule used, capable of being used, or for use in, generating amplicons from a template nucleic acid molecule. Under transcription-permissive conditions (e.g., in the presence of nucleotides and a DNA polymerase, and at a suitable temperature and pH), an oligonucleotide primer can provide a point of initiation of transcription from a template to which the oligonucleotide primer hybridizes. Typically, an oligonucleotide primer is a single-stranded nucleic acid between 5 and 200 nucleotides in length. Those of skill in the art will appreciate that optimal primer length for generating amplicons from a template nucleic acid molecule can vary with conditions including temperature parameters, primer composition, and transcription or amplification method. A pair of oligonucleotide primers, as used herein, refers to a set of two oligonucleotide primers that are respectively complementary to a first strand and a second strand of a template double-stranded nucleic acid molecule. First and second members of a pair of oligonucleotide primers may be referred to as a “forward” oligonucleotide primer and a “reverse” oligonucleotide primer, respectively, with respect to a template nucleic acid strand, in that the forward oligonucleotide primer is capable of hybridizing with a nucleic acid strand complementary to the template nucleic acid strand, the reverse oligonucleotide primer is capable of hybridizing with the template nucleic acid strand, and the position of the forward oligonucleotide primer with respect to the template nucleic acid strand is 5′ of the position of the reverse oligonucleotide primer sequence with respect to the template nucleic acid strand. It will be understood by those of skill in the art that the identification of a first and second oligonucleotide primer as forward and reverse oligonucleotide primers, respectively, is arbitrary inasmuch as these identifiers depend upon whether a given nucleic acid strand or its complement is utilized as a template nucleic acid molecule.

Overlapping: The term “overlapping” is used herein in reference to two regions of DNA, each of which contains a sub-sequence that is substantially identical to a sub-sequence of the same length in the other region (e.g., the two regions of DNA have a common sub-sequence). “Substantially identical” means that the two identically-long sub-sequences differ by fewer than a given number of base pairs. In certain instances, e.g., as set forth herein, each sub-sequence has a length of at least 20 base pairs that differ by fewer than 4, 3, 2, or 1 base pairs from each other (e.g., the two sub-sequences having at least 80%, at least 85%, at least 90%, at least 95% similarity, at least 97% similarity, at least 98% similarity, at least 99% similarity, or at least 99.5% similarity). In certain instances, e.g., as set forth herein, each sub-sequence has a length of at least 24 base pairs that differ by fewer than 5, 4, 3, 2, or 1 base pairs (e.g., the two sub-sequences having at least 80%, at least 85%, at least 90%, at least 95% similarity, at least 97% similarity, at least 98% similarity, at least 99% similarity, or at least 99.5% similarity). In certain instances, e.g., as set forth herein, each sub-sequence has a length of at least 50 base pairs that differ by fewer than 10, 9, 8, 7, 6, 5, 4, 3, 2, or 1 base pairs (e.g., the two sub-sequences having at least 80%, at least 85%, at least 90%, at least 95% similarity, at least 97% similarity, at least 98% similarity, at least 99% similarity, or at least 99.5% similarity). In certain instances, e.g., as set forth herein, each sub-sequence has a length of at least 100 base pairs that differ by fewer than 20, 15, 10, 9, 8, 7, 6, 5, 4, 3, 2, or 1 base pairs (e.g., the two sub-sequences having at least 80%, at least 85%, at least 90%, at least 95% similarity, at least 97% similarity, at least 98% similarity, at least 99% similarity, or at least 99.5% similarity). In certain instances, e.g., as set forth herein, each sub-sequence has a length of at least 200 base pairs that differ by fewer than 40, 30, 20, 15, 10, 9, 8, 7, 6, 5, 4, 3, 2, or 1 base pairs (e.g., the two sub-sequences having at least 80%, at least 85%, at least 90%, at least 95% similarity, at least 97% similarity, at least 98% similarity, at least 99% similarity, or at least 99.5% similarity). In certain instances, e.g., as set forth herein, each sub-sequence has a length of at least 250 base pairs that differ by fewer than 50, 40, 30, 20, 15, 10, 9, 8, 7, 6, 5, 4, 3, 2, or 1 base pairs (e.g., the two sub-sequences having at least 80%, at least 85%, at least 90%, at least 95% similarity, at least 97% similarity, at least 98% similarity, at least 99% similarity, or at least 99.5% similarity). In certain instances, e.g., as set forth herein, each sub-sequence has a length of at least 300 base pairs that differ by fewer than 60, 50, 40, 30, 20, 15, 10, 9, 8, 7, 6, 5, 4, 3, 2, or 1 base pairs (e.g., the two sub-sequences having at least 80%, at least 85%, at least 90%, at least 95% similarity, at least 97% similarity, at least 98% similarity, at least 99% similarity, or at least 99.5% similarity). In certain instances, e.g., as set forth herein, each sub-sequence has a length of at least 500 base pairs that differ by fewer than 100, 60, 50, 40, 30, 20, 15, 10, 9, 8, 7, 6, 5, 4, 3, 2, or 1 base pairs (e.g., the two sub-sequences having at least 80%, at least 85%, at least 90%, at least 95% similarity, at least 97% similarity, at least 98% similarity, at least 99% similarity, or at least 99.5% similarity). In certain instances, e.g., as set forth herein, each sub-sequence has a length of at least 1000 base pairs that differ by fewer than 200, 100, 60, 50, 40, 30, 20, 15, 10, 9, 8, 7, 6, 5, 4, 3, 2, or 1 base pairs (e.g., the two sub-sequences having at least 80%, at least 85%, at least 90%, at least 95% similarity, at least 97% similarity, at least 98% similarity, at least 99% similarity, or at least 99.5% similarity). In certain instances, e.g., as set forth herein, the subsequence of a first region of the two regions of DNA may comprise the entirety of the second region of the two regions of DNA (or vice versa) (e.g., the common sub-sequence may contain the whole of either or both regions). In certain embodiments, where a methylation locus has a sequence that comprises “at least a portion of” a DMR sequence listed herein (e.g., at least 20%, at least 30%, at least 40%, at least 50%, at least 60%, at least 70%, at least 80%, or at least 90% of the DMR sequence), the overlapping portion of the methylation locus has at least 95% similarity, at least 98% similarity, or at least 99% similarity with the overlapping portion of the DMR sequence (e.g., if the overlapping portion is 100 bp, the portion of the methylation locus that overlaps with the portion of the DMR differs by no more than 1 bp, no more than 2 bp, or no more than 5 bp). In certain embodiments, where a methylation locus has a sequence that comprises “at least a portion of” a DMR sequence listed herein, this means the methylation locus has a subsequence in common with the DMR sequence that has a consecutive series of bases that covers at least 20%, at least 30%, at least 40%, at least 50%, at least 60%, at least 70%, at least 80%, or at least 90% of the DMR sequence, e.g., wherein the subsequence in common differs by no more than 1 bp, no more than 2 bp, or no more than 5 bp). In certain embodiments, where a methylation locus has a sequence that comprises “at least a portion of” a DMR sequence listed herein, this means the methylation locus contains at least a portion of (e.g., at least 20%, at least 30%, at least 40%, at least 50%, at least 60%, at least 70%, at least 80%, or at least 90% of) the CpG dinucleotides corresponding to the CpG dinucleotides within the DMR sequence.

Pharmaceutical composition: As used herein, the term “pharmaceutical composition” refers to a composition in which an active agent is formulated together with one or more pharmaceutically acceptable carriers. In some embodiments, e.g., as set forth herein, the active agent is present in a unit dose amount appropriate for administration to a subject, e.g., in a therapeutic regimen that shows a statistically significant probability of achieving a predetermined therapeutic effect when administered to a relevant population. In some embodiments, e.g., as set forth herein, a pharmaceutical composition can be formulated for administration in a particular form (e.g., in a solid form or a liquid form), and/or can be specifically adapted for, for example: oral administration (for example, as a drenche (aqueous or non-aqueous solutions or suspensions), tablet, capsule, bolus, powder, granule, paste, etc., which can be formulated specifically for example for buccal, sublingual, or systemic absorption); parenteral administration (for example, by subcutaneous, intramuscular, intravenous or epidural injection as, for example, a sterile solution or suspension, or sustained-release formulation, etc.); topical application (for example, as a cream, ointment, patch or spray applied for example to skin, lungs, or oral cavity); intravaginal or intrarectal administration (for example, as a pessary, suppository, cream, or foam); ocular administration; nasal or pulmonary administration, etc.

Pharmaceutically acceptable: As used herein, the term “pharmaceutically acceptable,” as applied to one or more, or all, component(s) for formulation of a composition as disclosed herein, means that each component must be compatible with the other ingredients of the composition and not deleterious to the recipient thereof.

Pharmaceutically acceptable carrier: As used herein, the term “pharmaceutically acceptable carrier” refers to a pharmaceutically-acceptable material, composition, or vehicle, such as a liquid or solid filler, diluent, excipient, or solvent encapsulating material, that facilitates formulation and/or modifies bioavailability of an agent, e.g., a pharmaceutical agent. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Prevent or prevention: The terms “prevent” and “prevention,” as used herein in connection with the occurrence of a disease, disorder, or condition, refers to reducing the risk of developing the disease, disorder, or condition; delaying onset of the disease, disorder, or condition; delaying onset of one or more characteristics or symptoms of the disease, disorder, or condition; and/or to reducing the frequency and/or severity of one or more characteristics or symptoms of the disease, disorder, or condition. Prevention can refer to prevention in a particular subject or to a statistical impact on a population of subjects. Prevention can be considered complete when onset of a disease, disorder, or condition has been delayed for a predefined period of time.

Probe: As used herein, the term “probe” refers to a single- or double-stranded nucleic acid molecule that is capable of hybridizing with a complementary target and includes a detectable moiety. In certain embodiments, e.g., as set forth herein, a probe is a restriction digest product or is a synthetically produced nucleic acid, e.g., a nucleic acid produced by recombination or amplification. In some instances, e.g., as set forth herein, a probe is a capture probe useful in detection, identification, and/or isolation of a target sequence, such as a gene sequence. In various instances, e.g., as set forth herein, a detectable moiety of probe can be, e.g., an enzyme (e.g., ELISA, as well as enzyme-based histochemical assays), fluorescent moiety, radioactive moiety, or moiety associated with a luminescence signal.

Prognosis: As used herein, the term “prognosis” refers to determining the qualitative of quantitative probability of at least one possible future outcome or event. As used herein, a prognosis can be a determination of the likely course of a disease, disorder, or condition such as cancer in a subject, a determination regarding the life expectancy of a subject, or a determination regarding response to therapy, e.g., to a particular therapy.

Prognostic information: As used herein, the term “prognostic information” refers to information useful in providing a prognosis. Prognostic information can include, without limitation, biomarker status information.

Promoter: As used herein, a “promoter” can refer to a DNA regulatory region that directly or indirectly (e.g., through promoter-bound proteins or substances) associates with an RNA polymerase and participates in initiation of transcription of a coding sequence.

Reference: As used herein describes a standard or control relative to which a comparison is performed. For example, in some embodiments, e.g., as set forth herein, an agent, subject, animal, individual, population, sample, sequence, or value of interest is compared with a reference or control agent, subject, animal, individual, population, sample, sequence, or value. In some embodiments, e.g., as set forth herein, a reference or characteristic thereof is tested and/or determined substantially simultaneously with the testing or determination of the characteristic in a sample of interest. In some embodiments, e.g., as set forth herein, a reference is a historical reference, optionally embodied in a tangible medium. Typically, as would be understood by those of skill in the art, a reference is determined or characterized under comparable conditions or circumstances to those under assessment, e.g., with regard to a sample. Those skilled in the art will appreciate when sufficient similarities are present to justify reliance on and/or comparison to a particular possible reference or control.

Risk: As used herein with respect to a disease, disorder, or condition, the term “risk” refers to the qualitative of quantitative probability (whether expressed as a percentage or otherwise) that a particular individual will develop the disease, disorder, or condition. In some embodiments, e.g., as set forth herein, risk is expressed as a percentage. In some embodiments, e.g., as set forth herein, a risk is a qualitative of quantitative probability that is equal to or greater than 0, 1, 2, 3, 4, 5, 6, 7, 8, 9, 10, 20, 30, 40, 50, 60, 70, 80, 90, or 100%. In some embodiments, e.g., as set forth herein, risk is expressed as a qualitative of quantitative level of risk relative to a reference risk or level or the risk of the same outcome attributed to a reference. In some embodiments, e.g., as set forth herein, relative risk is increased or decreased in comparison to the reference sample by a factor of 1.1, 1.2, 1.3, 1.4, 1.5, 1.6, 1.7, 1.8, 1.9, 2, 3, 4, 5, 6, 7, 8, 9, 10, or more.

Sample: As used herein, the term “sample” typically refers to an aliquot of material obtained or derived from a source of interest. In some embodiments, e.g., as set forth herein, a source of interest is a biological or environmental source. In some embodiments, e.g., as set forth herein, a sample is a “primary sample” obtained directly from a source of interest. In some embodiments, e.g., as set forth herein, as will be clear from context, the term “sample” refers to a preparation that is obtained by processing of a primary sample (e.g., by removing one or more components of and/or by adding one or more agents to a primary sample). Such a “processed sample” can include, for example cells, nucleic acids, or proteins extracted from a sample or obtained by subjecting a primary sample to techniques such as amplification or reverse transcription of nucleic acids, isolation and/or purification of certain components, etc.

In certain instances, e.g., as set forth herein, a processed sample can be a DNA sample that has been amplified (e.g., pre-amplified). Thus, in various instances, e.g., as set forth herein, an identified sample can refer to a primary form of the sample or to a processed form of the sample. In some instances, e.g., as set forth herein, a sample that is enzyme-digested DNA can refer to primary enzyme-digested DNA (the immediate product of enzyme digestion) or a further processed sample such as enzyme-digested DNA that has been subject to an amplification step (e.g., an intermediate amplification step, e.g., pre-amplification) and/or to a filtering step, purification step, or step that modifies the sample to facilitate a further step, e.g., in a process of determining methylation status (e.g., methylation status of a primary sample of DNA and/or of DNA as it existed in its original source context).

Screening: As used herein, the term “screening” refers to any method, technique, process, or undertaking intended to generate diagnostic information and/or prognostic information. Accordingly, those of skill in the art will appreciate that the term screening encompasses method, technique, process, or undertaking that determines whether an individual has, is likely to have or develop, or is at risk of having or developing a disease, disorder, or condition, e.g., colorectal cancer.

Specificity: As used herein, the “specificity” of a biomarker refers to the percentage of samples that are characterized by absence of the event or state of interest for which measurement of the biomarker accurately indicates absence of the event or state of interest (true negative rate). In various embodiments, e.g., as set forth herein, characterization of the negative samples is independent of the biomarker, and can be achieved by any relevant measure, e.g., any relevant measure known to those of skill in the art. Thus, specificity reflects the probability that the biomarker would detect the absence of the event or state of interest when measured in a sample not characterized that event or state of interest. In particular embodiments in which the event or state of interest is colorectal cancer, e.g., as set forth herein, specificity refers to the probability that a biomarker would detect the absence of colorectal cancer in a subject lacking colorectal cancer. Lack of colorectal cancer can be determined, e.g., by histology.

Sensitivity: As used herein, the “sensitivity” of a biomarker refers to the percentage of samples that are characterized by the presence of the event or state of interest for which measurement of the biomarker accurately indicates presence of the event or state of interest (true positive rate). In various embodiments, e.g., as set forth herein, characterization of the positive samples is independent of the biomarker, and can be achieved by any relevant measure, e.g., any relevant measure known to those of skill in the art. Thus, sensitivity reflects the probability that a biomarker would detect the presence of the event or state of interest when measured in a sample characterized by presence of that event or state of interest. In particular embodiments in which the event or state of interest is colorectal cancer, e.g., as set forth herein, sensitivity refers to the probability that a biomarker would detect the presence of colorectal cancer in a subject that has colorectal cancer. Presence of colorectal cancer can be determined, e.g., by histology.

Solid Tumor: As used herein, the term “solid tumor” refers to an abnormal mass of tissue including cancer cells. In various embodiments, e.g., as set forth herein, a solid tumor is or includes an abnormal mass of tissue that does not contain cysts or liquid areas. In some embodiments, e.g., as set forth herein, a solid tumor can be benign; in some embodiments, a solid tumor can be malignant. Examples of solid tumors include carcinomas, lymphomas, and sarcomas. In some embodiments, e.g., as set forth herein, solid tumors can be or include adrenal, bile duct, bladder, bone, brain, breast, cervix, colon, endometrium, esophagum, eye, gall bladder, gastrointestinal tract, kidney, larynx, liver, lung, nasal cavity, nasopharynx, oral cavity, ovary, penis, pituitary, prostate, retina, salivary gland, skin, small intestine, stomach, testis, thymus, thyroid, uterine, vaginal, and/or vulval tumors.

Stage of cancer: As used herein, the term “stage of cancer” refers to a qualitative or quantitative assessment of the level of advancement of a cancer. In some embodiments, e.g., as set forth herein, criteria used to determine the stage of a cancer can include, but are not limited to, one or more of where the cancer is located in a body, tumor size, whether the cancer has spread to lymph nodes, whether the cancer has spread to one or more different parts of the body, etc. In some embodiments, e.g., as set forth herein, cancer can be staged using the so-called TNM System, according to which T refers to the size and extent of the main tumor, usually called the primary tumor; N refers to the number of nearby lymph nodes that have cancer; and M refers to whether the cancer has metastasized. In some embodiments, e.g., as set forth herein, a cancer can be referred to as Stage 0 (abnormal cells are present but have not spread to nearby tissue, also called carcinoma in situ, or CIS; CIS is not cancer, but it can become cancer), Stage I-III (cancer is present; the higher the number, the larger the tumor and the more it has spread into nearby tissues), or Stage IV (the cancer has spread to distant parts of the body). In some embodiments, e.g., as set forth herein, a cancer can be assigned to a stage selected from the group consisting of: in situ (abnormal cells are present but have not spread to nearby tissue); localized (cancer is limited to the place where it started, with no sign that it has spread); regional (cancer has spread to nearby lymph nodes, tissues, or organs): distant (cancer has spread to distant parts of the body); and unknown (there is not enough information to identify cancer stage).

Susceptible to: An individual who is “susceptible to” a disease, disorder, or condition is at risk for developing the disease, disorder, or condition. In some embodiments, e.g., as set forth herein, an individual who is susceptible to a disease, disorder, or condition does not display any symptoms of the disease, disorder, or condition. In some embodiments, e.g., as set forth herein, an individual who is susceptible to a disease, disorder, or condition has not been diagnosed with the disease, disorder, and/or condition. In some embodiments, e.g., as set forth herein, an individual who is susceptible to a disease, disorder, or condition is an individual who has been exposed to conditions associated with, or presents a biomarker status (e.g., a methylation status) associated with, development of the disease, disorder, or condition. In some embodiments, e.g., as set forth herein, a risk of developing a disease, disorder, and/or condition is a population-based risk (e.g., family members of individuals suffering from the disease, disorder, or condition).

Subject: As used herein, the term “subject” refers to an organism, typically a mammal (e.g., a human). In some embodiments, e.g., as set forth herein, a subject is suffering from a disease, disorder or condition. In some embodiments, e.g., as set forth herein, a subject is susceptible to a disease, disorder, or condition. In some embodiments, e.g., as set forth herein, a subject displays one or more symptoms or characteristics of a disease, disorder or condition. In some embodiments, e.g., as set forth herein, a subject is not suffering from a disease, disorder or condition. In some embodiments, e.g., as set forth herein, a subject does not display any symptom or characteristic of a disease, disorder, or condition. In some embodiments, e.g., as set forth herein, a subject is someone with one or more features characteristic of susceptibility to or risk of a disease, disorder, or condition. In some embodiments, e.g., as set forth herein, a subject is a patient. In some embodiments, e.g., as set forth herein, a subject is an individual to whom diagnosis has been performed and/or to whom therapy has been administered. In some instances, e.g., as set forth herein, a human subject can be interchangeably referred to as an “individual.”

Therapeutic agent: As used herein, the term “therapeutic agent” refers to any agent that elicits a desired pharmacological effect when administered to a subject. In some embodiments, e.g., as set forth herein, an agent is considered to be a therapeutic agent if it demonstrates a statistically significant effect across an appropriate population. In some embodiments, e.g., as set forth herein, the appropriate population can be a population of model organisms or a human population. In some embodiments, e.g., as set forth herein, an appropriate population can be defined by various criteria, such as a certain age group, gender, genetic background, preexisting clinical conditions, etc. In some embodiments, e.g., as set forth herein, a therapeutic agent is a substance that can be used for treatment of a disease, disorder, or condition. In some embodiments, e.g., as set forth herein, a therapeutic agent is an agent that has been or is required to be approved by a government agency before it can be marketed for administration to humans. In some embodiments, e.g., as set forth herein, a therapeutic agent is an agent for which a medical prescription is required for administration to humans.

Therapeutically effective amount: As used herein, the term “therapeutically effective amount” refers to an amount that produces a desired effect for which it is administered. In some embodiments, e.g., as set forth herein, the term refers to an amount that is sufficient, when administered to a population suffering from or susceptible to a disease, disorder, or condition, in accordance with a therapeutic dosing regimen, to treat the disease, disorder, or condition. Those of ordinary skill in the art will appreciate that the term therapeutically effective amount does not in fact require successful treatment be achieved in a particular individual. Rather, a therapeutically effective amount can be an amount that provides a particular desired pharmacological response in a significant number of subjects when administered to individuals in need of such treatment. In some embodiments, e.g., as set forth herein, reference to a therapeutically effective amount can be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in some embodiments, a therapeutically effective amount of a particular agent can be formulated and/or administered in a single dose. In some embodiments, e.g., as set forth herein, a therapeutically effective agent can be formulated and/or administered in a plurality of doses, for example, as part of a multi-dose dosing regimen.

Treatment: As used herein, the term “treatment” (also “treat” or “treating”) refers to administration of a therapy that partially or completely alleviates, ameliorates, relieves, inhibits, delays onset of, reduces severity of, and/or reduces incidence of one or more symptoms, features, and/or causes of a particular disease, disorder, or condition, or is administered for the purpose of achieving any such result. In some embodiments, e.g., as set forth herein, such treatment can be of a subject who does not exhibit signs of the relevant disease, disorder, or condition and/or of a subject who exhibits only early signs of the disease, disorder, or condition. Alternatively or additionally, such treatment can be of a subject who exhibits one or more established signs of the relevant disease, disorder and/or condition. In some embodiments, e.g., as set forth herein, treatment can be of a subject who has been diagnosed as suffering from the relevant disease, disorder, and/or condition. In some embodiments, e.g., as set forth herein, treatment can be of a subject known to have one or more susceptibility factors that are statistically correlated with increased risk of development of the relevant disease, disorder, or condition. In various examples, treatment is of a cancer.

Upstream: As used herein, the term “upstream” means a first DNA region is closer, relative to a second DNA region, to the N-terminus of a nucleic acid that includes the first DNA region and the second DNA region.

Unit dose: As used herein, the term “unit dose” refers to an amount administered as a single dose and/or in a physically discrete unit of a pharmaceutical composition. In many embodiments, e.g., as set forth herein, a unit dose contains a predetermined quantity of an active agent. In some embodiments, e.g., as set forth herein, a unit dose contains an entire single dose of the agent. In some embodiments, e.g., as set forth herein, more than one unit dose is administered to achieve a total single dose. In some embodiments, e.g., as set forth herein, administration of multiple unit doses is required, or expected to be required, in order to achieve an intended effect. A unit dose can be, for example, a volume of liquid (e.g., an acceptable carrier) containing a predetermined quantity of one or more therapeutic moieties, a predetermined amount of one or more therapeutic moieties in solid form, a sustained release formulation or drug delivery device containing a predetermined amount of one or more therapeutic moieties, etc. It will be appreciated that a unit dose can be present in a formulation that includes any of a variety of components in addition to the therapeutic agent(s). For example, acceptable carriers (e.g., pharmaceutically acceptable carriers), diluents, stabilizers, buffers, preservatives, etc., can be included. It will be appreciated by those skilled in the art, in many embodiments, e.g., as set forth herein, a total appropriate daily dosage of a particular therapeutic agent can comprise a portion, or a plurality, of unit doses, and can be decided, for example, by a medical practitioner within the scope of sound medical judgment. In some embodiments, e.g., as set forth herein, the specific effective dose level for any particular subject or organism can depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts

Unmethylated: As used herein, the terms “unmethylated” and “non-methylated” are used interchangeable and mean that an identified DNA region includes no methylated nucleotides.

Variant: As used herein, the term “variant” refers to an entity that shows significant structural identity with a reference entity but differs structurally from the reference entity in the presence, absence, or level of one or more chemical moieties as compared with the reference entity. In some embodiments, e.g., as set forth herein, a variant also differs functionally from its reference entity. In general, whether a particular entity is properly considered to be a “variant” of a reference entity is based on its degree of structural identity with the reference entity. A variant can be a molecule comparable, but not identical to, a reference. For example, a variant nucleic acid can differ from a reference nucleic acid at one or more differences in nucleotide sequence. In some embodiments, e.g., as set forth herein, a variant nucleic acid shows an overall sequence identity with a reference nucleic acid that is at least 85%, 86%, 87%, 88%, 89%, 90%, 91%, 92%, 93%, 94%, 95%, 96%, 97%, or 99%. In many embodiments, e.g., as set forth herein, a nucleic acid of interest is considered to be a “variant” of a reference nucleic acid if the nucleic acid of interest has a sequence that is identical to that of the reference but for a small number of sequence alterations at particular positions. In some embodiments, e.g., as set forth herein, a variant has 10, 9, 8, 7, 6, 5, 4, 3, 2, or 1 substituted residues as compared with a reference. In some embodiments, e.g., as set forth herein, a variant has not more than 5, 4, 3, 2, or 1 residue additions, substitutions, or deletions as compared with the reference. In various embodiments, e.g., as set forth herein, the number of additions, substitutions, or deletions is fewer than about 25, about 20, about 19, about 18, about 17, about 16, about 15, about 14, about 13, about 10, about 9, about 8, about 7, about 6, and commonly are fewer than about 5, about 4, about 3, or about 2 residues.

### BRIEF DESCRIPTION OF THE FIGURES

The foregoing and other objects, aspects, features, and advantages of the present disclosure will become more apparent and better understood by referring to the following description taken in conjunction with the accompanying drawings, in which:

FIG. 1 is a schematic showing an MSRE-qPCR approach, according to an illustrative embodiment.

FIG. 2 is a PCA (Principle Component Analysis) plot over an initial marker set indicating separation between disease groups.

FIGS. 3A-L are graphs representing 45-Ct values from MSRE-qPCR of DNA from plasma samples of subjects with advanced adenoma (AA) and colorectal cancer (CRC) as compared to control subjects (CNT; healthy subjects and subjects with hyperplastic polyps and GID). Higher 45-Ct values correspond to a higher degree of methylation in AA+CRC samples.

FIGS. 4A-C are graphs representing Ct values from MSRE-qPCR of DNA for subjects with advanced adenoma (AA) as compared to control subjects (CNT; healthy subjects and subjects with hyperplastic polyps and GID).

FIGS. 5A-R are graphs representing Ct values from MSRE-qPCR of DNA for subjects with colorectal cancer (CRC) as compared to control subjects (CNT; healthy subjects and subjects with hyperplastic polyps and GID).

### DETAILED DESCRIPTION

It is contemplated that systems, architectures, devices, methods, and processes of the claimed invention encompass variations and adaptations developed using information from the embodiments described herein. Adaptation and/or modification of the systems, architectures, devices, methods, and processes described herein may be performed, as contemplated by this description.

Throughout the description, where articles, devices, systems, and architectures are described as having, including, or comprising specific components, or where processes and methods are described as having, including, or comprising specific steps, it is contemplated that, additionally, there are articles, devices, systems, and architectures of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim.

Documents are incorporated herein by reference as noted. Where there is any discrepancy in the meaning of a particular term, the meaning provided in the Definition section above is controlling.

Screening for Advanced Adenoma and/or Colorectal Cancer (e.g., Early Stage Colorectal Cancer)

There is a need for improved methods of detecting (e.g., screening for) advanced adenoma and/or colorectal cancer, including screening for diagnosis of early-stage colorectal cancer. Despite recommendations for screening of individuals, e.g., over age 50, colorectal cancer screening programs are often ineffective or unsatisfactory. Improved colorectal cancer screening improves diagnosis and reduces colorectal cancer mortality.

DNA methylation (e.g., hypermethylation or hypomethylation) can activate or inactivate genes, including genes that impact cancer development. Thus, for example, hypermethylation can inactivate one or more genes that typically act to suppress cancer, causing or contributing to development of cancer in a sample or subject.

The present disclosure includes the discovery that determination of the methylation status of one or more methylation loci provided herein, and/or the methylation status of one or more DMRs provided herein, and/or the methylation status of one or more methylation sites provided herein, provides screening for advanced adenoma and/or colorectal cancer (e.g., early stage colorectal cancer), e.g., with a high degree of sensitivity and/or specificity. The present disclosure provides compositions and methods including or relating to advanced adenoma and/or colorectal cancer methylation biomarkers that, individually or in various panels comprising two or more methylation biomarkers, provide for screening of advanced adenoma and/or colorectal cancer (e.g., early stage colorectal cancer), e.g., with a high degree of specificity and/or sensitivity.

In various embodiments, a colorectal cancer methylation biomarker of the present disclosure is selected from a methylation locus that is or includes the DMRs listed in Table 1.

TABLE 1
List of DMRs found to have significantly
altered methylation pattern in the blood
of colorectal cancer and/or advanced adenoma
patients compared to controls.

| | | | | SEQ | | SEQ | | SEQ | |
|---|---|---|---|---|---|---|---|---|---|
| | | | | ID | | ID | | ID | associated_ |
| chr | start | end | prim_f | NO: | prim_r | NO: | Seq (DMR) | NO: | genes |
| 19 | 57499812 | 57499919 | CGAT | 27 | TGAGT | 91 | CGATGCTTGGC | 156 | ZNF773, |
| | | | GCTT | | TCTCTT | | CAATGAAAAG | | ZNF419 |
| | | | GGCC | | GAGGG | | AGGTCTACCC | | |
| | | | AATG | | CAGCG | | GAGAGTGCGA | | |
| | | | AAA | | AAA | | CGCGCAATGG | | |
| | | | AGA | | | | GCGGGACTTC | | |
| | | | GG | | | | CGGCGTCTCCC | | |
| | | | | | | | CTCGGCGGTTG | | |
| | | | | | | | CTTTCGCTGCC | | |
| | | | | | | | CTCAAGAGAA | | |
| | | | | | | | CTCA | | |
| 3 | 42686373 | 42686488 | GCAA | 28 | GGCTT | 92 | GCAAGGTGCA | 157 | KLHL40 |
| | | | GGTG | | TGCTT | | GATGGTGAAG | | |
| | | | CAGA | | GTGCC | | GATGCACACG | | |
| | | | TGGT | | CTTAT | | AGGGCCGCAT | | |
| | | | GAA | | CAGC | | CACCACGCTG | | |
| | | | GGAT | | | | CGGAAGAAAA | | |
| | | | G | | | | AGAAGGGGAA | | |
| | | | | | | | GGATGGAGCC | | |
| | | | | | | | GGGGCCAAGG | | |
| | | | | | | | AGGCTGATAA | | |
| | | | | | | | GGGCACAAGC | | |
| | | | | | | | AAAGCC | | |
| 20 | 58840200 | 58840314 | CTCC | 29 | GGTGG | 93 | CTCCTCTGGCT | 158 | GNAS, |
| | | | TCTG | | TGGGC | | CTCCTGCTCCA | | GNAS-AS1 |
| | | | GCTC | | GTTAA | | TCGCGCTCCTC | | |
| | | | TCCT | | GGAAG | | CGCGCCCTTGC | | |
| | | | GCTC | | CTC | | CACCTCCAAC | | |
| | | | CATC | | | | GCCCGTGCCC | | |
| | | | | | | | AGCAGCGCGC | | |
| | | | | | | | GGCTGCCCAA | | |
| | | | | | | | CAGCGCCGGA | | |
| | | | | | | | GCTTCCTTAAC | | |
| | | | | | | | GCCCACCACC | | |
| 22 | 39255457 | 39255580 | GAG | 30 | GTACC | 94 | GAGGCAAAAA | 159 | |
| | | | GCAA | | CTTCA | | GGACAATCGG | | |
| | | | AAA | | CCCAC | | CAAGTAAATA | | |
| | | | GGAC | | CCAGG | | GTAAATGAAC | | |
| | | | AATC | | GTTT | | AAGAAGACCC | | |
| | | | GGCA | | | | CGGTTGTGAG | | |
| | | | AG | | | | AAAATGTTAT | | |
| | | | | | | | AAAGCAAATA | | |
| | | | | | | | AATCAGAGAA | | |
| | | | | | | | ATGTGATCAC | | |
| | | | | | | | AAACCCTGGG | | |
| | | | | | | | TGGGTGAAGG | | |
| | | | | | | | GTAC | | |
| 3 | 75609726 | 75609832 | CCTG | 31 | CCCCT | 95 | CCTGCGACGT | 21 | |
| | | | CGAC | | ATCTC | | GAATCGTCAT | | |
| | | | GTGA | | CCCCT | | ATCCAGAGGG | | |
| | | | ATCG | | GGCTC | | GGGTGATATG | | |
| | | | TCAT | | TTAG | | ACTCCCCGCAT | | |
| | | | ATCC | | | | CGCGGGGGCC | | |
| | | | | | | | TCACCCCATTG | | |
| | | | | | | | CGATGGGGGT | | |
| | | | | | | | CCTAAGAGCC | | |
| | | | | | | | AGGGGGAGAT | | |
| | | | | | | | AGGGG | | |
| 1 | 2978882 | 2978962 | AGCT | 32 | GAACC | 96 | AGCTGAGGGA | 160 | |
| | | | GAG | | CCAGT | | AAGGGGGAAG | | |
| | | | GGA | | GGACC | | TCACTGGGCTG | | |
| | | | AAG | | CCTCA | | GGGGCCGGGG | | |
| | | | GGG | | GA | | CCGCTCACTCT | | |
| | | | GAA | | | | GGCCTCCTCTG | | |
| | | | GTCA | | | | AGGGGTCCAC | | |
| | | | C | | | | TGGGGTTC | | |
| 3 | 113441434 | 113441539 | TGCT | 33 | AGGGG | 97 | TGCTGAGGTCC | 5 | CFAP44 |
| | | | GAG | | TGGCG | | AAACTCACCG | | |
| | | | GTCC | | TTGTC | | AAGGTACTGA | | |
| | | | AAAC | | TCGGT | | CCGCCGCGGC | | |
| | | | TCAC | | AAC | | TCCTCTCTTCA | | |
| | | | CGAA | | | | CAGCGTCTGCC | | |
| | | | G | | | | GGAGGCCTCC | | |
| | | | | | | | GTTTACTCCGG | | |
| | | | | | | | TTACCGAGAC | | |
| | | | | | | | AACGCCACCC | | |
| | | | | | | | CT | | |
| 9 | 126688327 | 126688455 | CCTT | 34 | GGCCT | 98 | CCTTCCCCAGC | 161 | LMX1B |
| | | | CCCC | | CTGAG | | CTAAGAAGGT | | |
| | | | AGCC | | TGGAC | | TTCCTCTCCGG | | |
| | | | TAAG | | AGACA | | GAGTCACCCA | | |
| | | | AAG | | CTGG | | AGGTGTGCTG | | |
| | | | GTTT | | | | ACCCTGGCCTG | | |
| | | | CC | | | | GGACCCTGGG | | |
| | | | | | | | ACCGTGGCGC | | |
| | | | | | | | TCCCACGCTAG | | |
| | | | | | | | CAGCGACACG | | |
| | | | | | | | GCCAGTGTCTG | | |
| | | | | | | | TCCACTCAGA | | |
| | | | | | | | GGCC | | |
| 9 | 135927101 | 135927190 | GCCA | 35 | CTCGA | 99 | GCCAGTGAGT | 162 | |
| | | | GTGA | | GTCCT | | CAGAGGCAGA | | |
| | | | GTCA | | GGAGG | | GGTGCCAGAG | | |
| | | | GAG | | AGCCT | | ACCCCGCCCG | | |
| | | | GCAG | | GTG | | AAGGGAGGAG | | |
| | | | AGGT | | | | ATCTGAGAGC | | |
| | | | G | | | | CTGCAGCCAC | | |
| | | | | | | | AGGCTCCTCCA | | |
| | | | | | | | GGACTCGAG | | |
| 3 | 113441519 | 113441620 | TACC | 36 | GATAG | 100 | TACCGAGACA | 6 | CFAP44 |
| | | | GAG | | GTACC | | ACGCCACCCCT | | |
| | | | ACAA | | GGGTT | | CTTCCAGGGA | | |
| | | | CGCC | | CCCGG | | GGCGGAACCA | | |
| | | | ACCC | | AGAG | | GGGCGGGCCG | | |
| | | | CTCT | | | | TGGGGCGCAT | | |
| | | | T | | | | GCGCGGCCGG | | |
| | | | | | | | CGTCCAGCTCT | | |
| | | | | | | | CCGGGAACCC | | |
| | | | | | | | GGTACCTATC | | |
| 17 | 78124443 | 78124551 | GAGC | 37 | CACAG | 101 | GAGCTGGGAC | 163 | TMC6 |
| | | | TGGG | | GCCTG | | AAGAAGGGAA | | |
| | | | ACAA | | GAGCT | | CACGGTACCA | | |
| | | | GAA | | CCTCA | | GGGTAGCAGA | | |
| | | | GGG | | CA | | AGACAGGCAC | | |
| | | | AACA | | | | CCCCCGTCCCC | | |
| | | | CG | | | | CAGTCCTAGG | | |
| | | | | | | | GCTTCCTCACC | | |
| | | | | | | | GCGCCTGTGA | | |
| | | | | | | | GGAGCTCCAG | | |
| | | | | | | | GCCTGTG | | |
| 3 | 113441596 | 113441690 | GCTC | 38 | AAACT | 102 | GCTCTCCGGG | 7 | CFAP44 |
| | | | TCCG | | CGTCG | | AACCCGGTAC | | |
| | | | GGA | | GGCTT | | CTATCCGCCCT | | |
| | | | ACCC | | TTCTG | | TTGGTCGGGCC | | |
| | | | GGTA | | TTTGG | | TTCTCCGCCTC | | |
| | | | CCTA | | | | ATGACACTGG | | |
| | | | T | | | | TTCAAAGCCA | | |
| | | | | | | | AACAGAAAAG | | |
| | | | | | | | CCCGACGAGT | | |
| | | | | | | | TT | | |
| 6 | 34514653 | 34514751 | GCGT | 39 | GACAG | 103 | GCGTCTCTGTG | 164 | PACSIN1 |
| | | | CTCT | | CCTCC | | GCCGTGAAGT | | |
| | | | GTGG | | CTCCC | | GTATGCATGC | | |
| | | | CCGT | | ATGTA | | GTGCCCATGTT | | |
| | | | GAA | | CAGC | | GATGCGGCGC | | |
| | | | GTGT | | | | CGTGCGGGAG | | |
| | | | A | | | | GCGGGCATCC | | |
| | | | | | | | CCTGCTGTACA | | |
| | | | | | | | TGGGAGGGAG | | |
| | | | | | | | GCTGTC | | |
| 7 | 150996901 | 150997007 | CCGG | 40 | CCCGG | 104 | CCGGATCCAG | 11 | NOS3 |
| | | | ATCC | | GAAGC | | TGGGGGAAGC | | |
| | | | AGTG | | CTTAG | | TGCAGGTGCG | | |
| | | | GGG | | GAACT | | GCTGGCCAGC | | |
| | | | GAA | | GC | | GACTGAGAGA | | |
| | | | GCTG | | | | CCCGGGCGCT | | |
| | | | | | | | ACCAAAAGGG | | |
| | | | | | | | GAGCGGGGTG | | |
| | | | | | | | GCGGGGCAGT | | |
| | | | | | | | TCCTAAGGCTT | | |
| | | | | | | | CCCGGG | | |
| 10 | 75407300 | 75407400 | AATA | 41 | GGGCA | 105 | AATACATCCA | 18 | LOC101929234, |
| | | | CATC | | CTAGA | | GCTCGCAGGC | | ZNF503- |
| | | | CAGC | | AACCA | | ATCCTGCAAG | | AS2 |
| | | | TCGC | | CCTCG | | AAACGGCTCC | | |
| | | | AGGC | | AGTCC | | CGGCTCGCGT | | |
| | | | ATCC | | | | GTACGCCGAC | | |
| | | | | | | | ACCTCGGCCC | | |
| | | | | | | | AACGCAGGAC | | |
| | | | | | | | TCGAGGTGGTT | | |
| | | | | | | | TCTAGTGCCC | | |
| 11 | 129618087 | 129618193 | GGG | 42 | CCCAG | 106 | GGGAGCCTGG | 9 | LINC01395 |
| | | | AGCC | | CCCCG | | AGGGGTTGAC | | |
| | | | TGGA | | GGTAG | | ACCGCCTGCTC | | |
| | | | GGG | | TTACA | | CACCGCAAGC | | |
| | | | GTTG | | CCT | | CCCTGGAGGA | | |
| | | | ACAC | | | | AGAGCCCCGC | | |
| | | | | | | | TGTGCCCGAG | | |
| | | | | | | | AGCGAGCGCG | | |
| | | | | | | | GGCAGGTGTA | | |
| | | | | | | | ACTACCCGGG | | |
| | | | | | | | GCTGGG | | |
| 11 | 129618345 | 129618455 | CCTC | 43 | GCAGA | 107 | CCTCTGCTTCA | 10 | LINC01395 |
| | | | TGCT | | CACAC | | GGTGCTTGGCT | | |
| | | | TCAG | | ATGCG | | AGAGAAAGGG | | |
| | | | GTGC | | CTCAC | | CGGCAAGACG | | |
| | | | TTGG | | ATAA | | GGGCAGTGCG | | |
| | | | CTAG | | | | TGTGCGCGCG | | |
| | | | A | | | | CGGGCAAGTG | | |
| | | | | | | | CATGTGAGTG | | |
| | | | | | | | CACACTTATGT | | |
| | | | | | | | GAGCGCATGT | | |
| | | | | | | | GTGTCTGC | | |
| 13 | 24328192 | 24328315 | GCCC | 44 | CCAGT | 108 | GCCCAAGGGC | 165 | LINC00566 |
| | | | AAG | | TCCAG | | CACAAGAGTA | | |
| | | | GGCC | | GTGTG | | TGACGGGGCT | | |
| | | | ACAA | | GAACC | | GTACGAGCTG | | |
| | | | GAGT | | GAAC | | CTGTGACGGG | | |
| | | | ATGA | | | | TGCTGCATGCG | | |
| | | | C | | | | CTGCTCCGTCT | | |
| | | | | | | | GCACCGCACG | | |
| | | | | | | | CTCACCTCCTG | | |
| | | | | | | | GCTCCGCGTTC | | |
| | | | | | | | GGTTCCACACC | | |
| | | | | | | | TGGAACTGG | | |
| 13 | 24328295 | 24328404 | CGGT | 45 | GTGGT | 109 | CGGTTCCACAC | 166 | LINC00566 |
| | | | TCCA | | GAGGG | | CTGGAACTGG | | |
| | | | CACC | | CTGTT | | ATTTGGCGGC | | |
| | | | TGGA | | CCATG | | GCTGCTGCCGC | | |
| | | | ACTG | | CTT | | GCCGCCTCTGC | | |
| | | | GATT | | | | CGCGGTCCTA | | |
| | | | | | | | GAGCCGCTTG | | |
| | | | | | | | GCTTCACGCTC | | |
| | | | | | | | CGCAAGCATG | | |
| | | | | | | | GAACAGCCCT | | |
| | | | | | | | CACCAC | | |
| 13 | 24328295 | 24328375 | CGGT | 45 | CGTGA | 110 | CGGTTCCACAC | 167 | LINC00566 |
| | | | TCCA | | AGCCA | | CTGGAACTGG | | |
| | | | CACC | | AGCGG | | ATTTGGCGGC | | |
| | | | TGGA | | CTCTA | | GCTGCTGCCGC | | |
| | | | ACTG | | GGAC | | GCCGCCTCTGC | | |
| | | | GATT | | | | CGCGGTCCTA | | |
| | | | | | | | GAGCCGCTTG | | |
| | | | | | | | GCTTCACG | | |
| 13 | 24328351 | 24328441 | GGTC | 46 | GGGCT | 111 | GGTCCTAGAG | 168 | LINC00566 |
| | | | CTAG | | GCGGG | | CCGCTTGGCTT | | |
| | | | AGCC | | CAGGC | | CACGCTCCGC | | |
| | | | GCTT | | ACTAC | | AAGCATGGAA | | |
| | | | GGCT | | | | CAGCCCTCACC | | |
| | | | TCAC | | | | ACACGCACCC | | |
| | | | | | | | GCGCGGGGGG | | |
| | | | | | | | TAGTGCCTGCC | | |
| | | | | | | | CGCAGCCC | | |
| 13 | 114111799 | 114111878 | TGTC | 47 | ATGAT | 112 | TGTCAAACCTC | 12 | RASA3 |
| | | | AAAC | | CCTCT | | CATCTGTGGTC | | |
| | | | CTCC | | GGAAG | | AGGAGTTAGG | | |
| | | | ATCT | | CGCCG | | ACATCCCCAG | | |
| | | | GTGG | | TCT | | CTGCAATTTGA | | |
| | | | TCAG | | | | GCAAAGACGG | | |
| | | | G | | | | CGCTTCCAGA | | |
| | | | | | | | GGATCAT | | |
| 16 | 46844362 | 46844467 | TGGG | 48 | CATCC | 113 | TGGGGCCGAA | 169 | |
| | | | GCCG | | AGACC | | GAGATCCTTG | | |
| | | | AAG | | CGCGT | | AACACGTCGT | | |
| | | | AGAT | | GGACA | | AGGACTCCTC | | |
| | | | CCTT | | GC | | GTCGGCCGCC | | |
| | | | GAAC | | | | ACGCGGCCCA | | |
| | | | A | | | | CGGCCCTGAG | | |
| | | | | | | | TACGGGTGGC | | |
| | | | | | | | CCGGGCTGTCC | | |
| | | | | | | | ACGCGGGTCT | | |
| | | | | | | | GGATG | | |
| 16 | 85238430 | 85238559 | GCTG | 49 | AGGCA | 114 | GCTGCAGTTTC | 170 | GSE1 |
| | | | CAGT | | GCCAA | | GTCAGCCCTTG | | |
| | | | TTCG | | GACAA | | GCTCCGGGCTC | | |
| | | | TCAG | | GCAGA | | TGCAGGCGGA | | |
| | | | CCCT | | GAGG | | ATCCCGAGCCT | | |
| | | | TG | | | | GCGTGAGGGC | | |
| | | | | | | | CGCCCTGGCCT | | |
| | | | | | | | CGGCGTGTGTC | | |
| | | | | | | | CTGGGAAGGG | | |
| | | | | | | | GCGTTGGAAG | | |
| | | | | | | | CCTCTCTGCTT | | |
| | | | | | | | GTCTTGGCTGC | | |
| | | | | | | | CT | | |
| 17 | 78304805 | 78304921 | CAGC | 50 | ACCTC | 115 | CAGCCCTAGG | 26 | |
| | | | CCTA | | TTGAC | | GAGACAGCAG | | |
| | | | GGG | | CCCAG | | GATGGTTCCA | | |
| | | | AGAC | | CTCTG | | GGAAGCCTGG | | |
| | | | AGCA | | ATGC | | GCCGCTCCCCA | | |
| | | | GGAT | | | | GATCAATGCA | | |
| | | | G | | | | GGGACGGACA | | |
| | | | | | | | GCAGCCAGCA | | |
| | | | | | | | GGCTGGGCCA | | |
| | | | | | | | CGGCATCAGA | | |
| | | | | | | | GCTGGGGTCA | | |
| | | | | | | | AGAGGT | | |
| 19 | 4328790 | 4328882 | GGCT | 51 | GGAGG | 116 | GGCTCACCTTC | 171 | STAP2 |
| | | | CACC | | CAGGG | | AGGAAGCACC | | |
| | | | TTCA | | TTTAC | | TGTGGCGGGC | | |
| | | | GGA | | GTGCA | | CGCGTCACCC | | |
| | | | AGCA | | GAAG | | ACTCGGGACC | | |
| | | | CCTG | | | | CCGGAGACCA | | |
| | | | T | | | | AGTCCGCTCTT | | |
| | | | | | | | CTGCACGTAA | | |
| | | | | | | | ACCCTGCCTCC | | |
| 19 | 22709270 | 22709382 | GGGC | 52 | CCCTC | 117 | GGGCCAGTTC | 3 | |
| | | | CAGT | | CTCAT | | CTCCTACCAGC | | |
| | | | TCCT | | TCCCC | | TTCCTGCTGCC | | |
| | | | CCTA | | TGGAC | | ACCTCGGCTTC | | |
| | | | CCAG | | TCTT | | CATCAGAGGG | | |
| | | | CTTC | | | | ACGCTTAGGA | | |
| | | | | | | | TGGCGCAGGG | | |
| | | | | | | | GCCCGGAGAC | | |
| | | | | | | | ACTGTGAAGA | | |
| | | | | | | | GTCCAGGGGA | | |
| | | | | | | | ATGAGGAGGG | | |
| 19 | 37334711 | 37334817 | GGTC | 53 | CAGAG | 118 | GGTCTTTCCCA | 172 | ZNF875 |
| | | | TTTC | | GGGCA | | CACCTCTGCAC | | |
| | | | CCAC | | TGCTG | | CTTGTTACCTG | | |
| | | | ACCT | | ACTGC | | ACTTTCGGCTT | | |
| | | | CTGC | | CTAT | | CAGGATCCGC | | |
| | | | ACCT | | | | AGCGTGCACC | | |
| | | | | | | | CGCGTTCCGTG | | |
| | | | | | | | AGTGCCCTATA | | |
| | | | | | | | GGCAGTCAGC | | |
| | | | | | | | ATGCCCCTCTG | | |
| 19 | 47754827 | 47754942 | GCCT | 54 | AAGGA | 119 | GCCTGGCCAC | 173 | NOP53, |
| | | | GGCC | | AGGGG | | CACAGAGAAG | | SNORD23 |
| | | | ACCA | | GCGAG | | AAGACGGAGC | | |
| | | | CAGA | | GCATC | | AGCAGCGGCG | | |
| | | | GAA | | AG | | GCGGGAGAAG | | |
| | | | GAA | | | | GCTGTGCACA | | |
| | | | GA | | | | GGCTGGTGAG | | |
| | | | | | | | CGCCTGGGCC | | |
| | | | | | | | AGCGGGGCCT | | |
| | | | | | | | GCCTCTGATGC | | |
| | | | | | | | CTCGCCCCCTT | | |
| | | | | | | | CCTT | | |
| 19 | 48568713 | 48568802 | TGCT | 55 | CGGAC | 120 | TGCTCTCTCTC | 174 | SULT2B1 |
| | | | CTCT | | AGCAG | | CAAAGGCGAG | | |
| | | | CTCC | | GAGGG | | TTGATCACAG | | |
| | | | AAA | | ATTTC | | ACGCTGGCAG | | |
| | | | GGCG | | TCAG | | TGAGTCAGCG | | |
| | | | AGTT | | | | GCACCGCCAG | | |
| | | | G | | | | GGCTGCTGAG | | |
| | | | | | | | AAATCCCTCCT | | |
| | | | | | | | GCTGTCCG | | |
| 9 | 95313318 | 95313405 | CCTG | 56 | TGACT | 121 | CCTGGGAACC | 175 | FANCC |
| | | | GGA | | CCTCC | | AGTGCTGGAG | | |
| | | | ACCA | | AGAGC | | AAAGTATGTG | | |
| | | | GTGC | | GAGGT | | GAAGCTGGCG | | |
| | | | TGGA | | TGTG | | ATGGAGAAGG | | |
| | | | GAA | | | | CGCGCGCATG | | |
| | | | AG | | | | TGTGCACAAC | | |
| | | | | | | | CTCGCTCTGGA | | |
| | | | | | | | GGAGTCA | | |
| 9 | 127828322 | 127828421 | GCCC | 57 | CCAGA | 122 | GCCCCTGTAA | 8 | ENG |
| | | | CTGT | | GGGGA | | AATGGGGATA | | |
| | | | AAA | | CAAAG | | CAGCAGGGCA | | |
| | | | ATGG | | AGGCC | | CGACGTCTGTT | | |
| | | | GGAT | | AAG | | GGTCGCCTGG | | |
| | | | ACAG | | | | CACTGGGTCG | | |
| | | | CA | | | | GCCACCGAGG | | |
| | | | | | | | CCGCGCCTTGG | | |
| | | | | | | | CCTCTTTGTCC | | |
| | | | | | | | CCTCTGG | | |
| 11 | 2656072 | 2656156 | TGGG | 58 | TGCAT | 123 | TGGGCACTTGT | 17 | KCNQ1OT1, |
| | | | CACT | | CACTT | | CATCATGGGT | | KCNQ1 |
| | | | TGTC | | GTATG | | GTTTGGAAAG | | |
| | | | ATCA | | TAGAA | | CAACTCTACGT | | |
| | | | TGGG | | GGAAC | | TCTAGCCTGTG | | |
| | | | TGTT | | GATGG | | CTCCATCGTTC | | |
| | | | | | | | CTTCTACATAC | | |
| | | | | | | | AAGTGATGCA | | |
| 14 | 68628944 | 68629052 | ACAC | 59 | GGCCT | 124 | ACACTTTGAA | 176 | LOC100996664, |
| | | | TTTG | | TCCTG | | AAGCGTGGCG | | RAD51B |
| | | | AAA | | CAGCC | | TTCCAGCGCA | | |
| | | | AGCG | | GTCTC | | AACCAACCCG | | |
| | | | TGGC | | TC | | AACGGGTTGG | | |
| | | | GTTC | | | | AAGGGGGCAG | | |
| | | | C | | | | TCCTTTCTTCC | | |
| | | | | | | | CGCAAGTTCG | | |
| | | | | | | | GGGCTCGAGA | | |
| | | | | | | | GACGGCTGCA | | |
| | | | | | | | GGAAGGCC | | |
| 1 | 159200826 | 159200935 | AAA | 60 | TGGGC | 125 | AAAAGGCTCC | 177 | ACKR1, |
| | | | AGGC | | AGGCG | | GACGATGCTC | | CADM3, |
| | | | TCCG | | CCTCT | | CAGACGCGGA | | CADM3- |
| | | | ACGA | | AGATG | | CACGGCCATC | | AS1 |
| | | | TGCT | | AAAT | | ATCAATGCAG | | |
| | | | CCAG | | | | AAGGCGGGCA | | |
| | | | A | | | | GTCAGGAGGG | | |
| | | | | | | | GACGACAAGA | | |
| | | | | | | | AGGAATATTTC | | |
| | | | | | | | ATCTAGAGGC | | |
| | | | | | | | GCCTGCCCA | | |
| 11 | 128685299 | 128685448 | GCAC | 61 | GAACA | 126 | GCACCAAGAA | 16 | FLI1, |
| | | | CAAG | | AGCAG | | CTAACACATCC | | LOC101929538 |
| | | | AACT | | CGAGA | | TGGAGCTGCC | | |
| | | | AACA | | CACAC | | CGGAGTTCCG | | |
| | | | CATC | | TGAG | | CTCCTGCGGGC | | |
| | | | CTGG | | | | TTAGCAGGAA | | |
| | | | AG | | | | AGGGTGCCTA | | |
| | | | | | | | AGGTGAGTGC | | |
| | | | | | | | CCACTTGCGTC | | |
| | | | | | | | CGATCCTCTGG | | |
| | | | | | | | GGGCGATGCA | | |
| | | | | | | | GGGTCGGGGC | | |
| | | | | | | | GCCTCAGTGTG | | |
| | | | | | | | TCTCGCTGCTT | | |
| | | | | | | | GTTC | | |
| 1 | 114855187 | 114855327 | GAA | 62 | AATCC | 127 | GAAGGGAACG | 13 | SYCP1 |
| | | | GGG | | ACGGG | | GGCTTTCTTTT | | |
| | | | AACG | | GCTCT | | CAGGCCAGCG | | |
| | | | GGCT | | CGCTA | | TGGCAGCGGG | | |
| | | | TTCT | | CAGT | | CGGTAGGGCG | | |
| | | | TTTC | | | | AAAGGGAGAA | | |
| | | | AGG | | | | GGAAACGAGG | | |
| | | | | | | | GTTTATTCCGT | | |
| | | | | | | | TGCCCACTCCG | | |
| | | | | | | | CGGTAAGCGA | | |
| | | | | | | | CGTTGTAGGG | | |
| | | | | | | | CTCCACTGTAG | | |
| | | | | | | | CGAGAGCCCC | | |
| | | | | | | | GTGGATT | | |
| 3 | 45036157 | 45036279 | CGAG | 63 | CGGGG | 128 | CGAGAAGGGA | 178 | |
| | | | AAG | | CGTTT | | GGAGGTGAAG | | |
| | | | GGA | | TCATC | | GAGGGCGAGC | | |
| | | | GGA | | TCCCT | | TGAGCACACG | | |
| | | | GGTG | | AT | | CGCTTCATGCC | | |
| | | | AAG | | | | ACAGGAGGGT | | |
| | | | GAG | | | | GGGAATGAGC | | |
| | | | | | | | GGAGGACTGA | | |
| | | | | | | | GGAGAGGAAG | | |
| | | | | | | | GAGGGAAAGA | | |
| | | | | | | | ATAGGGAGAT | | |
| | | | | | | | GAAAACGCCC | | |
| | | | | | | | CG | | |
| 3 | 45036223 | 45036316 | TGAG | 64 | CCTGG | 129 | TGAGCGGAGG | 22 | |
| | | | CGGA | | CTTTG | | ACTGAGGAGA | | |
| | | | GGAC | | GTAAC | | GGAAGGAGGG | | |
| | | | TGAG | | TGTGC | | AAAGAATAGG | | |
| | | | GAG | | TGTGC | | GAGATGAAAA | | |
| | | | AGG | | | | CGCCCCGGTCT | | |
| | | | AA | | | | GCTGCTAAGC | | |
| | | | | | | | ACAGCACAGT | | |
| | | | | | | | TACCAAAGCC | | |
| | | | | | | | AGG | | |
| 1 | 54331507 | 54331625 | GCTC | 65 | GGCAC | 130 | GCTCTGATGCC | 179 | SSBP3 |
| | | | TGAT | | AGGCA | | TCTCCCTCCAC | | |
| | | | GCCT | | AATGC | | ACCACACCTGT | | |
| | | | CTCC | | CAAAT | | GATCTACTGTG | | |
| | | | CTCC | | CCT | | CATAGGATCTC | | |
| | | | ACAC | | | | ACAGGCCCAA | | |
| | | | | | | | TAACAGAGCT | | |
| | | | | | | | GGAGTTCCTCT | | |
| | | | | | | | TACGTGACAC | | |
| | | | | | | | AGGATTTGGC | | |
| | | | | | | | ATTTGCCTGTG | | |
| | | | | | | | CC | | |
| 13 | 49503072 | 49503187 | GACA | 66 | AAAGG | 131 | GACATCCTCCT | 180 | PHF11 |
| | | | TCCT | | CCCAG | | TGGCAGCCTTT | | |
| | | | CCTT | | AGATC | | CAACACGTTTC | | |
| | | | GGCA | | GGAGC | | TCAAATCCTTT | | |
| | | | GCCT | | TGAG | | CCCAGCTTCCT | | |
| | | | TTCA | | | | GTGCAGCCTTT | | |
| | | | | | | | CCTCCTCAGCC | | |
| | | | | | | | TGGCTGCCTTA | | |
| | | | | | | | CTGTCTCAGCT | | |
| | | | | | | | CCGATCTCTGG | | |
| | | | | | | | GCCTTT | | |
| 1 | 39515773 | 39515878 | TGAG | 67 | CATGC | 132 | TGAGCGCTTA | 181 | OXCT2P, |
| | | | CGCT | | CGTCC | | ACGATCCGGA | | BMP8A |
| | | | TAAC | | TCAAA | | AAGAGGAAGA | | |
| | | | GATC | | TTCCA | | TGGAGACGCT | | |
| | | | CGGA | | GAGC | | GGAAAGGAAG | | |
| | | | AAG | | | | AGGACGCCAG | | |
| | | | A | | | | GACGCGCATC | | |
| | | | | | | | ATCAGACGCG | | |
| | | | | | | | CAGCTCTGGA | | |
| | | | | | | | ATTTGAGGAC | | |
| | | | | | | | GGCATG | | |
| 7 | 100785886 | 100786015 | TGAC | 68 | AGACC | 133 | TGACCTCAGGT | 14 | ZAN |
| | | | CTCA | | CAGCT | | GATCCACCCGT | | |
| | | | GGTG | | CTTGC | | CTCGGCCTCCC | | |
| | | | ATCC | | CCAGC | | AAAGTGTTGG | | |
| | | | ACCC | | TGTA | | GATTACAGGC | | |
| | | | GTCT | | | | GTGAGCCGCC | | |
| | | | | | | | GCGCCCAGCC | | |
| | | | | | | | CCCTCCTCACT | | |
| | | | | | | | CTCTTTCTCTT | | |
| | | | | | | | CCTGTAACTTC | | |
| | | | | | | | TACAGCTGGG | | |
| | | | | | | | CAAGAGCTGG | | |
| | | | | | | | GTCT | | |
| 9 | 126676778 | 126676857 | CCCC | 69 | CTTTA | 134 | CCCCATAGGG | 182 | LMX1B |
| | | | ATAG | | ACCCT | | AGGACTTGCG | | |
| | | | GGA | | TTCCC | | CACAGTTGGC | | |
| | | | GGAC | | CTCGC | | GCTGGGTAAA | | |
| | | | TTGC | | CCGCA | | TGCTGGGAGA | | |
| | | | GCAC | | GCA | | ACTGCTGCGG | | |
| | | | AGTT | | | | GCGAGGGGAA | | |
| | | | GG | | | | AGGGTTAAAG | | |
| 14 | 104736436 | 104736562 | CACA | 70 | GTCCA | 135 | CACAGACACC | 4 | ADSSL1 |
| | | | GACA | | GACGA | | CTGAGCTTGCA | | |
| | | | CCCT | | GAGTG | | ACACTCCGGG | | |
| | | | GAGC | | ACCCG | | CCTCTGCCGCG | | |
| | | | TTGC | | GAGA | | TGTTTATTTCA | | |
| | | | AACA | | | | GGATGCCGTG | | |
| | | | | | | | GCATTTGGGTG | | |
| | | | | | | | ACCTTTTGTGC | | |
| | | | | | | | TCACCATGGCT | | |
| | | | | | | | TGCGTCGTCTC | | |
| | | | | | | | CGGGTCACTCT | | |
| | | | | | | | CGTCTGGAC | | |
| 1 | 27369167 | 27369316 | GCAA | 71 | GCCCA | 136 | GCAAAGGCAA | 19 | MAP3K6, |
| | | | AGGC | | CAAAT | | GGTGGCTGAC | | FCN3 |
| | | | AAG | | ATGGC | | GATCCGGAAG | | |
| | | | GTGG | | ATTGA | | CTGTACAGGA | | |
| | | | CTGA | | CTGG | | GAGATAAGGG | | |
| | | | CG | | | | CACTGGCTGCC | | |
| | | | | | | | AGAGTGCCCT | | |
| | | | | | | | ATCGAAGCAT | | |
| | | | | | | | CATCCGAACC | | |
| | | | | | | | CTGCGGTAGG | | |
| | | | | | | | GGTGGCCCAC | | |
| | | | | | | | ACCACGGCCT | | |
| | | | | | | | GAGGCCCAGT | | |
| | | | | | | | CAATGCCATAT | | |
| | | | | | | | TTGTGGGC | | |
| 1 | 27369224 | 27369347 | TGCC | 72 | CAATG | 137 | TGCCAGAGTG | 20 | MAP3K6, |
| | | | AGA | | GTCGC | | CCCTATCGAA | | FCN3 |
| | | | GTGC | | TATGC | | GCATCATCCG | | |
| | | | CCTA | | AGTGT | | AACCCTGCGG | | |
| | | | TCGA | | CTGAG | | TAGGGGTGGC | | |
| | | | AGCA | | G | | CCACACCACG | | |
| | | | T | | | | GCCTGAGGCC | | |
| | | | | | | | CAGTCAATGC | | |
| | | | | | | | CATATTTGTGG | | |
| | | | | | | | GCGGCAGCCT | | |
| | | | | | | | CAGACACTGC | | |
| | | | | | | | ATAGCGACCA | | |
| | | | | | | | TTG | | |
| 1 | 235011867 | 235011944 | GTCA | 73 | GTGCA | 138 | GTCATCAGTG | 183 | |
| | | | TCAG | | GGGGA | | AATCGACCAC | | |
| | | | TGAA | | CAGCA | | AAAGAGCCTT | | |
| | | | TCGA | | GACAT | | TGCGGAGGTG | | |
| | | | CCAC | | CAGA | | ATTTACAGGA | | |
| | | | AAA | | | | GAGCTCTGAT | | |
| | | | GAGC | | | | GTCTGCTGTCC | | |
| | | | | | | | CCTGCAC | | |
| 7 | 19772652 | 19772800 | GGA | 74 | TTGGG | 139 | GGAGAGCACC | 2 | TMEM196 |
| | | | GAGC | | ATCTG | | AAGAGGCTCC | | |
| | | | ACCA | | GTAGG | | CAATAATCTG | | |
| | | | AGA | | GGGTG | | ACCGCTGGTG | | |
| | | | GGCT | | GAAG | | CACATCCTTCC | | |
| | | | CCCA | | | | TCGGTCATCTT | | |
| | | | AT | | | | CCTTCCAGATC | | |
| | | | | | | | AGAGAGGGAA | | |
| | | | | | | | ATCAACCATCT | | |
| | | | | | | | ACCTTTTTTTC | | |
| | | | | | | | TTCCACTATCC | | |
| | | | | | | | TCCTTACCCCT | | |
| | | | | | | | TCCACCCCCTA | | |
| | | | | | | | CCAGATCCCA | | |
| | | | | | | | A | | |
| 7 | 129720565 | 129720676 | TAAC | 75 | GGAGC | 140 | TAACCACCTGC | 1 | NRF1 |
| | | | CACC | | CCCTC | | ACCTCTGCTGC | | |
| | | | TGCA | | TGCCT | | AATGTAAACA | | |
| | | | CCTC | | CCTTA | | GCAGATGTGG | | |
| | | | TGCT | | TTCC | | GCGCAGGGTG | | |
| | | | GCAA | | | | AGAAGGGAGA | | |
| | | | | | | | GGAAGCTACG | | |
| | | | | | | | TGCAATGGCA | | |
| | | | | | | | GGTTGGGGAA | | |
| | | | | | | | TAAGGAGGCA | | |
| | | | | | | | GAGGGGCTCC | | |
| 8 | 98951679 | 98951812 | ACAC | 76 | GGGAA | 141 | ACACCCCGCG | 184 | OSR2 |
| | | | CCCG | | GAGCT | | GCAGGACTTCT | | |
| | | | CGGC | | GGGGT | | AGAGAAGCCC | | |
| | | | AGG | | CAGTG | | AGGATCTGTCC | | |
| | | | ACTT | | AAGG | | CGTGCCGCCG | | |
| | | | CTA | | | | CTGCTCCCCTC | | |
| | | | | | | | CCCAGACACC | | |
| | | | | | | | TCTCCACGTCT | | |
| | | | | | | | CCTACCCAGG | | |
| | | | | | | | GGGTCGCATC | | |
| | | | | | | | CCTAGCCCTTC | | |
| | | | | | | | ACTGACCCCA | | |
| | | | | | | | GCTCTTCCC | | |
| 12 | 53694915 | 53695058 | CATC | 77 | AAGGA | 142 | CATCTCCTCCT | 23 | |
| | | | TCCT | | GAGCC | | CGCAAACCCC | | |
| | | | CCTC | | GGGAG | | AAGCCAAGGC | | |
| | | | GCAA | | GAGTG | | AAGCTGGATG | | |
| | | | ACCC | | AGTG | | AAGCGCTCCCT | | |
| | | | CAAG | | | | GGGCAGGCCC | | |
| | | | | | | | GGCTCTCCGTG | | |
| | | | | | | | TCCCTCCATCA | | |
| | | | | | | | CCTGACCCCGC | | |
| | | | | | | | TGGCTCTCGCA | | |
| | | | | | | | GACCCCTTCCT | | |
| | | | | | | | CCACACTCACT | | |
| | | | | | | | CCTCCCGGCTC | | |
| | | | | | | | TCCTT | | |
| 12 | 53695032 | 53695180 | CCAC | 78 | CCTGC | 143 | CCACACTCACT | 24 | |
| | | | ACTC | | GGAGG | | CCTCCCGGCTC | | |
| | | | ACTC | | CCGAA | | TCCTTCTATAA | | |
| | | | CTCC | | GGATA | | TCTCCTGACAT | | |
| | | | CGGC | | AA | | CTCTTCAAATC | | |
| | | | TCT | | | | CAATTATTGAA | | |
| | | | | | | | TTAATTGACGT | | |
| | | | | | | | ACGAACCCAG | | |
| | | | | | | | AGGCAAACAG | | |
| | | | | | | | AAAGGGGCGG | | |
| | | | | | | | CAAACACTGG | | |
| | | | | | | | GCGGCTCAGA | | |
| | | | | | | | TTTATCCTTCG | | |
| | | | | | | | GCCTCCGCAG | | |
| | | | | | | | G | | |
| 12 | 53695146 | 53695232 | TGGG | 79 | AGGTT | 144 | TGGGCGGCTC | 25 | |
| | | | CGGC | | TGAAC | | AGATTTATCCT | | |
| | | | TCAG | | TGTCG | | TCGGCCTCCGC | | |
| | | | ATTT | | CCGTG | | AGGGCCCGGC | | |
| | | | ATCC | | TTCG | | CGGACGAGAT | | |
| | | | TTCG | | | | TTACTGGGCCT | | |
| | | | | | | | CGAACACGGC | | |
| | | | | | | | GACAGTTCAA | | |
| | | | | | | | ACCT | | |
| 15 | 96947824 | 96947954 | TCGG | 80 | GCGCC | 145 | TCGGCAGTGA | 185 | |
| | | | CAGT | | CAGAC | | AAAGCGGGAG | | |
| | | | GAA | | CACCG | | ATTAGAAAAT | | |
| | | | AAGC | | AGGAC | | GTTTCATGCTA | | |
| | | | GGG | | | | ATTTCCATGGA | | |
| | | | AGAT | | | | GATTTCTTTAA | | |
| | | | TA | | | | TTTAGCGAAG | | |
| | | | | | | | ACTGCTTCCCG | | |
| | | | | | | | GGCTCCGCCTG | | |
| | | | | | | | GCCCGCGCCG | | |
| | | | | | | | GCCCGCGTCCT | | |
| | | | | | | | CGGTGGTCTG | | |
| | | | | | | | GGCGC | | |
| 15 | 96948043 | 96948167 | GGCT | 81 | GCCAG | 146 | GGCTCTCGGG | 186 | |
| | | | CTCG | | GCAGG | | CTCTCGCTTTT | | |
| | | | GGCT | | AGAAA | | TTTTTTTTTTTT | | |
| | | | CTCG | | GAGCT | | TCTTTCCGCGG | | |
| | | | CTTT | | TGAAA | | CAGTCTTAGG | | |
| | | | TT | | | | ATTCTTGTCAC | | |
| | | | | | | | ATGATGGCTTC | | |
| | | | | | | | ATCGGGCCCTT | | |
| | | | | | | | CTCCTCCTGAT | | |
| | | | | | | | CCTTTCAAGCT | | |
| | | | | | | | CTTTCTCCTGC | | |
| | | | | | | | CTGGC | | |
| 4 | 40198434 | 40198576 | CTGG | 82 | AGAAA | 147 | CTGGGGAGAA | 187 | RHOH |
| | | | GGA | | GCAGC | | GTGACCCCATT | | |
| | | | GAA | | CCCAA | | CAATAGTCCTT | | |
| | | | GTGA | | GTGGG | | GGTCTCCTTCT | | |
| | | | CCCC | | AAGA | | GCCCTGCGGCT | | |
| | | | ATTC | | | | GCGCTTCCTCG | | |
| | | | AA | | | | GCTCTCACGGC | | |
| | | | | | | | ACCAGCAGAA | | |
| | | | | | | | TTCCATGTGAG | | |
| | | | | | | | AGGGAGCTTG | | |
| | | | | | | | TCGAGCGTGG | | |
| | | | | | | | CCTCTTCCCAC | | |
| | | | | | | | TTGGGGCTGCT | | |
| | | | | | | | TTCT | | |
| 9 | 124007846 | 124007966 | GCAC | 83 | ATTTG | 148 | GCACCATCCTC | 188 | LHX2, |
| | | | CATC | | GGAGC | | AGAGCTTCAG | | LOC100505588 |
| | | | CTCA | | CACCA | | ACCATACATTG | | |
| | | | GAGC | | GGGAA | | ACAGTGAGCA | | |
| | | | TTCA | | GGA | | AAGGGGGCCC | | |
| | | | GACC | | | | CAGGCAGGCG | | |
| | | | A | | | | GGTCTGGGGC | | |
| | | | | | | | CAAGGAGGGC | | |
| | | | | | | | GGCTCCCCTGC | | |
| | | | | | | | GCGGATCCTTC | | |
| | | | | | | | CCTGGTGGCTC | | |
| | | | | | | | CCAAAT | | |
| 2 | 86862416 | 86862559 | GCTG | 84 | AATAG | 149 | GCTGGGAACT | 15 | CD8B, |
| | | | GGA | | AGCCA | | GGAGGTGCAG | | ANAPC1P1 |
| | | | ACTG | | GCCCC | | AGAAGGCCCC | | |
| | | | GAG | | AGGGC | | GACGCTGTTTG | | |
| | | | GTGC | | ACTC | | TAGGTTGTGG | | |
| | | | AGA | | | | GGGTGCAGCA | | |
| | | | GAA | | | | AGACCTAGAT | | |
| | | | | | | | CTTAAGAATTT | | |
| | | | | | | | CGAAGGACTG | | |
| | | | | | | | TGACGATCAC | | |
| | | | | | | | CGGCTGCGCC | | |
| | | | | | | | CTGCCGGCGA | | |
| | | | | | | | GTGCCCTGGG | | |
| | | | | | | | GCTGGCTCTAT | | |
| | | | | | | | T | | |
| 2 | 176002883 | 176002999 | GCTT | 85 | GGGTC | 150 | GCTTCAAACG | 189 | LNPK |
| | | | CAAA | | CACCT | | CCGTATCATGT | | |
| | | | CGCC | | ATCAG | | TGCTTTAAAAC | | |
| | | | GTAT | | GGCCT | | CTGCGGGTAA | | |
| | | | CATG | | GTG | | CAGCATAAGC | | |
| | | | TTGC | | | | TGAGTTTTCTA | | |
| | | | T | | | | TCTTAGAACTC | | |
| | | | | | | | TTAACCCCAA | | |
| | | | | | | | GAACACTCTTC | | |
| | | | | | | | ACAGGCCCTG | | |
| | | | | | | | ATAGGTGGAC | | |
| | | | | | | | CC | | |
| 3 | 196947318 | 196947391 | TGGT | 86 | CACCA | 151 | TGGTGCCAGG | 190 | NCBP2, |
| | | | GCCA | | GACAA | | GGTTACCACA | | PIGZ |
| | | | GGG | | CCAGC | | AAGAGGCGGC | | |
| | | | GTTA | | CTGCC | | AGAGCCATGG | | |
| | | | CCAC | | AAGT | | CCCACCAGCC | | |
| | | | AAA | | | | ACTTGGCAGG | | |
| | | | GA | | | | CTGGTTGTCTG | | |
| | | | | | | | GTG | | |
| 13 | 27267902 | 27268013 | GAG | 87 | CCTGA | 152 | GAGACAACAG | 191 | RASL11A |
| | | | ACAA | | TGGGG | | CCCAGACCCC | | |
| | | | CAGC | | GAAAA | | CATCACGGAG | | |
| | | | CCAG | | GGCAC | | CTGCACGTGA | | |
| | | | ACCC | | CATA | | CCCTGGAACTT | | |
| | | | CCAT | | | | AACAGCTTCC | | |
| | | | C | | | | AGTTGTTCCCT | | |
| | | | | | | | AGACAGTCAT | | |
| | | | | | | | TGTCTTTATGG | | |
| | | | | | | | TGCCTTTTCCC | | |
| | | | | | | | CCATCAGG | | |
| 13 | 109791131 | 109791246 | TGTA | 88 | CACAG | 153 | TGTAAGATGA | 192 | IRS2 |
| | | | AGAT | | GCTGG | | CACAGCTATAT | | |
| | | | GACA | | TCTTC | | TTTCTGGGAGA | | |
| | | | CAGC | | CCCAC | | GGGCGGGAGG | | |
| | | | TATA | | TTCA | | ATGCTCAGCG | | |
| | | | TTTT | | | | AGGGTGGCCC | | |
| | | | CTGG | | | | GGAGTGTCCTT | | |
| | | | GAG | | | | GTACAGAGTA | | |
| | | | AGG | | | | CAGATGTTATG | | |
| | | | | | | | AAGTGGGGAA | | |
| | | | | | | | GACCAGCCTG | | |
| | | | | | | | TG | | |
| 14 | 68628811 | 68628918 | GGA | 89 | TGTAC | 154 | GGAGGAGCTG | 193 | LOC100996664, |
| | | | GGA | | AGCGA | | AGGTTTCGGCT | | RAD51B |
| | | | GCTG | | TGGCC | | GAGCCCCCAG | | |
| | | | AGGT | | TGATA | | CCTCCCCCGAC | | |
| | | | TTCG | | AGCAA | | CGCACAGCCT | | |
| | | | GCTG | | | | CGGGCATGAA | | |
| | | | AG | | | | CCCGCGAAGC | | |
| | | | | | | | CAGACGCTTA | | |
| | | | | | | | GTTGCTTATCA | | |
| | | | | | | | GGCCATCGCT | | |
| | | | | | | | GTACA | | |
| 17 | 15966505 | 15966624 | AGG | 90 | TCACA | 155 | AGGAGGAGAC | 194 | ADORA2B |
| | | | AGG | | CGGAG | | CCTGCCCCAG | | |
| | | | AGAC | | GGAAT | | AAATAGGCCA | | |
| | | | CCTG | | CAACA | | GTGCTTGTTAT | | |
| | | | CCCC | | AAAGG | | GCAGGCCTTG | | |
| | | | AGA | | | | GCGGTTCCCCG | | |
| | | | AAT | | | | TTTCCTTACGT | | |
| | | | | | | | AACCTCAGTGT | | |
| | | | | | | | TCACGCTGTTT | | |
| | | | | | | | CCTTTTGTTGA | | |
| | | | | | | | TTCCCTCCGTG | | |
| | | | | | | | TGA | | |

Each row of Table 1 shows, for a particular DMR, the associated (human) chromosome, the start and end location, the forward and reverse oligonucleotide primers for amplification of the DMR sequence, the DMR sequence, and the associated gene(s) (where identified).

The DMRs in Table 1 are also presented below. The below listing shows (human) nucleic acid sequences that include DMR sequences of Table 1, presented in the order they appear in Table 1—the capitalized portion of the sequence provided is the DMR that is amplified by the forward and reverse oligonucleotide pairs shown in Table 1:

>reg101_102
(SEQ ID NO: 195)
catctccatcttctccatcatctccatcttctccatcatctccatcttcatcatctccatttccatcatctccatctccatcatcatctctatctccatcat
ctccatctccatcatctccatcatctccatctccatctccatctccatcatctaccgtctccaatctccatctccgaagttatgcccacttcctcgaa
gtttggagccacgcgaactacactgcccagaaggcgccgcgccgtgagccgCGATGCTTGGCCAATGAAAAGAG
GTCTACCCGAGAGTGCGACGCGCAATGGGCGGGACTTCCGGCGTCTCCCCTCGGCG
GTTGCTTTCGCTGCCCTCAAGAGAACTCAgcttgccggaagctggttgttcgctgcggcgacc
agctccggaaagcgcggtggggacgcgctgtgttctcgcagctcagaggcgggtctgaggctcggtggcggcgcccagggtggcccg
ggccctttcctcggtcgttgtctcaccgccacaggctccgatggcggcggccacgctgagggaccccgctcaggtgagcgccgcgtcctc
ccggcctcccccgaatcctaaagccctgtgagggccgcc
>reg11
(SEQ ID NO: 196)
cttcctcggactctcggccgacgagctcatcgccatcatctccagcgacggccttaacgtggagaaggaggaggcagtgttcgaggcggt
gatgcggtgggcgggtagcggcgacgccgaggcgcaggctgagcgccagcgcgcgctgcccaccgtcttcgagagcgtgcgctgcc
gcttgctgccgcgcgcctttctggaaagccgcgtggagcgccaccctctcgtgcgtgcccagcccgagttgctgcGCAAGGTGC
AGATGGTGAAGGATGCACACGAGGGCCGCATCACCACGCTGCGGAAGAAAAAGAA
GGGGAAGGATGGAGCCGGGGCCAAGGAGGCTGATAAGGGCACAAGCAAAGCCaaagc
agaggaggatgaggaggccgaacgtatccttcctgggatcctcaatgacaccctgcgcttcggcatgttcctgcaggatctcatcttcatgat
cagtgaggagggcgctgtggcctacgatccagcagccaacgagtgctactgtgcttccctctccaaccaggtccccaagaaccacgtcag
cctggttaccaaggagaaccaggtcttcgtggctggaggcctcttctacaacgaagacaaca
>reg110
(SEQ ID NO: 197)
ttccctttttctcctcacaaggaggtgaggctgggacctccgggccagcttctcacctcatagggtgtacctttcccggctccagcagccaat
gtgcttcggagccactctctgcagagccagagggcaggccggcttctcggtgtgtgcctaagaggatggatcggaggtcccgggctcag
cagtggcgccgagctcgccataattacaacgacctgtgcccgcccataggccgccgggcagccaccgcgCTCCTCTGGCTCT
CCTGCTCCATCGCGCTCCTCCGCGCCCTTGCCACCTCCAACGCCCGTGCCCAGCAGC
GCGCGGCTGCCCAACAGCGCCGGAGCTTCCTTAACGCCCACCACCgctccggcgcccaggtatt
ccctgagtcccccgaatcggaatctgaccacgagcacgaggaggcagaccttgagctgtccctccccgagtgcctagagtacgaggaag
agttcgactacgagaccgagagcgagaccgagtccgaaatcgagtccgagaccgacttcgagaccgagcctgagaccgcccccaccac
tgagcccgagaccgagcctgaagacgatcgcggcccggtggtgcccaagcactc
>reg119
(SEQ ID NO: 198)
tggtcagggcctgaggcaaccctgtcccagcgctgaggacccaggaacatgccaccagcctgggatgggggaggccacggagggag
ggagcagtgagcccccagggaggaatctcgagctgagggaccaggagttcgggcttgttctgagaaacgcacagtgtcagagtcactca
ttcagaaagactgagagagcctgccgagagctgggtaccggagacgcgtccctgccctctcagagttgacagtccaGAGGCAAA
AAGGACAATCGGCAAGTAAATAGTAAATGAACAAGAAGACCCCGGTTGTGAGAAA
ATGTTATAAAGCAAATAAATCAGAGAAATGTGATCACAAACCCTGGGTGGGTGAAG
GGTACaagtttaggaaacgggtcagggaaggcctctctgacatttgagctgagccttggatgaccagaaagaactattgaaagatctgg
gtggggccagaggaggggtgagtggcagatgccccaggagagaagaaagttgtccaggaggggcccgtgcactggaggcaggggac
ggggcaggcacaggggcagggggacgaagccagaggcactccctcccccagggtgctgagcaggggagccccctgactca
>reg12
(SEQ ID NO: 199)
ccaaagaatgcagagaatgtgcacccgtctgtgacatagttggtaatttccagaggcggagaagatattattgacaataaggtgaacacgct
gtgtgaccaccgtggatcgtcatatccagggggggagatgggggtgatatgactccccgcatcgcggggggcgcccgcccccctgcgat
gtggatcatcatatccagggggggagaggggggtgatatgactcccctcatcgcggtgggcgcccgccccCCTGCGACGTGA
ATCGTCATATCCAGAGGGGGGTGATATGACTCCCCGCATCGCGGGGGCCTCACCCC
ATTGCGATGGGGGTCCTAAGAGCCAGGGGGAGATAGGGGctggctcttactccccgtaccgccggg
ggggggggcctcaccgccctgcgacggggctccttagagccagggagggggaggggctggctcttactcccggtatcgcaggaggtgt
gtacaacccctgcgatattgggagtaatatcatcctctccccctgaatataagaaacaatatcacaggagcatgtacaccccctgcgatattg
gaagtaacatcattttctccccctagggatattcggaacaat
>reg124.2
(SEQ ID NO: 200)
ggggggcaaggacacggggccctccccaggctcgctggcagcccattgtgctgggctggaaggtctcccaacctgaggacacctaggg
gcaagggagccactggcctgagcctgagatctctgagcgggggcaggcagccctcgccatgccaagggcatccctaatccacccctaca
caccagcggaagccactggcagtgagggcccagggccaccaagcagggctggggcaggaaagaccagcaggtgcAGCTGAG
GGAAAGGGGGAAGTCACTGGGCTGGGGGCCGGGGCCGCTCACTCTGGCCTCCTCTG
AGGGGTCCACTGGGGTTCcggctcctcagaccctggctctgcagcctcagggccaacttcccgcttggagaaagggcag
cgcttgtccggggacccaccacatccatcctcgtagggggctgtctccacccagggtcccccccccaccccctcattcctcccagtggtga
aaggacagtgaaggaggagggcagcccaggagtggacatggagtgaccaggagcttcctggggggtccgggaggtggggcacaccc
tatcgcacacca
>reg14
(SEQ ID NO: 201)
tggccgggtctaagctgtgctcctgctgcctggctggcttccgcccggtcagactgacagggtcttgcaggcaggaaccgtgcacacagtg
tctagctccgagcctgagaatactcgtggcttcaaaagtttgctgagctaccgcagggaggacgaaggctataacactggtccagcctgag
agaagcccaagtggggttcactgccctctgagccacagatttaagggggagggtgtggaaactgccggcTGCTGAGGTCCAA
ACTCACCGAAGGTACTGACCGCCGCGGCTCCTCTCTTCACAGCGTCTGCCGGAGGCC
TCCGTTTACTCCGGTTACCGAGACAACGCCACCCCTcttccagggaggcggaaccagggcgggccgtg
gggcgcatgcgcggccggcgtccagctctccgggaacccggtacctatccgccctttggtcgggccttctccgcctcatgacactggttca
aagccaaacagaaaagcccgacgagtttattatcccctaaaggacgtcatgtagataattaaatgacatgaataccgtcgaagatacctgcct
gatattccaaaatggcccaacggagccctgatcg
>reg144
(SEQ ID NO: 202)
tataaagtgcgcgcagtttgttttatttcctgagtttttgcaatctagataacagatgataccctgagtggctggcgctgcctctgtaatggcggc
actgagcctttggagaagtattaataatagattgtgttgatgagtttggagaaagtagcaatcgaccccctgctgccaaggcattagcgcggc
tgttctgagcacagccagcactgtggctttgactgcaaatgcaggtcacccgccctgctgccCCTTCCCCAGCCTAAGAA
GGTTTCCTCTCCGGGAGTCACCCAAGGTGTGCTGACCCTGGCCTGGGACCCTGGGAC
CGTGGCGCTCCCACGCTAGCAGCGACACGGCCAGTGTCTGTCCACTCAGAGGCCgcag
aggtcaggctgcagaccttagtgtggccactaggtcaggtggagtgtggggaggggacagaggggcagtaggggttgggggaggacc
accctccatgtcagagcaccgggttctacaaacccaggctccttcctcagcccctcgggagagctggacagccagccagattcctagggc
ctctgcctaaagctgtcactgacagttgggtaggttgtgccctgaacaaggggattcagccagagggcc
>reg145.3
(SEQ ID NO: 203)
agtgtgttcctcacttccacctgtggcggtcgcttctggctgtcaccctgagcacatccatgtggcctcttggagtggcctctccacgtggcct
aggcttcctggcaacgcagccgcctcagggcagtgtgacttcctgatggtggtgactcaggacaacaaaagcgagaggccctgagagtc
aggcgggcaccacagggccttgctgaggcagccggggactcccgctccctctgctgacaccattggtgGCCAGTGAGTCAG
AGGCAGAGGTGCCAGAGACCCCGCCCGAAGGGAGGAGATCTGAGAGCCTGCAGCC
ACAGGCTCCTCCAGGACTCGAGcaccggggccgcacagagagccctttctctcctgggcaggccaggcggggatcc
cccagcgccctaacctgctctgtgaccacggcaatgtggccttggggatgtgccctgcctctctgggttccagtgcaggactcagggctgg
ccacctgagaagcatctctaggacattccaaagcctggaacagggacagcattgtggccctgctctggaaggctgcgtggaagccaagaa
gttgtcctggcctgt
>reg15_16
(SEQ ID NO: 204)
acagtgtctagctccgagcctgagaatactcgtggcttcaaaagtttgctgagctaccgcagggaggacgaaggctataacactggtccag
cctgagagaagcccaagtggggttcactgccctctgagccacagatttaagggggagggtgtggaaactgccggctgctgaggtccaaa
ctcaccgaaggtactgaccgccgcggctcctctcttcacagcgtctgccggaggcctccgtttactccggtTACCGAGACAACG
CCACCCCTCTTCCAGGGAGGCGGAACCAGGGCGGGCCGTGGGGCGCATGCGCGGCC
GGCGTCCAGCTCTCCGGGAACCCGGTACCTATCcgccctttggtcgggccttctccgcctcatgacactggtt
caaagccaaacagaaaagcccgacgagtttattatcccctaaaggacgtcatgtagataattaaatgacatgaataccgtcgaagatacctg
cctgatattccaaaatggcccaacggagccctgatcgcggcgttcctatgttgaggttttaacttcgattttaagaggggtcctgggagatagt
aggcagcttgccggcaacatcaac
>reg150
(SEQ ID NO: 205)
gatccagccgagtcagggactttccccacgccccacccgaccctcaggcctgacagccacaggggcagagcaggaggaggccaggca
ggggcagtgagggaaacagggaggggccttggccacagcaatcttgggcctccagccccatgggaaccccagcacgatgagcatcca
gggtcattgagggggaggcggggagctggctgtggccacctggagtcacagcggggcaagggttgggggccccagggGAGCTG
GGACAAGAAGGGAACACGGTACCAGGGTAGCAGAAGACAGGCACCCCCCGTCCCC
CAGTCCTAGGGCTTCCTCACCGCGCCTGTGAGGAGCTCCAGGCCTGTGcagacgggggcag
ggcccggcagggcgggtgggaaggcgacctgagggcccatgatgaaggccaccagcagcagcagcaggagggcattgaaagccag
cagggtcttgagaaagaggaagtaggagagcacgctggagccgaactggcccccgatgcgcttcagggcgtagcgccacggcatcag
ggcctgcagggcggagagcagcgccaggcccaggctgtgcaaggcctgcgggcacaggcagagag
>reg17
(SEQ ID NO: 206)
taacactggtccagcctgagagaagcccaagtggggttcactgccctctgagccacagatttaagggggagggtgtggaaactgccggct
gctgaggtccaaactcaccgaaggtactgaccgccgcggctcctctcttcacagcgtctgccggaggcctccgtttactccggttaccgag
acaacgccacccctcttccagggaggcggaaccagggcgggccgtggggcgcatgcgcggccggcgtccaGCTCTCCGGG
AACCCGGTACCTATCCGCCCTTTGGTCGGGCCTTCTCCGCCTCATGACACTGGTTCA
AAGCCAAACAGAAAAGCCCGACGAGTTTattatcccctaaaggacgtcatgtagataattaaatgacatgaatac
cgtcgaagatacctgcctgatattccaaaatggcccaacggagccctgatcgcggcgttcctatgttgaggttttaacttcgattttaagaggg
gtcctgggagatagtaggcagcttgccggcaacatcaacaacaaagatacatcgtgggatttttgttatttttaaaactatattatctctgttggct
tttaagagtaaa
>reg23
(SEQ ID NO: 207)
cacactcagcctggcctagaaaaaactcaaaattttgaattttcatcaaatgagagaataaatgattaaacaaatagaaatgcttcacccagca
gcaagcgcttagattttaaggacccaagcaaagtgcatggaaaggtgcagctgtctggaaggacgattgggaggtgggatcttggggaga
aagggaagaaaggggatggagcagggcttcccagtcgagggcggcggccgagcctgtgtccccaccaGCGTCTCTGTGGC
CGTGAAGTGTATGCATGCGTGCCCATGTTGATGCGGCGCCGTGCGGGAGGCGGGCA
TCCCCTGCTGTACATGGGAGGGAGGCTGTCtgtgcagagcattgcccagttgccatagaaacgagcagaagg
aggtgggtggctggagaaggaggcgggtcgggatcggggagtggggaggaggcagcggtggagggagctggctcctgcagttctgg
cgctgctgccttcctgagtgagcggtggagggaaccctagaggacagagcccccagcccggcagcaggccccctctccgcccgccacc
acggaggagaaggaggacagccagcccctccagc
>reg32
(SEQ ID NO: 208)
acaccacgtgggcccctcccgccctcccccagcacttgcacaaagcctggaggagggcctccctgtcccacacaacttcctgcttgtcccc
ttcccacccctctcctccccaggagcggctcccaggcccacgaacagcggcttcaagaggtggaagccgaggtggcagccacaggcac
ctaccagcttagggagagcgagctggtgttcggggctaagcaggcctggcgcaacgctccccgctgcgtgggCCGGATCCAG
TGGGGGAAGCTGCAGGTGCGGCTGGCCAGCGACTGAGAGACCCGGGCGCTACCAAA
AGGGGAGCGGGGTGGCGGGGCAGTTCCTAAGGCTTCCCGGGggctgggaggtcccaaactgtgg
gggagatccttgccttttcccttagagactggaaaggtagggggactgccccaccctcagcacccaggggaacctcagcccagtagtgaa
gacctggttatcaggccctatggtagtgccttggctggaggaggggaaagaagtctagacctgctgcaggggtgaggaagtctagacctg
ctgcaggggtgaggaagtctagacctgctgcaggggtgaggaagtct
>reg49
(SEQ ID NO: 209)
tgaaatatatgtccacacacggagaatttaagagtatttttatatttctctctagatctaaatattcagatgtgttaattacatgccctagaagctgg
aagcgatcagtggtgttcacactggacgtggagctgtttgtataattttcatctccctgcacttaaacatgactctcagtctaataaattcaacctt
gtcatttttagaatcgacgggatttctctggctgtcgtttgcgctgcatttatccgAATACATCCAGCTCGCAGGCATCCT
GCAAGAAACGGCTCCCGGCTCGCGTGTACGCCGACACCTCGGCCCAACGCAGGACT
CGAGGTGGTTTCTAGTGCCCgggtggctgcaagtctgccctccgagggaggctggacaagcggcgcccccaggtcga
gcggcctctcgctgcctggcagtgcctggcagcccccacctctgccagtgcttcggaaacccgcctggccaggttcgcccgcggtgaaaa
atgaaagcaaattccccaacagaggtagccggaactttcctcgacgaaggctccctcctgcgcctgtgtctggagaacccccagagcgct
gcaagttagcaag
>reg58
(SEQ ID NO: 210)
agtccaagtttctgccacagttccagggccgaggctgtttccaaagagccctgtaattgttttccacctgtgtctcacccaaacaccaaggctg
gcgcaggtggacaccttcccacttttctccctccaggctgggccccagaaatcagtagaggagggaggaatcagtcagcgtggccatgcc
tgggaggagaggcccgtgtgggtctgtggggctaagaggcaaaggcgggtggcggatgtgggccagcGGGAGCCTGGAG
GGGTTGACACCGCCTGCTCCACCGCAAGCCCCTGGAGGAAGAGCCCCGCTGTGCCC
GAGAGCGAGCGCGGGCAGGTGTAACTACCCGGGGCTGGGgctccgggggctccgcgcagcctcctt
ccctcccagggacaccgcccagctgcgccccgcgccccgccgactgcgcgggccttgagacgctggtggctgcctcggggttggcctg
ctcctcgcgcacatgttcagggtcatccgcgctgcgcctctgcttcaggtgcttggctagagaaagggcggcaagacggggcagtgcgtg
tgcgcgcgcgggcaagtgcatgtgagtgcacacttatgtgagcgc
>reg60
(SEQ ID NO: 211)
tggaggggttgacaccgcctgctccaccgcaagcccctggaggaagagccccgctgtgcccgagagcgagcgcgggcaggtgtaact
acccggggctggggctccgggggctccgcgcagcctccttccctcccagggacaccgcccagctgcgccccgcgccccgccgactgc
gcgggccttgagacgctggtggctgcctcggggttggcctgctcctcgcgcacatgttcagggtcatccgcgctgcgCCTCTGCTT
CAGGTGCTTGGCTAGAGAAAGGGCGGCAAGACGGGGCAGTGCGTGTGCGCGCGCGG
GCAAGTGCATGTGAGTGCACACTTATGTGAGCGCATGTGTGTCTGCgcttgtgcgtgtccaggg
gaaccacagggagcaccctcattctaagcctccagaggactgcctgaagccgctagatagaaactcccctagaatgtaagctccgggggg
gagggagctttgtttgatggctgctgtattcccagtgcccattgaagtactggggacacattagatgcttaataaacagctgttgagttaatcaa
cggactctaggaatggaggcagaccggcccttctggaactggagaaa
>reg62
(SEQ ID NO: 212)
ttgaaaccccgtctctactaaaaatacagaaaaaaaaaaaatagccgggcgtggtggcgggagcctgtagtctcagctactcgggaggctg
aggcaggagaatgtcgtgaacctgggaggcggagattgcagtgagcccagatcgcaccactgcactccagcctgggtgacagagcgag
actccgtctcaaaaaaaaaaaaaaaaaaaaaaagccgtcgcgcctcgggagtgggctggggggagagggggtGCCCAAGGGC
CACAAGAGTATGACGGGGCTGTACGAGCTGCTGTGACGGGTGCTGCATGCGCTGCT
CCGTCTGCACCGCACGCTCACCTCCTGGCTCCGCGTTCGGTTCCACACCTGGAACTG
Gatttggcggcgctgctgccgcgccgcctctgccgcggtcctagagccgcttggcttcacgctccgcaagcatggaacagccctcaccac
acgcacccgcgcggggggtagtgcctgcccgcagcccaccaccgaatgcgctggcgcgcggacggcccttccctggagaagctgcct
gtgcgcatgggcctggtgatcaccgaggtggagcaggaacccagcttctcggacatcgcgagcctcgtggtgtg
>reg63.1
(SEQ ID NO: 213)
gtcgtgaacctgggaggcggagattgcagtgagcccagatcgcaccactgcactccagcctgggtgacagagcgagactccgtctcaaa
aaaaaaaaaaaaaaaaaaaagccgtcgcgcctcgggagtgggctggggggagagggggtgcccaagggccacaagagtatgacggg
gctgtacgagctgctgtgacgggtgctgcatgcgctgctccgtctgcaccgcacgctcacctcctggctccgcgttCGGTTCCACA
CCTGGAACTGGATTTGGCGGCGCTGCTGCCGCGCCGCCTCTGCCGCGGTCCTAGAGC
CGCTTGGCTTCACGCTCCGCAAGCATGGAACAGCCCTCACCACacgcacccgcgcggggggtag
tgcctgcccgcagcccaccaccgaatgcgctggcgcgcggacggcccttccctggagaagctgcctgtgcgcatgggcctggtgatcac
cgaggtggagcaggaacccagcttctcggacatcgcgagcctcgtggtgtggtgtatggccgtgggcatctcctacattagcatctacgac
caccaaggtattttcaaaagaaataattccagattgatggatggaattt
>reg63.2
(SEQ ID NO: 214)
gtcgtgaacctgggaggcggagattgcagtgagcccagatcgcaccactgcactccagcctgggtgacagagcgagactccgtctcaaa
aaaaaaaaaaaaaaaaaaaagccgtcgcgcctcgggagtgggctggggggagagggggtgcccaagggccacaagagtatgacggg
gctgtacgagctgctgtgacgggtgctgcatgcgctgctccgtctgcaccgcacgctcacctcctggctccgcgttCGGTTCCACA
CCTGGAACTGGATTTGGCGGCGCTGCTGCCGCGCCGCCTCTGCCGCGGTCCTAGAGC
CGCTTGGCTTCACGctccgcaagcatggaacagccctcaccacacgcacccgcgcggggggtagtgcctgcccgcagccc
accaccgaatgcgctggcgcgcggacggcccttccctggagaagctgcctgtgcgcatgggcctggtgatcaccgaggtggagcagga
acccagcttctcggacatcgcgagcctcgtggtgtggtgtatggccgtgggcatctcctacattagcatctacgaccaccaaggtattttcaa
aag
>reg63.3
(SEQ ID NO: 215)
agcctgggtgacagagcgagactccgtctcaaaaaaaaaaaaaaaaaaaaaaagccgtcgcgcctcgggagtgggctggggggagag
ggggtgcccaagggccacaagagtatgacggggctgtacgagctgctgtgacgggtgctgcatgcgctgctccgtctgcaccgcacgct
cacctcctggctccgcgttcggttccacacctggaactggatttggcggcgctgctgccgcgccgcctctgccgcGGTCCTAGAG
CCGCTTGGCTTCACGCTCCGCAAGCATGGAACAGCCCTCACCACACGCACCCGCGCG
GGGGGTAGTGCCTGCCCGCAGCCCaccaccgaatgcgctggcgcgcggacggcccttccctggagaagctgcctg
tgcgcatgggcctggtgatcaccgaggtggagcaggaacccagcttctcggacatcgcgagcctcgtggtgtggtgtatggccgtgggca
tctcctacattagcatctacgaccaccaaggtattttcaaaagaaataattccagattgatggatggaattttaaaacaacagcaagaacttctg
ggcctagattgttc
>reg65.3
(SEQ ID NO: 216)
acgggctgagcctcaaacgagctgcaggccgagttctaacgggctgagcctcaaacgagctgcaggccgagttctaacgggctgagcct
ctaaggagggaaacgtcacttcctgcctcacacagagcccagcgtctccatgtccactgatagccttggtatttgcaactatgtccatgaccat
ctctgtttctccaaaacagcctctagctacataaactgtttagaaaacctcatgcgtaaagcagagtaTGTCAAACCTCCATCT
GTGGTCAGGAGTTAGGACATCCCCAGCTGCAATTTGAGCAAAGACGGCGCTTCCAG
AGGATCATcggatcctgtgtcttggttggggttggggcccatcaacttaaaatagcttctgtttatgctggtgaaggaggcacagactt
caccctatctaattccaaggaacaggcgagggtgggagctgtagcggaagagacaaaagcaaaaggcagattcgcccctttgtgtggtcc
cgtaagtgacactgtccctccctctccctggaaacagcagcccccaggcaccccccccagcaactgggacaagggcaca
>reg72
(SEQ ID NO: 217)
tggctgtcgaggagctgctgttgctgttccgcgtcggtcctgctcctgcgcgcgtcgtccaggcccgccaggtcgccgaccagtctctagg
gcgtccatcgcgggacccacgggaggcagaagtggaggccgtgcgcaccgcgagctcaacacagttgggggccaggtggccgcctc
ccagcaggttgtcggggttgagctgggtcttgtgctcatcgctgggcttgtagtgcggtgccggtcctcaaggaTGGGGCCGAAG
AGATCCTTGAACACGTCGTAGGACTCCTCGTCGGCCGCCACGCGGCCCACGGCCCTG
AGTACGGGTGGCCCGGGCTGTCCACGCGGGTCTGGATGgcgcctccagcgcgaagccacccctggc
gcgcagctccgcgttcagctggggcagcgcctcggccactgggtcttggtggccgctcaggtcgggaaactcgtcctgcgccgggaggc
gagcttcagcgccccgcggctgtcggagaagggcatgtgcgggcgctcggtgggtccgcagctctgagcgtggccactttttaactgttat
aaataattctgctatcaacattcatatgtacacttttcttat
>reg74
(SEQ ID NO: 218)
ctgctattgatgttttctctgcccaggttctccccacacacggggttagggagggtgtgccagcctgccctcacatccccagacagagtcccc
ctccagcatctgctgcctacctccttctccctcagtgcctgtttgtttttcttccagaaccatcgcctctcaccaaggcagccatccaagggggg
cggtgttccggagacatcctctgccccccgcacccctgcagcggtagcctggtgggggctggtGCTGCAGTTTCGTCAGC
CCTTGGCTCCGGGCTCTGCAGGCGGAATCCCGAGCCTGCGTGAGGGCCGCCCTGGCC
TCGGCGTGTGTCCTGGGAAGGGGCGTTGGAAGCCTCTCTGCTTGTCTTGGCTGCCTctg
ctcgctcagctctgcccccactggggccgccagcctctgcactcccccttggaggagccaggcagggtttgggtcggagctggggtaga
ggaaggctccaggcggcttgccgcaggatctccctgctgtagccagcccttggggcgctcagcagggtgggggaccatcagtcagggtg
ggggaccctcagtcaggataggggggctcctgttctttccactgccaccaagctacccttcccctaact
>reg77
(SEQ ID NO: 219)
taccccagctgcctgaccgggagagcatcctgttcttcccctctggaattccgggtccacagctgtcttcctactcacatctggcctcggcatt
cccgccaagccctccccttgaagcacaaggatgttttgtccaggatcctgagcccagggccttccaggtggcagagagagatccggatgt
ccagccagctctgggggttcccccatcctgccagtgtggggacctccttgctgtagccaggtcaggcCAGCCCTAGGGAGA
CAGCAGGATGGTTCCAGGAAGCCTGGGCCGCTCCCCAGATCAATGCAGGGACGGAC
AGCAGCCAGCAGGCTGGGCCACGGCATCAGAGCTGGGGTCAAGAGGTttctagccctcttgtg
gctctcagccccgggtcctggctgcttcctgctgggcagtgacctccccagtccatttccctccctccttcctcccctggcctgagctcagctc
atggaaggaggccctgtgtgcaggaaccttgatctgcacctctgaaggatgtcagggcagctttttctctgggcctgtatgactcagcgcag
gatttagggcaggtggctccaccgtggagcctcagtttcctcatctgg
>reg85
(SEQ ID NO: 220)
cccgtctcggctctggctccgtcccctggcctacccactagcgggtcggactccgcccctgcttctgaccacgcccccgcgcccaccctctt
cccaccctcctcccacccagggctctccagacgcgcatgcgcacccgttgtgcatctgccgcgtggtgaccgacacgccgtcggcgccgt
ccccgctgggccgcagcagcaggttcccgcactcggggtagcgctccaggagcagttgtgcctccagccGGCTCACCTTCA
GGAAGCACCTGTGGCGGGCCGCGTCACCCACTCGGGACCCCGGAGACCAAGTCCGC
TCTTCTGCACGTAAACCCTGCCTCCtctgagacccagccccatccccatcccctaggcccaggagaccctgccctg
ctctccagacccaggcccctcccacggagacccagtccggccttccaggctcctagtttttgtggggttttttgttttttttttgagacagggtttc
gctcttgttgcccaggctggagtgcaatggcgctatctcggctcaccgcaacctccgcctcccgggttcaagcgattccccttcctcacagg
cccggctaat
>reg86
(SEQ ID NO: 221)
gattttcaggaaccatgcatggctatcgcctcctcccgcctggagggctgctcctgcgcctctgaccggcgctggttccagccgcggccca
gctgagcacagcaggaaccgcagtagcagccggagcgcccacgcccggggtcgcctagcccaggaacgccttagttgcaaccctgcgt
cgaggcccagctccgtgcgcagaaagccgaggccaaccagagcatttcctggacgagtcctctcggcctgcgGGGCCAGTTC
CTCCTACCAGCTTCCTGCTGCCACCTCGGCTTCCATCAGAGGGACGCTTAGGATGGC
GCAGGGGCCCGGAGACACTGTGAAGAGTCCAGGGGAATGAGGAGGGgctgggccgggcag
cctcaggcccagcgcaggttagcgcttctcacgcctgagcagagatcagctactgccactgcggggaggacagaaggacccaggctcc
ccagcctccctctgcaccgggagtgtaggaaactatttaaaaataataataataataataataataagtatggaatagaacttgcagatctaac
ccaaccaagttttcattctttttccttttccttttcttttttttaatgtatttt
>reg90
(SEQ ID NO: 222)
tcacttgggcatcttaagagtgggttcgtaaacttggttgtgtgcgctgtgcagatgtcagtcaccctgtgtggtgggcaaagccgacttctcc
gcctctgtagctccgaaactacaatccccagaggcctctgcggtcacttccgctcccctccctacccttcagtgtgtagcgttgacgtcagaa
acacttccggtcggtggcccaggcgcgttaagctggttgggacccgggaaggcctccctcttaaGGTCTTTCCCACACCTC
TGCACCTTGTTACCTGACTTTCGGCTTCAGGATCCGCAGCGTGCACCCGCGTTCCGT
GAGTGCCCTATAGGCAGTCAGCATGCCCCTCTGcgtgtccctgtgttacggggacgccggctgggagccg
cagagctatctcagaactagggcgctctcctttgggcacctccaggccattttcctttcattcgagcccacagggttagagataaaccctcact
ccgttgcttggggacaagggcttcactccctgtcccgagcttgcggctgagcttgagggtggctgggtcatcctggccccccactggatgg
gaattggctgctctggtgatttctgtga
>reg92
(SEQ ID NO: 223)
cggagaagctggagcggcagctggccctgcccgccacggagcaggccgccacccaggtgagccccgcacctgcccactccctcccct
ccccgggcctcctacccacccctgacactgcaccccgcctccccaggagtccacattccaggagctgtgcgaggggctgctggaggagt
cggatggtgagggggagccaggccagggcgaggggccggaggctggggatgccgaggtctgtcccacgcccgcccGCCTGGC
CACCACAGAGAAGAAGACGGAGCAGCAGCGGCGGCGGGAGAAGGCTGTGCACAGG
CTGGTGAGCGCCTGGGCCAGCGGGGCCTGCCTCTGATGCCTCGCCCCCTTCCTTccttcc
tcccaccatgggctgccctgggtgctgcgggcagcctgcacaccccaagccccgcatgtggcctgtggtttgggctgtttgggatcctcac
agctgagactcatttcccagcctatccaggcagggctcgggctggggtgggacagggtccctggcgcttctgtttgaggggcggggtgg
ggggaggtttctgcaccgcagaccaggggagatggatgacaaaaggggcttcagcaaacagct
>reg96
(SEQ ID NO: 224)
gatctccctaagaggttatgccagtcacactcctgccaagagagtatctctgcgccacggccaagggtgagtcatcctgctgagaggttgag
ctggggacgcctgcccagatgggctccaagtgagggagagcctggcggggagaacagcccggacagaggcagggcagggcgccgg
gacactgcttggcgcgtcctgggagtgaagcgcattgaacccagctcaggctggtggtgggggagtcttggcaaTGCTCTCTCT
CCAAAGGCGAGTTGATCACAGACGCTGGCAGTGAGTCAGCGGCACCGCCAGGGCTG
CTGAGAAATCCCTCCTGCTGTCCGatcgcattcctggaagggtgggccgctcagggccccccagctccagtcccac
tcaggccccagaatcccagcagcccaccactcacttctttgcgctcactcttccttctggtccccacacaccgctccctctctcgctaccttca
gtctttgctcagatgtcgagttcccagaggggcctccctgacgccaccgttctagcagcatttagcatttagataaatgacaaattttagattaa
atgttagat
>reg41.1_41.2
(SEQ ID NO: 225)
agagcctgcactggggaagatacacaccacagaagccggcgctgcagaagcacatatgccaaggacctagcgctggagacgtgcaca
cgcctgggaacaggtgctggggcggcacccaagcacgggagccagcgttggggaagcggcacaccccagggagctagcgctggcaa
agcacacccaccaagagtgagcgctagaaagccgcacacactatgggagctccgccctggagaagcgtcacgtgtgtgCCTGGG
AACCAGTGCTGGAGAAAGTATGTGGAAGCTGGCGATGGAGAAGGCGCGCGCATGTG
TGCACAACCTCGCTCTGGAGGAGTCAcggccaggtgcgcgcacgacaaccttcaccggagaagtcacacgcat
gcgtgcgctggagaacctgaatttgtaatttcaaatttccctataaagaaatatccacgaactgatgactttgtgagtgaattctatcaaatatttg
aagaaaaaaaaataccaatccttcacaaactctgaaaaaataggagggaacacttcccaactcattctaagatgccactattacggtaatacc
aaagccagacaga
>reg42_43
(SEQ ID NO: 226)
aagtgttgggattacagacataagccaggtcgcctggcccaagctagatattgaggactgccagatggcaacagtagacaagacacccta
gaatggcccatctagaaggaagtagataccttctctgtagggattcaacaagggggcaaagtgatgggcatcttgggtgaggaacccagc
ccgcggaatgggaaagggctgggacactggctttacagctgggttgggaaagggatctgatccttgagtcaGCCCCTGTAAAA
TGGGGATACAGCAGGGCACGACGTCTGTTGGTCGCCTGGCACTGGGTCGGCCACCG
AGGCCGCGCCTTGGCCTCTTTGTCCCCTCTGGccaccggccccagggagcccgctcgggaagcagcgcgg
ccccaggaggaaggcggcgcggccgaggccagagccggcggctactgcgaccttccggctggcgggcgcgtttcatgttcctgcctca
ccctgggctgcacggactcagatcgggaagggggaggatccatggtaaaggccacgccccctctgggacctcgattccccttctgggcg
gccgagggatgggctgcaaggagtcaaatcctctt
>reg2.45
(SEQ ID NO: 227)
gccagcagacaataacctctcctcttgaattggaaaaacaaatctgtggaggccttctctgccccttgttataggggccccatatgccgttccc
aggattaaaccaaaaagcacacattcctctctggcaggtgcaggtcccacccactctgggcagccaactgatgtgcacattttaatttcctaa
aacaccaggacagaaccttcctcaggggtcaggtggctcacccttggccctcaggctttggagaTGGGCACTTGTCATCAT
GGGTGTTTGGAAAGCAACTCTACGTTCTAGCCTGTGCTCCATCGTTCCTTCTACATAC
AAGTGATGCAaacatcaaaatatgttttttctttcttccttcctttaaaaaaaattgaatcctggatgaagttttagctctgtcacttgacaa
ctgcattatataacctagggtacttgaatctcagccccaaatctctaaaatgggggcagtaacatgcttctgccaggtcccagacctgtgggtc
tccaaatctgcacattcttcaagccttacaggtccttccctggtctctctaaggatgtcatgggcacagagcc
>reg2.53_B
(SEQ ID NO: 228)
gaggcagagggtagggggtgaggaggtgtttcttgtcttcttcttccaatctcagaagtaaacattggaaagtggggcccccagcagtgtac
agcccgtttccaaaccaggcctgtaaggaggagctgaggtttcggctgagcccccagcctcccccgaccgcacagcctcgggcatgaac
ccgcgaagccagacgcttagttgcttatcaggccatcgctgtacatatttagaaagtacctatcactcagACACTTTGAAAAGC
GTGGCGTTCCAGCGCAAACCAACCCGAACGGGTTGGAAGGGGGCAGTCCTTTCTTC
CCGCAAGTTCGGGGCTCGAGAGACGGCTGCAGGAAGGCCatcacccctggcttcctgcagccacagc
ttccagccccacacgatgcccaacttcattttagcagtggcccccaggggaaatcacaccattcttggttttgtccctccctcctgaggttggg
acattgttcaaacaaaagtaagccttcagctgacagagaagctgccccgcctcttccctgcccttgtcttgctggcattcattgggactaccag
gtagctttccttcccagctcaggtgtttacctgc
>reg2.19
(SEQ ID NO: 229)
ttatagatgagattctacttaggggtaggattcattattcatgaagggtgtggtcaggtgaggcatgttggaagcaaaatgcgaattaggtaag
gtggagtagaagagagctattggcaagagaaaaattacttgagcagtgtgtgagtgggtgggtgagaaagtgggcagggtggactcaga
ggttgggaagctgctcctgagaggagaagcctctgtctctacacaggaacctacctgacacatgaggcAAAAGGCTCCGACG
ATGCTCCAGACGCGGACACGGCCATCATCAATGCAGAAGGCGGGCAGTCAGGAGGG
GACGACAAGAAGGAATATTTCATCTAGAGGCGCCTGCCCActtcctgcgccccccaggggccctgt
ggggactgctggggccgtcaccaacccggacttgtacagagcaaccgcagggccgcccctcccgcttgctccccagcccacccacccc
cctgtacagaatgtctgctttgggtgcggttttgtactcggtttggaatggggagggaggagggcggggggaggggagggttgccctcag
ccctttccgtggatctctgcatttgggttattattatttttgtaa
>reg2.46
(SEQ ID NO: 230)
aatcaaattacaagaaagggaaagagaaaggaagagggagtgggacccagagagctggcgggaggcagcgaaggggaaagcttcag
tgcacgcatagctcctgcacagcggctcctgcagccccccaggatgcgcctgagctgaggctgcttgtgggcaggccctagagagaggc
aaactttgactccaggcacgcagcaggtttaactcctcactggctgggttctgggagctctgggcacacaggatagGCACCAAGA
ACTAACACATCCTGGAGCTGCCCGGAGTTCCGCTCCTGCGGGCTTAGCAGGAAAGG
GTGCCTAAGGTGAGTGCCCACTTGCGTCCGATCCTCTGGGGGCGATGCAGGGTCGGG
GCGCCTCAGTGTGTCTCGCTGCTTGTTCtggttgcagtcgggaaatgtgggactttggggtcttctcctttctccggc
tttcttttttctccttctttcctctctgttttcttgtaaattacacttcgactttcaaaaaaaaaaatgtaggggaccggtggggtcgctggggttggg
ggagagactgaagaaagtgcgcctgggcggaggcggcgaagggaatctctgggcccgaggaatataccttgtccctgcactagtgtgtg
ttctcttgtggc
>reg2.17
(SEQ ID NO: 231)
tggttctttctgttgccctcatagaccgtatgtagcagttcgcgtgggcacagaacccacggtttcccgctagttcttcaaaggtgagggcagg
tgccccgagttattttcctggggactgagcccagagcggggcgatgttgtgctactgcacctccccgccgcagccctccgctgttttcttttgg
gtagtggtccaggaacttaagacagttcctcctggcgatgtgatggaatttaatgggacaggaGAAGGGAACGGGCTTTCT
TTTCAGGCCAGCGTGGCAGCGGGCGGTAGGGCGAAAGGGAGAAGGAAACGAGGGT
TTATTCCGTTGCCCACTCCGCGGTAAGCGACGTTGTAGGGCTCCACTGTAGCGAGAG
CCCCGTGGATTcctttttttttagccatttagtttgtaaacatcactttaaagaatacatagtgtattcatgacactcggtgaaaaaaaact
ttccttcccctcccgcccccccggggcagtagatatttacaaccgtaacagagaaaatggaaaagcaaaagccctttgcattgttcgtaccac
cgagatcaagcagcagtcaggtgtctgcggtgaaacctcagaccctgggaggcgattccactttcttcaaggtaaa
>reg2.29_A
(SEQ ID NO: 232)
ccagttcgggatcgtgtagccggcggggcgggggccgtggggggcctggaggagggcaggggccgcgggaggccgggaggaggg
tggggaccttgcagcccccatcctctccgtgcgcttggagcctctttttgcaaataaagttggtgcagcttcgcggagaggagaggcgctgc
agtctgtgctgtgtccgcggggcggggaggaggtcccaggagccggttcgaaagctccctccgtgatgaagtaggCGAGAAGG
GAGGAGGTGAAGGAGGGCGAGCTGAGCACACGCGCTTCATGCCACAGGAGGGTGG
GAATGAGCGGAGGACTGAGGAGAGGAAGGAGGGAAAGAATAGGGAGATGAAAAC
GCCCCGgtctgctgctaagcacagcacagttaccaaagccaggaaactaacactgacacgatattttatttacgttacagctctattcaaa
gctccaggcttctttttgtagaatcgtttccatctgctggaatccagcatcgcccccaccccccgccccatttctagggggatgcccccactgc
tgacctctcctgctgtagatctatttctgggaggcactgacatgctgactcttgctatggggtcggcgggg
>reg2.29_B
(SEQ ID NO: 233)
gggaggccgggaggagggtggggaccttgcagcccccatcctctccgtgcgcttggagcctctttttgcaaataaagttggtgcagcttcg
cggagaggagaggcgctgcagtctgtgctgtgtccgcggggcggggaggaggtcccaggagccggttcgaaagctccctccgtgatga
agtaggcgagaagggaggaggtgaaggagggcgagctgagcacacgcgcttcatgccacaggagggtgggaaTGAGCGGAG
GACTGAGGAGAGGAAGGAGGGAAAGAATAGGGAGATGAAAACGCCCCGGTCTGCT
GCTAAGCACAGCACAGTTACCAAAGCCAGGaaactaacactgacacgatattttatttacgttacagctctattca
aagctccaggcttctttttgtagaatcgtttccatctgctggaatccagcatcgcccccaccccccgccccatttctagggggatgcccccact
gctgacctctcctgctgtagatctatttctgggaggcactgacatgctgactcttgctatggggtcggcggggagtggggagctgggcattcc
ccttcttcctcaggaca
>reg2.15
(SEQ ID NO: 234)
acacacactccctcagaccagatgcccaaccacttccagatgctacagtctcggatatccttggttaaggaagaggaagaaaaagctcgcc
cttcacgtccagatacttgggttcgggttacatgaaacaggattagttcagaaaatcgtgccacttcacagccaagacaaaaacccaagaat
gaaaaccatgtatacagccaacacaatagcaagactgaagacagtgacaaagagagttttctggttctGCTCTGATGCCTCTC
CCTCCACACCACACCTGTGATCTACTGTGCATAGGATCTCACAGGCCCAATAACAGA
GCTGGAGTTCCTCTTACGTGACACAGGATTTGGCATTTGCCTGTGCCgggctatcactcctgcc
ctgcaacacgctggtcagctggagaagcctgctgctcacacactcaccagcaacttctctaccctggatggtcaccaaaaaggaagagca
atgtctgtgcccccagcattggtgcaaaggaagtggcagagaagcaacaaggagggtggtgccttgccccaactgcccgccagcaccca
cagccaaggcaactgttctctggtgaaggcagagctggaaatgcatgcctgagc
>reg2.51
(SEQ ID NO: 235)
caagggattctcctgcctcagcctcccgagtagctgggataacaggcatgcaccaccacgcctagctaatttttttttttaatgtagtagagatg
gggtttcaccatgttagccaggatggtctcgatctcctgatcttgtgatccacccacctcggcctcccaaagtgcagggattacaggcgtgag
caccgtgcccgaccaagattgaccttcttaaacaactttgtcatcatgtgcttctcctgctcaGACATCCTCCTTGGCAGCCT
TTCAACACGTTTCTCAAATCCTTTCCCAGCTTCCTGTGCAGCCTTTCCTCCTCAGCCT
GGCTGCCTTACTGTCTCAGCTCCGATCTCTGGGCCTTTtcccatatggctgcttccctctacagtgttcctc
ctagcccataccccaacccaccccacctttccctcctctccaggttgtaccagttccaggcccctgcccttgacaatactccttcccacgagg
agcacttcctcggctacctccttagcgtgtattggaattcccactcacttggcagttgcactttgtgacacttaattctgccattttattttcctaact
gctatttagtctccctgtttattt
>reg200
(SEQ ID NO: 236)
gagggagttcaacggcgaccacttccttttggagcgcgccatccgggcagacttcgccctggtgaaagggtggaaggccgaccgggca
ggaaacgtggtcttcaggagaagcgcccgcaatttcaacgtgcccatgtgcaaagctgcagacgtcacggcggtggaggtgggggcttc
cccccagaagacatccacgttcctaacatttatgtaggtcgcgtgataaaggggcagaaatacgagaaacgaatTGAGCGCTTAA
CGATCCGGAAAGAGGAAGATGGAGACGCTGGAAAGGAAGAGGACGCCAGGACGCG
CATCATCAGACGCGCAGCTCTGGAATTTGAGGACGGCATGtacgccaatctgggcataggcatcccc
ctgctggccagcaacttcatcagtcccagcatgactgtccatcttcacagtgagaacgggatcctgggcctgggcccgtttcccacggaag
atgaggtggatgccgacctcatcaatgcaggcaagcagacggtcacggtgcttcccgggggctgcttcttcgccagcgacgactccttcgc
catgatccgagggggacacatccaactaaccatgcttggag
>reg206
(SEQ ID NO: 237)
agaactggccctcccctcttcactcttttttttttttttcttgagacagagtctcgctctgttgcccaggctggaatgcagtggtgcgatcttggctc
actgcaacctctgcctcctgggttcaagcaattctcctgcctcagcctcttgagtagctgggattacaagtgtgtgctaccacacctggctaatt
tttgtatttttagtagagacagggtttcaccatgttggctgggctcgtcacaaactccTGACCTCAGGTGATCCACCCGTC
TCGGCCTCCCAAAGTGTTGGGATTACAGGCGTGAGCCGCCGCGCCCAGCCCCCTCCT
CACTCTCTTTCTCTTCCTGTAACTTCTACAGCTGGGCAAGAGCTGGGTCTccagcggttgca
cggagaagtgtgtctgcacgggaggagccattcagtgcggggacttccgatgcccctctgggtcccactgccagctcacttccgacaaca
gcaacagcaattgtgtctcagacagtaaggggagcgaccggggaggttggagaggggagcacctgtggccagggcggaggtggagg
aagaggcagggtgggaaggggcttagcctgaaccccagcacagtcaggggttggggcgggcg
>reg208
(SEQ ID NO: 238)
gaaggatgagaagcattttgccagactcacagcgggacagtattccaagtagagcatggacaaggtacagagaccaggtggctggctttct
ctagcaacctgtctgctaccccagttcttccatgaccaaactgggtgtattgaacaagttacctcccctctctgagcctcagtttgctcatcagc
aaaatgggggtgttggcagagacctttcagacctttcggagttaccaggggtggggtccgcagatCCCCATAGGGAGGACT
TGCGCACAGTTGGCGCTGGGTAAATGCTGGGAGAACTGCTGCGGGCGAGGGGAAAG
GGTTAAAGctaggcgctttttaattgtcaaatgactgcgggcgattagcactggcagcttcctcaataatcgctcttctctgtacctgctg
ggagcttaattaaaaaacaaagaggctcaatttaaaggccattactatgctaatgcggccgggcgggcggtgattaagcggctcaggcagg
cagcgggcggctggggcggggcatggggcgatcagttacccactaaatgggcgggctgcgtgccctgcctgtcccg
>reg211.2
(SEQ ID NO: 239)
caggacagtgtgggggcggtccctagttcacagcagggaggccctaagcagtgaggtggcctgcccgccatgtcgcaggagcccctag
ccctggcagactgggcagtagcgctggctggccgctccgggttgtgctgcaggaagcccttctccagccgccagcctcgtcctgcccagc
cctgggccccacatggcaggaaacaaggccaaagaggcaccgcttagcaagcggcaggacgtgccagggctcaCACAGACA
CCCTGAGCTTGCAACACTCCGGGCCTCTGCCGCGTGTTTATTTCAGGATGCCGTGGC
ATTTGGGTGACCTTTTGTGCTCACCATGGCTTGCGTCGTCTCCGGGTCACTCTCGTCT
GGACtgaagtcccgtctccctcagctgagcctgtgccatggccagcctcagcgggaactggcaaagggaaaggggttccttggggag
gcagcaggggtttctgaaggatcatcttcaagcaaggggtttagagctcaggagtgtatttgtgtttttttgttttttgtttattttgagacagggtc
tggctgtgtcactcaggctggaatgcaatggcacagtcctggctcactgcagcctcaacctactgggc
>reg302.1
(SEQ ID NO: 240)
cttgaatctcctccagtccttccactatatgcccctctggccttgtcttgcacacctccagagacagaagcctcactactttagaggctgtccttt
ccatcttcctgactggaaggaaattcttccctgaatggagctgaaacttgtgtccctgctctgccctctagggttttttcccagtaactggaaga
gtcttgaaagggctaaaatgattttatttttaaatgtggacaggcaagcagaggtggttgGCAAAGGCAAGGTGGCTGAC
GATCCGGAAGCTGTACAGGAGAGATAAGGGCACTGGCTGCCAGAGTGCCCTATCGA
AGCATCATCCGAACCCTGCGGTAGGGGTGGCCCACACCACGGCCTGAGGCCCAGTC
AATGCCATATTTGTGGGCggcagcctcagacactgcatagcgaccattgagatttgatcggtaacaggatgcataccacca
ggcaccgtggacaatcactgcacagttgctgttgcttgaatcgtggtcagcgtcataggtggtaaagggcctcccactgtggaggctcagg
gaatcccctagcagggaagggatggaaagcaccttggtgcccagcaccacgcctggcacctttggagatataatgccatgggagtctcag
agcaac
>reg302.2
(SEQ ID NO: 241)
ccagagacagaagcctcactactttagaggctgtcctttccatcttcctgactggaaggaaattcttccctgaatggagctgaaacttgtgtccc
tgctctgccctctagggttttttcccagtaactggaagagtcttgaaagggctaaaatgattttatttttaaatgtggacaggcaagcagaggtg
gttggcaaaggcaaggtggctgacgatccggaagctgtacaggagagataagggcactggcTGCCAGAGTGCCCTATC
GAAGCATCATCCGAACCCTGCGGTAGGGGTGGCCCACACCACGGCCTGAGGCCCAG
TCAATGCCATATTTGTGGGCGGCAGCCTCAGACACTGCATAGCGACCATTGagatttgatc
ggtaacaggatgcataccaccaggcaccgtggacaatcactgcacagttgctgttgcttgaatcgtggtcagcgtcataggtggtaaaggg
cctcccactgtggaggctcagggaatcccctagcagggaagggatggaaagcaccttggtgcccagcaccacgcctggcacctttggag
atataatgccatgggagtctcagagcaactaagagttgaattttatcaggccccacgagc
>reg305.1
(SEQ ID NO: 242)
ttccatggcccagaagtctgcaggacccacagcaggtattcgggactatttgttcaatccacacctgagtcgttgcacgattatgctcaagtcc
ctcggaacacctcgcctgccatctgacagcttcccatccagaaaccacacagtacagtaaaaaacagaaaaaagaaagccgttagacccc
agtgaatgttatttttaatgaaagtggtgcattttgactcacaatgttgaaaccagattataaatgaGTCATCAGTGAATCGACC
ACAAAGAGCCTTTGCGGAGGTGATTTACAGGAGAGCTCTGATGTCTGCTGTCCCCTG
CACacgcttcacagagatgctgtcagacgcagagctggtctggggcatctgttgccgcgtcagctcaaaaggatgctgtgttgtcaccaa
tgggattccccagcccaggcggtgttgcggtcccacccacacaaggaaggcggccatcactgaataatgcttgtggttacatcatcattgct
ggtttccaggtagtgactagcagatactggagagagacaggccatctgctcttcctgtgcgcctcagctcc
>reg314.3
(SEQ ID NO: 243)
tagcttgtcagcatgaacctacatgcaagccagagatctatgattttgtttcccagggagggagtgactaatgcgcgcaccctgaccatcacc
gtaaagaggtaaagagagagtgaaatggctcaacgtacacacaccccgctccataccggggacgagtctccgagctgcggcttgtgctct
cggagggccaggctgaagctgaccgcccccacggccacgctggacacccccagccctatctccagcacGGAGAGCACCAA
GAGGCTCCCAATAATCTGACCGCTGGTGCACATCCTTCCTCGGTCATCTTCCTTCCAG
ATCAGAGAGGGAAATCAACCATCTACCTTTTTTTCTTCCACTATCCTCCTTACCCCTT
CCACCCCCTACCAGATCCCAAaacttttctttcttcaagagcgaggcattatccacaagggctggatttccagaaacgaag
accttccctggctgggccagaggcaaaggagctgctccaccccctggcacgttcagatagggatcgtagaaggatcttcctgggttcggtg
gtgcgaagattgcacaccggtaccggggcttttaagcagcggaaaacctggaggagcccagggagctccgagccttgctccccaggcg
ctgtccagagt
>reg315
(SEQ ID NO: 244)
tgtcttagtcaggagggttggatgtaagaaacaagcccttaacattcgcttctttgtggacgagatgcagtagaatcatttagtccttgcactctg
agcctctccacagaatttgctgttggaagtcatctcagtaagaaatacacagagaaatctggtctttgttcctatgatgacaaagcagtttcataa
tctgccctcttgcagcttgctctgttttgggtgcagataaaacaagcatggttctctaaTAACCACCTGCACCTCTGCTGC
AATGTAAACAGCAGATGTGGGCGCAGGGTGAGAAGGGAGAGGAAGCTACGTGCAA
TGGCAGGTTGGGGAATAAGGAGGCAGAGGGGCTCCttcatcttttacagggtaaaatgggatcaggacag
ttgcaggacagacttgtttctcaaccacgctgttaagagaatttcatactgcaagtcacaagggcccagggctccacggcctttagcccaccc
tggctttctaacaacccaaagtgggtatggagaaattgtcctttaaaaacctaaaaactatgttaattttcattttcaaaataaagaataaatcagc
acttttggaaaggaggtgggaaggg
>reg316.2
(SEQ ID NO: 245)
acgcttgtgataacgataagacagaaactattgaaaagggtgcagtggtggtgtgaaggattaatcctttgcttgcttcacatctgaacaggaa
tctccacacaaatgtcccacatgtggaagaacctttaatcagagaagtaatctgaaaactcaccttctcacccatacagacatcaagccctac
agctgcgagcagtgcggcaaagtgttcaggcgaaactgtgatctgcggcggcacagcctgactcACACCCCGCGGCAGGA
CTTCTAGAGAAGCCCAGGATCTGTCCCGTGCCGCCGCTGCTCCCCTCCCCAGACACC
TCTCCACGTCTCCTACCCAGGGGGTCGCATCCCTAGCCCTTCACTGACCCCAGCTCTT
CCCttgctgcagccgcacctgcagctccagggagttaactcttcttctgggggactgagaactgtagaaagccacacactactacatccct
tcacaaagagtatatgctagtttcttgtagatattcacagctcattttagagctctgtacataatgttgtgggtctttgttttgttgttttgtttgctttgg
gatcttgttggatgcacttagatatggaaaatggaagccaaattttatctttaaagactg
>reg318.2
(SEQ ID NO: 246)
ggggaaaactccaggtcagatggggtgaaccagagggaacaatgcacttcttcacaaaccaacatataaacacttgcgaatgaaatcacg
cagagacattcatcagcttcaaaaggagagcggaactgggaaaggagtcggcagaattgagagaggagaatttgggaaagcttctccatg
agagcggtgcctggagaggtgggttgggaaccgtcgctgagaataaggcacaggtcagccacctttcccagCATCTCCTCCTC
GCAAACCCCAAGCCAAGGCAAGCTGGATGAAGCGCTCCCTGGGCAGGCCCGGCTCT
CCGTGTCCCTCCATCACCTGACCCCGCTGGCTCTCGCAGACCCCTTCCTCCACACTCA
CTCCTCCCGGCTCTCCTTctataatctcctgacatctcttcaaatccaattattgaattaattgacgtacgaacccagaggcaaa
cagaaaggggcggcaaacactgggcggctcagatttatccttcggcctccgcagggcccggccggacgagatttactgggcctcgaaca
cggcgacagttcaaacctttgattaatcatgtttttctgcctaccccataatttagttgctctttttccctccctgccttttttttttttttta
>reg318.3
(SEQ ID NO: 247)
gagcggaactgggaaaggagtcggcagaattgagagaggagaatttgggaaagcttctccatgagagcggtgcctggagaggtgggttg
ggaaccgtcgctgagaataaggcacaggtcagccacctttcccagcatctcctcctcgcaaaccccaagccaaggcaagctggatgaagc
gctccctgggcaggcccggctctccgtgtccctccatcacctgaccccgctggctctcgcagaccccttcctCCACACTCACTC
CTCCCGGCTCTCCTTCTATAATCTCCTGACATCTCTTCAAATCCAATTATTGAATTAA
TTGACGTACGAACCCAGAGGCAAACAGAAAGGGGCGGCAAACACTGGGCGGCTCA
GATTTATCCTTCGGCCTCCGCAGGgcccggccggacgagatttactgggcctcgaacacggcgacagttcaaacctt
tgattaatcatgtttttctgcctaccccataatttagttgctctttttccctccctgcctttttttttttttttatcagcggaaacagagacggagtcctca
tcagcttcaattacaaatattaaggtcccggacagcactttgacagagaggcggccagccccccacttcgtaccacccccctaaatcatctcc
ga
>reg318.4
(SEQ ID NO: 248)
aggtcagccacctttcccagcatctcctcctcgcaaaccccaagccaaggcaagctggatgaagcgctccctgggcaggcccggctctcc
gtgtccctccatcacctgaccccgctggctctcgcagaccccttcctccacactcactcctcccggctctccttctataatctcctgacatctctt
caaatccaattattgaattaattgacgtacgaacccagaggcaaacagaaaggggcggcaaacacTGGGCGGCTCAGATTT
ATCCTTCGGCCTCCGCAGGGCCCGGCCGGACGAGATTTACTGGGCCTCGAACACGG
CGACAGTTCAAACCTttgattaatcatgtttttctgcctaccccataatttagttgctctttttccctccctgcctttttttttttttttatcag
cggaaacagagacggagtcctcatcagcttcaattacaaatattaaggtcccggacagcactttgacagagaggcggccagccccccactt
cgtaccacccccctaaatcatctccgaattaacatcacatcggcggctggcgcgtgttcagatttaaatggtggcatat
>reg319.2
(SEQ ID NO: 249)
agctagcgtgttcatgctggatgtggtgataataacagtaacagcagcaacagcaataataatactgtcctatcttttttttttttttttttttttttttttc
agaaaagatagcctaaaagggttaagaatcccagcaagacacaacatagatgggctgaaaactcgtggcaggatggaagggtataaaga
cgccggggaagtggctggggaataataaaataagagggaagctaaaccagtgacccttgTCGGCAGTGAAAAGCGGG
AGATTAGAAAATGTTTCATGCTAATTTCCATGGAGATTTCTTTAATTTAGCGAAGAC
TGCTTCCCGGGCTCCGCCTGGCCCGCGCCGGCCCGCGTCCTCGGTGGTCTGGGCGCcc
cggctgagccgctagcgggtcactcgggcggctccgacgtctctatcagccgcgcccgcgccgcccgcctccccgcgctgctgcccgg
ctctcgggctctcgctttttttttttttttttctttccgcggcagtcttaggattcttgtcacatgatggcttcatcgggcccttctcctcctgatcctttc
aagctctttctcctgcctggcatatcaaaggagatttgtgggtcaccgagccgggacg
>reg319.4
(SEQ ID NO: 250)
aaataagagggaagctaaaccagtgacccttgtcggcagtgaaaagcgggagattagaaaatgtttcatgctaatttccatggagatttcttta
atttagcgaagactgcttcccgggctccgcctggcccgcgccggcccgcgtcctcggtggtctgggcgccccggctgagccgctagcgg
gtcactcgggcggctccgacgtctctatcagccgcgcccgcgccgcccgcctccccgcgctgctgcccGGCTCTCGGGCTCT
CGCTTTTTTTTTTTTTTTTTCTTTCCGCGGCAGTCTTAGGATTCTTGTCACATGATGGC
TTCATCGGGCCCTTCTCCTCCTGATCCTTTCAAGCTCTTTCTCCTGCCTGGCatatcaaagga
gatttgtgggtcaccgagccgggacgcagcatataaagtcatcagcctggccggcaccacctcgatcatttgccgcattgttcttgcaagga
gcccaggatggctgtggctttttaataactagcttagtagttagccgaaaaatcttagtttttaaaaatacaaaaaaaaaaaaaaaaaaaaaag
agacagtctgatagtttatttgtttttccatacactcttaattgaaactcagt
>reg324
(SEQ ID NO: 251)
ctgggggcaaactggagttgtcaggaagatctgggctttggaagaatgcgaagtgtcggtagaaggagaaggggcaggtgatttcagact
gggaggaccttgtgggcaaaggcacaaaggcgagactgacctggagatgataaggccagttgaagagacactggagaagagaagaca
gtttgttttacacattgcaggaaatcagattagacagttagggtgtggacacaaaagcgaggaccttgcaggcaCTGGGGAGAAG
TGACCCCATTCAATAGTCCTTGGTCTCCTTCTGCCCTGCGGCTGCGCTTCCTCGGCTC
TCACGGCACCAGCAGAATTCCATGTGAGAGGGAGCTTGTCGAGCGTGGCCTCTTCCC
ACTTGGGGCTGCTTTCTgcatccctgtgcctggctgtgggcctccatttgccctctactgtcttcccttaggacatcatttatgca
gagaaaggttcgtgtggctcggggtaccagtaagacctccacctctggtttcttcattttaaggaggcccttcaattatccaggaattaaagtg
gccttcctcttgggagaacgagttggttgatgaatgataagcaagtctctattcctcaaaagccagtccccaaattccatgaaatat
>reg328
(SEQ ID NO: 252)
acacacacacacttccctgagcattcccactttggtaaggaaggagtataatttgctgaatggtgcaagcaagccaggaggacagaagatgt
tacactttactcagggaacagaggcgggcaactggccctgtgactgcagccaacagctttaagaacacagtcctttctgcttcaaggttagg
gagacgttctcgcctctttcttctttgcagttattattcaagaggcttcccccgaccccagtccccaGCACCATCCTCAGAGCT
TCAGACCATACATTGACAGTGAGCAAAGGGGGCCCCAGGCAGGCGGGTCTGGGGCC
AAGGAGGGCGGCTCCCCTGCGCGGATCCTTCCCTGGTGGCTCCCAAATccggcgttttctctg
ccgcctctccctcgggggagactcggaaaggctgcaaaaatctgggcgcccgttcgctcgcttgtcaagaagcaaactgtcttcacattctc
caagagcaacatccctgcctaggaagaggaaggaagaggcaaaataaataaaaccagttaatgttgtagttaacttgcaaatcaagtaaatc
tgttggtgccgtatttgagaaataaaccatcacagcgtcacagcaaacaca
>reg2.23
(SEQ ID NO: 253)
ccctgccccgggaggtgctcaggaaagggttgtgaccccgagtgacagtagaggctcagagaggtcaggatgtgtagtgcatggtggag
ctggccactaactcgggccgcttcttgtcttgtttgagtagcaattgaggggctcctgggtgccccgggctgggctgggcctggagtcagca
agccccaagtcttgccctcccttgccagggaggaaggaaaggtaaccggctgtgacactgagggaggtgaGCTGGGAACTGG
AGGTGCAGAGAAGGCCCCGACGCTGTTTGTAGGTTGTGGGGGTGCAGCAAGACCTA
GATCTTAAGAATTTCGAAGGACTGTGACGATCACCGGCTGCGCCCTGCCGGCGAGTG
CCCTGGGGCTGGCTCTATTtgttgcgcgatccagccctggtggggagatttgtgaggggagacctggctcaggctgtgtct
tcctgttcaaacgggggttagtagagagggggttggggaggtccagggagaactgggcatgcagcctgcaggggagagggaccccttg
gagggctgcgggagaggctccttgtaaatgtcaacaaagacccagccaggcaggtccatgggttaccctaagagcttagagtttatcgga
gaggaaatgg
>reg2.27_B
(SEQ ID NO: 254)
aggttggggagttgagaaggatggagatgggtgcatctggaagggagtccgtcctgaggagtcccccatcagctgtcagccagccagca
gcaaagcaaattaagactacacagctccgaagaagccagttcccaaccaagccagtggagaaaagtcagcccggtccccaggagtgctt
gaggctctgtcactcttggacgtcaaaaagggtcatttgatgactggacgcttacctcaccggtgtgaggtaaGCTTCAAACGCC
GTATCATGTTGCTTTAAAACCTGCGGGTAACAGCATAAGCTGAGTTTTCTATCTTAG
AACTCTTAACCCCAAGAACACTCTTCACAGGCCCTGATAGGTGGACCCacaaaaaaaccact
caggctatatttgactcggatttgaaacgctgccgaaacggtattaagtgtcctcctcaactggaaaagacaaataacaaatgatgcctgaat
gagaaaaagactagacgtgcacacagtaatgtgtgagcagggaaacttcagcgaaggtttttatcatgctttaccccttttacatgctttacccc
atgtgcaaacatttttcatgggtttttttctattttttatttattttt
>reg2.31
(SEQ ID NO: 255)
gtcccagcgtcccagcccagctacccaagtagaaggtggggcggcatcttctatggctgagtcttgggcagtgggtgctctgtcatattgtc
aggtttcttcccccagctccacaatgtgaagactgaggtggtccctccaagccccactcagcaaggaggacagggctggtggatcctccag
ggtcagatggggaaataaaagtgtttcattcttgaaggggaagctgcacttctccacggcacgcctggTGGTGCCAGGGGTTA
CCACAAAGAGGCGGCAGAGCCATGGCCCACCAGCCACTTGGCAGGCTGGTTGTCTG
GTGaagatttcagggtttggcacagggcccagtcctcagtcccccccatgtccaccacctccactggtgcccccaggcctgggaggtgt
aggaggtgccgggggggcatgtaggtgtgagtgaagaggagtgtgtagtgggtgggtgtgcttgggagcacaggggcatggaccacct
gctccaggtactccaggccaggcaccaggcccccctgatgcaggcagccgaagaggagggcaccgagggcgttga
>reg2.50
(SEQ ID NO: 256)
gaggaatcatgattgtctaaactagtcatggccatcccagtcttctttgctagatacttgagtaatctctttcccagtttctctgttctggccagtga
gatgtaagagagagtctgctgagttatgtgagaactgattatttcataaagagaacaactaagagagtacttccttcccttcctgtttgggtaa
ggacttgatgctgcaggagccaccctgcatccataaaggagaagtcaaaaggaatcaGAGACAACAGCCCAGACCCC
CATCACGGAGCTGCACGTGACCCTGGAACTTAACAGCTTCCAGTTGTTCCCTAGACA
GTCATTGTCTTTATGGTGCCTTTTCCCCCATCAGGgaggaaggtgccttgatcaagtcttttcaattaaactg
cagtttaacagagaaaattgccttatgaacagtcaaaagtcagtacaacttaaatatggcagtttatctcaggacatctcccagcacacgtcttc
ctgagagcaagctgctctcaaaaccaaggcaatgggaaaatggtcagaaacatgacttctgtattttccagttcatacactgaagacagcatt
cattcattcatttggaaagtcag
>reg2.52
(SEQ ID NO: 257)
tgactgtataacactagtagatatttttaaaatgcaagagcatcttatagatcattacttttccttggaatgcttggcccctgtaatataatacaccg
gtattttgcatgatgaaattgatgtcctgtgtgttgatcatgttgctatcctagctgccgattaaaacgttttttttttttcatgccagagcagaacaa
aattgtctgcttctcaatctgcacatcataagcagatgacattaaaaatgtcTGTAAGATGACACAGCTATATTTTCTG
GGAGAGGGCGGGAGGATGCTCAGCGAGGGTGGCCCGGAGTGTCCTTGTACAGAGTA
CAGATGTTATGAAGTGGGGAAGACCAGCCTGTGttcattgattcacctattgattccaggagcaagctcaccc
tgtttcatacactgctcaggaggtaaacaggaggaagggagccagcctggcttttttgccacatgctctgctgtttggtagaactgtattatagt
cagaaaccttccgcttttctgcagttgtttgcatgctgtttccaaggctagccctctgagtctgttttctagagttgttttgaaattcaacctaaagat
aacagaggaaatgtga
>reg2.53_A
(SEQ ID NO: 258)
gcgcggcacaacggcgcattgtggggccaagcgaggggcgaagggggctgggggtggccggcgcgatggggacgctccggttgcg
ccaagttgactctgccgtttgggtcacctgggctgagtcgcgggcgtggaggcagagggtagggggtgaggaggtgtttcttgtcttcttctt
ccaatctcagaagtaaacattggaaagtggggcccccagcagtgtacagcccgtttccaaaccaggcctgtaaGGAGGAGCTGA
GGTTTCGGCTGAGCCCCCAGCCTCCCCCGACCGCACAGCCTCGGGCATGAACCCGCG
AAGCCAGACGCTTAGTTGCTTATCAGGCCATCGCTGTACAtatttagaaagtacctatcactcagacac
tttgaaaagcgtggcgttccagcgcaaaccaacccgaacgggttggaagggggcagtcctttcttcccgcaagttcggggctcgagagac
ggctgcaggaaggccatcacccctggcttcctgcagccacagcttccagccccacacgatgcccaacttcattttagcagtggcccccagg
ggaaatcacaccattcttggttttgtccctccctcctgag
>reg2.58
(SEQ ID NO: 259)
cttagcaatctgcatttctaaagtgattgtacacattccgtctatgttaaaagcctcagcagcaggttggaggcgggttctggggctagtgtttc
cgatgggaagctcaggctccatccagcctgtggctggactggccaggctcaatgtcactccccaggtagctgcccttgatctatactaggag
caccttgagagctgggaattgatttctaagcctggtttgagctgagggccacagagccagtgcAGGAGGAGACCCTGCCC
CAGAAATAGGCCAGTGCTTGTTATGCAGGCCTTGGCGGTTCCCCGTTTCCTTACGTA
ACCTCAGTGTTCACGCTGTTTCCTTTTGTTGATTCCCTCCGTGTGActgtttttctgtcaatctcctta
gctaatgagctccttataaggagaatggatggatcagagcacagctccgtacacagtggtggggcatagccatttcccagagtgtggacttt
cccagaactcccctgttgtgtgggcctgcaaaggctgggattgtttctgccttgtttggaataataaagctgcctgtgtttcctgtgttcacttttc
agtcgcctgttattcactctcctacatttggggcggtt

For the avoidance of any doubt, any methylation biomarker provided herein can be, or be included in, among other things, an advanced adenoma and/or colorectal cancer (e.g., early stage colorectal cancer) methylation biomarker.

In some embodiments, a said methylation biomarker can be or include a single methylation locus. In some embodiments, a methylation biomarker can be or include two or more methylation loci. In some embodiments, a methylation biomarker can be or include a single differentially methylated region (DMR) (e.g., (i) a DMR selected from those listed in Table 1, (ii) a DMR that encompasses a DMR selected from those listed in Table 1, (iii) a DMR that overlaps with one or more DMRs selected from those listed in Table 1, or (iv) a DMR that is a portion of a DMR selected from those listed in Table 1, e.g., at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the DMR). In some embodiments, a methylation locus can be or include two or more DMRs (e.g., two, three, four, five, six, seven, eight, nine, ten, eleven, twelve, thirteen, fourteen, fifteen, sixteen, seventeen, eighteen, nineteen, twenty, or more DMRs selected from those listed in Table 1, or two, three, four, five, six, seven, eight, nine, ten, eleven, twelve, thirteen, fourteen, fifteen, sixteen, seventeen, eighteen, nineteen, twenty, or more DMRs each of which overlap with encompass a DMR selected from those listed in Table 1). In some embodiments, a methylation biomarker can be or include a single methylation site. In other embodiments, a methylation biomarker can be or include two or more methylation sites. In some embodiments, a methylation locus can include two or more DMRs and further include DNA regions adjacent to one or more of the included DMRs.

In some instances, a methylation locus is or includes a gene, such as a gene provided in Table 1. In some instances a methylation locus is or includes a portion of a gene, e.g., a portion of a gene provided in Table 1. In some instances, a methylation locus includes but is not limited to identified nucleic acid boundaries of a gene.

In some instances, a methylation locus is or includes a coding region of a gene, such as a coding region of a gene provided in Table 1. In some instances a methylation locus is or includes a portion of the coding region of gene, e.g., a portion of the coding region a gene provided in Table 1. In some instances, a methylation locus includes but is not limited to identified nucleic acid boundaries of a coding region of gene.

In some instances, a methylation locus is or includes a promoter and/or other regulatory region of a gene, such as a promoter and/or other regulatory region of a gene provided in Table 1. In some instances a methylation locus is or includes a portion of the promoter and/or regulatory region of gene, e.g., a portion of promoter and/or regulatory region a gene provided in Table 1. In some instances, a methylation locus includes but is not limited to identified nucleic acid boundaries of a promoter and/or other regulatory region of gene. In some embodiments a methylation locus is or includes a high CpG density promoter, or a portion thereof.

In some embodiments, a methylation locus is or includes non-coding sequence. In some embodiments, a methylation locus is or includes one or more exons, and/or one or more introns.

In some embodiments, a methylation locus includes a DNA region extending a predetermined number of nucleotides upstream of a coding sequence, and/or a DNA region extending a predetermined number of nucleotides downstream of a coding sequence. In various instances, a predetermined number of nucleotides upstream and/or downstream and be or include, e.g., 500 bp, 1 kb, 2 kb, 3 kb, 4 kb, 5 kb, 10 kb, 20 kb, 30 kb, 40 kb, 50 kb, 75 kb, or 100 kb. Those of skill in the art will appreciate that methylation biomarkers capable of impacting expression of a coding sequence may typically be within any of these distances of the coding sequence, upstream and/or downstream.

Those of skill in the art will appreciate that a methylation locus identified as a methylation biomarker need not necessarily be assayed in a single experiment, reaction, or amplicon. A single methylation locus identified as a colorectal cancer methylation biomarker can be assayed, e.g., in a method including separate amplification (or providing oligonucleotide primers and conditions sufficient for amplification of) of one or more distinct or overlapping DNA regions within a methylation locus, e.g., one or more distinct or overlapping DMRs. Those of skill in the art will further appreciate that a methylation locus identified as a methylation biomarker need not be analyzed for methylation status of each nucleotide, nor each CpG, present within the methylation locus. Rather, a methylation locus that is a methylation biomarker may be analyzed, e.g., by analysis of a single DNA region within the methylation locus, e.g., by analysis of a single DMR within the methylation locus.

DMRs of the present disclosure can be a methylation locus or include a portion of a methylation locus. In some instances, a DMR is a DNA region with a methylation locus that is, e.g., 1 to 5,000 bp in length. In various embodiments, a DMR is a DNA region with a methylation locus that is equal to or less than 5000 bp, 4,000 bp, 3,000 bp, 2,000 bp, 1,000 bp, 950 bp, 900 bp, 850 bp, 800 bp, 750 bp, 700 bp, 650 bp, 600 bp, 550 bp, 500 bp, 450 bp, 400 bp, 350 bp, 300 bp, 250 bp, 200 bp, 150 bp, 100 bp, 50 bp, 40 bp, 30 bp, 20 bp, or 10 bp in length. In some embodiments, a DMR is 1, 2, 3, 4, 5, 6, 7, 8 or 9 bp in length.

Methylation biomarkers, including without limitation methylation loci and DMRs provided herein, can include at least one methylation site that is an advanced adenoma and/or colorectal cancer (e.g., early stage colorectal cancer) methylation biomarker.

For clarity, those of skill in the art will appreciate that term methylation biomarker is used broadly, such that a methylation locus can be a methylation biomarker that includes one or more DMRs, each of which DMRs is also itself a methylation biomarker, and each of which DMRs can include one or more methylation sites, each of which methylation sites is also itself a methylation biomarker. Moreover, a methylation biomarker can include two or more methylation loci. Accordingly, status as a methylation biomarker does not turn on the contiguousness of nucleic acids included in a biomarker, but rather on the existence of a change in methylation status for included DNA region(s) between a first state and a second state, such as between colorectal cancer and controls.

As provided herein, a methylation locus can be any of one or more methylation loci each of which methylation loci is, includes, or is a portion of a gene (or a specific DMR) identified in Table 1. In some particular embodiments, an advanced adenoma and/or colorectal cancer (e.g., early stage colorectal cancer) methylation biomarker includes a single methylation locus that is, includes, or is a portion of a gene identified in Table 1.

In some particular embodiments, a methylation biomarker includes two or more methylation loci, each of which is, includes, or is a portion of a gene identified in Table 1. In some embodiments, a colorectal cancer methylation biomarker includes a plurality of methylation loci, each of which is, includes, or is a portion of a gene identified in Table 1.

In various embodiments, a methylation biomarker can be or include one or more individual nucleotides (e.g., a single individual cysteine residue in the context of CpG) or a plurality of individual cysteine residues (e.g., of a plurality of CpGs) present within one or more methylation loci (e.g, one or more DMRs) provided herein. Thus, in certain embodiments a methylation biomarker is or includes methylation status of a plurality of individual methylation sites.

In various embodiments, a methylation biomarker is, includes, or is characterized by change in methylation status that is a change in the methylation of one or more methylation sites within one or more methylation loci (e.g., one or more DMRs). In various embodiments, a methylation biomarker is or includes a change in methylation status that is a change in the number of methylated sites within one or more methylation loci (e.g., one or more DMRs). In various embodiments, a methylation biomarker is or includes a change in methylation status that is a change in the frequency of methylation sites within one or more methylation loci (e.g., one or more DMRs). In various embodiments, a methylation biomarker is or includes a change in methylation status that is a change in the pattern of methylation sites within one or more methylation loci (e.g., one or more DMRs).

In various embodiments, methylation status of one or more methylation loci (e.g., one or more DMRs) is expressed as a fraction or percentage of the one or more methylation loci (e.g., the one or more DMRs) present in a sample that are methylated, e.g., as a fraction of the number of individual DNA strands of DNA in a sample that are methylated at one or more particular methylation loci (e.g., one or more particular DMRs). Those of skill in the art will appreciate that, in some instances, the fraction or percentage of methylation can be calculated from the ratio of methylated DMRs to unmethylated DMRs for one or more analyzed DMRs, e.g., within a sample.

In various embodiments, methylation status of one or more methylation loci (e.g., one or more DMRs) is compared to a reference methylation status value and/or to methylation status of the one or more methylation loci (e.g., one or more DMRs) in a reference sample or a group of reference samples. For example, in certain embodiments, the group of reference samples is a plurality of samples obtained from individuals where said samples are known to represent a particular state (e.g., a “normal” non-cancer state, or a cancer state). In certain instances, a reference is a non-contemporaneous sample from the same source, e.g., a prior sample from the same source, e.g., from the same subject. In certain instances, a reference for the methylation status of one or more methylation loci (e.g., one or more DMRs) is the methylation status of the one or more methylation loci (e.g., one or more DMRs) in a sample (e.g., a sample from a subject), or a plurality of samples, known to represent a particular state (e.g., a cancer state or a non-cancer state). Thus, a reference can be or include one or more predetermined thresholds, which thresholds can be quantitative (e.g., a methylation value) or qualitative. Those of skill in the art will appreciate that a reference measurement is typically produced by measurement using a methodology identical to, similar to, or comparable to that by which the non-reference measurement was taken.

The DMRs provided in Tables 4-6, 9-10 and 12-15 are selected regions that consist of, overlap with, or contain portions of DMRs of Table 1.

In various embodiments, methylation status of one or more methylation loci (e.g., one or more DMRs) is compared to a reference methylation status value and/or to methylation status of the one or more methylation loci (e.g., one or more DMRs) in a reference sample. In certain instances, a reference is a non-contemporaneous sample from the same source, e.g., a prior sample from the same source, e.g., from the same subject. In certain instances, a reference for the methylation status of one or more methylation loci (e.g., one or more DMRs) is the methylation status of the one or more methylation loci (e.g., one or more DMRs) in a sample (e.g., a sample from a subject), or a plurality of samples, known to represent a particular state (e.g., a cancer state or a non-cancer state). Thus, a reference can be or include one or more predetermined thresholds, which thresholds can be quantitative (e.g., a methylation value) or qualitative. Those of skill in the art will appreciate that a reference measurement is typically produced by measurement using a methodology identical to, similar to, or comparable to that by which the non-reference measurement was taken.

### Cancers

In certain embodiments, methods and compositions of the present disclosure are useful for screening for advanced adenoma and/or cancer, particularly colorectal cancer. Colorectal cancers include, without limitation, colon cancer, rectal cancer, and combinations thereof. Colorectal cancers include metastatic colorectal cancers and non-metastatic colorectal cancers. Colorectal cancers include cancer located in the proximal part of the colon cancer and cancer located the distal part of the colon.

Colorectal cancers include colorectal cancers at any of the various possible stages known in the art, including, e.g., Stage I, Stage II, Stage III, and Stage IV colorectal cancers (e.g., stages 0, I, IIA, IIB, IIC, IIIA, IIIB, IIIC, IVA, IVB, and IVC). Colorectal cancers include all stages of the Tumor/Node/Metastasis (TNM) staging system. With respect to colorectal cancer, T can refer to whether the tumor grown into the wall of the colon or rectum, and if so by how many layers; N can refer to whether the tumor has spread to lymph nodes, and if so how many lymph nodes and where they are located; and M can refer to whether the cancer has spread to other parts of the body, and if so which parts and to what extent. Particular stages of T, N, and M are known in the art. T stages can include TX, T0, Tis, T1, T2, T3, T4a, and T4b; N stages can include NX, N0, N1a, N1b, N1c, N2a, and N2b; M stages can include M0, M1a, and M1b. Moreover, grades of colorectal cancer can include GX, G1, G2, G3, and G4. Various means of staging cancer, and colorectal cancer in particular, are well known in the art summarized, e.g., on the world wide web at cancer.net/cancer-types/colorectal-cancer/stages.

In certain instances, the present disclosure includes screening of early stage colorectal cancer. Early stage colorectal cancers can include, e.g., colorectal cancers localized within a subject, e.g., in that they have not yet spread to lymph nodes of the subject, e.g., lymph nodes near to the cancer (stage NO), and have not spread to distant sites (stage M0). Early stage cancers include colorectal cancers corresponding to, e.g., Stages 0 to II C.

Thus, colorectal cancers of the present disclosure include, among other things, pre-malignant colorectal cancer and malignant colorectal cancer. Methods and compositions of the present disclosure are useful for screening of colorectal cancer in all of its forms and stages, including without limitation those named herein or otherwise known in the art, as well as all subsets thereof. Accordingly, the person of skill in art will appreciate that all references to colorectal cancer provided here include, without limitation, colorectal cancer in all of its forms and stages, including without limitation those named herein or otherwise known in the art, as well as all subsets thereof.

### Subjects and Samples

A sample analyzed using methods and compositions provided herein can be any biological sample and/or any sample including nucleic acid. In various particular embodiments, a sample analyzed using methods and compositions provided herein can be a sample from a mammal. In various particular embodiments, a sample analyzed using methods and compositions provided herein can be a sample from a human subject. In various particular embodiments, a sample analyzed using methods and compositions provided herein can be a sample form a mouse, rat, pig, horse, chicken, or cow.

In various instances, a human subject is a subject diagnosed or seeking diagnosis as having, diagnosed as or seeking diagnosis as at risk of having, and/or diagnosed as or seeking diagnosis as at immediate risk of having, a cancer such as a colorectal cancer. In various instances, a human subject is a subjected identified as a subject in need of colorectal cancer screening. In certain instances, a human subject is a subjected identified as in need of colorectal cancer screening by a medical practitioner. In various instances, a human subject is identified as in need of colorectal cancer screening due to age, e.g., due to an age equal to or greater than 50 years, e.g., an age equal to or greater than 50, 55, 60, 65, 70, 75, 80, 85, or 90 years. In various instances, a human subject is a subject not diagnosed as having, not at risk of having, not at immediate risk of having, not diagnosed as having, and/or not seeking diagnosis for a cancer such as a colorectal cancer, or any combination thereof.

A sample from a subject, e.g., a human or other mammalian subject, can be a sample of, e.g., blood, blood component, cfDNA, ctDNA, stool, or colorectal tissue. In some particular embodiments, a sample is an excretion or bodily fluid of a subject (e.g., stool, blood, lymph, or urine of a subject) or a colorectal cancer tissue sample. A sample from a subject can be a cell or tissue sample, e.g., a cell or tissue sample that is of a cancer or includes cancer cells, e.g., of a tumor or of a metastatic tissue. In various embodiments, a sample from a subject, e.g., a human or other mammalian subject, can be obtained by biopsy (e.g., fine needle aspiration or tissue biopsy) or surgery.

In various particular embodiments, a sample is a sample of cell-free DNA (cfDNA). cfDNA is typically found in human biofluids (e.g., plasma, serum, or urine) in short, double-stranded fragments. The concentration of cfDNA is typically low, but can significantly increase under particular conditions, including without limitation pregnancy, autoimmune disorder, myocardial infraction, and cancer. Circulating tumor DNA (ctDNA) is the component of circulating DNA specifically derived from cancer cells. ctDNA can be present in human biofluids bound to leukocytes and erythrocytes or not bound to leukocytes and erythrocytes. Various tests for detection of tumor-derived cfDNA are based on detection of genetic or epigenetic modifications that are characteristic of cancer (e.g., of a relevant cancer). Genetic or epigenetic modifications characteristic of cancer can include, without limitation, oncogenic or cancer-associated mutations in tumor-suppressor genes, activated oncogenes, hypermethylation, and/or chromosomal disorders. Detection of genetic or epigenetic modifications characteristic of cancer can confirm that detected cfDNA is ctDNA.

cfDNA and ctDNA provide a real-time or nearly real time metric of the methylation status of a source tissue. cfDNA and ctDNA demonstrate a half-life in blood of about 2 hours, such that a sample taken at a given time provides a relatively timely reflection of the status of a source tissue.

Various methods of isolating nucleic acids from a sample (e.g., of isolating cfDNA from blood or plasma) are known in the art. Nucleic acids can be isolated, e.g., without limitation, standard DNA purification techniques, by direct gene capture (e.g., by clarification of a sample to remove assay-inhibiting agents and capturing a target nucleic acid, if present, from the clarified sample with a capture agent to produce a capture complex, and isolating the capture complex to recover the target nucleic acid).

### Methods of Measuring Methylation Status

Methylation status can be measured by a variety of methods known in the art and/or by methods provided herein. Those of skill in the art will appreciate that a method for measuring methylation status can generally be applied to samples from any source and of any kind, and will further be aware of processing steps available to modify a sample into a form suitable for measurement by a given methodology. Methods of measuring methylation status include, without limitation, methods including whole genome bisulfite sequencing, targeted enzymatic methylation sequencing, methylation-status-specific polymerase chain reaction (PCR), methods including nucleic acid sequencing, methods including mass spectrometry, methods including methylation-specific nucleases, methylation arrays, methods including methylation-specific nucleases, methods including mass-based separation, methods including target-specific capture, and methods including methylation-specific oligonucleotide primers. Certain particular assays for methylation utilize a bisulfite reagent (e.g., hydrogen sulfite ions) or enzymatic conversion reagents (e.g., Tet methylcytosine dioxygenase 2).

Bisulfite reagents can include, among other things, bisulfite, disulfite, hydrogen sulfite, or combinations thereof, which reagents can be useful in distinguishing methylated and unmethylated nucleic acids. Bisulfite interacts differently with cytosine and 5-methylcytosine. In typical bisulfite-based methods, contacting of DNA with bisulfite deaminates unmethylated cytosine to uracil, while methylated cytosine remains unaffected; methylated cytosines, but not unmethylated cytosines, are selectively retained. Thus, in a bisulfite processed sample, uracil residues stand in place of, and thus provide an identifying signal for, unmethylated cytosine residues, while remaining (methylated) cytosine residues thus provide an identifying signal for methylated cytosine residues. Bisulfite processed samples can be analyzed, e.g., by PCR.

Enzymatic conversion reagents can include Tet methylcytosine dioxygenase 2 (TET2). TET2 oxidizes 5-methylcytosine and thus protects it from the consecutive deamination by APOBEC. APOBEC deaminates unmethylated cytosine to uracile, while oxidizes 5-mthylcytosine remains unaffected. Thus, in a TET2 processed sample, uracil residues stand in place of, and thus provide an identifying signal for, unmethylated cytosine residues, while remaining (methylated) cytosine residues thus provide an identifying signal for methylated cytosine residues. TET2 processed samples can be analyzed, e.g., by next generation sequencing (NGS).

Various methylation assay procedures can be used in conjunction with bisulfite treatment or enzymatic treatment to determine methylation status of a target sequence such as a DMR. Such assays can include, among others, whole genome sequencing, targeted sequencing, Methylation-Specific Restriction Enzyme qPCR, sequencing of bisulfite-treated nucleic acid, PCR (e.g., with sequence-specific amplification), Methylation Specific Nuclease-assisted Minor-allele Enrichment PCR, and Methylation-Sensitive High Resolution Melting. In some embodiments, DMRs are amplified from a bisulfite-treated DNA sample and a DNA sequencing library is prepared for sequencing according to, e.g., an Illumina protocol or transpose-based Nextera XT protocol. In certain embodiments, high-throughput and/or next-generation sequencing techniques are used to achieve base-pair level resolution of DNA sequence, permitting analysis of methylation status.

In various embodiments, methylation status is detected by a method including PCR amplification with methylation-specific oligonucleotide primers (MSP methods), e.g., as applied to bisulfite-treated sample (see, e.g., Herman 1992 Proc. Natl. Acad. Sci. USA 93: 9821-9826, which is herein incorporated by reference with respect to methods of determining methylation status). Use of methylation-status-specific oligonucleotide primers for amplification of bisulfite-treated DNA allows differentiation between methylated and unmethylated nucleic acids. Oligonucleotide primer pairs for use in MSP methods include at least one oligonucleotide primer capable of hybridizing with sequence that includes a methylation cite, e.g., a CpG. An oligonucleotide primer that includes a T residue at a position complementary to a cytosine residue will selectively hybridize to templates in which the cytosine was unmethylated prior to bisulfite treatment, while an oligonucleotide primer that includes a G residue at a position complementary to a cytosine residue will selectively hybridize to templates in which the cytosine was methylated cytosine prior to bisulfite treatment. MSP results can be obtained with or without sequencing amplicons, e.g., using gel electrophoresis. MSP (methylation-specific PCR) allows for highly sensitive detection (detection level of 0.1% of the alleles, with full specificity) of locus-specific DNA methylation, using PCR amplification of bisulfite-converted DNA.

Another method that can be used to determine methylation status after bisulfite treatment of a sample is Methylation-Sensitive High Resolution Melting (MS-HRM) PCR (see, e.g., Hussmann 2018 Methods Mol Biol. 1708:551-571, which is herein incorporated by reference with respect to methods of determining methylation status). MS-HRM is an in-tube, PCR-based method to detect methylation levels at specific loci of interest based on hybridization melting. Bisulfite treatment of the DNA prior to performing MS-HRM ensures a different base composition between methylated and unmethylated DNA, which is used to separate the resulting amplicons by high resolution melting. A unique primer design facilitates a high sensitivity of the assays enabling detection of down to 0.1-1% methylated alleles in an unmethylated background. Oligonucleotide primers for MS-HRM assays are designed to be complementary to the methylated allele, and a specific annealing temperature enables these primers to anneal both to the methylated and the unmethylated alleles thereby increasing the sensitivity of the assays.

Another method that can be used to determine methylation status after bisulfite treatment of a sample is Quantitative Multiplex Methylation-Specific PCR (QM-MSP). QM-MSP uses methylation specific primers for sensitive quantification of DNA methylation (see, e.g., Fackler 2018 Methods Mol Biol. 1708:473-496, which is herein incorporated by reference with respect to methods of determining methylation status). QM-MSP is a two-step PCR approach, where in the first step, one pair of gene-specific primers (forward and reverse) amplifies the methylated and unmethylated copies of the same gene simultaneously and in multiplex, in one PCR reaction. This methylation-independent amplification step produces amplicons of up to 10⁹ copies per L after 36 cycles of PCR. In the second step, the amplicons of the first reaction are quantified with a standard curve using real-time PCR and two independent fluorophores to detect methylated/unmethylated DNA of each gene in the same well (e.g., 6FAM and VIC). One methylated copy is detectable in 100,000 reference gene copies.

Another method that can be used to determine methylation status after bisulfite treatment of a sample is Methylation Specific Nuclease-assisted Minor-allele Enrichment (MS-NaME) (see, e.g., Liu 2017 Nucleic Acids Res. 45(6):e39, which is herein incorporated by reference with respect to methods of determining methylation status). Ms-NaME is based on selective hybridization of probes to target sequences in the presence of DNA nuclease specific to double-stranded (ds) DNA (DSN), such that hybridization results in regions of double-stranded DNA that are subsequently digested by the DSN. Thus, oligonucleotide probes targeting unmethylated sequences generate local double stranded regions resulting to digestion of unmethylated targets; oligonucleotide probes capable of hybridizing to methylated sequences generate local double-stranded regions that result in digestion of methylated targets, leaving methylated targets intact. Moreover, oligonucleotide probes can direct DSN activity to multiple targets in bisulfite-treated DNA, simultaneously. Subsequent amplification can enrich non-digested sequences. Ms-NaME can be used, either independently or in combination with other techniques provided herein.

Another method that can be used to determine methylation status after bisulfite treatment of a sample is Methylation-sensitive Single Nucleotide Primer Extension (Ms-SNuPE™) (see, e.g., Gonzalgo 2007 Nat Protoc. 2(8):1931-6, which is herein incorporated by reference with respect to methods of determining methylation status). In Ms-SNuPE, strand-specific PCR is performed to generate a DNA template for quantitative methylation analysis using Ms-SNuPE. SNuPE is then performed with oligonucleotide(s) designed to hybridize immediately upstream of the CpG site(s) being interrogated. Reaction products can be electrophoresed on polyacrylamide gels for visualization and quantitation by phosphor-image analysis. Amplicons can also carry a directly or indirectly detectable labels such as a fluorescent label, radionuclide, or a detachable molecule fragment or other entity having a mass that can be distinguished by mass spectrometry. Detection may be carried out and/or visualized by means of, e.g., matrix assisted laser desorption/ionization mass spectrometry (MALDI) or using electron spray mass spectrometry (ESI).

Certain methods that can be used to determine methylation status after bisulfite treatment of a sample utilize a first oligonucleotide primer, a second oligonucleotide primer, and an oligonucleotide probe in an amplification-based method. For instance, the oligonucleotide primers and probe can be used in a method of real-time polymerase chain reaction (PCR) or droplet digital PCR (ddPCR). In various instances, the first oligonucleotide primer, the second oligonucleotide primer, and/or the oligonucleotide probe selectively hybridize methylated DNA and/or unmethylated DNA, such that amplification or probe signal indicate methylation status of a sample.

Other bisulfite-based methods for detecting methylation status (e.g., the presence of level of 5-methylcytosine) are disclosed, e.g., in Frommer (1992 Proc Natl Acad Sci USA. 1; 89(5):1827-31, which is herein incorporated by reference with respect to methods of determining methylation status).

Certain methods that can be used to determine methylation status do not include bisulfite treatment of a sample. For instance, changes in methylation status can be detected by a PCR-based process in which DNA is digested with one or more methylation-sensitive restriction enzymes (MSREs) prior to PCR amplification (e.g., by MSRE-qPCR). Typically, MSREs have recognition sites that include at least one CpG motif, such that activity of the MSRE is blocked from cleaving a possible recognition site if the site includes 5-methylcytosine. (see, e.g., Beikircher 2018 Methods Mol Biol. 1708:407-424, which is herein incorporated by reference with respect to methods of determining methylation status). Thus, MSREs selectively digest nucleic acids based upon methylation status of the recognition site of the MSRE; they can digest DNA at MSRE recognition sites that are unmethylated, but not digest DNA in MSRE recognition sites that are methylated. In certain embodiments, an aliquot of sample can be digested with MSREs, generating a processed sample in which unmethylated DNA has been cleaved by the MSREs, such that, the proportion of uncleaved and/or amplifiable DNA with at least one methylated site within MSRE recognition sites (e.g., at least one methylated site within each MSRE recognition site of the DNA molecule) is increased relative to uncleaved and/or amplifiable DNA that did not include at least one methylated site within MSRE recognition sites (e.g., did not include at least one methylated site within each MSRE recognition site of the DNA molecule). Uncleaved sequences of a restriction-enzyme-digested sample can then be preamplified, e.g, in PCR, and quantified e.g. by qPCR, real-time PCR, or digital PCR. Oligonucleotide primers for MSRE-qPCR amplify regions that include one or more MSRE cleavage sites, and/or a plurality of MSRE cleavage sites. Amplicons including a plurality of MSRE cleavage sites are typically more likely to yield robust results. The number of cleavage sites within a DMR amplicon, and in some instances the resulting robustness of methylation status determination for the DMR, can be increased by design of DMRs that include a plurality of MSRE recognition sites (as opposed to a single recognition site) in a DMR amplicon. In various instances, a plurality of MSREs can be applied to the same sample, including, e.g., two or more of AciI, Hin6I, HpyCH4IV, and HpaII (e.g., including AciI, Hin6I, and HpyCH4IV). A plurality of MSREs (e.g., the combination of AciI, Hin6I, HpyCH4IV, and HpaII, or the combination of AciI, Hin6I, and HpyCH4IV) can provide improved frequency of MSRE recognition sites within DMR amplicons.

MSRE-qPCR can also include a pre-amplification step following sample digestion by MSREs but before qPCR in order to improve the amount of available sample, given the low prevalence of cfDNA in blood.

In certain MSRE-qPCR embodiments, the amount of total DNA is measured in an aliquot of sample in native (e.g., undigested) form using, e.g., real-time PCR or digital PCR.

Various amplification technologies can be used alone or in conjunction with other techniques described herein for detection of methylation status. Those of skill in the art, having reviewed the present specification, will understand how to combine various amplification technologies known in the art and/or described herein together with various other technologies for methylation status determination known in the art and/or provided herein. Amplification technologies include, without limitation, PCR, e.g., quantitative PCR (qPCR), real-time PCR, and/or digital PCR. Those of skill in the art will appreciate that polymerase amplification can multiplex amplification of multiple targets in a single reaction. PCR amplicons are typically 100 to 2000 base pairs in length. In various instances, an amplification technology is sufficient to determine methylations status.

Digital PCR (dPCR) based methods involve dividing and distributing a sample across wells of a plate with 96-, 384-, or more wells, or in individual emulsion droplets (ddPCR) e.g., using a microfluidic device, such that some wells include one or more copies of template and others include no copies of template. Thus, the average number of template molecules per well is less than one prior to amplification. The number of wells in which amplification of template occurs provides a measure of template concentration. If the sample has been contacted with MSRE, the number of wells in which amplification of template occurs provides a measure of the concentration of methylated template.

In various embodiments a fluorescence-based real-time PCR assay, such as MethyLight™, can be used to measure methylation status (see, e.g., Campan 2018 Methods Mol Biol. 1708:497-513, which is herein incorporated by reference with respect to methods of determining methylation status). MethyLight is a quantitative, fluorescence-based, real-time PCR method to sensitively detect and quantify DNA methylation of candidate regions of the genome. MethyLight is uniquely suited for detecting low-frequency methylated DNA regions against a high background of unmethylated DNA, as it combines methylation-specific priming with methylation-specific fluorescent probing. Additionally, MethyLight can be combined with Digital PCR, for the highly sensitive detection of individual methylated molecules, with use in disease detection and screening.

Real-time PCR-based methods for use in determining methylation status typically include a step of generating a standard curve for unmethylated DNA based on analysis of external standards. A standard curve can be constructed from at least two points and can permit comparison of a real-time Ct value for digested DNA and/or a real-time Ct value for undigested DNA to known quantitative standards. In particular instances, sample Ct values can be determined for MSRE-digested and/or undigested samples or sample aliquots, and the genomic equivalents of DNA can be calculated from the standard curve. Ct values of MSRE-digested and undigested DNA can be evaluated to identify amplicons digested (e.g., efficiently digested; e.g., yielding a Ct value of 45). Amplicons not amplified under either digested or undigested conditions can also be identified. Corrected Ct values for amplicons of interest can then be directly compared across conditions to establish relative differences in methylation status between conditions. Alternatively or additionally, delta-difference between the Ct values of digested and undigested DNA can be used to establish relative differences in methylation status between conditions.

Methods of measuring methylation status can include, without limitation, massively parallel sequencing (e.g., next-generation sequencing) to determine methylation state, e.g., sequencing by-synthesis, real-time (e.g., single-molecule) sequencing, bead emulsion sequencing, nanopore sequencing, or other sequencing techniques known in the art. In some embodiments, a method of measuring methylation status can include whole-genome sequencing, e.g., measuring whole genome methylation status from bisulfite or enzymatically treated material with base-pair resolution.

In some embodiments, methods of measuring methylation status include, without limitation, targeted bisulfite sequencing, targeted enzymatic methylation sequencing, and reduced representation bisulfite sequencing e.g., utilizing use of restriction enzymes to measure methylation status of high CpG content regions from bisulfite or enzymatically treated material with base-pair resolution.

In certain particular embodiments, MSRE-qPCR, among other techniques, can be used to determine the methylation status of an advanced adenoma and/or colorectal cancer (e.g., early stage colorectal cancer) methylation biomarker that is or includes a single methylation locus. In certain particular embodiments, MSRE-qPCR, among other techniques, can be used to determine the methylation status of a methylation biomarker that is or includes two or more methylation loci. In certain particular embodiments, MSRE-qPCR, among other techniques, can be used to determine the methylation status of a methylation biomarker that is or includes a single differentially methylated region (DMR). In certain particular embodiments, MSRE-qPCR, among other techniques, can be used to determine the methylation status of a methylation biomarker that is or includes two or more DMRs. In certain particular embodiments, MSRE-qPCR, among other techniques, can be used to determine the methylation status of a methylation biomarker that is or includes a single methylation site. In certain particular embodiments, MSRE-qPCR, among other techniques, can be used to determine the methylation status of a methylation biomarker that is or includes two or more methylation sites. In various embodiments, a methylation biomarker can be any methylation biomarker provided herein. The present disclosure includes, among other things, oligonucleotide primer pairs for amplification of DMRs, e.g., for amplification of DMRs identified in Table 1.

In certain particular embodiments, a cfDNA sample is derived from subject plasma and contacted with MSREs that are or include one or more of AciI, Hin6I, HpyCH4IV, and HpaII (e.g., AciI, Hin6I, and HpyCH4IV). The digested sample can be preamplified with oligonucleotide primer pairs of one or more DMRs, e.g., with one or more oligonucleotide primer pairs provided in Table 1. Digested DNA, e.g., preamplified digested DNA, can be quantified with qPCR with oligonucleotide primer pairs of one or more DMRs, e.g., with one or more oligonucleotide primer pairs provided in Table 1. qPCR ct values can then be determined and used to determine methylation status of each DMR amplicon.

It will be appreciated by those of skill in the art that oligonucleotide primer pairs provided in Table 1 can be used in accordance with any combination of colorectal cancer methylation biomarkers identified herein. The skilled artisan will be aware that the oligonucleotide primer pairs of Table 1 may be individually included or not included in a given analysis in order to analyze a particular desire combination of DMRs.

The person of skill in the art will further appreciate that while other oligonucleotide primer pairs may be used, selection and pairing of oligonucleotide primers to produce useful DMR amplicons is non-trivial and represents a substantial contribution.

Those of skill in the art will further appreciate that methods, reagents, and protocols for qPCR are well-known in the art. Unlike traditional PCR, qPCR is able to detect the production of amplicons over time in amplification (e.g., at the end of each amplification cycle), often by use of an amplification-responsive fluorescence system, e.g., in combination with a thermocycler with fluorescence-detection capability. Two common types of fluorescent reporters used in qPCR include (i) double-stranded DNA binding dyes that fluoresce substantially more brightly when bound than when unbound; and (ii) labeled oligonucleotides (e.g., labeled oligonucleotide primers or labeled oligonucleotide probes).

Those of skill in the art will appreciate that in embodiments in which a plurality of methylation loci (e.g., a plurality of DMRs) are analyzed for methylation status in a method of screening for colorectal cancer provided herein, methylation status of each methylation locus can be measured or represented in any of a variety of forms, and the methylation statuses of a plurality of methylation loci (preferably each measured and/or represented in a same, similar, or comparable manner) be together or cumulatively analyzed or represented in any of a variety of forms. In various embodiments, methylation status of each methylation locus can be measured as a ct value. In various embodiments, methylation status of each methylation locus can be represented as the difference in ct value between a measured sample and a reference. In various embodiments, methylation status of each methylation locus can be represented as a qualitative comparison to a reference, e.g., by identification of each methylation locus as hypermethylated or not hypermethyated.

In some embodiments in which a single methylation locus is analyzed, hypermethylation of the single methylation locus constitutes a diagnosis that a subject is suffering from or possibly suffering from a condition (e.g., advanced adenoma and/or colorectal cancer, e.g., early stage colorectal cancer), while absence of hypermethylation of the single methylation locus constitutes a diagnosis that the subject is likely not suffering from the condition. In some embodiments, hypermethylation of a single methylation locus (e.g., a single DMR) of a plurality of analyzed methylation loci constitutes a diagnosis that a subject is suffering from or possibly suffering from the condition, while the absence of hypermethylation at any methylation locus of a plurality of analyzed methylation loci constitutes a diagnosis that a subject is likely not suffering from the condition. In some embodiments, hypermethylation of a determined percentage (e.g., a predetermined percentage) of methylation loci (e.g., at least 10% (e.g., at least 10%, at least 20%, at least 30%, at least 40%, at least 50%, at least 60%, at least 70%, at least 80%, at least 90%, or 100%)) of a plurality of analyzed methylation loci constitutes a diagnosis that a subject is suffering from or possibly suffering from the condition, while the absence of hypermethylation of a determined percentage (e.g., a predetermined percentage) of methylation loci (e.g., at least 10% (e.g., at least 10%, at least 20%, at least 30%, at least 40%, at least 50%, at least 60%, at least 70%, at least 80%, at least 90%, or 100%)) of a plurality of analyzed methylation loci constitutes a diagnosis that a subject is not likely suffering from the condition. In some embodiments, hypermethylation of a determined number (e.g., a predetermined number) of methylation loci (e.g., at least 1, 2, 3, 4, 5, 6, 7, 8, 9, 10, 11, 12, 13, 14, 15, 16, 17, 18, 19, or 20 DMRs) of a plurality of analyzed methylation loci (e.g., 2, 3, 4, 5, 6, 7, 8, 9, 10, 11, 12, 13, 14, 15, 16, 17, 18, 19, or 20 DMRs) constitutes a diagnosis that a subject is suffering from or possibly suffering from the condition, while the absence of hypermethylation of a determined number (e.g., a predetermined number) of methylation loci (e.g., at least 1, 2, 3, 4, 5, 6, 7, 8, 9, 10, 11, 12, 13, 14, 15, 16, 17, 18, 19, or 20 DMRs) of a plurality of analyzed methylation loci (e.g., 2, 3, 4, 5, 6, 7, 8, 9, 10, 11, 12, 13, 14, 15, 16, 17, 18, 19, or 20 DMRs) constitutes a diagnosis that a subject is not likely suffering from the condition.

In some embodiments, methylation status of a plurality of methylation loci (e.g., a plurality of DMRs) is measured qualitatively or quantitatively and the measurement for each of the plurality of methylation loci are combined to provide a diagnosis. In some embodiments, the qualitative of quantitatively measured methylation status of each of a plurality of methylation loci is individually weighted, and weighted values are combined to provide a single value that can be comparative to a reference in order to provide a diagnosis. To provide but one example of such an approach, support vector machine (SVM) algorithm can be used to analyze the methylation statuses of a plurality of methylation loci of the present disclosure to produce a diagnosis. At least one objective of the support vector machine algorithm is to identify a hyperplane in an N-dimensional space (N—the number of features) that distinctly classifies the data points with the objective to find a plane that has the maximum margin, i.e. the maximum distance between data points of both classes. As discussed in the present Examples, an SVM model is built on marker values (e.g., ct values) derived from a training sample set (e.g., the first subject group and/or the second subject group) that are transformed to support vector values upon which a prediction is made. In application of the SVM model to new samples, samples will be mapped onto vectoral space the model and categorized as having a probability of belonging to the first condition or the second condition, e.g., based on each new sample's location relative to the gap between the two conditions. Those of skill in the art will appreciate that, once relevant compositions and methods have been identified, vector values can be used in conjunction with an SVM algorithm defined by predict ( ) function of R-package (see Hypertext Transfer Protocol Secure (HTTPS)://cran.r-project.org/web/packages/e1071/index.html, the SVM of which is hereby incorporated by reference) to easily generate a prediction on a new sample. Accordingly, with compositions and methods for advanced adenoma and/or colorectal cancer diagnosis disclosed herein in hand (and only then), generation of a predictive model utilizing algorithm input information in combination to predict ( ) function of R-package (see Hypertext Transfer Protocol Secure (HTTPS)://cran.r-project.org/web/packages/e1071/index.html, the SVM of which is hereby incorporated by reference) to provide condition diagnosis would be straightforward.

### Applications

Methods and compositions of the present disclosure can be used in any of a variety of applications. For example, methods and compositions of the present disclosure can be used to screen, or aid in screening for, advanced adenoma and/or colorectal cancer (e.g., early stage colorectal cancer). In various instances, screening using methods and compositions of the present disclosure can detect any stage of colorectal cancer, including without limitation early-stage colorectal cancer. In some embodiments, screening using methods and compositions of the present disclosure is applied to individuals 50 years of age or older, e.g., 50, 55, 60, 65, 70, 75, 80, 85, or 90 years or older. In some embodiments, screening using methods and compositions of the present disclosure is applied to individuals 20 years of age or older, e.g., 20, 25, 30, 35, 40, 45, 50, 55, 60, 65, 70, 75, 80, 85, or 90 years or older. In some embodiments, screening using methods and compositions of the present disclosure is applied to individuals 20 to 50 years of age, e.g., 20 to 30 years of age, 20 to 40 years of age, 20 to 50 years of age, 30 to 40 years of age, 30 to 50 years of age, or 40 to 50 years of age. In various embodiments, screening using methods and compositions of the present disclosure is applied to individuals experiencing abdominal pain or discomfort, e.g., experiencing undiagnosed or incompletely diagnosed abdominal pain or discomfort. In various embodiments, screening using methods and compositions of the present disclosure is applied to individuals experiencing no symptoms likely to be associated with advanced adenoma and/or colorectal cancer. Thus, in certain embodiments, screening using methods and compositions of the present disclosure is fully or partially preventative or prophylactic, at least with respect to later or non-early stages of colorectal cancer.

In various embodiments, screening using methods and compositions of the present disclosure can be applied to an asymptomatic human subject. As used herein, a subject can be referred to as “asymptomatic” if the subject does not report, and/or demonstrate by non-invasively observable indicia (e.g., without one, several, or all of device-based probing, tissue sample analysis, bodily fluid analysis, surgery, or colorectal cancer screening), sufficient characteristics of the condition to support a medically reasonable suspicion that the subject is likely suffering from the condition. Detection of advanced adenoma and/or early stage colorectal cancer is particularly likely in asymptomatic individuals screened in accordance with methods and compositions of the present disclosure.

Those of skill in the art will appreciate that regular, preventative, and/or prophylactic screening for advanced adenoma and/or colorectal cancer improves diagnosis. As noted above, early stage cancers include, according to at least one system of cancer staging, Stages 0 to II C of colorectal cancer. Thus, the present disclosure provides, among other things, methods and compositions particularly useful for the diagnosis and treatment of advanced adenoma and/or early stage colorectal cancer. Generally, and particularly in embodiments in which screening in accordance with the present disclosure is carried out annually, and/or in which a subject is asymptomatic at time of screening, methods and compositions of the present invention are especially likely to detect early stage colorectal cancer.

In various embodiments colorectal cancer screening in accordance with the present disclosure is performed once for a given subject or multiple times for a given subject. In various embodiments, colorectal cancer screening in accordance with the present disclosure is performed on a regular basis, e.g., every six months, annually, every two years, every three years, every four years, every five years, or every ten years.

In various embodiments, screening using methods and compositions disclosed herein will be provide a diagnosis of cancer condition. In other instances, screening for colorectal cancer using methods and compositions disclosed herein will be indicative of condition diagnosis but not definitive for condition diagnosis. In various instances, screening using methods and compositions of the present disclosure can be followed by a further diagnosis-confirmatory assay, which further assay can confirm, support, undermine, or reject a diagnosis resulting from prior screening, e.g., screening in accordance with the present disclosure. As used herein, a diagnosis-confirmatory assay can be a colorectal cancer assay that provides a diagnosis recognized as definitive by medical practitioners, e.g., a colonoscopy-based diagnosed, or a colorectal cancer assay that substantially increases or decreases the likelihood that a prior diagnosis was correct, e.g., a diagnosis resulting from screening in accordance with the present disclosure. Diagnosis-confirmatory assays could include existing screening technologies, which are generally in need of improvement with respect to one or more of sensitivity, specificity, and non-invasiveness, particularly in the detection of early stage colorectal cancers.

In some instances, a diagnosis-confirmatory assay is a test that is or includes a visual or structural inspection of subject tissues, e.g., by colonoscopy. In some embodiments, colonoscopy includes or is followed by histological analysis. Visual and/or structural assays for colorectal cancer can include inspection of the structure of the colon and/or rectum for any abnormal tissues and/or structures. Visual and/or structural inspection can be conducted, for example, by use of a scope via the rectum or by CT-scan. In some instances, a diagnosis-confirmatory assay is a colonoscopy, e.g., including or followed by histological analysis. According to some reports, colonoscopy is currently the predominant and/or most relied upon diagnosis-confirmatory assay.

Another visual and/or structural diagnosis confirmatory assay based on computer tomography (CT) is CT colonography, sometimes referred to as virtual colonoscopy. A CT scan utilizes numerous x-ray images of the colon and/or rectum to produce dimensional representations of the colon. Although useful as a diagnosis-confirmatory assay, some reports suggest that CT colonography is not sufficient for replacement of colonoscopy, at least in part because a medical practitioner has not physically accessed the subject's colon to obtain tissue for histological analysis.

Another diagnosis-confirmatory assay can be a sigmoidoscopy. In sigmoidoscopy, a sigmoidoscope is used via the rectum to image portions of the colon and/or rectum. According to some reports, sigmoidoscopy is not widely used.

In some instances, a diagnosis-confirmatory assay is a stool-based assay. Typically, stool-based assays, when used in place of visual or structural inspection, are recommended to be utilized at a greater frequency than would be required if using visual or structural inspection. In some instances, a diagnosis-confirmatory assay is a guiac-based fecal occult blood test or a fecal immunochemical test (gFOBTs/FITs) (see, e.g., Navarro 2017 World J Gastroenterol. 23(20):3632-3642, which is herein incorporated by reference with respect to colorectal cancer assays). FOBTs and FITs are sometimes used for diagnosis of colorectal cancer (see, e.g., Nakamura 2010 J Diabetes Investig. October 19; 1(5):208-11, which is herein incorporated by reference with respect to colorectal cancer assays). FIT is based on detection of occult blood in stool, the presence of which is often indicative of colorectal cancer but is often not in sufficient volume to permit identification by the unaided eye. For example, in a typical FIT, the test utilizes hemoglobin-specific reagent to test for occult blood in a stool sample. In various instances, FIT kits are suitable for use by individuals in their own homes. When used in the absence of other diagnosis-confirmatory assays, FIT may be recommended for use on an annual basis. FIT is generally not relied upon to provide sufficient diagnostic information for conclusive diagnosis of colorectal cancer.

Diagnosis-confirmatory assays also include gFOBT, which is designed to detect occult blood in stool by chemical reaction. Like FIT, when used in the absence of other diagnosis-confirmatory assays, gFOBT may be recommended for use on an annual basis. gFOBT is generally not relied upon to provide sufficient diagnostic information for conclusive diagnosis of colorectal cancer.

Diagnosis-confirmatory assays can also include stool DNA testing. Stool DNA testing for colorectal cancer can be designed to identify DNA sequences characteristic of cancer in stool samples. When used in the absence of other diagnosis-confirmatory assays, stool DNA testing may be recommended for use every three years. Stool DNA testing is generally not relied upon to provide sufficient diagnostic information for conclusive diagnosis of colorectal cancer.

One particular screening technology is a stool-based screening test (Cologuard® (Exact Sciences Corporation, Madison, Wis., United States), which combines an FIT assay with analysis of DNA for abnormal modifications, such as mutation and methylation. The Cologuard® test demonstrates improved sensitivity as compared to FIT assay alone, but can be clinically impracticable or ineffective due to low compliance rates, which low compliance rates are at least in part due to subject dislike of using stool-based assays (see, e.g., doi: 10.1056/NEJMc1405215 (e.g., 2014 N Engl J Med. 371(2):184-188)). The Cologuard® test appears to leave almost half of the eligible population out of the screening programs (see, e.g., van der Vlugt 2017 Br J Cancer. 116(1):44-49). Use of screening as provided herein, e.g., by a blood-based analysis, would increase the number of individuals electing to screen for colorectal cancer (see, e.g., Adler 2014 BMC Gastroenterol. 14:183; Liles 2017 Cancer Treatment and Research Communications 10: 27-31). To present knowledge, only one existing screening technology for colorectal cancer, Epiprocolon, is FDA-approved and CE-IVD marked and is blood-based. Epiprocolon is based on hypermethylation of SEPT9 gene. The Epiprocolon test suffers from low accuracy for colorectal cancer detection with sensitivity of 68% and advanced adenoma sensitivity of only 22% (see, e.g., Potter 2014 Clin Chem. 60(9):1183-91). There is need in the art for, among other things, a non-invasive colorectal cancer screen that will likely achieve high subject adherence with high and/or improved specificity and/or sensitivity.

In various embodiments, screening in accordance with methods and compositions of the present disclosure reduces colorectal cancer mortality, e.g., by early colorectal cancer diagnosis. Data supports that colorectal cancer screening reduces colorectal cancer mortality, which effect persisted for over 30 years (see, e.g., Shaukat 2013 N Engl J Med. 369(12):1106-14). Moreover, colorectal cancer is particularly difficult to treat at least in part because colorectal cancer, absent timely screening, may not be detected until cancer is past early stages. For at least this reason, treatment of colorectal cancer is often unsuccessful. To maximize population-wide improvement of colorectal cancer outcomes, utilization of screening in accordance with the present disclosure can be paired with, e.g., recruitment of eligible subjects to ensure widespread screening.

In various embodiments, screening of colorectal cancer including one or more methods and/or composition s disclosed herein is followed by treatment of colorectal cancer, e.g., treatment of early stage colorectal cancer. In various embodiments, treatment of colorectal cancer, e.g., early stage colorectal cancer, includes administration of a therapeutic regimen including one or more of surgery, radiation therapy, and chemotherapy. In various embodiments, treatment of colorectal cancer, e.g., early stage colorectal cancer, includes administration of a therapeutic regimen including one or more of treatments provided herein for treatment of stage 0 colorectal cancer, stage I colorectal cancer, and/or stage II colorectal cancer.

In various embodiments, treatment of colorectal cancer includes treatment of early stage colorectal cancer, e.g., stage 0 colorectal cancer or stage I colorectal cancer, by one or more of surgical removal of cancerous tissue e.g., by local excision (e.g., by colonoscope), partial colectomy, or complete colectomy.

In various embodiments, treatment of colorectal cancer includes treatment of early stage colorectal cancer, e.g., stage II colorectal cancer, by one or more of surgical removal of cancerous tissue (e.g., by local excision (e.g., by colonoscope), partial colectomy, or complete colectomy), surgery to remove lymph nodes near to identified colorectal cancer tissue, and chemotherapy (e.g., administration of one or more of 5-FU and leucovorin, oxaliplatin, or capecitabine).

In various embodiments, treatment of colorectal cancer includes treatment of stage III colorectal cancer, by one or more of surgical removal of cancerous tissue (e.g., by local excision (e.g., by colonoscopy-based excision), partial colectomy, or complete colectomy), surgical removal of lymph nodes near to identified colorectal cancer tissue, chemotherapy (e.g., administration of one or more of 5-FU, leucovorin, oxaliplatin, capecitabine, e.g., in a combination of (i) 5-FU and leucovorin, (ii) 5-FU, leucovorin, and oxaliplatin (e.g., FOLFOX), or (iii) capecitabine and oxaliplatin (e.g., CAPEOX)), and radiation therapy.

In various embodiments, treatment of colorectal cancer includes treatment of stage IV colorectal cancer, by one or more of surgical removal of cancerous tissue (e.g., by local excision (e.g., by colonoscope), partial colectomy, or complete colectomy), surgical removal of lymph nodes near to identified colorectal cancer tissue, surgical removal of metastases, chemotherapy (e.g., administration of one or more of 5-FU, leucovorin, oxaliplatin, capecitabine, irinotecan, VEGF-targeted therapeutic agent (e.g., bevacizumab, ziv-aflibercept, or ramucirumab), EGFR-targeted therapeutic agent (e.g., cetuximab or panitumumab), Regorafenib, trifluridine, and tipiracil, e.g., in a combination of or including (i) 5-FU and leucovorin, (ii) 5-FU, leucovorin, and oxaliplatin (e.g., FOLFOX), (iii) capecitabine and oxaliplatin (e.g., CAPEOX), (iv) leucovorin, 5-FU, oxaliplatin, and irinotecan (FOLFOXIRI), and (v) trifluridine and tipiracil (Lonsurf)), radiation therapy, hepatic artery infusion (e.g., if cancer has metastasized to liver), ablation of tumors, embolization of tumors, colon stent, colorectomy, colostomy (e.g., diverting colostomy), and immunotherapy (e.g., pembrolizumab).

Those of skill in the art that treatments of colorectal cancer provided herein can be utilized, e.g., as determined by a medical practitioner, alone or in any combination, in any order, regimen, and/or therapeutic program. Those of skill in the art will further appreciate that advanced treatment options may be appropriate for earlier stage cancers in subjects previously having suffered a cancer or colorectal cancer, e.g., subjects diagnosed as having a recurrent colorectal cancer.

In some embodiments, methods and compositions for colorectal cancer screening provided herein can inform treatment and/or payment (e.g., reimbursement for or reduction of cost of medical care, such as screening or treatment) decisions and/or actions, e.g., by individuals, healthcare facilities, healthcare practitioners, health insurance providers, governmental bodies, or other parties interested in healthcare cost.

In some embodiments, methods and compositions for colorectal cancer screening provided herein can inform decision making relating to whether health insurance providers reimburse a healthcare cost payer or recipient (or not), e.g., for (1) screening itself (e.g., reimbursement for screening otherwise unavailable, available only for periodic/regular screening, or available only for temporally- and/or incidentally-motivated screening); and/or for (2) treatment, including initiating, maintaining, and/or altering therapy, e.g., based on screening results. For example, in some embodiments, methods and compositions for colorectal cancer screening provided herein are used as the basis for, to contribute to, or support a determination as to whether a reimbursement or cost reduction will be provided to a healthcare cost payer or recipient. In some instances, a party seeking reimbursement or cost reduction can provide results of a screen conducted in accordance with the present specification together with a request for such reimbursement or cost reduction of a healthcare cost. In some instances, a party making a determination as to whether or not to provide a reimbursement or cost reduction of a healthcare cost will reach a determination based in whole or in part upon receipt and/or review of results of a screen conducted in accordance with the present specification.

For the avoidance of any doubt, those of skill in the art will appreciate from the present disclosure that methods and compositions for colorectal cancer diagnosis of the present specification are at least for in vitro use. Accordingly, all aspects and embodiments of the present disclosure can be performed and/or used at least in vitro.

### Kits

The present disclosure includes, among other things, kits including one or more compositions for use in screening as provided herein, optionally in combination with instructions for use thereof in screening (e.g., screening for advanced adenoma and/or colorectal cancer, e.g., early-stage colorectal cancer). In various embodiments, a kit for screening can include one or more of: one or more oligonucleotide primers (e.g., one or more oligonucleotide primer pairs, e.g., as found in Table 1), one or more MSREs, one or more reagents for qPCR (e.g., reagents sufficient for a complete qPCR reaction mixture, including without limitation dNTP and polymerase), and instructions for use of one or more components of the kit for colorectal cancer screening. In various embodiments, a kit for screening of colorectal cancer can include one or more of: one or more oligonucleotide primers (e.g., one or more oligonucleotide primer pairs, e.g., as found in Table 1), one or more bisulfite reagents, one or more reagents for qPCR (e.g., reagents sufficient for a complete qPCR reaction mixture, including without limitation dNTP and polymerase), and instructions for use of one or more components of the kit for colorectal cancer screening.

In certain embodiments, a kit of the present disclosure includes at least one oligonucleotide primer pair for amplification of a methylation locus and/or DMR as disclosed herein.

In some instances, a kit of the present disclosure includes one or more oligonucleotide primer pairs for amplification of one or more methylation loci of the present disclosure. In some instances, kit of the present disclosure includes one or more oligonucleotide primer pairs for amplification of one or more methylation loci that are or include all or a portion of one or more genes provided in Table 1. In some particular instances, a kit of the present disclosure includes oligonucleotide primer pairs for a plurality of methylation loci that each are or include all or a portion of a gene identified in Table 1, the plurality of methylation loci including, e.g. 3, 4, 5, 6, 7, 8, 9, 10, 11, 12, 13, 14, 15, 16, or more methylation loci, e.g., as provided Table 1.

In some instances, a kit of the present disclosure includes one or more oligonucleotide primer pairs for amplification of one or more DMRs of the present disclosure. In some instances, kit of the present disclosure includes one or more oligonucleotide primer pairs for amplification of one or more DMRs that are, include all or a portion of, or are within a gene identified in Table 1. In some particular embodiments, a kit of the present disclosure includes oligonucleotide primer pairs for a plurality of DMRs each of which is, includes all or a portion of, or is within a gene identified in Table 1, e.g., 2, 3, 4, 5, 6, 7, 8, 9, 10, 11, 12, 13, 14, 15, 16 or more DMRs.

A kit of the present disclosure can further include one or more MSREs individually or in a single solution. In various embodiments, one or more MSREs are selected from the set of MSREs including AciI, Hin6I, HpyCH4IV, and HpaII (e.g., such that the kit includes AciI, Hin6I, and HpyCH4IV, either individually or in a single solution). In certain embodiments, a kit of the present disclosure includes one or more reagents for qPCR (e.g., reagents sufficient for a complete qPCR reaction mixture, including without limitation dNTP and polymerase).

### EXAMPLES

### Biomarker Discovery

The present example includes identification of biomarkers (e.g., CpG loci) that are hypermethylated in one or more of colorectal cancer and advanced adenoma as compared to healthy control subjects. Healthy controls subjects are colonoscopy verified controls who do not have any significant findings, such as advanced adenomas and/or colorectal cancer. In the present example, methods and systems for identifying DMRs, CpG loci and CpGs for further analysis is disclosed in an initial phase of biomarker selection.

In the initial phase of potential marker selection, plasma was gathered from (i) healthy patients, (ii) patients with advanced adenomas, and (iii) patients with colorectal cancer. Plasma was pooled into 4 separate groups: (i) healthy patient controls (CTR), (ii) advanced adenoma (AA), (iii) stage 1 cancer (CRC1), and (iv) mixed stage cancer (CRC). CTR, AA and CRC pools were divided into 3 technical replicates and each of the technical replicates were treated as individual samples.

Cell-free DNA (cfDNA) was extracted from each pool with QIAamp Circulating Nucleic Acid Kit. Extracted cfDNA was bisulfide-converted with EZ DNA Methylation-Lightning kit (ZymoResearch). Sequencing libraries were prepared from the bisulfite converted cfDNA by using Accel-NGS Methyl-seq DNA library kit (Swift Biosciences) and consequently sequenced with average depth of 37.5× with NovaSeq6000 (Illumina) equipment, using paired-end sequencing (2×150 bp). The sequenced reads were aligned to a bisulfite-converted human genome (Ensembl 91 assembly), using Bisulfite Read Mapper with Bowtie 2 following standard steps:
Evaluate the sequencing quality
Align to reference genome (hg38)
Deduplication and cleaning from adapter dimers
Methylation calling

CpG counts were normalized by their library size and the mean coverage of technical replicas was calculated. Strand coverage (for each CpG) also showed that the majority of reads align fully to either forward or reverse strand, or half the coverage coming from both strands. The counts from the forward and reverse strands were summed and the methylation proportion was calculated. Coverage distribution of methylated CpGs shows that the majority of CpGs (out of all CpGs in the genome) were covered in plasma, with a peak around coverage 9 in all groups. Coverage was slightly higher in the late stage CRC group.

CpG dense regions were defined by adjusting the maximum distance between neighboring CpGs. In the current example, the hg38 assembly of the human genome is used. Based on this distance threshold, CpGs were grouped into loci. The maximum distance thresholds between neighboring CpGs was adjusted from 4 to 395 bps (base pairs). From these loci, the length and the mean distance between CpGs were calculated for each of the distance thresholds.

The size of cfDNA fragments in blood primarily depends on the length of DNA wrapped around nucleosomes, which protect the fragments from degradation. The two main structures which protect cfDNA fragments are nucleosomes and chromatosomes (i.e., nucleosome+linker histone). These structures protect ˜147 bp and ˜167 bp of DNA, respectively. A distance of 77 bp, which represents the 65th percentile of cfDNA fragment lengths, was selected. In this instance, the median locus size is 140 bp and the median distance between CpGs within a locus is 43 bp.

First, CpG counts (e.g., a read coverage) lower than 4 were filtered and CpGs with a coverage higher than 90 were also filtered out, since these primarily represent misalignments. For all defined loci, mean Methylation and Coverage were calculated from all CpGs and the top 5 CpGs (e.g., CpGs having the highest methylation difference between conditions) within a locus.

Marker selection was focused on maximizing the difference in methylation between plasma from control subjects (CTR) and advanced adenoma subjects (AA). For each locus, the following values were calculated: AA (advanced adenoma) CpG coverage, AA methylation difference (compared to CTR), CTR fraction of methylation, CTR coverage, and number of CpGs within a locus. Top loci were selected using the following filters: AA methylation difference >0.2, CTR fraction of methylation <0.1 (e.g., fraction of methylated CpGs being less than 0.1% of CpGs). All regions were also inspected visually. The final filtering and selection identified 147 differentially methylated regions (DMRs) with 250 single CpGs.

### MSRE-qPCR Validation of Selected Regions

For screening purposes, it is important to allow diagnostic marker detection from a readily obtainable biospecimen (e.g., a biological sample). In certain embodiments, the biological sample may be blood, a blood product (e.g., plasma), urine, tissue, or stool. Confirming tissue markers in blood or plasma however is challenging due to low concentration of circulating tumor-derived DNA (0.1-1%) as compared to the non-tumor DNA background of the sample. For blood-based confirmatory testing, Methylation-Sensitive Restriction Enzyme (MSRE)-qPCR allows for detection of <10 copies of targets in highly multiplexed format, making it suitable for use in a context where circulating DNA (e.g., cfDNA) is found in low amounts.

CpG-rich regions, which are also candidate regions for methylation differences, are targets for MSRE-qPCR assay design, as they usually contain a large number of MSRE cleavage sites. MSRE-qPCR assays can utilize multiple restriction enzymes to enhance the range of colorectal cancer and/or advanced adenoma methylation biomarker sites that can be assayed by a single MSRE-qPCR reaction, as a single MSRE is unlikely to cleave sites that together include all methylation biomarker sites of interest. MSRE-qPCR assays of the present Examples utilize the MSREs AciI, Hin6I, HpyCH4IV, and HpaII, which together are presently found to provide sufficient coverage.

In MSRE-qPCR, “native” DNA is targeted with no prior chemical alterations required. However, primer selection requires coverage of a target region that presents at least one advanced adenoma and/or colorectal cancer MSRE cleavage site (i.e., an MSRE cleavage site that covers at least one colorectal cancer and/or advanced adenoma methylation biomarker site, such that cleavage of the MSRE cleavage site is permitted in nucleic acid molecules where all of the at least one colorectal cancer and/or advanced adenoma methylation biomarker sites are unmethylated and blocked in nucleic acid molecules where at least one of the at least one colorectal cancer methylation biomarker sites is methylated).

From the initial 250 CpG targets, 147 assays were developed with primer-pairs covering at least 1 restriction-enzyme cut-site (e.g., a MSRE cleavage site). Additionally, methylation of 4 established control genes (JUB, H19, SNRPN, IRF4) was measured to assure the robustness and reproducibility of each assay run. All assays were then evaluated for their utility for plasma-based marker detection and clinical prediction power by using DNA extracted from plasma of patients found to have advanced adenomas, colorectal cancer and control (including colonoscopy negative patients, patients with hyperplastic polyps and patients with non-malignant gastrointestinal diseases) patients.

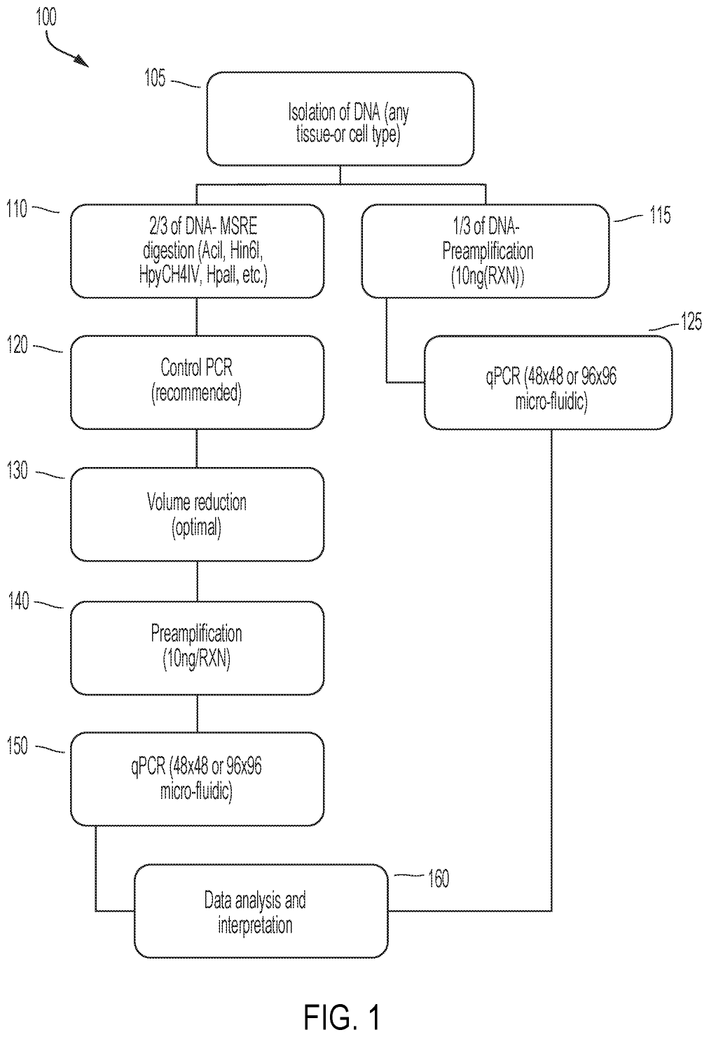

A general assay work-flow (100) can be schematically seen in FIG. 1. As performed in the present examples, cfDNA was extracted from blood of a subject (about 4 ml of a plasma sample).

As shown in FIG. 1, isolated cfDNA was divided into two aliquots, a first of which aliquots is utilized in a qPCR quality control analysis (115), and a second of which aliquots is used in MSRE-qPCR (110). cfDNA (cell free DNA) was extracted (105) from the sample with QIAamp MinElute ccfDNA Kit for manual isolation of the samples following a protocol defined by the manufacturer (QIAamp MinElute ccfDNA Handbook 08/2018, Qiagene). As shown in FIG. 1, ⅓ of the eluted cfDNA volume (115) was directly used for PCR amplification of the target regions and consecutive qPCR (125) analysis. This reaction functions as a quality control, showing whether a target of interest is detectable and quantifiable from plasma in its “native” DNA format.

The remaining ⅔ of the initially eluted cfDNA volume was used for digestion with methylation specific restriction enzymes (MSREs) (110). In the present example, AciI, Hin6I or HpyCH4IV were selected as the methylation specific restriction enzymes. DMRs typically include 1-15 MSRE cleavage sites to enrich for the methylation-derived signal. Methylation sensitive restriction enzymes detect unmethylated DNA regions and consecutively the DNA strand is digested and thus eliminated from the sample, leaving only the methylated regions intact and quantifiable. A control PCR assay is recommended following the disgestion of DNA with MSREs (120). Following volume reduction (optimally) (130), preamplification (140) of the sample occurs. Next, a qPCR assay is employed on the sample (150). Finally, data analysis and interpretation of results obtained from qPCR (160) and the quality control qPCR (125) occurs (e.g., as described herein).

### Experimental Methods

To probe clinical diagnostic and prognostic power of identified methylation biomarkers, the DMRs amplified by the MSRE-qPCR oligonucleotide primer pairs covering 147 methylation biomarker sites (e.g., DMRs), and appropriate controls, were assayed in cfDNA extracted from plasma of human subjects.

Samples were collected from 150 participants attending colorectal cancer screening centers and oncology clinics in Spain and US during 2017-2019. Sample cohorts are described in Table 2. Table 2 contains a table which describes characteristics of the pilot cohort and validation cohort used in the training and validation studies described herein. The pilot cohort samples (or a portion thereof) were used for initial marker evaluation and prediction model development. The validation cohort samples (or a portion thereof) were used for validation of the prediction model.

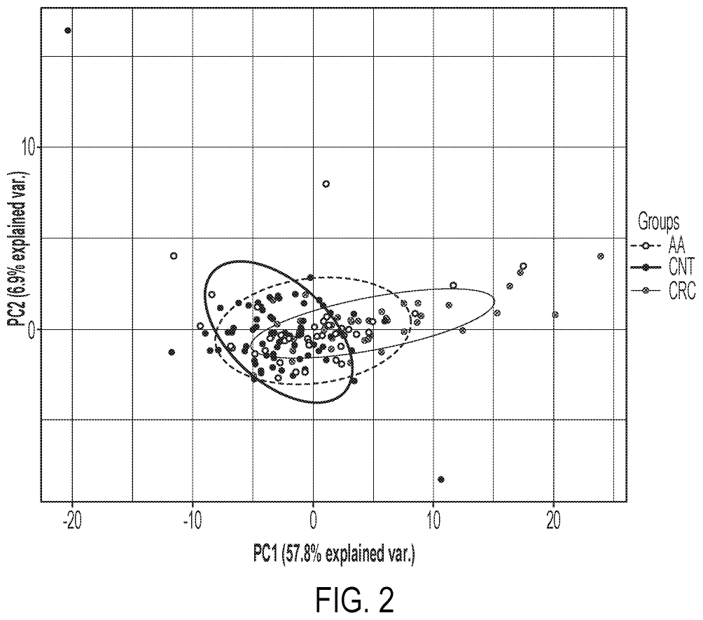

The qPCR cycle threshold (ct) values were used for data analyses. For simplification of visualization, all ct-values were subtracted from the maximum threshold value of 45.01. R version 3.3.2 software was used for data analysis. Unsupervised analysis by principal component analysis (PCA) with the function prcomp was first used for evaluating the general discriminative power of methylation markers. As can be seen from FIG. 2, clear separation could be seen between colorectal cancer (CRC) and healthy controls+ patients with hyperplastic polyps+GID (CNT) group. Good separation could also be seen between advanced adenoma (AA) and healthy controls+ patients with hyperplastic polyps+GID (CNT) group, which indicates that there are components (e.g., markers) that have potential for predicting (e.g., diagnosing) advanced adenoma.

TABLE 2
Describing sample cohort used in this study, indicating samples used in Pilot
cohort for initial marker evaluation and prediction model development and Validation
cohort samples that were used for prediction algorithm validation

| Controls (healthy + hyperplastic polyps + gastrointestinal disease) | | | Cases (CRC + AA) | |
|---|---|---|---|---|
| | Pilot cohort | Validation cohort | Pilot cohort | Validation cohort |
| Characteristics | (n = 30) | (n = 40) | (n = 48) | (n = 32) |
| Age (years, average (IQR)) | 62 (51-76) | 60 (47-82) | 62 (44-78) | 58 (47-68) |
| Gender (n (%)) | | | | |
| Female | 15 (50%) | 20 (50%) | 24 (50%) | 14 (44%) |
| Male | 15 (50%) | 20 (50%) | 24 (50%) | 18 (56%) |
| Healthy controls | 21 | 18 | | |
| Gastrointestinal disease | / | 10 | | |
| Hyperplastic polyps | 9 | 12 | | |
| Stage | | | | |
| Stage I | | | 4 | 3 |
| Stage II | | | 8 | 5 |
| Stage III | | | 6 | 8 |
| Stage IV | | | 6 | / |
| Location in colon | | | | |
| Proximal colon | | | 14 | 6 |
| Distal colon | | | 10 | 9 |
| Unknown | | | | 1 |
| Adenoma characteristics | | | | |
| High grade dysplasia | | | 2 | 8 |
| Size >=10 mm | | | 22 | 8 |
| Location in colon | | | | |
| Proximal colon | | | 10 | 8 |
| Distal colon | | | 12 | 8 |
| Unknown | | | 2 | |

### Evaluation of Models for Marker Performance

In the present example, performance of the markers is evaluated in order to identify panels DMRs useful for diagnosis and/or classification of colorectal cancer and/or advanced adenoma in subjects.

Further analysis was performed to evaluate the performance of combinations of markers (e.g., combinations of DMRs) for building a prediction model. The model allows for detection of colorectal cancer (CRC) and/or advanced adenomas (AA) in cfDNA of plasma of subjects. Control samples (CNT) includes patients with hyperplastic polyps and with gastrointestinal diseases (GIDs).

78 plasma samples were obtained from 30 control subjects and 48 subjects having CRC or AA. These subjects were selected from the pilot cohort and used for training the algorithm. The algorithm was trained using Monte-Carlo cross-validation over 50 runs by sub-setting iterations on the training set to rank the pre-selected markers. A random forest algorithm was used for feature selection and a SBS method for ranking the markers for each run. Finally, support-vector machine (SVM) algorithm was used to build the classification model over best performing markers according to SBS. The SVM-model was then applied on the remaining samples (Table 2: Validation cohort).

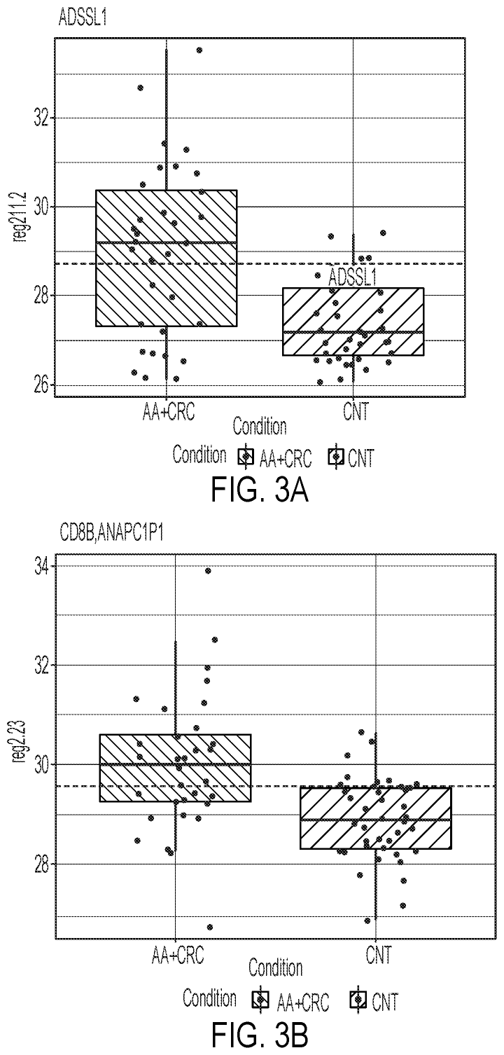

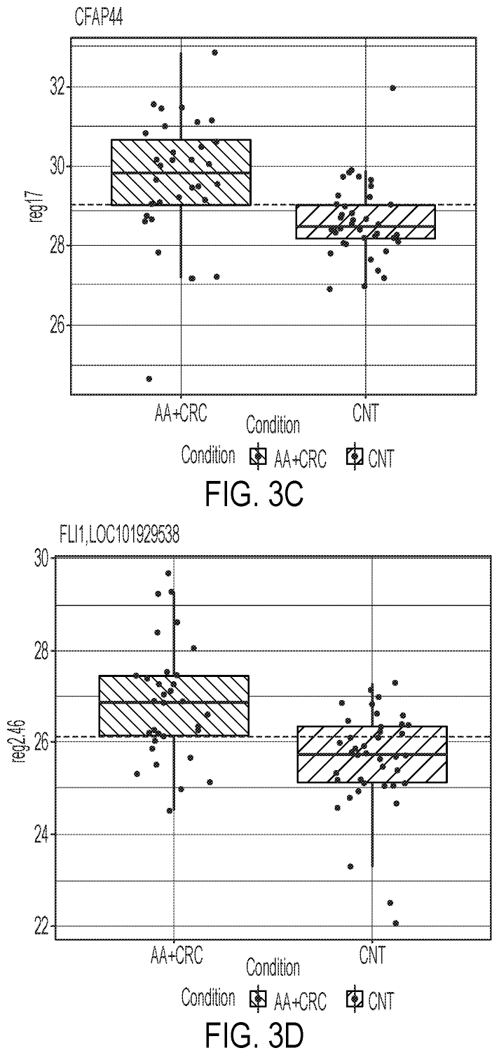

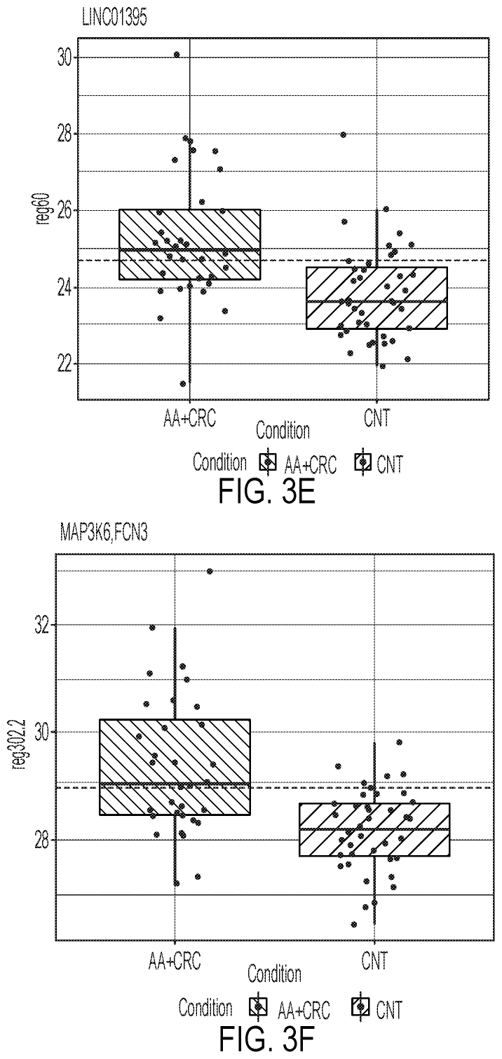

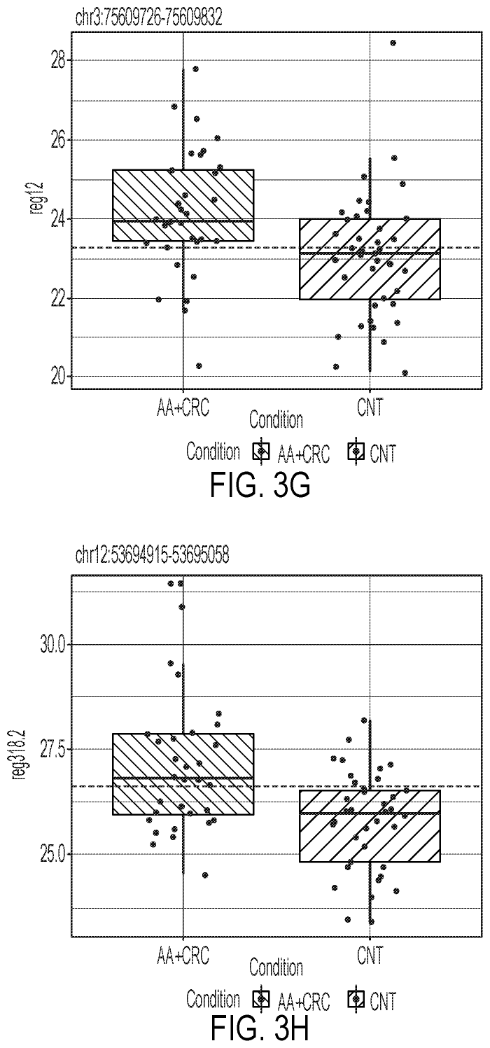

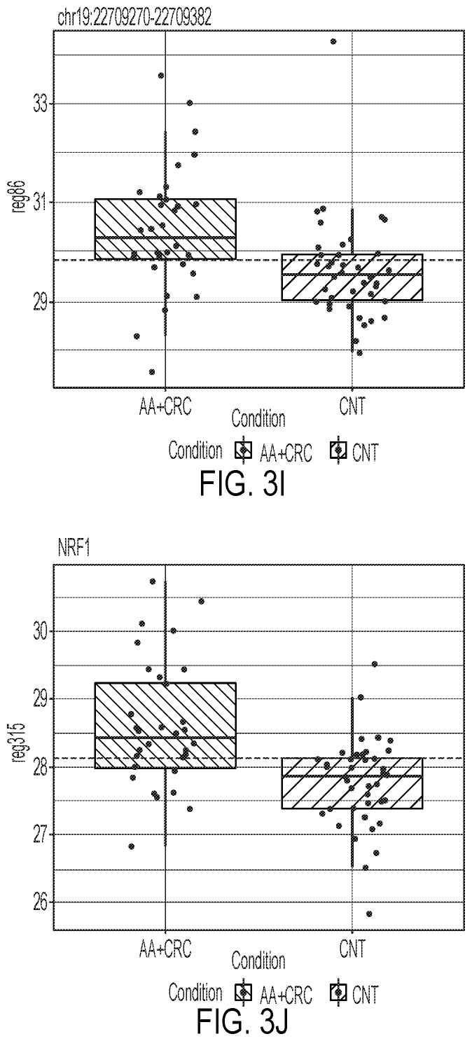

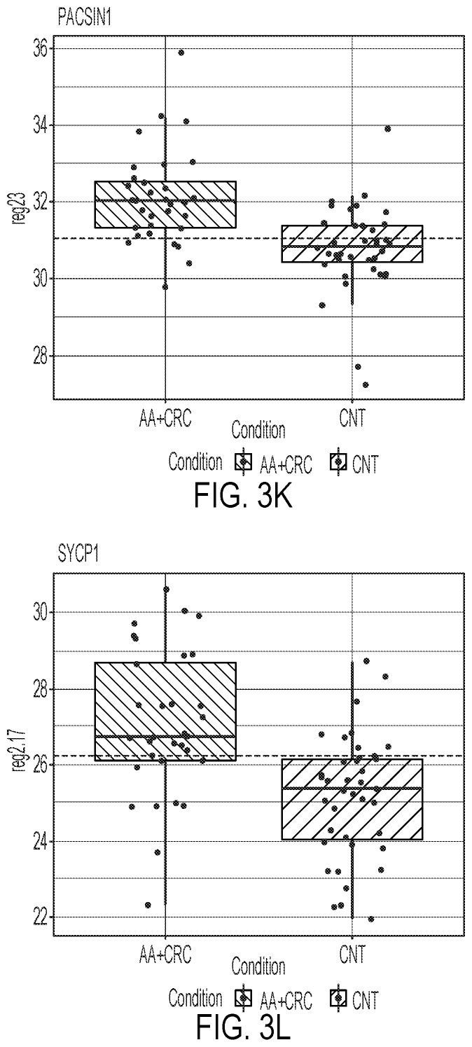

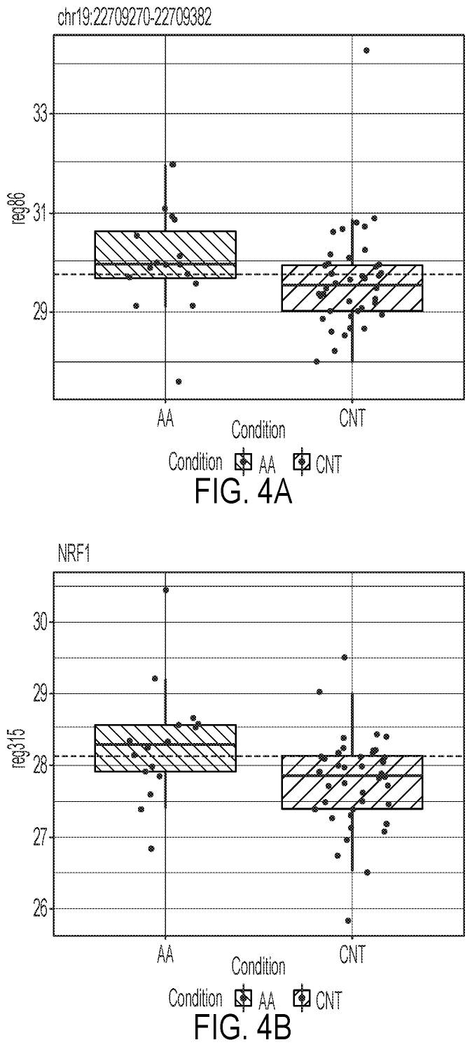

Evaluating different combinations of markers on the validation set of 72 samples (Table 2: Validation cohort) showed combinations of two DMRs (Table 4), six DMRs (Table 5), and twelve DMRs (Table 6 and FIGS. 3A-L), which all performed well in distinguishing AA and/or CRC patient samples from controls subject samples (CNT). Combinations of 2 DMRs could achieve an AUC of 75% as seen in Table 3 below. Additional DMRs (e.g., 6 and 12 markers) increased accuracy of the models. The most accurate results were achieved with a 12 DMR panel, where AUC was 79%, sensitivity for detection of AA+CRC was 78%, and Specificity was 73%. Using the 12 DMR panel, sensitivity for AA was 62.5%. The same sensitivity could be obtained both for patients with advanced adenomas with high grade dysplasia as well as with low grade dysplasia but size >=10 mm. The sensitivity for colorectal cancer was as high as 87.5% with 67% of the stage I cancers, 100% of the stage II cancers and 87.5% of the stage III cancer being correctly identified.

In certain embodiments, any one or more of the markers disclosed in Tables 4, 5, and 6 may be useful in detecting advanced adenoma and/or colorectal cancer. Furthermore, combinations of any 2, 3, 4, 5, 6, 7, 8, 9, 10, 11, or 12 DMRs as disclosed in Tables 4-6 may be useful in detecting advanced adenoma and/or colorectal cancer.

TABLE 3
Prediction algorithm accuracy estimates according to different marker-
combinations for AA + CRC vs CNT + GID group

| | 2 | 6 | 12 |
|---|---|---|---|
| AUC | 0.75 | 0.76 | 0.79 |
| AUC_CI_LOW | 0.64 | 0.65 | 0.68 |
| AUC_CI_HIGH | 0.86 | 0.87 | 0.90 |
| Sensitivity | 0.69 | 0.75 | 0.78 |
| Specificity | 0.73 | 0.73 | 0.73 |
| Accuracy | 0.69 | 0.72 | 0.74 |
| Kappa | 0.40 | 0.45 | 0.48 |

TABLE 4
2 DMR Combination

| associated_genes | chr | start | end |
|---|---|---|---|
| NA | 3 | 75609726 | 75609832 |
| NA | 19 | 22709270 | 22709382 |

TABLE 5
6 DMR Combination

| associated_genes | chr | start | end |
|---|---|---|---|
| NA | 3 | 75609726 | 75609832 |
| NA | 19 | 22709270 | 22709382 |
| NRF1 | 7 | 129720565 | 129720676 |
| CD8B, ANAPC1P1 | 2 | 86862416 | 86862559 |
| LINC01395 | 11 | 129618345 | 129618455 |
| MAP3K6, FCN3 | 1 | 27369224 | 27369347 |

TABLE 6
12 DMR Combination

| associated_genes | chr | start | end |
|---|---|---|---|
| ADSSL1 | 14 | 104736436 | 104736562 |
| CD8B, ANAPC1P1 | 2 | 86862416 | 86862559 |
| CFAP44 | 3 | 113441596 | 113441690 |
| FLI1, LOC101929538 | 11 | 128685299 | 128685448 |
| LINC01395 | 11 | 129618345 | 129618455 |
| MAP3K6, FCN3 | 1 | 27369224 | 27369347 |
| NA | 3 | 75609726 | 75609832 |
| NA | 19 | 22709270 | 22709382 |
| NA | 12 | 53694915 | 53695058 |
| NRF1 | 7 | 129720565 | 129720676 |
| PACSIN1 | 6 | 34514653 | 34514751 |
| SYCP1 | 1 | 114855187 | 114855327 |

### Sub-Group Analysis for Advanced Adenoma Groups Shows Panels of DMRs Useful in Diagnosing Advanced Adenoma

In the present example, performance of DMRs is evaluated in order to identify panels of DMRs useful for diagnosis and/or classification of advanced adenoma in subjects. For the avoidance of doubt, the exemplified DMRs may also be useful in diagnosis and/or classification of colorectal cancer.

The 65 DMRs of Table 1 were further analyzed for their potential for distinguishing advanced adenomas from control group that included patients with colonoscopy negative findings, hyperplastic polyps and gastrointestinal diseases (GIDs). As described in Table 2, 24 advanced adenoma cases from a pilot cohort and 30 control cases from the pilot cohort were used train the model. 16 advanced adenoma and 40 control cases (Table 3, the “Validation cohorts”) were used for validation of the model.

As described above, the classification algorithm was trained by using Monte-Carlo cross-validation over 50 runs by sub-setting the training set to testing and training of the model. A random forest algorithm was used for feature selection and SBS (sequential backward selection) was used to rank the markers for each run. Finally, a support-vector machine (SVM) algorithm was used to build the classification model over the best performing DMRs according to SBS. Evaluating different combinations of markers on a validation set of 56 samples showed combinations of 2 (Table 9) and 3 DMRs (Table 10) that performed well. With the 2 DMRs of Table 9, a sensitivity of 50% at specificity of 80% was achieved as set forth in Table 8. Increasing the marker panel to 3 markers reached optimal accuracy, where AUC was 78% and sensitivity for detection of AA was 69% at specificity of 80% (Table 8).

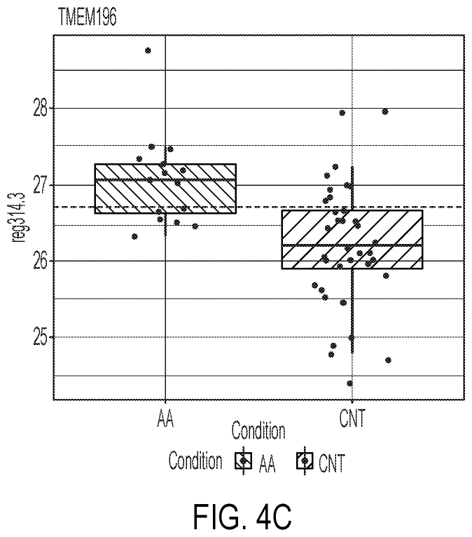

FIGS. 4A-C show detection of hyper-methylated markers in plasma. 45-Ct values plotted for 3 DMRs (the DMRs of Table 10) for control (CNT; healthy+ hyperplastic polyps+GID) samples (right) and AA samples (left). Higher 45-Ct values correspond to higher degrees of hypermethylation in AA samples.

Any one of the markers disclosed in Tables 9 and 10 are useful in detecting advanced adenoma. Furthermore, combinations of any 2 or 3 DMRs as disclosed in Tables 9-10 are useful in detecting advanced adenoma.

TABLE 8
Prediction algorithm accuracy estimates according to different
marker-combinations for AA vs CNT + GID group

| | 2 | 3 |
|---|---|---|
| AUC | 0.67 | 0.78 |
| AUC_CI_LOW | 0.50 | 0.63 |
| AUC_CI_HIGH | 0.84 | 0.92 |
| Sensitivity | 0.50 | 0.69 |
| Specificity | 0.80 | 0.80 |
| Accuracy | 0.71 | 0.77 |
| Kappa | 0.30 | 0.46 |

TABLE 9
2-DMR combination

| associatcd_genes | chr | start | end |
|---|---|---|---|
| NA | 19 | 22709270 | 22709382 |
| NRF1 | 7 | 129720565 | 129720676 |

TABLE 10
3-DMR combination

| associated_genes | chr | start | end |
|---|---|---|---|
| NA | 19 | 22709270 | 22709382 |
| NRF1 | 7 | 129720565 | 129720676 |
| TMEM196 | 7 | 19772652 | 19772800 |

### Sub-Analysis of Colorectal Cancer Samples Shows DMR Panels Useful in Detecting Colorectal Cancer

In the present example, performance of DMRs is evaluated in order to identify panels of DMRs useful for diagnosis and/or classification of colorectal cancer in subjects. For the avoidance of doubt, the exemplified DMRs may also be useful in diagnosis and/or classification advanced adenoma.

The 65 DMRs of Table 1 were further examined for their potential for distinguishing subjects having colorectal cancer from a control group. The control group included patients with colonoscopy negative findings, hyperplastic polyps and gastrointestinal diseases (GIDs). For ranking of DMRs and prediction algorithm development, 24 colorectal cancer cases and 30 control cases were used as pilot cohorts for the present example. 16 colorectal cancer and 40 control cases (Table 2. 2: Validation cohort) were used for validation of the developed model.

As previously described herein, the algorithm was trained by using Monte-Carlo cross-validation over 50 runs by sub-setting the training set to test and train the model. A random forest algorithm was used for feature selection and SBS (Sequential Back Selection) was used to ranking the markers for each run. Finally, a support-vector machine (SVM) algorithm was used to build the classification model over the best performing DMRs according to SBS ranking. Evaluating different combinations of DMRs on a validation set of 56 samples (e.g., the validation cohort) showed combinations of two, three, nine, and eighteen DMRs performed well. With only, two DMRs, Table 11 shows a sensitivity of 69% at specificity of 78%. Increasing the number of DMRs in the panel from two to three, nine, or eighteen DMRs resulted in improved AUC and accuracy. The eighteen DMR panel had the highest AUC. The AUC of the eighteen DMR panel was 95% and sensitivity for detection of CRC was 94% at specificity of 83%.

For the further avoidance of doubt, any one of the markers disclosed in Tables 12-15 are useful in detecting colorectal cancer. Furthermore, combinations of any 2, 3, 4, 5, 6, 7, 8, 9, 10, 11, 12, 13, 14, 15, 16, 17, or 18 DMRs as disclosed in Tables 12-15 are useful in detecting colorectal cancer.

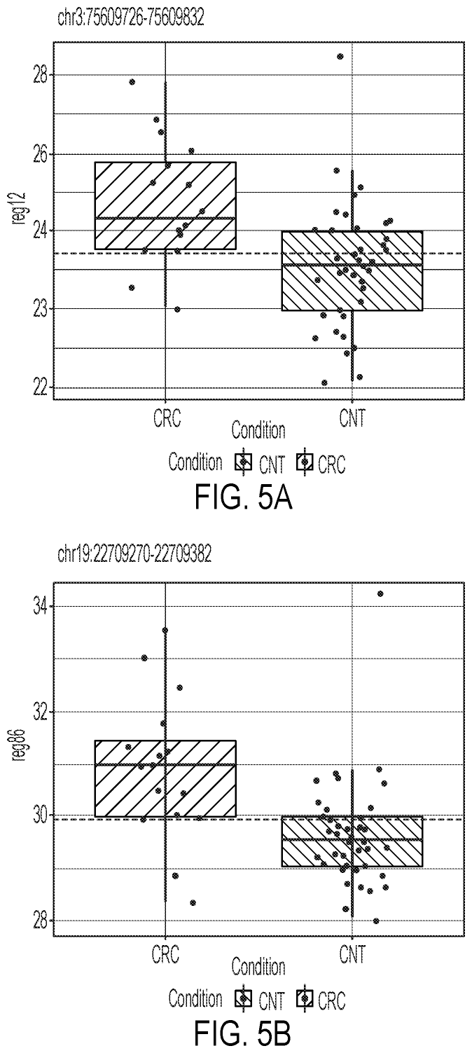

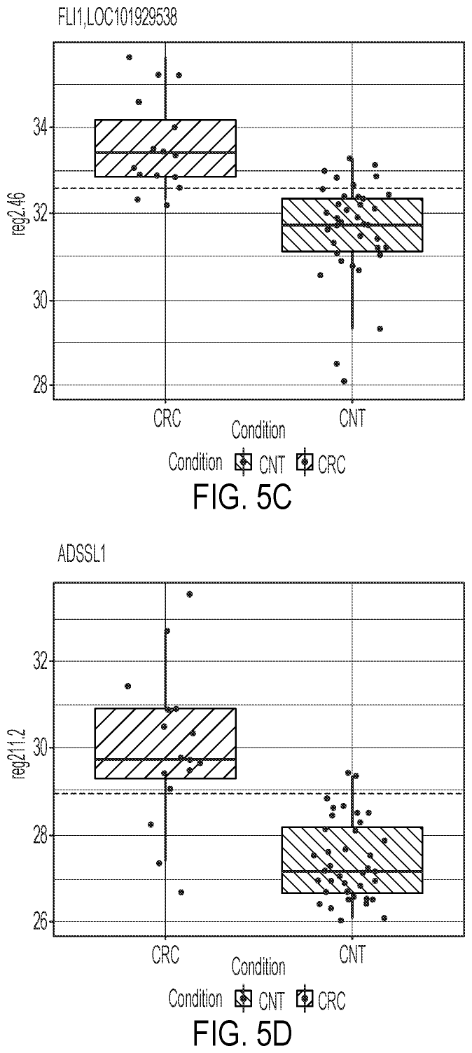

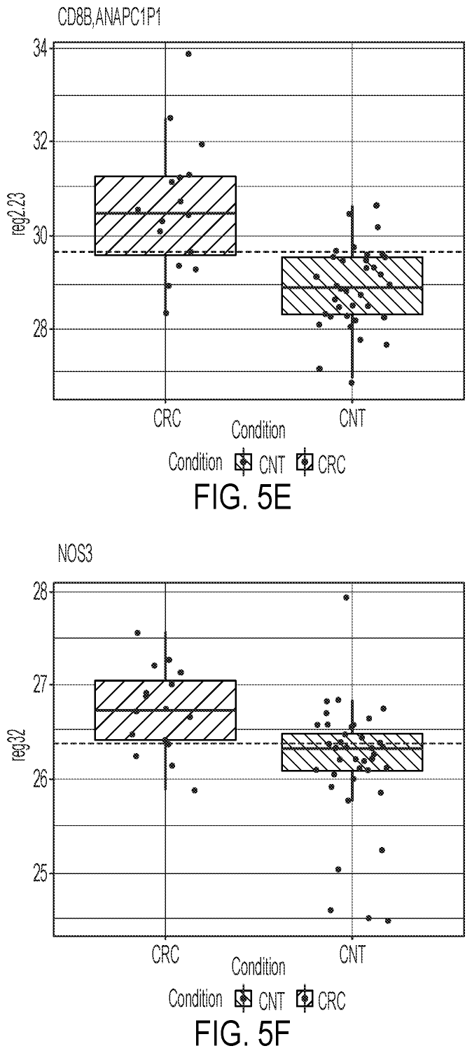

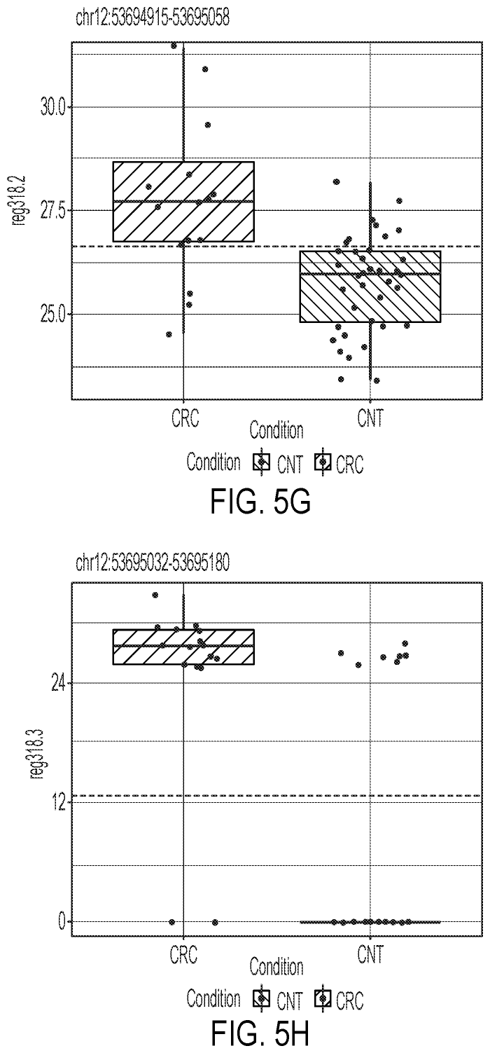

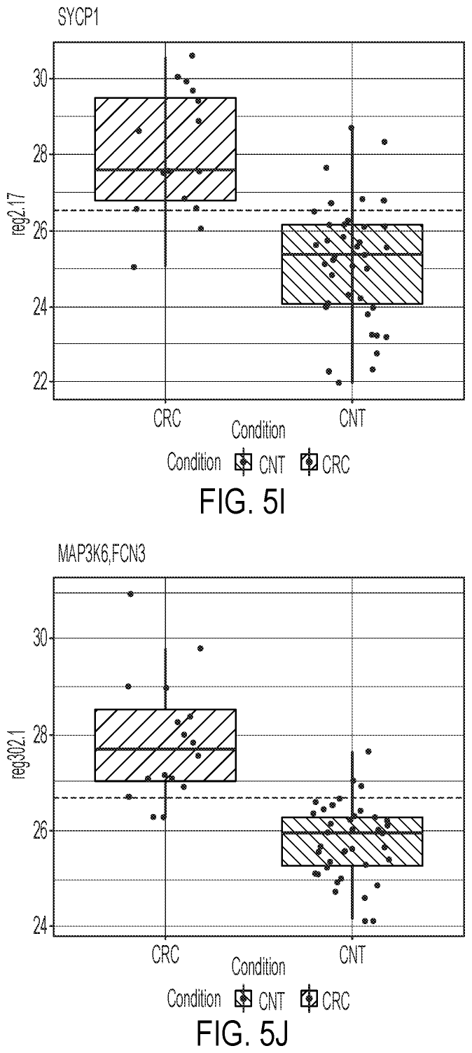

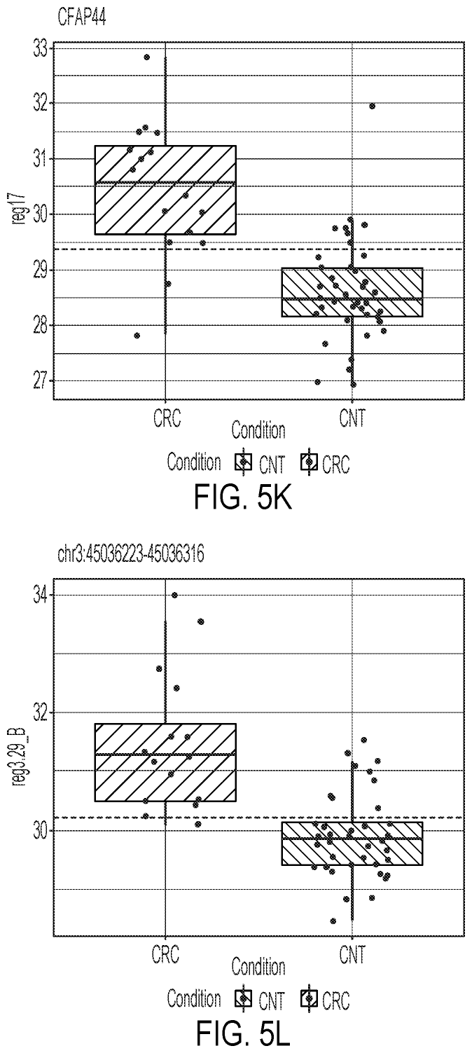

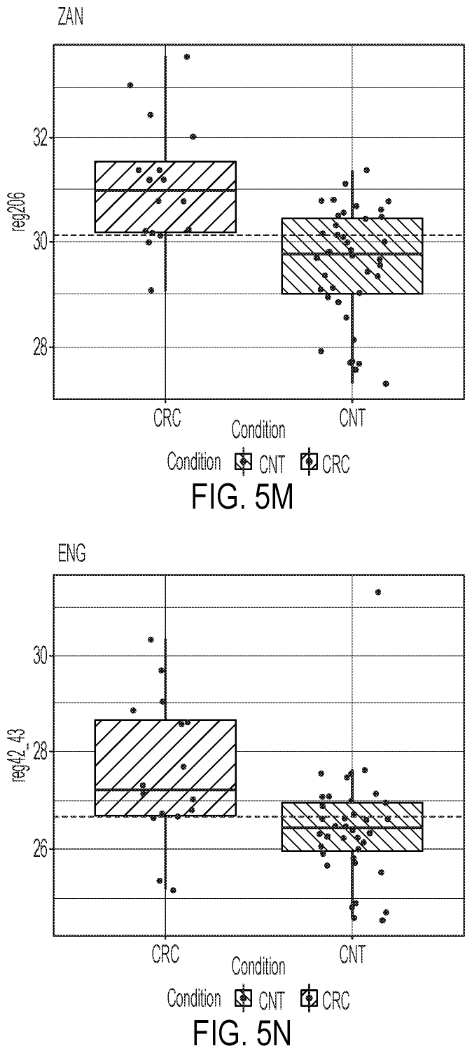

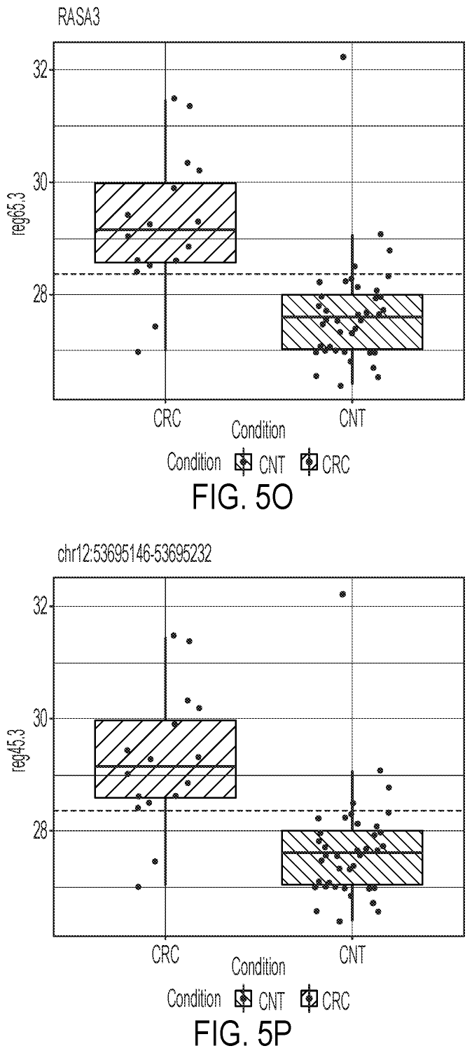

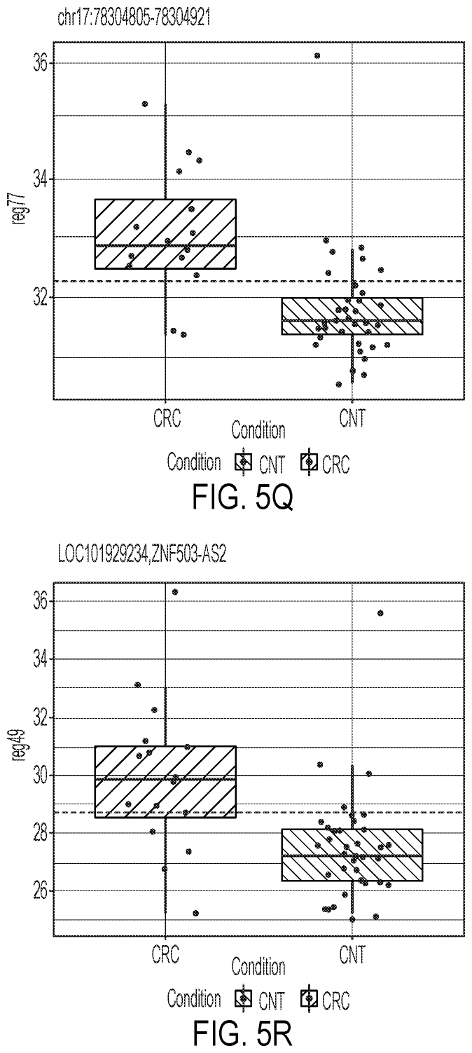

FIGS. 5A-R are graphs representing Ct values from MSRE-qPCR of DNA for subjects with colorectal cancer (CRC) as compared to control subjects (CNT; healthy subjects and subjects with hyperplastic polyps and GID).

TABLE 11
Prediction algorithm accuracy estimates according to
different DMR-combinations for CRC vs CNT + GID group.

| | 2 | 3 | 9 | 18 |
|---|---|---|---|---|
| AUC | 0.83 | 0.89 | 0.93 | 0.95 |
| AUC_CI_LOW | 0.71 | 0.80 | 0.86 | 0.89 |
| AUC_CI_HIGH | 0.94 | 0.98 | 0.99 | 1.00 |
| Sensitivity | 0.69 | 0.94 | 0.81 | 0.94 |
| Specificity | 0.78 | 0.70 | 0.75 | 0.83 |
| Accuracy | 0.75 | 0.77 | 0.77 | 0.86 |
| Kappa | 0.43 | 0.53 | 0.50 | 0.69 |

TABLE 12
2-DMR combination

| associated_genes | chr | start | end |
|---|---|---|---|
| NA | 3 | 75609726 | 75609832 |
| NA | 19 | 22709270 | 22709382 |

TABLE 13
3- DMR combination

| associatcd_genes | chr | start | end |
|---|---|---|---|
| NA | 3 | 75609726 | 75609832 |
| NA | 19 | 22709270 | 22709382 |
| FLI1, LOC101929538 | 11 | 128685299 | 128685448 |

TABLE 14
9- DMR combination

| associated_genes | chr | start | end |
|---|---|---|---|
| NA | 3 | 75609726 | 75609832 |
| NA | 19 | 22709270 | 22709382 |
| FLI1, LOC101929538 | 11 | 128685299 | 128685448 |
| ADSSL1 | 14 | 104736436 | 104736562 |
| CD8B, ANAPC1P1 | 2 | 86862416 | 86862559 |
| NOS3 | 7 | 150996901 | 150997007 |
| NA | 12 | 53694915 | 53695058 |
| NA | 12 | 53695032 | 53695180 |
| SYCP1 | 1 | 114855187 | 114855327 |

TABLE 15
18- DMR combination

| associated_genes | chr | start | end |
|---|---|---|---|
| NA | 3 | 75609726 | 75609832 |
| NA | 19 | 22709270 | 22709382 |
| FLI1, LOC101929538 | 11 | 128685299 | 128685448 |
| ADSSL1 | 14 | 104736436 | 104736562 |
| CD8B, ANAPC1P1 | 2 | 86862416 | 86862559 |
| NOS3 | 7 | 150996901 | 150997007 |
| NA | 12 | 53694915 | 53695058 |
| NA | 12 | 53695032 | 53695180 |
| SYCP1 | 1 | 114855187 | 114855327 |
| MAP3K6, FCN3 | 1 | 27369167 | 27369316 |
| CFAP44 | 3 | 113441519 | 113441620 |
| NA | 3 | 45036223 | 45036316 |
| ZAN | 7 | 100785886 | 100786015 |
| ENG | 9 | 127828322 | 127828421 |
| RASA3 | 13 | 114111799 | 114111878 |
| NA | 12 | 53695146 | 53695232 |
| NA | 17 | 78304805 | 78304921 |
| LOC101929234, | 10 | 75407300 | 75407400 |
| ZNF503-AS2 | | | |

SEQUENCES
SEQ ID NO: 1
taaccacctg cacctctgct gcaatgtaaa cagcagatgt gggcgcaggg tgagaaggga
gaggaagcta cgtgcaatgg caggttgggg aataaggagg cagaggggct cc
SEQ ID NO: 2
ggagagcacc aagaggctcc caataatctg accgctggtg cacatccttc ctcggtcatc
ttccttccag atcagagagg gaaatcaacc atctaccttt ttttcttcca ctatcctcct
taccccttcc accccctacc agatcccaa
SEQ ID NO: 3
gggccagttc ctcctaccag cttcctgctg ccacctcggc ttccatcaga gggacgctta
ggatggcgca ggggcccgga gacactgtga agagtccagg ggaatgagga ggg
SEQ ID NO: 4
cacagacacc ctgagcttgc aacactccgg gcctctgccg cgtgtttatt tcaggatgcc
gtggcatttg ggtgaccttt tgtgctcacc atggcttgcg tcgtctccgg gtcactctcg
tctggac
SEQ ID NO: 5
tgctgaggtc caaactcacc gaaggtactg accgccgcgg ctcctctctt cacagcgtct
gccggaggcc tccgtttact ccggttaccg agacaacgcc acccct
SEQ ID NO: 6
taccgagaca acgccacccc tcttccaggg aggcggaacc agggcgggcc gtggggcgca
tgcgcggccg gcgtccagct ctccgggaac ccggtaccta tc
SEQ ID NO: 7
gctctccggg aacccggtac ctatccgccc tttggtcggg ccttctccgc ctcatgacac
tggttcaaag ccaaacagaa aagcccgacg agttt
SEQ ID NO: 8
gcccctgtaa aatggggata cagcagggca cgacgtctgt tggtcgcctg gcactgggtc
ggccaccgag gccgcgcctt ggcctctttg tcccctctgg
SEQ ID NO: 9
gggagcctgg aggggttgac accgcctgct ccaccgcaag cccctggagg aagagccccg
ctgtgcccga gagcgagcgc gggcaggtgt aactacccgg ggctggg
SEQ ID NO: 10
cctctgcttc aggtgcttgg ctagagaaag ggcggcaaga cggggcagtg cgtgtgcgcg
cgcgggcaag tgcatgtgag tgcacactta tgtgagcgca tgtgtgtctg c
SEQ ID NO: 11
ccggatccag tgggggaagc tgcaggtgcg gctggccagc gactgagaga cccgggcgct
accaaaaggg gagcggggtg gcggggcagt tcctaaggct tcccggg
SEQ ID NO: 12
tgtcaaacct ccatctgtgg tcaggagtta ggacatcccc agctgcaatt tgagcaaaga
cggcgcttcc agaggatcat
SEQ ID NO: 13
gaagggaacg ggctttcttt tcaggccagc gtggcagcgg gcggtagggc gaaagggaga
aggaaacgag ggtttattcc gttgcccact ccgcggtaag cgacgttgta gggctccact
gtagcgagag ccccgtggat t
SEQ ID NO: 14
tgacctcagg tgatccaccc gtctcggcct cccaaagtgt tgggattaca ggcgtgagcc
gccgcgccca gccccctcct cactctcttt ctcttcctgt aacttctaca gctgggcaag
agctgggtct
SEQ ID NO: 15
gctgggaact ggaggtgcag agaaggcccc gacgctgttt gtaggttgtg ggggtgcagc
aagacctaga tcttaagaat ttcgaaggac tgtgacgatc accggctgcg ccctgccggc
gagtgccctg gggctggctc tatt
SEQ ID NO: 16
gcaccaagaa ctaacacatc ctggagctgc ccggagttcc gctcctgcgg gcttagcagg
aaagggtgcc taaggtgagt gcccacttgc gtccgatcct ctgggggcga tgcagggtcg
gggcgcctca gtgtgtctcg ctgcttgttc
SEQ ID NO: 17
tgggcacttg tcatcatggg tgtttggaaa gcaactctac gttctagcct gtgctccatc
gttccttcta catacaagtg atgca
SEQ ID NO: 18
aatacatcca gctcgcaggc atcctgcaag aaacggctcc cggctcgcgt gtacgccgac
acctcggccc aacgcaggac tcgaggtggt ttctagtgcc c
SEQ ID NO: 19
gcaaaggcaa ggtggctgac gatccggaag ctgtacagga gagataaggg cactggctgc
cagagtgccc tatcgaagca tcatccgaac cctgcggtag gggtggccca caccacggcc
tgaggcccag tcaatgccat atttgtgggc
SEQ ID NO: 20
tgccagagtg ccctatcgaa gcatcatccg aaccctgcgg taggggtggc ccacaccacg
gcctgaggcc cagtcaatgc catatttgtg ggcggcagcc tcagacactg catagcgacc
attg
SEQ ID NO: 21
cctgcgacgt gaatcgtcat atccagaggg gggtgatatg actccccgca tcgcgggggc
ctcaccccat tgcgatgggg gtcctaagag ccagggggag atagggg
SEQ ID NO: 22
tgagcggagg actgaggaga ggaaggaggg aaagaatagg gagatgaaaa cgccccggtc
tgctgctaag cacagcacag ttaccaaagc cagg
SEQ ID NO: 23
catctcctcc tcgcaaaccc caagccaagg caagctggat gaagcgctcc ctgggcaggc
ccggctctcc gtgtccctcc atcacctgac cccgctggct ctcgcagacc ccttcctcca
cactcactcc tcccggctct cctt
SEQ ID NO: 24
ccacactcac tcctcccggc tctccttcta taatctcctg acatctcttc aaatccaatt
attgaattaa ttgacgtacg aacccagagg caaacagaaa ggggcggcaa acactgggcg
gctcagattt atccttcggc ctccgcagg
SEQ ID NO: 25
tgggcggctc agatttatcc ttcggcctcc gcagggcccg gccggacgag atttactggg
cctcgaacac ggcgacagtt caaacct
SEQ ID NO: 26
cagccctagg gagacagcag gatggttcca ggaagcctgg gccgctcccc agatcaatgc
agggacggac agcagccagc aggctgggcc acggcatcag agctggggtc aagaggt
SEQ ID NO: 27
cgatgcttgg ccaatgaaaa gagg
SEQ ID NO: 28
gcaaggtgca gatggtgaag gatg
SEQ ID NO: 29
ctcctctggc tctcctgctc catc
SEQ ID NO: 30
gaggcaaaaa ggacaatcgg caag
SEQ ID NO: 31
cctgcgacgt gaatcgtcat atcc
SEQ ID NO: 32
agctgaggga aagggggaag tcac
SEQ ID NO: 33
tgctgaggtc caaactcacc gaag
SEQ ID NO: 34
ccttccccag cctaagaagg tttcc
SEQ ID NO: 35
gccagtgagt cagaggcaga ggtg
SEQ ID NO: 36
taccgagaca acgccacccc tctt
SEQ ID NO: 37
gagctgggac aagaagggaa cacg
SEQ ID NO: 38
gctctccggg aacccggtac ctat
SEQ ID NO: 39
gcgtctctgt ggccgtgaag tgta
SEQ ID NO: 40
ccggatccag tgggggaagc tg
SEQ ID NO: 41
aatacatcca gctcgcaggc atcc
SEQ ID NO: 42
gggagcctgg aggggttgac ac
SEQ ID NO: 43
cctctgcttc aggtgcttgg ctaga
SEQ ID NO: 44
gcccaagggc cacaagagta tgac
SEQ ID NO: 45
cggttccaca cctggaactg gatt
SEQ ID NO: 46
ggtcctagag ccgcttggct tcac
SEQ ID NO: 47
tgtcaaacct ccatctgtgg tcagg
SEQ ID NO: 48
tggggccgaa gagatccttg aaca
SEQ ID NO: 49
gctgcagttt cgtcagccct tg
SEQ ID NO: 50
cagccctagg gagacagcag gatg
SEQ ID NO: 51
ggctcacctt caggaagcac ctgt
SEQ ID NO: 52
gggccagttc ctcctaccag cttc
SEQ ID NO: 53
ggtctttccc acacctctgc acct
SEQ ID NO: 54
gcctggccac cacagagaag aaga
SEQ ID NO: 55
tgctctctct ccaaaggcga gttg
SEQ ID NO: 56
cctgggaacc agtgctggag aaag
SEQ ID NO: 57
gcccctgtaa aatggggata cagca
SEQ ID NO: 58
tgggcacttg tcatcatggg tgtt
SEQ ID NO: 59
acactttgaa aagcgtggcg ttcc
SEQ ID NO: 60
aaaaggctcc gacgatgctc caga
SEQ ID NO: 61
gcaccaagaa ctaacacatc ctggag
SEQ ID NO: 62
gaagggaacg ggctttcttt tcagg
SEQ ID NO: 63
cgagaaggga ggaggtgaag gag
SEQ ID NO: 64
tgagcggagg actgaggaga ggaa
SEQ ID NO: 65
gctctgatgc ctctccctcc acac
SEQ ID NO: 66
gacatcctcc ttggcagcct ttca
SEQ ID NO: 67
tgagcgctta acgatccgga aaga
SEQ ID NO: 68
tgacctcagg tgatccaccc gtct
SEQ ID NO: 69
ccccataggg aggacttgcg cacagttgg
SEQ ID NO: 70
cacagacacc ctgagcttgc aaca
SEQ ID NO: 71
gcaaaggcaa ggtggctgac g
SEQ ID NO: 72
tgccagagtg ccctatcgaa gcat
SEQ ID NO: 73
gtcatcagtg aatcgaccac aaagagc
SEQ ID NO: 74
ggagagcacc aagaggctcc caat
SEQ ID NO: 75
taaccacctg cacctctgct gcaa
SEQ ID NO: 76
acaccccgcg gcaggacttc ta
SEQ ID NO: 77
catctcctcc tcgcaaaccc caag
SEQ ID NO: 78
ccacactcac tcctcccggc tct
SEQ ID NO: 79
tgggcggctc agatttatcc ttcg
SEQ ID NO: 80
tcggcagtga aaagcgggag atta
SEQ ID NO: 81
ggctctcggg ctctcgcttt tt
SEQ ID NO: 82
ctggggagaa gtgaccccat tcaa
SEQ ID NO: 83
gcaccatcct cagagcttca gacca
SEQ ID NO: 84
gctgggaact ggaggtgcag agaa
SEQ ID NO: 85
gcttcaaacg ccgtatcatg ttgct
SEQ ID NO: 86
tggtgccagg ggttaccaca aaga
SEQ ID NO: 87
gagacaacag cccagacccc catc
SEQ ID NO: 88
tgtaagatga cacagctata ttttctggga gagg
SEQ ID NO: 89
ggaggagctg aggtttcggc tgag
SEQ ID NO: 90
aggaggagac cctgccccag aaat
SEQ ID NO: 91
tgagttctct tgagggcagc gaaa
SEQ ID NO: 92
ggctttgctt gtgcccttat cagc
SEQ ID NO: 93
ggtggtgggc gttaaggaag ctc
SEQ ID NO: 94
gtacccttca cccacccagg gttt
SEQ ID NO: 95
cccctatctc cccctggctc ttag
SEQ ID NO: 96
gaaccccagt ggacccctca ga
SEQ ID NO: 97
aggggtggcg ttgtctcggt aac
SEQ ID NO: 98
ggcctctgag tggacagaca ctgg
SEQ ID NO: 99
ctcgagtcct ggaggagcct gtg
SEQ ID NO: 100
gataggtacc gggttcccgg agag
SEQ ID NO: 101
cacaggcctg gagctcctca ca
SEQ ID NO: 102
aaactcgtcg ggcttttctg tttgg
SEQ ID NO: 103
gacagcctcc ctcccatgta cagc
SEQ ID NO: 104
cccgggaagc cttaggaact gc
SEQ ID NO: 105
gggcactaga aaccacctcg agtcc
SEQ ID NO: 106
cccagccccg ggtagttaca cct
SEQ ID NO: 107
gcagacacac atgcgctcac ataa
SEQ ID NO: 108
ccagttccag gtgtggaacc gaac
SEQ ID NO: 109
gtggtgaggg ctgttccatg ctt
SEQ ID NO: 110
cgtgaagcca agcggctcta ggac
SEQ ID NO: 111
gggctgcggg caggcactac
SEQ ID NO: 112
atgatcctct ggaagcgccg tct
SEQ ID NO: 113
catccagacc cgcgtggaca gc
SEQ ID NO: 114
aggcagccaa gacaagcaga gagg
SEQ ID NO: 115
acctcttgac cccagctctg atgc
SEQ ID NO: 116
ggaggcaggg tttacgtgca gaag
SEQ ID NO: 117
ccctcctcat tcccctggac tctt
SEQ ID NO: 118
cagaggggca tgctgactgc ctat
SEQ ID NO: 119
aaggaagggg gcgaggcatc ag
SEQ ID NO: 120
cggacagcag gagggatttc tcag
SEQ ID NO: 121
tgactcctcc agagcgaggt tgtg
SEQ ID NO: 122
ccagagggga caaagaggcc aag
SEQ ID NO: 123
tgcatcactt gtatgtagaa ggaacgatgg
SEQ ID NO: 124
ggccttcctg cagccgtctc tc
SEQ ID NO: 125
tgggcaggcg cctctagatg aaat
SEQ ID NO: 126
gaacaagcag cgagacacac tgag
SEQ ID NO: 127
aatccacggg gctctcgcta cagt
SEQ ID NO: 128
cggggcgttt tcatctccct at
SEQ ID NO: 129
cctggctttg gtaactgtgc tgtgc
SEQ ID NO: 130
ggcacaggca aatgccaaat cct
SEQ ID NO: 131
aaaggcccag agatcggagc tgag
SEQ ID NO: 132
catgccgtcc tcaaattcca gagc
SEQ ID NO: 133
agacccagct cttgcccagc tgta
SEQ ID NO: 134
ctttaaccct ttcccctcgc ccgcagca
SEQ ID NO: 135
gtccagacga gagtgacccg gaga
SEQ ID NO: 136
gcccacaaat atggcattga ctgg
SEQ ID NO: 137
caatggtcgc tatgcagtgt ctgagg
SEQ ID NO: 138
gtgcagggga cagcagacat caga
SEQ ID NO: 139
ttgggatctg gtagggggtg gaag
SEQ ID NO: 140
ggagcccctc tgcctcctta ttcc
SEQ ID NO: 141
gggaagagct ggggtcagtg aagg
SEQ ID NO: 142
aaggagagcc gggaggagtg agtg
SEQ ID NO: 143
cctgcggagg ccgaaggata aa
SEQ ID NO: 144
aggtttgaac tgtcgccgtg ttcg
SEQ ID NO: 145
gcgcccagac caccgaggac
SEQ ID NO: 146
gccaggcagg agaaagagct tgaaa
SEQ ID NO: 147
agaaagcagc cccaagtggg aaga
SEQ ID NO: 148
atttgggagc caccagggaa gga
SEQ ID NO: 149
aatagagcca gccccagggc actc
SEQ ID NO: 150
gggtccacct atcagggcct gtg
SEQ ID NO: 151
caccagacaa ccagcctgcc aagt
SEQ ID NO: 152
cctgatgggg gaaaaggcac cata
SEQ ID NO: 153
cacaggctgg tcttccccac ttca
SEQ ID NO: 154
tgtacagcga tggcctgata agcaa
SEQ ID NO: 155
tcacacggag ggaatcaaca aaagg
SEQ ID NO: 156
cgatgcttgg ccaatgaaaa gaggtctacc cgagagtgcg acgcgcaatg ggcgggactt
ccggcgtctc ccctcggcgg ttgctttcgc tgccctcaag agaactca
SEQ ID NO: 157
gcaaggtgca gatggtgaag gatgcacacg agggccgcat caccacgctg cggaagaaaa
agaaggggaa ggatggagcc ggggccaagg aggctgataa gggcacaagc aaagcc
SEQ ID NO: 158
ctcctctggc tctcctgctc catcgcgctc ctccgcgccc ttgccacctc caacgcccgt
gcccagcagc gcgcggctgc ccaacagcgc cggagcttcc ttaacgccca ccacc
SEQ ID NO: 159
gaggcaaaaa ggacaatcgg caagtaaata gtaaatgaac aagaagaccc cggttgtgag
aaaatgttat aaagcaaata aatcagagaa atgtgatcac aaaccctggg tgggtgaagg
gtac
SEQ ID NO: 160
agctgaggga aagggggaag tcactgggct gggggccggg gccgctcact ctggcctcct
ctgaggggtc cactggggtt c
SEQ ID NO: 161
ccttccccag cctaagaagg tttcctctcc gggagtcacc caaggtgtgc tgaccctggc
ctgggaccct gggaccgtgg cgctcccacg ctagcagcga cacggccagt gtctgtccac
tcagaggcc
SEQ ID NO: 162
gccagtgagt cagaggcaga ggtgccagag accccgcccg aagggaggag atctgagagc
ctgcagccac aggctcctcc aggactcgag
SEQ ID NO: 163
gagctgggac aagaagggaa cacggtacca gggtagcaga agacaggcac cccccgtccc
ccagtcctag ggcttcctca ccgcgcctgt gaggagctcc aggcctgtg
SEQ ID NO: 164
gcgtctctgt ggccgtgaag tgtatgcatg cgtgcccatg ttgatgcggc gccgtgcggg
aggcgggcat cccctgctgt acatgggagg gaggctgtc
SEQ ID NO: 165
gcccaagggc cacaagagta tgacggggct gtacgagctg ctgtgacggg tgctgcatgc
gctgctccgt ctgcaccgca cgctcacctc ctggctccgc gttcggttcc acacctggaa
ctgg
SEQ ID NO: 166
cggttccaca cctggaactg gatttggcgg cgctgctgcc gcgccgcctc tgccgcggtc
ctagagccgc ttggcttcac gctccgcaag catggaacag ccctcaccac
SEQ ID NO: 167
cggttccaca cctggaactg gatttggcgg cgctgctgcc gcgccgcctc tgccgcggtc
ctagagccgc ttggcttcac g
SEQ ID NO: 168
ggtcctagag ccgcttggct tcacgctccg caagcatgga acagccctca ccacacgcac
ccgcgcgggg ggtagtgcct gcccgcagcc c
SEQ ID NO: 169
tggggccgaa gagatccttg aacacgtcgt aggactcctc gtcggccgcc acgcggccca
cggccctgag tacgggtggc ccgggctgtc cacgcgggtc tggatg
SEQ ID NO: 170
gctgcagttt cgtcagccct tggctccggg ctctgcaggc ggaatcccga gcctgcgtga
gggccgccct ggcctcggcg tgtgtcctgg gaaggggcgt tggaagcctc tctgcttgtc
ttggctgcct
SEQ ID NO: 171
ggctcacctt caggaagcac ctgtggcggg ccgcgtcacc cactcgggac cccggagacc
aagtccgctc ttctgcacgt aaaccctgcc tcc
SEQ ID NO: 172
ggtctttccc acacctctgc accttgttac ctgactttcg gcttcaggat ccgcagcgtg
cacccgcgtt ccgtgagtgc cctataggca gtcagcatgc ccctctg
SEQ ID NO: 173
gcctggccac cacagagaag aagacggagc agcagcggcg gcgggagaag gctgtgcaca
ggctggtgag cgcctgggcc agcggggcct gcctctgatg cctcgccccc ttcctt
SEQ ID NO: 174
tgctctctct ccaaaggcga gttgatcaca gacgctggca gtgagtcagc ggcaccgcca
gggctgctga gaaatccctc ctgctgtccg
SEQ ID NO: 175
cctgggaacc agtgctggag aaagtatgtg gaagctggcg atggagaagg cgcgcgcatg
tgtgcacaac ctcgctctgg aggagtca
SEQ ID NO: 176
acactttgaa aagcgtggcg ttccagcgca aaccaacccg aacgggttgg aagggggcag
tcctttcttc ccgcaagttc ggggctcgag agacggctgc aggaaggcc
SEQ ID NO: 177
aaaaggctcc gacgatgctc cagacgcgga cacggccatc atcaatgcag aaggcgggca
gtcaggaggg gacgacaaga aggaatattt catctagagg cgcctgccca
SEQ ID NO: 178
cgagaaggga ggaggtgaag gagggcgagc tgagcacacg cgcttcatgc cacaggaggg
tgggaatgag cggaggactg aggagaggaa ggagggaaag aatagggaga tgaaaacgcc
ccg
SEQ ID NO: 179
gctctgatgc ctctccctcc acaccacacc tgtgatctac tgtgcatagg atctcacagg
cccaataaca gagctggagt tcctcttacg tgacacagga tttggcattt gcctgtgcc
SEQ ID NO: 180
gacatcctcc ttggcagcct ttcaacacgt ttctcaaatc ctttcccagc ttcctgtgca
gcctttcctc ctcagcctgg ctgccttact gtctcagctc cgatctctgg gccttt
SEQ ID NO: 181
tgagcgctta acgatccgga aagaggaaga tggagacgct ggaaaggaag aggacgccag
gacgcgcatc atcagacgcg cagctctgga atttgaggac ggcatg
SEQ ID NO: 182
ccccataggg aggacttgcg cacagttggc gctgggtaaa tgctgggaga actgctgcgg
gcgaggggaa agggttaaag
SEQ ID NO: 183
gtcatcagtg aatcgaccac aaagagcctt tgcggaggtg atttacagga gagctctgat
gtctgctgtc ccctgcac
SEQ ID NO: 184
acaccccgcg gcaggacttc tagagaagcc caggatctgt cccgtgccgc cgctgctccc
ctccccagac acctctccac gtctcctacc cagggggtcg catccctagc ccttcactga
ccccagctct tccc
SEQ ID NO: 185
tcggcagtga aaagcgggag attagaaaat gtttcatgct aatttccatg gagatttctt
taatttagcg aagactgctt cccgggctcc gcctggcccg cgccggcccg cgtcctcggt
ggtctgggcg c
SEQ ID NO: 186
ggctctcggg ctctcgcttt tttttttttt ttttctttcc gcggcagtct taggattctt
gtcacatgat ggcttcatcg ggcccttctc ctcctgatcc tttcaagctc tttctcctgc
ctggc
SEQ ID NO: 187
ctggggagaa gtgaccccat tcaatagtcc ttggtctcct tctgccctgc ggctgcgctt
cctcggctct cacggcacca gcagaattcc atgtgagagg gagcttgtcg agcgtggcct
cttcccactt ggggctgctt tct
SEQ ID NO: 188
gcaccatcct cagagcttca gaccatacat tgacagtgag caaagggggc cccaggcagg
cgggtctggg gccaaggagg gcggctcccc tgcgcggatc cttccctggt ggctcccaaa
t
SEQ ID NO: 189
gcttcaaacg ccgtatcatg ttgctttaaa acctgcgggt aacagcataa gctgagtttt
ctatcttaga actcttaacc ccaagaacac tcttcacagg ccctgatagg tggaccc
SEQ ID NO: 190
tggtgccagg ggttaccaca aagaggcggc agagccatgg cccaccagcc acttggcagg
ctggttgtct ggtg
SEQ ID NO: 191
gagacaacag cccagacccc catcacggag ctgcacgtga ccctggaact taacagcttc
cagttgttcc ctagacagtc attgtcttta tggtgccttt tcccccatca gg
SEQ ID NO: 192
tgtaagatga cacagctata ttttctggga gagggcggga ggatgctcag cgagggtggc
ccggagtgtc cttgtacaga gtacagatgt tatgaagtgg ggaagaccag cctgtg
SEQ ID NO: 193
ggaggagctg aggtttcggc tgagccccca gcctcccccg accgcacagc ctcgggcatg
aacccgcgaa gccagacgct tagttgctta tcaggccatc gctgtaca
SEQ ID NO: 194
aggaggagac cctgccccag aaataggcca gtgcttgtta tgcaggcctt ggcggttccc
cgtttcctta cgtaacctca gtgttcacgc tgtttccttt tgttgattcc ctccgtgtga
SEQ ID NO: 195
catctccatc ttctccatca tctccatctt ctccatcatc tccatcttca tcatctccat
ttccatcatc tccatctcca tcatcatctc tatctccatc atctccatct ccatcatctc
catcatctcc atctccatct ccatctccat catctaccgt ctccaatctc catctccgaa
gttatgccca cttcctcgaa gtttggagcc acgcgaacta cactgcccag aaggcgccgc
gccgtgagcc gcgatgcttg gccaatgaaa agaggtctac ccgagagtgc gacgcgcaat
gggcgggact tccggcgtct cccctcggcg gttgctttcg ctgccctcaa gagaactcag
cttgccggaa gctggttgtt cgctgcggcg accagctccg gaaagcgcgg tggggacgcg
ctgtgttctc gcagctcaga ggcgggtctg aggctcggtg gcggcgccca gggtggcccg
ggccctttcc tcggtcgttg tctcaccgcc acaggctccg atggcggcgg ccacgctgag
ggaccccgct caggtgagcg ccgcgtcctc ccggcctccc ccgaatccta aagccctgtg
agggccgcc
SEQ ID NO: 196
cttcctcgga ctctcggccg acgagctcat cgccatcatc tccagcgacg gccttaacgt
ggagaaggag gaggcagtgt tcgaggcggt gatgcggtgg gcgggtagcg gcgacgccga
ggcgcaggct gagcgccagc gcgcgctgcc caccgtcttc gagagcgtgc gctgccgctt
gctgccgcgc gcctttctgg aaagccgcgt ggagcgccac cctctcgtgc gtgcccagcc
cgagttgctg cgcaaggtgc agatggtgaa ggatgcacac gagggccgca tcaccacgct
gcggaagaaa aagaagggga aggatggagc cggggccaag gaggctgata agggcacaag
caaagccaaa gcagaggagg atgaggaggc cgaacgtatc cttcctggga tcctcaatga
caccctgcgc ttcggcatgt tcctgcagga tctcatcttc atgatcagtg aggagggcgc
tgtggcctac gatccagcag ccaacgagtg ctactgtgct tccctctcca accaggtccc
caagaaccac gtcagcctgg ttaccaagga gaaccaggtc ttcgtggctg gaggcctctt
ctacaacgaa gacaaca
SEQ ID NO: 197
ttcccttttt ctcctcacaa ggaggtgagg ctgggacctc cgggccagct tctcacctca
tagggtgtac ctttcccggc tccagcagcc aatgtgcttc ggagccactc tctgcagagc
cagagggcag gccggcttct cggtgtgtgc ctaagaggat ggatcggagg tcccgggctc
agcagtggcg ccgagctcgc cataattaca acgacctgtg cccgcccata ggccgccggg
cagccaccgc gctcctctgg ctctcctgct ccatcgcgct cctccgcgcc cttgccacct
ccaacgcccg tgcccagcag cgcgcggctg cccaacagcg ccggagcttc cttaacgccc
accaccgctc cggcgcccag gtattccctg agtcccccga atcggaatct gaccacgagc
acgaggaggc agaccttgag ctgtccctcc ccgagtgcct agagtacgag gaagagttcg
actacgagac cgagagcgag accgagtccg aaatcgagtc cgagaccgac ttcgagaccg
agcctgagac cgcccccacc actgagcccg agaccgagcc tgaagacgat cgcggcccgg
tggtgcccaa gcactc
SEQ ID NO: 198
tggtcagggc ctgaggcaac cctgtcccag cgctgaggac ccaggaacat gccaccagcc
tgggatgggg gaggccacgg agggagggag cagtgagccc ccagggagga atctcgagct
gagggaccag gagttcgggc ttgttctgag aaacgcacag tgtcagagtc actcattcag
aaagactgag agagcctgcc gagagctggg taccggagac gcgtccctgc cctctcagag
ttgacagtcc agaggcaaaa aggacaatcg gcaagtaaat agtaaatgaa caagaagacc
ccggttgtga gaaaatgtta taaagcaaat aaatcagaga aatgtgatca caaaccctgg
gtgggtgaag ggtacaagtt taggaaacgg gtcagggaag gcctctctga catttgagct
gagccttgga tgaccagaaa gaactattga aagatctggg tggggccaga ggaggggtga
gtggcagatg ccccaggaga gaagaaagtt gtccaggagg ggcccgtgca ctggaggcag
gggacggggc aggcacaggg gcagggggac gaagccagag gcactccctc ccccagggtg
ctgagcaggg gagccccctg actca
SEQ ID NO: 199
ccaaagaatg cagagaatgt gcacccgtct gtgacatagt tggtaatttc cagaggcgga
gaagatatta ttgacaataa ggtgaacacg ctgtgtgacc accgtggatc gtcatatcca
gggggggaga tgggggtgat atgactcccc gcatcgcggg gggcgcccgc ccccctgcga
tgtggatcat catatccagg gggggagagg ggggtgatat gactcccctc atcgcggtgg
gcgcccgccc ccctgcgacg tgaatcgtca tatccagagg ggggtgatat gactccccgc
atcgcggggg cctcacccca ttgcgatggg ggtcctaaga gccaggggga gataggggct
ggctcttact ccccgtaccg ccgggggggg gggcctcacc gccctgcgac ggggctcctt
agagccaggg agggggaggg gctggctctt actcccggta tcgcaggagg tgtgtacaac
ccctgcgata ttgggagtaa tatcatcctc tccccctgaa tataagaaac aatatcacag
gagcatgtac accccctgcg atattggaag taacatcatt ttctccccct agggatattc
ggaacaat
SEQ ID NO: 200
ggggggcaag gacacggggc cctccccagg ctcgctggca gcccattgtg ctgggctgga
aggtctccca acctgaggac acctaggggc aagggagcca ctggcctgag cctgagatct
ctgagcgggg gcaggcagcc ctcgccatgc caagggcatc cctaatccac ccctacacac
cagcggaagc cactggcagt gagggcccag ggccaccaag cagggctggg gcaggaaaga
ccagcaggtg cagctgaggg aaagggggaa gtcactgggc tgggggccgg ggccgctcac
tctggcctcc tctgaggggt ccactggggt tccggctcct cagaccctgg ctctgcagcc
tcagggccaa cttcccgctt ggagaaaggg cagcgcttgt ccggggaccc accacatcca
tcctcgtagg gggctgtctc cacccagggt ccccccccca ccccctcatt cctcccagtg
gtgaaaggac agtgaaggag gagggcagcc caggagtgga catggagtga ccaggagctt
cctggggggt ccgggaggtg gggcacaccc tatcgcacac ca
SEQ ID NO: 201
tggccgggtc taagctgtgc tcctgctgcc tggctggctt ccgcccggtc agactgacag
ggtcttgcag gcaggaaccg tgcacacagt gtctagctcc gagcctgaga atactcgtgg
cttcaaaagt ttgctgagct accgcaggga ggacgaaggc tataacactg gtccagcctg
agagaagccc aagtggggtt cactgccctc tgagccacag atttaagggg gagggtgtgg
aaactgccgg ctgctgaggt ccaaactcac cgaaggtact gaccgccgcg gctcctctct
tcacagcgtc tgccggaggc ctccgtttac tccggttacc gagacaacgc cacccctctt
ccagggaggc ggaaccaggg cgggccgtgg ggcgcatgcg cggccggcgt ccagctctcc
gggaacccgg tacctatccg ccctttggtc gggccttctc cgcctcatga cactggttca
aagccaaaca gaaaagcccg acgagtttat tatcccctaa aggacgtcat gtagataatt
aaatgacatg aataccgtcg aagatacctg cctgatattc caaaatggcc caacggagcc
ctgatcg
SEQ ID NO: 202
tataaagtgc gcgcagtttg ttttatttcc tgagtttttg caatctagat aacagatgat
accctgagtg gctggcgctg cctctgtaat ggcggcactg agcctttgga gaagtattaa
taatagattg tgttgatgag tttggagaaa gtagcaatcg accccctgct gccaaggcat
tagcgcggct gttctgagca cagccagcac tgtggctttg actgcaaatg caggtcaccc
gccctgctgc cccttcccca gcctaagaag gtttcctctc cgggagtcac ccaaggtgtg
ctgaccctgg cctgggaccc tgggaccgtg gcgctcccac gctagcagcg acacggccag
tgtctgtcca ctcagaggcc gcagaggtca ggctgcagac cttagtgtgg ccactaggtc
aggtggagtg tggggagggg acagaggggc agtaggggtt gggggaggac caccctccat
gtcagagcac cgggttctac aaacccaggc tccttcctca gcccctcggg agagctggac
agccagccag attcctaggg cctctgccta aagctgtcac tgacagttgg gtaggttgtg
ccctgaacaa ggggattcag ccagagggcc
SEQ ID NO: 203
agtgtgttcc tcacttccac ctgtggcggt cgcttctggc tgtcaccctg agcacatcca
tgtggcctct tggagtggcc tctccacgtg gcctaggctt cctggcaacg cagccgcctc
agggcagtgt gacttcctga tggtggtgac tcaggacaac aaaagcgaga ggccctgaga
gtcaggcggg caccacaggg ccttgctgag gcagccgggg actcccgctc cctctgctga
caccattggt ggccagtgag tcagaggcag aggtgccaga gaccccgccc gaagggagga
gatctgagag cctgcagcca caggctcctc caggactcga gcaccggggc cgcacagaga
gccctttctc tcctgggcag gccaggcggg gatcccccag cgccctaacc tgctctgtga
ccacggcaat gtggccttgg ggatgtgccc tgcctctctg ggttccagtg caggactcag
ggctggccac ctgagaagca tctctaggac attccaaagc ctggaacagg gacagcattg
tggccctgct ctggaaggct gcgtggaagc caagaagttg tcctggcctg t
SEQ ID NO: 204
acagtgtcta gctccgagcc tgagaatact cgtggcttca aaagtttgct gagctaccgc
agggaggacg aaggctataa cactggtcca gcctgagaga agcccaagtg gggttcactg
ccctctgagc cacagattta agggggaggg tgtggaaact gccggctgct gaggtccaaa
ctcaccgaag gtactgaccg ccgcggctcc tctcttcaca gcgtctgccg gaggcctccg
tttactccgg ttaccgagac aacgccaccc ctcttccagg gaggcggaac cagggcgggc
cgtggggcgc atgcgcggcc ggcgtccagc tctccgggaa cccggtacct atccgccctt
tggtcgggcc ttctccgcct catgacactg gttcaaagcc aaacagaaaa gcccgacgag
tttattatcc cctaaaggac gtcatgtaga taattaaatg acatgaatac cgtcgaagat
acctgcctga tattccaaaa tggcccaacg gagccctgat cgcggcgttc ctatgttgag
gttttaactt cgattttaag aggggtcctg ggagatagta ggcagcttgc cggcaacatc
aac
SEQ ID NO: 205
gatccagccg agtcagggac tttccccacg ccccacccga ccctcaggcc tgacagccac
aggggcagag caggaggagg ccaggcaggg gcagtgaggg aaacagggag gggccttggc
cacagcaatc ttgggcctcc agccccatgg gaaccccagc acgatgagca tccagggtca
ttgaggggga ggcggggagc tggctgtggc cacctggagt cacagcgggg caagggttgg
gggccccagg ggagctggga caagaaggga acacggtacc agggtagcag aagacaggca
ccccccgtcc cccagtccta gggcttcctc accgcgcctg tgaggagctc caggcctgtg
cagacggggg cagggcccgg cagggcgggt gggaaggcga cctgagggcc catgatgaag
gccaccagca gcagcagcag gagggcattg aaagccagca gggtcttgag aaagaggaag
taggagagca cgctggagcc gaactggccc ccgatgcgct tcagggcgta gcgccacggc
atcagggcct gcagggcgga gagcagcgcc aggcccaggc tgtgcaaggc ctgcgggcac
aggcagagag
SEQ ID NO: 206
taacactggt ccagcctgag agaagcccaa gtggggttca ctgccctctg agccacagat
ttaaggggga gggtgtggaa actgccggct gctgaggtcc aaactcaccg aaggtactga
ccgccgcggc tcctctcttc acagcgtctg ccggaggcct ccgtttactc cggttaccga
gacaacgcca cccctcttcc agggaggcgg aaccagggcg ggccgtgggg cgcatgcgcg
gccggcgtcc agctctccgg gaacccggta cctatccgcc ctttggtcgg gccttctccg
cctcatgaca ctggttcaaa gccaaacaga aaagcccgac gagtttatta tcccctaaag
gacgtcatgt agataattaa atgacatgaa taccgtcgaa gatacctgcc tgatattcca
aaatggccca acggagccct gatcgcggcg ttcctatgtt gaggttttaa cttcgatttt
aagaggggtc ctgggagata gtaggcagct tgccggcaac atcaacaaca aagatacatc
gtgggatttt tgttattttt aaaactatat tatctctgtt ggcttttaag agtaaa
SEQ ID NO: 207
cacactcagc ctggcctaga aaaaactcaa aattttgaat tttcatcaaa tgagagaata
aatgattaaa caaatagaaa tgcttcaccc agcagcaagc gcttagattt taaggaccca
agcaaagtgc atggaaaggt gcagctgtct ggaaggacga ttgggaggtg ggatcttggg
gagaaaggga agaaagggga tggagcaggg cttcccagtc gagggcggcg gccgagcctg
tgtccccacc agcgtctctg tggccgtgaa gtgtatgcat gcgtgcccat gttgatgcgg
cgccgtgcgg gaggcgggca tcccctgctg tacatgggag ggaggctgtc tgtgcagagc
attgcccagt tgccatagaa acgagcagaa ggaggtgggt ggctggagaa ggaggcgggt
cgggatcggg gagtggggag gaggcagcgg tggagggagc tggctcctgc agttctggcg
ctgctgcctt cctgagtgag cggtggaggg aaccctagag gacagagccc ccagcccggc
agcaggcccc ctctccgccc gccaccacgg aggagaagga ggacagccag cccctccagc
SEQ ID NO: 208
acaccacgtg ggcccctccc gccctccccc agcacttgca caaagcctgg aggagggcct
ccctgtccca cacaacttcc tgcttgtccc cttcccaccc ctctcctccc caggagcggc
tcccaggccc acgaacagcg gcttcaagag gtggaagccg aggtggcagc cacaggcacc
taccagctta gggagagcga gctggtgttc ggggctaagc aggcctggcg caacgctccc
cgctgcgtgg gccggatcca gtgggggaag ctgcaggtgc ggctggccag cgactgagag
acccgggcgc taccaaaagg ggagcggggt ggcggggcag ttcctaaggc ttcccggggg
ctgggaggtc ccaaactgtg ggggagatcc ttgccttttc ccttagagac tggaaaggta
gggggactgc cccaccctca gcacccaggg gaacctcagc ccagtagtga agacctggtt
atcaggccct atggtagtgc cttggctgga ggaggggaaa gaagtctaga cctgctgcag
gggtgaggaa gtctagacct gctgcagggg tgaggaagtc tagacctgct gcaggggtga
ggaagtct
SEQ ID NO: 209
tgaaatatat gtccacacac ggagaattta agagtatttt tatatttctc tctagatcta
aatattcaga tgtgttaatt acatgcccta gaagctggaa gcgatcagtg gtgttcacac
tggacgtgga gctgtttgta taattttcat ctccctgcac ttaaacatga ctctcagtct
aataaattca accttgtcat ttttagaatc gacgggattt ctctggctgt cgtttgcgct
gcatttatcc gaatacatcc agctcgcagg catcctgcaa gaaacggctc ccggctcgcg
tgtacgccga cacctcggcc caacgcagga ctcgaggtgg tttctagtgc ccgggtggct
gcaagtctgc cctccgaggg aggctggaca agcggcgccc ccaggtcgag cggcctctcg
ctgcctggca gtgcctggca gcccccacct ctgccagtgc ttcggaaacc cgcctggcca
ggttcgcccg cggtgaaaaa tgaaagcaaa ttccccaaca gaggtagccg gaactttcct
cgacgaaggc tccctcctgc gcctgtgtct ggagaacccc cagagcgctg caagttagca
ag
SEQ ID NO: 210
agtccaagtt tctgccacag ttccagggcc gaggctgttt ccaaagagcc ctgtaattgt
tttccacctg tgtctcaccc aaacaccaag gctggcgcag gtggacacct tcccactttt
ctccctccag gctgggcccc agaaatcagt agaggaggga ggaatcagtc agcgtggcca
tgcctgggag gagaggcccg tgtgggtctg tggggctaag aggcaaaggc gggtggcgga
tgtgggccag cgggagcctg gaggggttga caccgcctgc tccaccgcaa gcccctggag
gaagagcccc gctgtgcccg agagcgagcg cgggcaggtg taactacccg gggctggggc
tccgggggct ccgcgcagcc tccttccctc ccagggacac cgcccagctg cgccccgcgc
cccgccgact gcgcgggcct tgagacgctg gtggctgcct cggggttggc ctgctcctcg
cgcacatgtt cagggtcatc cgcgctgcgc ctctgcttca ggtgcttggc tagagaaagg
gcggcaagac ggggcagtgc gtgtgcgcgc gcgggcaagt gcatgtgagt gcacacttat
gtgagcgc
SEQ ID NO: 211
tggaggggtt gacaccgcct gctccaccgc aagcccctgg aggaagagcc ccgctgtgcc
cgagagcgag cgcgggcagg tgtaactacc cggggctggg gctccggggg ctccgcgcag
cctccttccc tcccagggac accgcccagc tgcgccccgc gccccgccga ctgcgcgggc
cttgagacgc tggtggctgc ctcggggttg gcctgctcct cgcgcacatg ttcagggtca
tccgcgctgc gcctctgctt caggtgcttg gctagagaaa gggcggcaag acggggcagt
gcgtgtgcgc gcgcgggcaa gtgcatgtga gtgcacactt atgtgagcgc atgtgtgtct
gcgcttgtgc gtgtccaggg gaaccacagg gagcaccctc attctaagcc tccagaggac
tgcctgaagc cgctagatag aaactcccct agaatgtaag ctccgggggg gagggagctt
tgtttgatgg ctgctgtatt cccagtgccc attgaagtac tggggacaca ttagatgctt
aataaacagc tgttgagtta atcaacggac tctaggaatg gaggcagacc ggcccttctg
gaactggaga aa
SEQ ID NO: 212
ttgaaacccc gtctctacta aaaatacaga aaaaaaaaaa atagccgggc gtggtggcgg
gagcctgtag tctcagctac tcgggaggct gaggcaggag aatgtcgtga acctgggagg
cggagattgc agtgagccca gatcgcacca ctgcactcca gcctgggtga cagagcgaga
ctccgtctca aaaaaaaaaa aaaaaaaaaa aagccgtcgc gcctcgggag tgggctgggg
ggagaggggg tgcccaaggg ccacaagagt atgacggggc tgtacgagct gctgtgacgg
gtgctgcatg cgctgctccg tctgcaccgc acgctcacct cctggctccg cgttcggttc
cacacctgga actggatttg gcggcgctgc tgccgcgccg cctctgccgc ggtcctagag
ccgcttggct tcacgctccg caagcatgga acagccctca ccacacgcac ccgcgcgggg
ggtagtgcct gcccgcagcc caccaccgaa tgcgctggcg cgcggacggc ccttccctgg
agaagctgcc tgtgcgcatg ggcctggtga tcaccgaggt ggagcaggaa cccagcttct
cggacatcgc gagcctcgtg gtgtg
SEQ ID NO: 213
gtcgtgaacc tgggaggcgg agattgcagt gagcccagat cgcaccactg cactccagcc
tgggtgacag agcgagactc cgtctcaaaa aaaaaaaaaa aaaaaaaaag ccgtcgcgcc
tcgggagtgg gctgggggga gagggggtgc ccaagggcca caagagtatg acggggctgt
acgagctgct gtgacgggtg ctgcatgcgc tgctccgtct gcaccgcacg ctcacctcct
ggctccgcgt tcggttccac acctggaact ggatttggcg gcgctgctgc cgcgccgcct
ctgccgcggt cctagagccg cttggcttca cgctccgcaa gcatggaaca gccctcacca
cacgcacccg cgcggggggt agtgcctgcc cgcagcccac caccgaatgc gctggcgcgc
ggacggccct tccctggaga agctgcctgt gcgcatgggc ctggtgatca ccgaggtgga
gcaggaaccc agcttctcgg acatcgcgag cctcgtggtg tggtgtatgg ccgtgggcat
ctcctacatt agcatctacg accaccaagg tattttcaaa agaaataatt ccagattgat
ggatggaatt t
SEQ ID NO: 214
gtcgtgaacc tgggaggcgg agattgcagt gagcccagat cgcaccactg cactccagcc
tgggtgacag agcgagactc cgtctcaaaa aaaaaaaaaa aaaaaaaaag ccgtcgcgcc
tcgggagtgg gctgggggga gagggggtgc ccaagggcca caagagtatg acggggctgt
acgagctgct gtgacgggtg ctgcatgcgc tgctccgtct gcaccgcacg ctcacctcct
ggctccgcgt tcggttccac acctggaact ggatttggcg gcgctgctgc cgcgccgcct
ctgccgcggt cctagagccg cttggcttca cgctccgcaa gcatggaaca gccctcacca
cacgcacccg cgcggggggt agtgcctgcc cgcagcccac caccgaatgc gctggcgcgc
ggacggccct tccctggaga agctgcctgt gcgcatgggc ctggtgatca ccgaggtgga
gcaggaaccc agcttctcgg acatcgcgag cctcgtggtg tggtgtatgg ccgtgggcat
ctcctacatt agcatctacg accaccaagg tattttcaaa ag
SEQ ID NO: 215
agcctgggtg acagagcgag actccgtctc aaaaaaaaaa aaaaaaaaaa aaagccgtcg
cgcctcggga gtgggctggg gggagagggg gtgcccaagg gccacaagag tatgacgggg
ctgtacgagc tgctgtgacg ggtgctgcat gcgctgctcc gtctgcaccg cacgctcacc
tcctggctcc gcgttcggtt ccacacctgg aactggattt ggcggcgctg ctgccgcgcc
gcctctgccg cggtcctaga gccgcttggc ttcacgctcc gcaagcatgg aacagccctc
accacacgca cccgcgcggg gggtagtgcc tgcccgcagc ccaccaccga atgcgctggc
gcgcggacgg cccttccctg gagaagctgc ctgtgcgcat gggcctggtg atcaccgagg
tggagcagga acccagcttc tcggacatcg cgagcctcgt ggtgtggtgt atggccgtgg
gcatctccta cattagcatc tacgaccacc aaggtatttt caaaagaaat aattccagat
tgatggatgg aattttaaaa caacagcaag aacttctggg cctagattgt tc
SEQ ID NO: 216
acgggctgag cctcaaacga gctgcaggcc gagttctaac gggctgagcc tcaaacgagc
tgcaggccga gttctaacgg gctgagcctc taaggaggga aacgtcactt cctgcctcac
acagagccca gcgtctccat gtccactgat agccttggta tttgcaacta tgtccatgac
catctctgtt tctccaaaac agcctctagc tacataaact gtttagaaaa cctcatgcgt
aaagcagagt atgtcaaacc tccatctgtg gtcaggagtt aggacatccc cagctgcaat
ttgagcaaag acggcgcttc cagaggatca tcggatcctg tgtcttggtt ggggttgggg
cccatcaact taaaatagct tctgtttatg ctggtgaagg aggcacagac ttcaccctat
ctaattccaa ggaacaggcg agggtgggag ctgtagcgga agagacaaaa gcaaaaggca
gattcgcccc tttgtgtggt cccgtaagtg acactgtccc tccctctccc tggaaacagc
agcccccagg cacccccccc agcaactggg acaagggcac a
SEQ ID NO: 217
tggctgtcga ggagctgctg ttgctgttcc gcgtcggtcc tgctcctgcg cgcgtcgtcc
aggcccgcca ggtcgccgac cagtctctag ggcgtccatc gcgggaccca cgggaggcag
aagtggaggc cgtgcgcacc gcgagctcaa cacagttggg ggccaggtgg ccgcctccca
gcaggttgtc ggggttgagc tgggtcttgt gctcatcgct gggcttgtag tgcggtgccg
gtcctcaagg atggggccga agagatcctt gaacacgtcg taggactcct cgtcggccgc
cacgcggccc acggccctga gtacgggtgg cccgggctgt ccacgcgggt ctggatggcg
cctccagcgc gaagccaccc ctggcgcgca gctccgcgtt cagctggggc agcgcctcgg
ccactgggtc ttggtggccg ctcaggtcgg gaaactcgtc ctgcgccggg aggcgagctt
cagcgccccg cggctgtcgg agaagggcat gtgcgggcgc tcggtgggtc cgcagctctg
agcgtggcca ctttttaact gttataaata attctgctat caacattcat atgtacactt
ttcttat
SEQ ID NO: 218
ctgctattga tgttttctct gcccaggttc tccccacaca cggggttagg gagggtgtgc
cagcctgccc tcacatcccc agacagagtc cccctccagc atctgctgcc tacctccttc
tccctcagtg cctgtttgtt tttcttccag aaccatcgcc tctcaccaag gcagccatcc
aaggggggcg gtgttccgga gacatcctct gccccccgca cccctgcagc ggtagcctgg
tgggggctgg tgctgcagtt tcgtcagccc ttggctccgg gctctgcagg cggaatcccg
agcctgcgtg agggccgccc tggcctcggc gtgtgtcctg ggaaggggcg ttggaagcct
ctctgcttgt cttggctgcc tctgctcgct cagctctgcc cccactgggg ccgccagcct
ctgcactccc ccttggagga gccaggcagg gtttgggtcg gagctggggt agaggaaggc
tccaggcggc ttgccgcagg atctccctgc tgtagccagc ccttggggcg ctcagcaggg
tgggggacca tcagtcaggg tgggggaccc tcagtcagga taggggggct cctgttcttt
ccactgccac caagctaccc ttcccctaac t
SEQ ID NO: 219
taccccagct gcctgaccgg gagagcatcc tgttcttccc ctctggaatt ccgggtccac
agctgtcttc ctactcacat ctggcctcgg cattcccgcc aagccctccc cttgaagcac
aaggatgttt tgtccaggat cctgagccca gggccttcca ggtggcagag agagatccgg
atgtccagcc agctctgggg gttcccccat cctgccagtg tggggacctc cttgctgtag
ccaggtcagg ccagccctag ggagacagca ggatggttcc aggaagcctg ggccgctccc
cagatcaatg cagggacgga cagcagccag caggctgggc cacggcatca gagctggggt
caagaggttt ctagccctct tgtggctctc agccccgggt cctggctgct tcctgctggg
cagtgacctc cccagtccat ttccctccct ccttcctccc ctggcctgag ctcagctcat
ggaaggaggc cctgtgtgca ggaaccttga tctgcacctc tgaaggatgt cagggcagct
ttttctctgg gcctgtatga ctcagcgcag gatttagggc aggtggctcc accgtggagc
ctcagtttcc tcatctgg
SEQ ID NO: 220
cccgtctcgg ctctggctcc gtcccctggc ctacccacta gcgggtcgga ctccgcccct
gcttctgacc acgcccccgc gcccaccctc ttcccaccct cctcccaccc agggctctcc
agacgcgcat gcgcacccgt tgtgcatctg ccgcgtggtg accgacacgc cgtcggcgcc
gtccccgctg ggccgcagca gcaggttccc gcactcgggg tagcgctcca ggagcagttg
tgcctccagc cggctcacct tcaggaagca cctgtggcgg gccgcgtcac ccactcggga
ccccggagac caagtccgct cttctgcacg taaaccctgc ctcctctgag acccagcccc
atccccatcc cctaggccca ggagaccctg ccctgctctc cagacccagg cccctcccac
ggagacccag tccggccttc caggctccta gtttttgtgg ggttttttgt tttttttttg
agacagggtt tcgctcttgt tgcccaggct ggagtgcaat ggcgctatct cggctcaccg
caacctccgc ctcccgggtt caagcgattc cccttcctca caggcccggc taat
SEQ ID NO: 221
gattttcagg aaccatgcat ggctatcgcc tcctcccgcc tggagggctg ctcctgcgcc
tctgaccggc gctggttcca gccgcggccc agctgagcac agcaggaacc gcagtagcag
ccggagcgcc cacgcccggg gtcgcctagc ccaggaacgc cttagttgca accctgcgtc
gaggcccagc tccgtgcgca gaaagccgag gccaaccaga gcatttcctg gacgagtcct
ctcggcctgc ggggccagtt cctcctacca gcttcctgct gccacctcgg cttccatcag
agggacgctt aggatggcgc aggggcccgg agacactgtg aagagtccag gggaatgagg
aggggctggg ccgggcagcc tcaggcccag cgcaggttag cgcttctcac gcctgagcag
agatcagcta ctgccactgc ggggaggaca gaaggaccca ggctccccag cctccctctg
caccgggagt gtaggaaact atttaaaaat aataataata ataataataa taagtatgga
atagaacttg cagatctaac ccaaccaagt tttcattctt tttccttttc cttttctttt
ttttaatgta tttt
SEQ ID NO: 222
tcacttgggc atcttaagag tgggttcgta aacttggttg tgtgcgctgt gcagatgtca
gtcaccctgt gtggtgggca aagccgactt ctccgcctct gtagctccga aactacaatc
cccagaggcc tctgcggtca cttccgctcc cctccctacc cttcagtgtg tagcgttgac
gtcagaaaca cttccggtcg gtggcccagg cgcgttaagc tggttgggac ccgggaaggc
ctccctctta aggtctttcc cacacctctg caccttgtta cctgactttc ggcttcagga
tccgcagcgt gcacccgcgt tccgtgagtg ccctataggc agtcagcatg cccctctgcg
tgtccctgtg ttacggggac gccggctggg agccgcagag ctatctcaga actagggcgc
tctcctttgg gcacctccag gccattttcc tttcattcga gcccacaggg ttagagataa
accctcactc cgttgcttgg ggacaagggc ttcactccct gtcccgagct tgcggctgag
cttgagggtg gctgggtcat cctggccccc cactggatgg gaattggctg ctctggtgat
ttctgtga
SEQ ID NO: 223
cggagaagct ggagcggcag ctggccctgc ccgccacgga gcaggccgcc acccaggtga
gccccgcacc tgcccactcc ctcccctccc cgggcctcct acccacccct gacactgcac
cccgcctccc caggagtcca cattccagga gctgtgcgag gggctgctgg aggagtcgga
tggtgagggg gagccaggcc agggcgaggg gccggaggct ggggatgccg aggtctgtcc
cacgcccgcc cgcctggcca ccacagagaa gaagacggag cagcagcggc ggcgggagaa
ggctgtgcac aggctggtga gcgcctgggc cagcggggcc tgcctctgat gcctcgcccc
cttccttcct tcctcccacc atgggctgcc ctgggtgctg cgggcagcct gcacacccca
agccccgcat gtggcctgtg gtttgggctg tttgggatcc tcacagctga gactcatttc
ccagcctctt ccaggcaggg ctcgggctgg ggtgggacag ggtccctggc gcttctgttt
gaggggcggg gtggggggag gtttctgcac cgcagaccag gggagatgga tgacaaaagg
ggcttcagca aacagct
SEQ ID NO: 224
gatctcccta agaggttatg ccagtcacac tcctgccaag agagtatctc tgcgccacgg
ccaagggtga gtcatcctgc tgagaggttg agctggggac gcctgcccag atgggctcca
agtgagggag agcctggcgg ggagaacagc ccggacagag gcagggcagg gcgccgggac
actgcttggc gcgtcctggg agtgaagcgc attgaaccca gctcaggctg gtggtggggg
agtcttggca atgctctctc tccaaaggcg agttgatcac agacgctggc agtgagtcag
cggcaccgcc agggctgctg agaaatccct cctgctgtcc gatcgcattc ctggaagggt
gggccgctca gggcccccca gctccagtcc cactcaggcc ccagaatccc agcagcccac
cactcacttc tttgcgctca ctcttccttc tggtccccac acaccgctcc ctctctcgct
accttcagtc tttgctcaga tgtcgagttc ccagaggggc ctccctgacg ccaccgttct
agcagcattt agcatttaga taaatgacaa attttagatt aaatgttaga t
SEQ ID NO: 225
agagcctgca ctggggaaga tacacaccac agaagccggc gctgcagaag cacatatgcc
aaggacctag cgctggagac gtgcacacgc ctgggaacag gtgctggggc ggcacccaag
cacgggagcc agcgttgggg aagcggcaca ccccagggag ctagcgctgg caaagcacac
ccaccaagag tgagcgctag aaagccgcac acactatggg agctccgccc tggagaagcg
tcacgtgtgt gcctgggaac cagtgctgga gaaagtatgt ggaagctggc gatggagaag
gcgcgcgcat gtgtgcacaa cctcgctctg gaggagtcac ggccaggtgc gcgcacgaca
accttcaccg gagaagtcac acgcatgcgt gcgctggaga acctgaattt gtaatttcaa
atttccctat aaagaaatat ccacgaactg atgactttgt gagtgaattc tatcaaatat
ttgaagaaaa aaaaatacca atccttcaca aactctgaaa aaataggagg gaacacttcc
caactcattc taagatgcca ctattacggt aataccaaag ccagacaga
SEQ ID NO: 226
aagtgttggg attacagaca taagccaggt cgcctggccc aagctagata ttgaggactg
ccagatggca acagtagaca agacacccta gaatggccca tctagaagga agtagatacc
ttctctgtag ggattcaaca agggggcaaa gtgatgggca tcttgggtga ggaacccagc
ccgcggaatg ggaaagggct gggacactgg ctttacagct gggttgggaa agggatctga
tccttgagtc agcccctgta aaatggggat acagcagggc acgacgtctg ttggtcgcct
ggcactgggt cggccaccga ggccgcgcct tggcctcttt gtcccctctg gccaccggcc
ccagggagcc cgctcgggaa gcagcgcggc cccaggagga aggcggcgcg gccgaggcca
gagccggcgg ctactgcgac cttccggctg gcgggcgcgt ttcatgttcc tgcctcaccc
tgggctgcac ggactcagat cgggaagggg gaggatccat ggtaaaggcc acgccccctc
tgggacctcg attccccttc tgggcggccg agggatgggc tgcaaggagt caaatcctct
t
SEQ ID NO: 227
gccagcagac aataacctct cctcttgaat tggaaaaaca aatctgtgga ggccttctct
gccccttgtt ataggggccc catatgccgt tcccaggatt aaaccaaaaa gcacacattc
ctctctggca ggtgcaggtc ccacccactc tgggcagcca actgatgtgc acattttaat
ttcctaaaac accaggacag aaccttcctc aggggtcagg tggctcaccc ttggccctca
ggctttggag atgggcactt gtcatcatgg gtgtttggaa agcaactcta cgttctagcc
tgtgctccat cgttccttct acatacaagt gatgcaaaca tcaaaatatg ttttttcttt
cttccttcct ttaaaaaaaa ttgaatcctg gatgaagttt tagctctgtc acttgacaac
tgcattatat aacctagggt acttgaatct cagccccaaa tctctaaaat gggggcagta
acatgcttct gccaggtccc agacctgtgg gtctccaaat ctgcacattc ttcaagcctt
acaggtcctt ccctggtctc tctaaggatg tcatgggcac agagcc
SEQ ID NO: 228
gaggcagagg gtagggggtg aggaggtgtt tcttgtcttc ttcttccaat ctcagaagta
aacattggaa agtggggccc ccagcagtgt acagcccgtt tccaaaccag gcctgtaagg
aggagctgag gtttcggctg agcccccagc ctcccccgac cgcacagcct cgggcatgaa
cccgcgaagc cagacgctta gttgcttatc aggccatcgc tgtacatatt tagaaagtac
ctatcactca gacactttga aaagcgtggc gttccagcgc aaaccaaccc gaacgggttg
gaagggggca gtcctttctt cccgcaagtt cggggctcga gagacggctg caggaaggcc
atcacccctg gcttcctgca gccacagctt ccagccccac acgatgccca acttcatttt
agcagtggcc cccaggggaa atcacaccat tcttggtttt gtccctccct cctgaggttg
ggacattgtt caaacaaaag taagccttca gctgacagag aagctgcccc gcctcttccc
tgcccttgtc ttgctggcat tcattgggac taccaggtag ctttccttcc cagctcaggt
gtttacctgc
SEQ ID NO: 229
ttatagatga gattctactt aggggtagga ttcattattc atgaagggtg tggtcaggtg
aggcatgttg gaagcaaaat gcgaattagg taaggtggag tagaagagag ctattggcaa
gagaaaaatt acttgagcag tgtgtgagtg ggtgggtgag aaagtgggca gggtggactc
agaggttggg aagctgctcc tgagaggaga agcctctgtc tctacacagg aacctacctg
acacatgagg caaaaggctc cgacgatgct ccagacgcgg acacggccat catcaatgca
gaaggcgggc agtcaggagg ggacgacaag aaggaatatt tcatctagag gcgcctgccc
acttcctgcg ccccccaggg gccctgtggg gactgctggg gccgtcacca acccggactt
gtacagagca accgcagggc cgcccctccc gcttgctccc cagcccaccc acccccctgt
acagaatgtc tgctttgggt gcggttttgt actcggtttg gaatggggag ggaggagggc
ggggggaggg gagggttgcc ctcagccctt tccgtggctt ctctgcattt gggttattat
tatttttgta a
SEQ ID NO: 230
aatcaaatta caagaaaggg aaagagaaag gaagagggag tgggacccag agagctggcg
ggaggcagcg aaggggaaag cttcagtgca cgcatagctc ctgcacagcg gctcctgcag
ccccccagga tgcgcctgag ctgaggctgc ttgtgggcag gccctagaga gaggcaaact
ttgactccag gcacgcagca ggtttaactc ctcactggct gggttctggg agctctgggc
acacaggata ggcaccaaga actaacacat cctggagctg cccggagttc cgctcctgcg
ggcttagcag gaaagggtgc ctaaggtgag tgcccacttg cgtccgatcc tctgggggcg
atgcagggtc ggggcgcctc agtgtgtctc gctgcttgtt ctggttgcag tcgggaaatg
tgggactttg gggtcttctc ctttctccgg ctttcttttt tctccttctt tcctctctgt
tttcttgtaa attacacttc gactttcaaa aaaaaaaatg taggggaccg gtggggtcgc
tggggttggg ggagagactg aagaaagtgc gcctgggcgg aggcggcgaa gggaatctct
gggcccgagg aatatacctt gtccctgcac tagtgtgtgt tctcttgtgg c
SEQ ID NO: 231
tggttctttc tgttgccctc atagaccgta tgtagcagtt cgcgtgggca cagaacccac
ggtttcccgc tagttcttca aaggtgaggg caggtgcccc gagttatttt cctggggact
gagcccagag cggggcgatg ttgtgctact gcacctcccc gccgcagccc tccgctgttt
tcttttgggt agtggtccag gaacttaaga cagttcctcc tggcgatgtg atggaattta
atgggacagg agaagggaac gggctttctt ttcaggccag cgtggcagcg ggcggtaggg
cgaaagggag aaggaaacga gggtttattc cgttgcccac tccgcggtaa gcgacgttgt
agggctccac tgtagcgaga gccccgtgga ttcctttttt tttagccatt tagtttgtaa
acatcacttt aaagaataca tagtgtattc atgacactcg gtgaaaaaaa actttccttc
ccctcccgcc cccccggggc agtagatatt tacaaccgta acagagaaaa tggaaaagca
aaagcccttt gcattgttcg taccaccgag atcaagcagc agtcaggtgt ctgcggtgaa
acctcagacc ctgggaggcg attccacttt cttcaaggta aa
SEQ ID NO: 232
ccagttcggg atcgtgtagc cggcggggcg ggggccgtgg ggggcctgga ggagggcagg
ggccgcggga ggccgggagg agggtgggga ccttgcagcc cccatcctct ccgtgcgctt
ggagcctctt tttgcaaata aagttggtgc agcttcgcgg agaggagagg cgctgcagtc
tgtgctgtgt ccgcggggcg gggaggaggt cccaggagcc ggttcgaaag ctccctccgt
gatgaagtag gcgagaaggg aggaggtgaa ggagggcgag ctgagcacac gcgcttcatg
ccacaggagg gtgggaatga gcggaggact gaggagagga aggagggaaa gaatagggag
atgaaaacgc cccggtctgc tgctaagcac agcacagtta ccaaagccag gaaactaaca
ctgacacgat attttattta cgttacagct ctattcaaag ctccaggctt ctttttgtag
aatcgtttcc atctgctgga atccagcatc gcccccaccc cccgccccat ttctaggggg
atgcccccac tgctgacctc tcctgctgta gatctatttc tgggaggcac tgacatgctg
actcttgcta tggggtcggc gggg
SEQ ID NO: 233
gggaggccgg gaggagggtg gggaccttgc agcccccatc ctctccgtgc gcttggagcc
tctttttgca aataaagttg gtgcagcttc gcggagagga gaggcgctgc agtctgtgct
gtgtccgcgg ggcggggagg aggtcccagg agccggttcg aaagctccct ccgtgatgaa
gtaggcgaga agggaggagg tgaaggaggg cgagctgagc acacgcgctt catgccacag
gagggtggga atgagcggag gactgaggag aggaaggagg gaaagaatag ggagatgaaa
acgccccggt ctgctgctaa gcacagcaca gttaccaaag ccaggaaact aacactgaca
cgatatttta tttacgttac agctctattc aaagctccag gcttcttttt gtagaatcgt
ttccatctgc tggaatccag catcgccccc accccccgcc ccatttctag ggggatgccc
ccactgctga cctctcctgc tgtagatcta tttctgggag gcactgacat gctgactctt
gctatggggt cggcggggag tggggagctg ggcattcccc ttcttcctca ggaca
SEQ ID NO: 234
acacacactc cctcagacca gatgcccaac cacttccaga tgctacagtc tcggatatcc
ttggttaagg aagaggaaga aaaagctcgc ccttcacgtc cagatacttg ggttcgggtt
acatgaaaca ggattagttc agaaaatcgt gccacttcac agccaagaca aaaacccaag
aatgaaaacc atgtatacag ccaacacaat agcaagactg aagacagtga caaagagagt
tttctggttc tgctctgatg cctctccctc cacaccacac ctgtgatcta ctgtgcatag
gatctcacag gcccaataac agagctggag ttcctcttac gtgacacagg atttggcatt
tgcctgtgcc gggctatcac tcctgccctg caacacgctg gtcagctgga gaagcctgct
gctcacacac tcaccagcaa cttctctacc ctggatggtc accaaaaagg aagagcaatg
tctgtgcccc cagcattggt gcaaaggaag tggcagagaa gcaacaagga gggtggtgcc
ttgccccaac tgcccgccag cacccacagc caaggcaact gttctctggt gaaggcagag
ctggaaatgc atgcctgagc
SEQ ID NO: 235
caagggattc tcctgcctca gcctcccgag tagctgggat aacaggcatg caccaccacg
cctagctaat tttttttttt aatgtagtag agatggggtt tcaccatgtt agccaggatg
gtctcgatct cctgatcttg tgatccaccc acctcggcct cccaaagtgc agggattaca
ggcgtgagca ccgtgcccga ccaagattga ccttcttaaa caactttgtc atcatgtgct
tctcctgctc agacatcctc cttggcagcc tttcaacacg tttctcaaat cctttcccag
cttcctgtgc agcctttcct cctcagcctg gctgccttac tgtctcagct ccgatctctg
ggccttttcc catatggctg cttccctcta cagtgttcct cctagcccat accccaaccc
accccacctt tccctcctct ccaggttgta ccagttccag gcccctgccc ttgacaatac
tccttcccac gaggagcact tcctcggcta cctccttagc gtgtattgga attcccactc
acttggcagt tgcactttgt gacacttaat tctgccattt tattttccta actgctattt
agtctccctg tttattt
SEQ ID NO: 236
gagggagttc aacggcgacc acttcctttt ggagcgcgcc atccgggcag acttcgccct
ggtgaaaggg tggaaggccg accgggcagg aaacgtggtc ttcaggagaa gcgcccgcaa
tttcaacgtg cccatgtgca aagctgcaga cgtcacggcg gtggaggtgg gggcttcccc
ccagaagaca tccacgttcc taacatttat gtaggtcgcg tgataaaggg gcagaaatac
gagaaacgaa ttgagcgctt aacgatccgg aaagaggaag atggagacgc tggaaaggaa
gaggacgcca ggacgcgcat catcagacgc gcagctctgg aatttgagga cggcatgtac
gccaatctgg gcataggcat ccccctgctg gccagcaact tcatcagtcc cagcatgact
gtccatcttc acagtgagaa cgggatcctg ggcctgggcc cgtttcccac ggaagatgag
gtggatgccg acctcatcaa tgcaggcaag cagacggtca cggtgcttcc cgggggctgc
ttcttcgcca gcgacgactc cttcgccatg atccgagggg gacacatcca actaaccatg
cttggag
SEQ ID NO: 237
agaactggcc ctcccctctt cactcttttt tttttttttc ttgagacaga gtctcgctct
gttgcccagg ctggaatgca gtggtgcgat cttggctcac tgcaacctct gcctcctggg
ttcaagcaat tctcctgcct cagcctcttg agtagctggg attacaagtg tgtgctacca
cacctggcta atttttgtat ttttagtaga gacagggttt caccatgttg gctgggctcg
tcacaaactc ctgacctcag gtgatccacc cgtctcggcc tcccaaagtg ttgggattac
aggcgtgagc cgccgcgccc agccccctcc tcactctctt tctcttcctg taacttctac
agctgggcaa gagctgggtc tccagcggtt gcacggagaa gtgtgtctgc acgggaggag
ccattcagtg cggggacttc cgatgcccct ctgggtccca ctgccagctc acttccgaca
acagcaacag caattgtgtc tcagacagta aggggagcga ccggggaggt tggagagggg
agcacctgtg gccagggcgg aggtggagga agaggcaggg tgggaagggg cttagcctga
accccagcac agtcaggggt tggggcgggc g
SEQ ID NO: 238
gaaggatgag aagcattttg ccagactcac agcgggacag tattccaagt agagcatgga
caaggtacag agaccaggtg gctggctttc tctagcaacc tgtctgctac cccagttctt
ccatgaccaa actgggtgta ttgaacaagt tacctcccct ctctgagcct cagtttgctc
atcagcaaaa tgggggtgtt ggcagagacc tttcagacct ttcggagtta ccaggggtgg
ggtccgcaga tccccatagg gaggacttgc gcacagttgg cgctgggtaa atgctgggag
aactgctgcg ggcgagggga aagggttaaa gctaggcgct ttttaattgt caaatgactg
cgggcgatta gcactggcag cttcctcaat aatcgctctt ctctgtacct gctgggagct
taattaaaaa acaaagaggc tcaatttaaa ggccattact atgctaatgc ggccgggcgg
gcggtgatta agcggctcag gcaggcagcg ggcggctggg gcggggcatg gggcgatcag
ttacccacta aatgggcggg ctgcgtgccc tgcctgtccc g
SEQ ID NO: 239
caggacagtg tgggggcggt ccctagttca cagcagggag gccctaagca gtgaggtggc
ctgcccgcca tgtcgcagga gcccctagcc ctggcagact gggcagtagc gctggctggc
cgctccgggt tgtgctgcag gaagcccttc tccagccgcc agcctcgtcc tgcccagccc
tgggccccac atggcaggaa acaaggccaa agaggcaccg cttagcaagc ggcaggacgt
gccagggctc acacagacac cctgagcttg caacactccg ggcctctgcc gcgtgtttat
ttcaggatgc cgtggcattt gggtgacctt ttgtgctcac catggcttgc gtcgtctccg
ggtcactctc gtctggactg aagtcccgtc tccctcagct gagcctgtgc catggccagc
ctcagcggga actggcaaag ggaaaggggt tccttgggga ggcagcaggg gtttctgaag
gcttcatctt caagcaaggg gtttagagct caggagtgta tttgtgtttt tttgtttttt
gtttattttg agacagggtc tggctgtgtc actcaggctg gaatgcaatg gcacagtcct
ggctcactgc agcctcaacc tactgggc
SEQ ID NO: 240
cttgaatctc ctccagtcct tccactatat gcccctctgg ccttgtcttg cacacctcca
gagacagaag cctcactact ttagaggctg tcctttccat cttcctgact ggaaggaaat
tcttccctga atggagctga aacttgtgtc cctgctctgc cctctagggt tttttcccag
taactggaag agtcttgaaa gggctaaaat gattttattt ttaaatgtgg acaggcaagc
agaggtggtt ggcaaaggca aggtggctga cgatccggaa gctgtacagg agagataagg
gcactggctg ccagagtgcc ctatcgaagc atcatccgaa ccctgcggta ggggtggccc
acaccacggc ctgaggccca gtcaatgcca tatttgtggg cggcagcctc agacactgca
tagcgaccat tgagatttga tcggtaacag gatgcatacc accaggcacc gtggacaatc
actgcacagt tgctgttgct tgaatcgtgg tcagcgtcat aggtggtaaa gggcctccca
ctgtggaggc tcagggaatc ccctagcagg gaagggatgg aaagcacctt ggtgcccagc
accacgcctg gcacctttgg agatataatg ccatgggagt ctcagagcaa c
SEQ ID NO: 241
ccagagacag aagcctcact actttagagg ctgtcctttc catcttcctg actggaagga
aattcttccc tgaatggagc tgaaacttgt gtccctgctc tgccctctag ggttttttcc
cagtaactgg aagagtcttg aaagggctaa aatgatttta tttttaaatg tggacaggca
agcagaggtg gttggcaaag gcaaggtggc tgacgatccg gaagctgtac aggagagata
agggcactgg ctgccagagt gccctatcga agcatcatcc gaaccctgcg gtaggggtgg
cccacaccac ggcctgaggc ccagtcaatg ccatatttgt gggcggcagc ctcagacact
gcatagcgac cattgagatt tgatcggtaa caggatgcat accaccaggc accgtggaca
atcactgcac agttgctgtt gcttgaatcg tggtcagcgt cataggtggt aaagggcctc
ccactgtgga ggctcaggga atcccctagc agggaaggga tggaaagcac cttggtgccc
agcaccacgc ctggcacctt tggagatata atgccatggg agtctcagag caactaagag
ttgaatttta tcaggcccca cgagc
SEQ ID NO: 242
ttccatggcc cagaagtctg caggacccac agcaggtatt cgggactatt tgttcaatcc
acacctgagt cgttgcacga ttatgctcaa gtccctcgga acacctcgcc tgccatctga
cagcttccca tccagaaacc acacagtaca gtaaaaaaca gaaaaaagaa agccgttaga
ccccagtgaa tgttattttt aatgaaagtg gtgcattttg actcacaatg ttgaaaccag
attataaatg agtcatcagt gaatcgacca caaagagcct ttgcggaggt gatttacagg
agagctctga tgtctgctgt cccctgcaca cgcttcacag agatgctgtc agacgcagag
ctggtctggg gcatctgttg ccgcgtcagc tcaaaaggat gctgtgttgt caccaatggg
attccccagc ccaggcggtg ttgcggtccc acccacacaa ggaaggcggc catcactgaa
taatgcttgt ggttacatca tcattgctgg tttccaggta gtgactagca gatactggag
agagacaggc catctgctct tcctgtgcgc ctcagctcc
SEQ ID NO: 243
tagcttgtca gcatgaacct acatgcaagc cagagatcta tgattttgtt tcccagggag
ggagtgacta atgcgcgcac cctgaccatc accgtaaaga ggtaaagaga gagtgaaatg
gctcaacgta cacacacccc gctccatacc ggggacgagt ctccgagctg cggcttgtgc
tctcggaggg ccaggctgaa gctgaccgcc cccacggcca cgctggacac ccccagccct
atctccagca cggagagcac caagaggctc ccaataatct gaccgctggt gcacatcctt
cctcggtcat cttccttcca gatcagagag ggaaatcaac catctacctt tttttcttcc
actatcctcc ttaccccttc caccccctac cagatcccaa aacttttctt tcttcaagag
cgaggcatta tccacaaggg ctggatttcc agaaacgaag accttccctg gctgggccag
aggcaaagga gctgctccac cccctggcac gttcagatag ggatcgtaga aggatcttcc
tgggttcggt ggtgcgaaga ttgcacaccg gtaccggggc ttttaagcag cggaaaacct
ggaggagccc agggagctcc gagccttgct ccccaggcgc tgtccagagt
SEQ ID NO: 244
tgtcttagtc aggagggttg gatgtaagaa acaagccctt aacattcgct tctttgtgga
cgagatgcag tagaatcatt tagtccttgc actctgagcc tctccacaga atttgctgtt
ggaagtcatc tcagtaagaa atacacagag aaatctggtc tttgttccta tgatgacaaa
gcagtttcat aatctgccct cttgcagctt gctctgtttt gggtgcagat aaaacaagca
tggttctcta ataaccacct gcacctctgc tgcaatgtaa acagcagatg tgggcgcagg
gtgagaaggg agaggaagct acgtgcaatg gcaggttggg gaataaggag gcagaggggc
tccttcatct tttacagggt aaaatgggat caggacagtt gcaggacaga cttgtttctc
aaccacgctg ttaagagaat ttcatactgc aagtcacaag ggcccagggc tccacggcct
ttagcccacc ctggctttct aacaacccaa agtgggtatg gagaaattgt cctttaaaaa
cctaaaaact atgttaattt tcattttcaa aataaagaat aaatcagcac ttttggaaag
gaggtgggaa ggg
SEQ ID NO: 245
acgcttgtga taacgataag acagaaacta ttgaaaaggg tgcagtggtg gtgtgaagga
ttaatccttt gcttgcttca catctgaaca ggaatctcca cacaaatgtc ccacatgtgg
aagaaccttt aatcagagaa gtaatctgaa aactcacctt ctcacccata cagacatcaa
gccctacagc tgcgagcagt gcggcaaagt gttcaggcga aactgtgatc tgcggcggca
cagcctgact cacaccccgc ggcaggactt ctagagaagc ccaggatctg tcccgtgccg
ccgctgctcc cctccccaga cacctctcca cgtctcctac ccagggggtc gcatccctag
cccttcactg accccagctc ttcccttgct gcagccgcac ctgcagctcc agggagttaa
ctcttcttct gggggactga gaactgtaga aagccacaca ctactacatc ccttcacaaa
gagtatatgc tagtttcttg tagatattca cagctcattt tagagctctg tacataatgt
tgtgggtctt tgttttgttg ttttgtttgc tttgggatct tgttggatgc acttagatat
ggaaaatgga agccaaattt tatctttaaa gactg
SEQ ID NO: 246
ggggaaaact ccaggtcaga tggggtgaac cagagggaac aatgcacttc ttcacaaacc
aacatataaa cacttgcgaa tgaaatcacg cagagacatt catcagcttc aaaaggagag
cggaactggg aaaggagtcg gcagaattga gagaggagaa tttgggaaag cttctccatg
agagcggtgc ctggagaggt gggttgggaa ccgtcgctga gaataaggca caggtcagcc
acctttccca gcatctcctc ctcgcaaacc ccaagccaag gcaagctgga tgaagcgctc
cctgggcagg cccggctctc cgtgtccctc catcacctga ccccgctggc tctcgcagac
cccttcctcc acactcactc ctcccggctc tccttctata atctcctgac atctcttcaa
atccaattat tgaattaatt gacgtacgaa cccagaggca aacagaaagg ggcggcaaac
actgggcggc tcagatttat ccttcggcct ccgcagggcc cggccggacg agatttactg
ggcctcgaac acggcgacag ttcaaacctt tgattaatca tgtttttctg cctaccccat
aatttagttg ctctttttcc ctccctgcct tttttttttt tttta
SEQ ID NO: 247
gagcggaact gggaaaggag tcggcagaat tgagagagga gaatttggga aagcttctcc
atgagagcgg tgcctggaga ggtgggttgg gaaccgtcgc tgagaataag gcacaggtca
gccacctttc ccagcatctc ctcctcgcaa accccaagcc aaggcaagct ggatgaagcg
ctccctgggc aggcccggct ctccgtgtcc ctccatcacc tgaccccgct ggctctcgca
gaccccttcc tccacactca ctcctcccgg ctctccttct ataatctcct gacatctctt
caaatccaat tattgaatta attgacgtac gaacccagag gcaaacagaa aggggcggca
aacactgggc ggctcagatt tatccttcgg cctccgcagg gcccggccgg acgagattta
ctgggcctcg aacacggcga cagttcaaac ctttgattaa tcatgttttt ctgcctaccc
cataatttag ttgctctttt tccctccctg cctttttttt tttttttatc agcggaaaca
gagacggagt cctcatcagc ttcaattaca aatattaagg tcccggacag cactttgaca
gagaggcggc cagcccccca cttcgtacca cccccctaaa tcatctccga
SEQ ID NO: 248
aggtcagcca cctttcccag catctcctcc tcgcaaaccc caagccaagg caagctggat
gaagcgctcc ctgggcaggc ccggctctcc gtgtccctcc atcacctgac cccgctggct
ctcgcagacc ccttcctcca cactcactcc tcccggctct ccttctataa tctcctgaca
tctcttcaaa tccaattatt gaattaattg acgtacgaac ccagaggcaa acagaaaggg
gcggcaaaca ctgggcggct cagatttatc cttcggcctc cgcagggccc ggccggacga
gatttactgg gcctcgaaca cggcgacagt tcaaaccttt gattaatcat gtttttctgc
ctaccccata atttagttgc tctttttccc tccctgcctt tttttttttt tttatcagcg
gaaacagaga cggagtcctc atcagcttca attacaaata ttaaggtccc ggacagcact
ttgacagaga ggcggccagc cccccacttc gtaccacccc cctaaatcat ctccgaatta
acatcacatc ggcggctggc gcgtgttcag atttaaatgg tggcatat
SEQ ID NO: 249
agctagcgtg ttcatgctgg atgtggtgat aataacagta acagcagcaa cagcaataat
aatactgtcc tatctttttt tttttttttt tttttttttt ttcagaaaag atagcctaaa
agggttaaga atcccagcaa gacacaacat agatgggctg aaaactcgtg gcaggatgga
agggtataaa gacgccgggg aagtggctgg ggaataataa aataagaggg aagctaaacc
agtgaccctt gtcggcagtg aaaagcggga gattagaaaa tgtttcatgc taatttccat
ggagatttct ttaatttagc gaagactgct tcccgggctc cgcctggccc gcgccggccc
gcgtcctcgg tggtctgggc gccccggctg agccgctagc gggtcactcg ggcggctccg
acgtctctat cagccgcgcc cgcgccgccc gcctccccgc gctgctgccc ggctctcggg
ctctcgcttt tttttttttt ttttctttcc gcggcagtct taggattctt gtcacatgat
ggcttcatcg ggcccttctc ctcctgatcc tttcaagctc tttctcctgc ctggcatatc
aaaggagatt tgtgggtcac cgagccggga cg
SEQ ID NO: 250
aaataagagg gaagctaaac cagtgaccct tgtcggcagt gaaaagcggg agattagaaa
atgtttcatg ctaatttcca tggagatttc tttaatttag cgaagactgc ttcccgggct
ccgcctggcc cgcgccggcc cgcgtcctcg gtggtctggg cgccccggct gagccgctag
cgggtcactc gggcggctcc gacgtctcta tcagccgcgc ccgcgccgcc cgcctccccg
cgctgctgcc cggctctcgg gctctcgctt tttttttttt tttttctttc cgcggcagtc
ttaggattct tgtcacatga tggcttcatc gggcccttct cctcctgatc ctttcaagct
ctttctcctg cctggcatat caaaggagat ttgtgggtca ccgagccggg acgcagcata
taaagtcatc agcctggccg gcaccacctc gatcatttgc cgcattgttc ttgcaaggag
cccaggatgg ctgtggcttt ttaataacta gcttagtagt tagccgaaaa atcttagttt
ttaaaaatac aaaaaaaaaa aaaaaaaaaa aagagacagt ctgatagttt atttgttttt
ccatacactc ttaattgaaa ctcagt
SEQ ID NO: 251
ctgggggcaa actggagttg tcaggaagat ctgggctttg gaagaatgcg aagtgtcggt
agaaggagaa ggggcaggtg atttcagact gggaggacct tgtgggcaaa ggcacaaagg
cgagactgac ctggagatga taaggccagt tgaagagaca ctggagaaga gaagacagtt
tgttttacac attgcaggaa atcagattag acagttaggg tgtggacaca aaagcgagga
ccttgcaggc actggggaga agtgacccca ttcaatagtc cttggtctcc ttctgccctg
cggctgcgct tcctcggctc tcacggcacc agcagaattc catgtgagag ggagcttgtc
gagcgtggcc tcttcccact tggggctgct ttctgcatcc ctgtgcctgg ctgtgggcct
ccatttgccc tctactgtct tcccttagga catcatttat gcagagaaag gttcgtgtgg
ctcggggtac cagtaagacc tccacctctg gtttcttcat tttaaggagg cccttcaatt
atccaggaat taaagtggcc ttcctcttgg gagaacgagt tggttgatga atgataagca
agtctctatt cctcaaaagc cagtccccaa attccatgaa atat
SEQ ID NO: 252
acacacacac acttccctga gcattcccac tttggtaagg aaggagtata atttgctgaa
tggtgcaagc aagccaggag gacagaagat gttacacttt actcagggaa cagaggcggg
caactggccc tgtgactgca gccaacagct ttaagaacac agtcctttct gcttcaaggt
tagggagacg ttctcgcctc tttcttcttt gcagttatta ttcaagaggc ttcccccgac
cccagtcccc agcaccatcc tcagagcttc agaccataca ttgacagtga gcaaaggggg
ccccaggcag gcgggtctgg ggccaaggag ggcggctccc ctgcgcggat ccttccctgg
tggctcccaa atccggcgtt ttctctgccg cctctccctc gggggagact cggaaaggct
gcaaaaatct gggcgcccgt tcgctcgctt gtcaagaagc aaactgtctt cacattctcc
aagagcaaca tccctgccta ggaagaggaa ggaagaggca aaataaataa aaccagttaa
tgttgtagtt aacttgcaaa tcaagtaaat ctgttggtgc cgtatttgag aaataaacca
tcacagcgtc acagcaaaca ca
SEQ ID NO: 253
ccctgccccg ggaggtgctc aggaaagggt tgtgaccccg agtgacagta gaggctcaga
gaggtcagga tgtgtagtgc atggtggagc tggccactaa ctcgggccgc ttcttgtctt
gtttgagtag caattgaggg gctcctgggt gccccgggct gggctgggcc tggagtcagc
aagccccaag tcttgccctc ccttgccagg gaggaaggaa aggtaaccgg ctgtgacact
gagggaggtg agctgggaac tggaggtgca gagaaggccc cgacgctgtt tgtaggttgt
gggggtgcag caagacctag atcttaagaa tttcgaagga ctgtgacgat caccggctgc
gccctgccgg cgagtgccct ggggctggct ctatttgttg cgcgatccag ccctggtggg
gagatttgtg aggggagacc tggctcaggc tgtgtcttcc tgttcaaacg ggggttagta
gagagggggt tggggaggtc cagggagaac tgggcatgca gcctgcaggg gagagggacc
ccttggaggg ctgcgggaga ggctccttgt aaatgtcaac aaagacccag ccaggcaggt
ccatgggtta ccctaagagc ttagagttta tcggagagga aatgg
SEQ ID NO: 254
aggttgggga gttgagaagg atggagatgg gtgcatctgg aagggagtcc gtcctgagga
gtcccccatc agctgtcagc cagccagcag caaagcaaat taagactaca cagctccgaa
gaagccagtt cccaaccaag ccagtggaga aaagtcagcc cggtccccag gagtgcttga
ggctctgtca ctcttggacg tcaaaaaggg tcatttgatg actggacgct tacctcaccg
gtgtgaggta agcttcaaac gccgtatcat gttgctttaa aacctgcggg taacagcata
agctgagttt tctatcttag aactcttaac cccaagaaca ctcttcacag gccctgatag
gtggacccac aaaaaaacca ctcaggctat atttgactcg gatttgaaac gctgccgaaa
cggtattaag tgtcctcctc aactggaaaa gacaaataac aaatgatgcc tgaatgagaa
aaagactaga cgtgcacaca gtaatgtgtg agcagggaaa cttcagcgaa ggtttttatc
atgctttacc ccttttacat gctttacccc atgtgcaaac atttttcatg ggtttttttc
tattttttat ttattttt
SEQ ID NO: 255
gtcccagcgt cccagcccag ctacccaagt agaaggtggg gcggcatctt ctatggctga
gtcttgggca gtgggtgctc tgtcatattg tcaggtttct tcccccagct ccacaatgtg
aagactgagg tggtccctcc aagccccact cagcaaggag gacagggctg gtggatcctc
cagggtcaga tggggaaata aaagtgtttc attcttgaag gggaagctgc acttctccac
ggcacgcctg gtggtgccag gggttaccac aaagaggcgg cagagccatg gcccaccagc
cacttggcag gctggttgtc tggtgaagct tttcagggtt tggcacaggg cccagtcctc
agtccccccc atgtccacca cctccactgg tgcccccagg cctgggaggt gtaggaggtg
ccgggggggc atgtaggtgt gagtgaagag gagtgtgtag tgggtgggtg tgcttgggag
cacaggggca tggaccacct gctccaggta ctccaggcca ggcaccaggc ccccctgatg
caggcagccg aagaggaggg caccgagggc gttga
SEQ ID NO: 256
gaggaatcat gattgtctaa actagtcatg gccatcccag tcttctttgc tagatacttg
agtaatctct ttcccagttt ctctgttctg gccagtgaga tgtaagagag agtctgctga
gttcttgtga gaactgcttt atttcataaa gagaacaact aagagagtac ttccttccct
tcctgtttgg gtaaggactt gatgctgcag gagccaccct gcatccataa aggagaagtc
aaaaggaatc agagacaaca gcccagaccc ccatcacgga gctgcacgtg accctggaac
ttaacagctt ccagttgttc cctagacagt cattgtcttt atggtgcctt ttcccccatc
agggaggaag gtgccttgct tcaagtcttt tcaattaaac tgcagtttaa cagagaaaat
tgccttatga acagtcaaaa gtcagtacaa cttaaatatg gcagtttatc tcaggacatc
tcccagcaca cgtcttcctg agagcaagct gctctcaaaa ccaaggcaat gggaaaatgg
tcagaaacat gacttctgta ttttccagtt catacactga agacagcatt cattcattca
tttggaaagt cag
SEQ ID NO: 257
tgactgtata acactagtag atatttttaa aatgcaagag catcttctta gatcattact
tttccttgga atgcttggcc cctgtaatat aatacaccgg tattttgcat gatgaaattg
atgtcctgtg tgttgcttca tgttgctatc ctagctgccg attaaaacgt tttttttttt
tcatgccaga gcagaacaaa attgtctgct tctcaatctg cacatcataa gcagatgaca
ttaaaaatgt ctgtaagatg acacagctat attttctggg agagggcggg aggatgctca
gcgagggtgg cccggagtgt ccttgtacag agtacagatg ttatgaagtg gggaagacca
gcctgtgttc attgattcac ctattgattc caggagcaag ctcaccctgt ttcatacact
gctcaggagg taaacaggag gaagggagcc agcctggctt ttttgccaca tgctctgctg
tttggtagaa ctgtattata gtcagaaacc ttccgctttt ctgcagttgt ttgcatgctg
tttccaaggc tagccctctg agtctgtttt ctagagttgt tttgaaattc aacctaaaga
taacagagga aatgtga
SEQ ID NO: 258
gcgcggcaca acggcgcatt gtggggccaa gcgaggggcg aagggggctg ggggtggccg
gcgcgatggg gacgctccgg ttgcgccaag ttgactctgc cgtttgggtc acctgggctg
agtcgcgggc gtggaggcag agggtagggg gtgaggaggt gtttcttgtc ttcttcttcc
aatctcagaa gtaaacattg gaaagtgggg cccccagcag tgtacagccc gtttccaaac
caggcctgta aggaggagct gaggtttcgg ctgagccccc agcctccccc gaccgcacag
cctcgggcat gaacccgcga agccagacgc ttagttgctt atcaggccat cgctgtacat
atttagaaag tacctatcac tcagacactt tgaaaagcgt ggcgttccag cgcaaaccaa
cccgaacggg ttggaagggg gcagtccttt cttcccgcaa gttcggggct cgagagacgg
ctgcaggaag gccatcaccc ctggcttcct gcagccacag cttccagccc cacacgatgc
ccaacttcat tttagcagtg gcccccaggg gaaatcacac cattcttggt tttgtccctc
cctcctgag
SEQ ID NO: 259
cttagcaatc tgcatttcta aagtgctttg tacacattcc gtctatgtta aaagcctcag
cagcaggttg gaggcgggtt ctggggctag tgtttccgat gggaagctca ggctccatcc
agcctgtggc tggactggcc aggctcaatg tcactcccca ggtagctgcc cttgatctat
actaggagca ccttgagagc tgggaattga tttctaagcc tggtttgagc tgagggccac
agagccagtg caggaggaga ccctgcccca gaaataggcc agtgcttgtt atgcaggcct
tggcggttcc ccgtttcctt acgtaacctc agtgttcacg ctgtttcctt ttgttgattc
cctccgtgtg actgtttttc tgtcaatctc cttagctaat gagctcctta taaggagaat
ggatggatca gagcacagct ccgtacacag tggtggggca tagccatttc ccagagtgtg
gactttccca gaactcccct gttgtgtggg cctgcaaagg ctgggattgt ttctgccttg
tttggaataa taaagctgcc tgtgtttcct gtgttcactt ttcagtcgcc tgttattcac
tctcctacat ttggggcggt t

### Other Embodiments

While we have described a number of embodiments, it is apparent that our basic disclosure and examples may provide other embodiments that utilize or are encompassed by the compositions and methods described herein. Therefore, it will be appreciated that the scope of is to be defined by that which may be understood from the disclosure and the appended claims rather than by the specific embodiments that have been represented by way of example.

All references cited herein are hereby incorporated by reference.

1. A method of detecting advanced adenoma, the method comprising:
determining a methylation status for each of one or both of the following, in deoxyribonucleic acid (DNA) of a human subject:
(i) a methylation locus within gene NRF1; and
(ii) a methylation locus within gene TMEM196; and
diagnosing advanced adenoma in the human subject based at least on said determined methylation status(es).

2. The method of claim 1, wherein the methylation locus of NRF1 comprises at least one CpG dinucleotide.

3. The method of claim 1, wherein the methylation locus of TMEM196 comprises at least one CpG dinucleotide.

4. The method of claim 1, wherein the methylation locus within gene NRF1 comprises at least a portion of NRF1 '565 (SEQ ID NO: 1).

5. The method of claim 4, wherein the methylation locus within gene NRF1 comprises at least 50% of NRF1 '565 and wherein the portion of the methylation locus that overlaps with NRF1 '565 has at least 98% similarity with the overlapping portion of NRF1 '565.

6. The method of claim 1, wherein the methylation locus within gene TMEM196 comprises at least a portion of TMEM196 '652 (SEQ ID NO: 2).

7. The method of claim 1, further comprising determining a methylation status for a methylation locus comprising at least a portion of chr19:22709270-22709382 (SEQ ID NO: 3), and wherein the diagnosing step comprises diagnosing advanced adenoma in the human subject based at least on the determined methylation status for the methylation locus comprising chr19:22709270-22709382 (SEQ ID NO: 3).

8. The method of claim 1, wherein the DNA is isolated from blood or plasma of the human subject.

9. The method of claim 1, wherein the DNA is cell-free DNA of the human subject.

10. The method of claim 1, wherein methylation status is determined using quantitative polymerase chain reaction (qPCR).

11. The method of claim 1, wherein the methylation status is determined using methylation sensitive restriction enzyme (MSRE) qPCR.

12. The method of claim 1, wherein methylation status is determined using massively parallel sequencing.

13. A method of detecting colorectal cancer, the method comprising:
determining a methylation status for each of one or more of the following, in deoxyribonucleic acid (DNA) of a human subject:
(i) a methylation locus within gene ADSSL1;
(ii) a methylation locus within gene CFAP44;
(iii) a methylation locus within gene ENG;
(iv) a methylation locus within gene LINC01395;
(v) a methylation locus within gene NOS3;
(vi) a methylation locus within gene RASA3;
(vii) a methylation locus within gene SYCP1;
(viii) a methylation locus within gene ZAN;
(ix) a methylation locus within a genetic region comprising a portion of either or both of genes CD8B & ANAPC1P1;
(x) a methylation locus within a genetic region comprising a portion of either or both of genes FLI1 & LOC101929538;
(xi) a methylation locus within a genetic region comprising a portion of either or both of genes KCNQ1OT1 & KCNQ;
(xii) a methylation locus within a genetic region comprising a portion of either or both of genes LOC101929234 & ZNF503-AS2;
(xiii) a methylation locus within a genetic region comprising a portion of either or both of genes MAP3K6 & FCN3;
(xiv) a methylation locus comprising at least a portion of ch3:75609726-75609832 (SEQ ID NO: 21);
(xv) a methylation locus comprising at least a portion of ch3:45036223-45036316 (SEQ ID NO: 22);
(xvi) a methylation locus comprising at least a portion of ch12:53694915-53695058 (SEQ ID NO: 23);
(xvii) a methylation locus comprising at least a portion of ch12:53695032-53695180 (SEQ ID NO: 24);
(xviii) a methylation locus comprising at least a portion of ch12:53695146-53695232 (SEQ ID NO: 25);
(xix) a methylation locus comprising at least a portion of ch17:78304805-78304921 (SEQ ID NO: 26); and
(xx) a methylation locus comprising at least a portion of ch19:22709270-22709382 (SEQ ID NO: 3); and
diagnosing colorectal cancer in the human subject based at least on said determined methylation status(es).

14. The method of claim 13, comprising determining a methylation status for a methylation locus within gene ADSSL1, wherein the methylation locus within gene ADSSL1 comprises at least a portion of ch14:104736436-104736562 (SEQ ID NO: 4).

15. The method of claim 13, comprising determining a methylation status for a methylation locus within gene CFAP44, wherein the methylation locus within gene CFAP44 comprises at least a portion of one or more of:
ch3:113441434-113441539 (SEQ ID NO: 5);
ch3:113441519-113441620 (SEQ ID NO: 6); and/or
ch3:113441596-113441690 (SEQ ID NO: 7).

16. The method of claim 13, comprising determining a methylation status for a methylation locus within gene ENG, wherein the methylation locus within gene ENG comprises at least a portion of ch9:127828322-127828421 (SEQ ID NO: 8).

17. The method of claim 13, comprising determining a methylation status for a methylation locus within gene LINC01395, wherein the methylation locus within gene LINC01395 comprises at least a portion of ch11:129618087-129618193 (SEQ ID NO: 9) and/or at least a portion of ch11:129618345-129618455 (SEQ ID NO: 10).

18. The method of claim 13, comprising determining a methylation status for a methylation locus within gene NOS3, wherein the methylation locus within gene NOS3 comprises at least a portion of ch7:150996901-150997007 (SEQ ID NO: 11).

19. The method of claim 13, comprising determining a methylation status for a methylation locus within gene RASA3, wherein the methylation locus within gene RASA3 comprises at least a portion of ch13:114111799-114111878 (SEQ ID NO: 12).

20. The method of claim 13, comprising determining a methylation status for a methylation locus within gene SYCP1, wherein the methylation locus within gene SYCP1 comprises at least a portion of ch1:114855187-114855327 (SEQ ID NO: 13).

21. The method of claim 13, comprising determining a methylation status for a methylation locus within gene ZAN, wherein the methylation locus within gene ZAN comprises at least a portion of ch7:100785886-100786015 (SEQ ID NO: 14).

22. The method of claim 13, comprising determining a methylation status for a methylation locus within a portion of either or both of genes GD8B & ANAPC1P1, wherein the methylation locus within the portion of either or both of genes CD8B & ANAPC1P1 comprises at least a portion of ch2:86862416-86862559 (SEQ ID NO: 15).

23. The method of claim 13, comprising determining a methylation status for a methylation locus within a portion of either or both of genes FLI1 & LOC101929538, wherein the methylation locus within the portion of either or both of genes FLI1 & LOC101929538 comprises at least a portion of ch11:128685299-128685448 (SEQ ID NO: 16).

24. The method of claim 13, comprising determining a methylation status for a methylation locus within a portion of either or both of genes KCNQ1OT1 & KCNQ, wherein the methylation locus within the portion of either or both of genes KCNQ1OT1 & KCNQ comprises at least a portion of ch11:2656072-2656156 (SEQ ID NO: 17).

25. The method of claim 13, comprising determining a methylation status for a methylation locus within a portion of either or both of genes MAP3K6 & FCN3, wherein the methylation locus within the portion of either or both of genes MAP3K6 & FCN3 comprises at least a portion of ch1:27369167-27369316 (SEQ ID NO: 19) and/or at least a portion of ch1:27369224-27369347 (SEQ ID NO: 20).

26. The method of claim 13, comprising determining a methylation status for a methylation locus within gene ADSSL1, wherein the methylation locus within gene ADSSL1 comprises at least one CpG dinucleotide.

27. The method of claim 13, comprising determining a methylation status for a methylation locus within gene CFAP44, wherein the methylation locus within gene CFAP44 comprises at least one CpG dinucleotide.

28. The method of claim 13, comprising determining a methylation status for a methylation locus within gene ENG, wherein the methylation locus within gene ENG comprises at least one CpG dinucleotide.

29. The method of claim 13, comprising determining a methylation status for a methylation locus within gene LINC01395, wherein the methylation locus within gene LINC01395 comprises at least one CpG dinucleotide.

30. The method of claim 13, comprising determining a methylation status for a methylation locus within gene NOS3, wherein the methylation locus within gene NOS3 comprises at least one CpG dinucleotide.

31. The method of claim 13, comprising determining a methylation status for a methylation locus within gene RASA3, wherein the methylation locus within gene RASA3 comprises at least one CpG dinucleotide.

32. The method of claim 13, comprising determining a methylation status for a methylation locus within gene SYCP1, wherein the methylation locus within gene SYCP1 comprises at least one CpG dinucleotide.

33. The method of claim 13, comprising determining a methylation status for a methylation locus within gene ZAN, wherein the methylation locus within gene ZAN comprises at least one CpG dinucleotide.

34. The method of claim 13, comprising determining a methylation status for a methylation locus within a genetic region comprising a portion of either or both of genes CD8B & ANAC1P1, wherein the methylation locus within a genetic region comprising a portion of either or both of genes CD8B & ANAPC1P1 comprises at least one CpG dinucleotide.

35. The method of claim 13, comprising determining a methylation status for a methylation locus within a genetic region comprising a portion of either or both of genes FLI1 & LOC101929538, wherein the methylation locus within a genetic region comprising a portion of either or both of genes FLI1 & LOC101929538 comprises at least one CpG dinucleotide.

36. The method of claim 13, comprising determining a methylation status for a methylation locus within a genetic region comprising a portion of either or both of genes KCNQ1OT1 & KCNQ, wherein the methylation locus within a genetic region comprising a portion of either or both of genes KCNQ1OT1 & KCNQ comprises at least one CpG dinucleotide.

37. The method of claim 13, comprising determining a methylation status for a methylation locus within a genetic region comprising a portion of either or both of genes LOC101929234 & ZNF503-AS2, wherein the methylation locus within a genetic region comprising a portion of either or both of genes LOC101929234 & ZNF503-AS2 comprises at least one CpG dinucleotide.

38. The method of claim 13, comprising determining a methylation status for a methylation locus within a genetic region comprising a portion of either or both of genes MAP3K6 & FCN3, wherein the methylation locus within a genetic region comprising a portion of either or both of genes MAP3K6 & FCN3 comprises at least one CpG dinucleotide.

39. The method of claim 13, wherein the DNA is isolated from blood or plasma of the human subject.

40. The method of claim 13, wherein the DNA is cell-free DNA of the human subject.

41. The method of claim 13, wherein the methylation status is determined using quantitative polymerase chain reaction (qPCR).

42. The method of claim 13, wherein the methylation status is determined using methylation sensitive restriction enzyme (MSRE)-qPCR.

43. The method of claim 13, wherein methylation status is determined using massively parallel sequencing.

44. A method of screening for a colorectal neoplasm in a sample obtained from a subject, the method comprising:
determining a methylation status of each of one or more markers identified in the sample; and
determining whether the subject has a colorectal neoplasm based at least in part on the determined methylation status of each of the one or more markers and a corresponding methylation status of said one or more markers representative of one or more subjects that do not have a colorectal neoplasm that is considered to be either malignant or pre-malignant,
wherein each of the one or more markers comprises a base in a differentially methylated region (DMR) selected from the DMRs listed in Table 1.

45. The method of claim 44, wherein the colorectal neoplasm comprises colorectal cancer and/or advanced adenoma.

46. The method of claim 44, wherein the sample comprises a stool sample, a colorectal tissue sample, a blood sample, or a blood product sample.

47. The method of claim 44, wherein each methylation locus is equal to or less than 5000 bp in length.

48. A kit for use in a method of screening for a colorectal neoplasm in a sample obtained from a subject, the kit comprising one or more oligonucleotide primer pairs for amplification of one or more corresponding methylation locus/loci of Table 1.

49. The kit of claim 48, wherein the one or more corresponding methylation loci each comprise at least one methylation sensitive restriction enzyme cleavage sites.

50. A diagnostic qPCR reaction for detection of colorectal cancer, the diagnostic qPCR reaction including
(a) human DNA,
(b) a polymerase,
(c) one or more oligonucleotide primer pairs for amplification of one or more corresponding methylation locus/loci of Table 1, and, optionally, at least one methylation sensitive restriction enzyme.

51. The diagnostic qPCR reaction of claim 50, wherein each of the one or more corresponding methylation loci is equal to or less than 5000 bp.

52. The diagnostic qPCR reaction of claim 50, wherein each of the one or more corresponding methylation locus/loci comprises at least one methylation sensitive restriction enzyme (MSRE) cleavage site.


SEQUENCE LISTING (259 sequences)

SEQ ID NO: 1 - DNA, 112 bp, Artificial Sequence
  Description of Artificial Sequence Synthetic polynucleotide
taaccacctgcacctctgctgcaatgtaaacagcagatgtgggcgcagggtgagaagggagaggaagctacgtgcaatggcaggttggggaataaggaggcagaggggctcc

SEQ ID NO: 2 - DNA, 149 bp, Artificial Sequence
  Description of Artificial Sequence Synthetic polynucleotide
ggagagcaccaagaggctcccaataatctgaccgctggtgcacatccttcctcggtcatcttccttccagatcagagagggaaatcaaccatctacctttttttcttccactatcctccttaccccttccaccccctaccagatcccaa

SEQ ID NO: 3 - DNA, 113 bp, Artificial Sequence
  Description of Artificial Sequence Synthetic polynucleotide
gggccagttcctcctaccagcttcctgctgccacctcggcttccatcagagggacgcttaggatggcgcaggggcccggagacactgtgaagagtccaggggaatgaggaggg

SEQ ID NO: 4 - DNA, 127 bp, Artificial Sequence
  Description of Artificial Sequence Synthetic polynucleotide
cacagacaccctgagcttgcaacactccgggcctctgccgcgtgtttatttcaggatgccgtggcatttgggtgaccttttgtgctcaccatggcttgcgtcgtctccgggtcactctcgtctggac

SEQ ID NO: 5 - DNA, 106 bp, Artificial Sequence
  Description of Artificial Sequence Synthetic polynucleotide
tgctgaggtccaaactcaccgaaggtactgaccgccgcggctcctctcttcacagcgtctgccggaggcctccgtttactccggttaccgagacaacgccacccct

SEQ ID NO: 6 - DNA, 102 bp, Artificial Sequence
  Description of Artificial Sequence Synthetic polynucleotide
taccgagacaacgccacccctcttccagggaggcggaaccagggcgggccgtggggcgcatgcgcggccggcgtccagctctccgggaacccggtacctatc

SEQ ID NO: 7 - DNA, 95 bp, Artificial Sequence
  Description of Artificial Sequence Synthetic oligonucleotide
gctctccgggaacccggtacctatccgccctttggtcgggccttctccgcctcatgacactggttcaaagccaaacagaaaagcccgacgagttt

SEQ ID NO: 8 - DNA, 100 bp, Artificial Sequence
  Description of Artificial Sequence Synthetic polynucleotide
gcccctgtaaaatggggatacagcagggcacgacgtctgttggtcgcctggcactgggtcggccaccgaggccgcgccttggcctctttgtcccctctgg

SEQ ID NO: 9 - DNA, 107 bp, Artificial Sequence
  Description of Artificial Sequence Synthetic polynucleotide
gggagcctggaggggttgacaccgcctgctccaccgcaagcccctggaggaagagccccgctgtgcccgagagcgagcgcgggcaggtgtaactacccggggctggg

SEQ ID NO: 10 - DNA, 111 bp, Artificial Sequence
  Description of Artificial Sequence Synthetic polynucleotide
cctctgcttcaggtgcttggctagagaaagggcggcaagacggggcagtgcgtgtgcgcgcgcgggcaagtgcatgtgagtgcacacttatgtgagcgcatgtgtgtctgc

SEQ ID NO: 11 - DNA, 107 bp, Artificial Sequence
  Description of Artificial Sequence Synthetic polynucleotide
ccggatccagtgggggaagctgcaggtgcggctggccagcgactgagagacccgggcgctaccaaaaggggagcggggtggcggggcagttcctaaggcttcccggg

SEQ ID NO: 12 - DNA, 80 bp, Artificial Sequence
  Description of Artificial Sequence Synthetic oligonucleotide
tgtcaaacctccatctgtggtcaggagttaggacatccccagctgcaatttgagcaaagacggcgcttccagaggatcat

SEQ ID NO: 13 - DNA, 141 bp, Artificial Sequence
  Description of Artificial Sequence Synthetic polynucleotide
gaagggaacgggctttcttttcaggccagcgtggcagcgggcggtagggcgaaagggagaaggaaacgagggtttattccgttgcccactccgcggtaagcgacgttgtagggctccactgtagcgagagccccgtggatt

SEQ ID NO: 14 - DNA, 130 bp, Artificial Sequence
  Description of Artificial Sequence Synthetic polynucleotide
tgacctcaggtgatccacccgtctcggcctcccaaagtgttgggattacaggcgtgagccgccgcgcccagccccctcctcactctctttctcttcctgtaacttctacagctgggcaagagctgggtct

SEQ ID NO: 15 - DNA, 144 bp, Artificial Sequence
  Description of Artificial Sequence Synthetic polynucleotide
gctgggaactggaggtgcagagaaggccccgacgctgtttgtaggttgtgggggtgcagcaagacctagatcttaagaatttcgaaggactgtgacgatcaccggctgcgccctgccggcgagtgccctggggctggctctatt

SEQ ID NO: 16 - DNA, 150 bp, Artificial Sequence
  Description of Artificial Sequence Synthetic polynucleotide
gcaccaagaactaacacatcctggagctgcccggagttccgctcctgcgggcttagcaggaaagggtgcctaaggtgagtgcccacttgcgtccgatcctctgggggcgatgcagggtcggggcgcctcagtgtgtctcgctgcttgttc

SEQ ID NO: 17 - DNA, 85 bp, Artificial Sequence
  Description of Artificial Sequence Synthetic oligonucleotide
tgggcacttgtcatcatgggtgtttggaaagcaactctacgttctagcctgtgctccatcgttccttctacatacaagtgatgca

SEQ ID NO: 18 - DNA, 101 bp, Artificial Sequence
  Description of Artificial Sequence Synthetic polynucleotide
aatacatccagctcgcaggcatcctgcaagaaacggctcccggctcgcgtgtacgccgacacctcggcccaacgcaggactcgaggtggtttctagtgccc

SEQ ID NO: 19 - DNA, 150 bp, Artificial Sequence
  Description of Artificial Sequence Synthetic polynucleotide
gcaaaggcaaggtggctgacgatccggaagctgtacaggagagataagggcactggctgccagagtgccctatcgaagcatcatccgaaccctgcggtaggggtggcccacaccacggcctgaggcccagtcaatgccatatttgtgggc

SEQ ID NO: 20 - DNA, 124 bp, Artificial Sequence
  Description of Artificial Sequence Synthetic polynucleotide
tgccagagtgccctatcgaagcatcatccgaaccctgcggtaggggtggcccacaccacggcctgaggcccagtcaatgccatatttgtgggcggcagcctcagacactgcatagcgaccattg

SEQ ID NO: 21 - DNA, 107 bp, Artificial Sequence
  Description of Artificial Sequence Synthetic polynucleotide
cctgcgacgtgaatcgtcatatccagaggggggtgatatgactccccgcatcgcgggggcctcaccccattgcgatgggggtcctaagagccagggggagatagggg

SEQ ID NO: 22 - DNA, 94 bp, Artificial Sequence
  Description of Artificial Sequence Synthetic oligonucleotide
tgagcggaggactgaggagaggaaggagggaaagaatagggagatgaaaacgccccggtctgctgctaagcacagcacagttaccaaagccagg

SEQ ID NO: 23 - DNA, 144 bp, Artificial Sequence
  Description of Artificial Sequence Synthetic polynucleotide
catctcctcctcgcaaaccccaagccaaggcaagctggatgaagcgctccctgggcaggcccggctctccgtgtccctccatcacctgaccccgctggctctcgcagaccccttcctccacactcactcctcccggctctcctt

SEQ ID NO: 24 - DNA, 149 bp, Artificial Sequence
  Description of Artificial Sequence Synthetic polynucleotide
ccacactcactcctcccggctctccttctataatctcctgacatctcttcaaatccaattattgaattaattgacgtacgaacccagaggcaaacagaaaggggcggcaaacactgggcggctcagatttatccttcggcctccgcagg

SEQ ID NO: 25 - DNA, 87 bp, Artificial Sequence
  Description of Artificial Sequence Synthetic oligonucleotide
tgggcggctcagatttatccttcggcctccgcagggcccggccggacgagatttactgggcctcgaacacggcgacagttcaaacct

SEQ ID NO: 26 - DNA, 117 bp, Artificial Sequence
  Description of Artificial Sequence Synthetic polynucleotide
cagccctagggagacagcaggatggttccaggaagcctgggccgctccccagatcaatgcagggacggacagcagccagcaggctgggccacggcatcagagctggggtcaagaggt

SEQ ID NO: 27 - DNA, 24 bp, Artificial Sequence
  Description of Artificial Sequence Synthetic primer
cgatgcttggccaatgaaaagagg

SEQ ID NO: 28 - DNA, 24 bp, Artificial Sequence
  Description of Artificial Sequence Synthetic primer
gcaaggtgcagatggtgaaggatg

SEQ ID NO: 29 - DNA, 24 bp, Artificial Sequence
  Description of Artificial Sequence Synthetic primer
ctcctctggctctcctgctccatc

SEQ ID NO: 30 - DNA, 24 bp, Artificial Sequence
  Description of Artificial Sequence Synthetic primer
gaggcaaaaaggacaatcggcaag

SEQ ID NO: 31 - DNA, 24 bp, Artificial Sequence
  Description of Artificial Sequence Synthetic primer
cctgcgacgtgaatcgtcatatcc

SEQ ID NO: 32 - DNA, 24 bp, Artificial Sequence
  Description of Artificial Sequence Synthetic primer
agctgagggaaagggggaagtcac

SEQ ID NO: 33 - DNA, 24 bp, Artificial Sequence
  Description of Artificial Sequence Synthetic primer
tgctgaggtccaaactcaccgaag

SEQ ID NO: 34 - DNA, 25 bp, Artificial Sequence
  Description of Artificial Sequence Synthetic primer
ccttccccagcctaagaaggtttcc

SEQ ID NO: 35 - DNA, 24 bp, Artificial Sequence
  Description of Artificial Sequence Synthetic primer
gccagtgagtcagaggcagaggtg

SEQ ID NO: 36 - DNA, 24 bp, Artificial Sequence
  Description of Artificial Sequence Synthetic primer
taccgagacaacgccacccctctt

SEQ ID NO: 37 - DNA, 24 bp, Artificial Sequence
  Description of Artificial Sequence Synthetic primer
gagctgggacaagaagggaacacg

SEQ ID NO: 38 - DNA, 24 bp, Artificial Sequence
  Description of Artificial Sequence Synthetic primer
gctctccgggaacccggtacctat

SEQ ID NO: 39 - DNA, 24 bp, Artificial Sequence
  Description of Artificial Sequence Synthetic primer
gcgtctctgtggccgtgaagtgta

SEQ ID NO: 40 - DNA, 22 bp, Artificial Sequence
  Description of Artificial Sequence Synthetic primer
ccggatccagtgggggaagctg

SEQ ID NO: 41 - DNA, 24 bp, Artificial Sequence
  Description of Artificial Sequence Synthetic primer
aatacatccagctcgcaggcatcc

SEQ ID NO: 42 - DNA, 22 bp, Artificial Sequence
  Description of Artificial Sequence Synthetic primer
gggagcctggaggggttgacac

SEQ ID NO: 43 - DNA, 25 bp, Artificial Sequence
  Description of Artificial Sequence Synthetic primer
cctctgcttcaggtgcttggctaga

SEQ ID NO: 44 - DNA, 24 bp, Artificial Sequence
  Description of Artificial Sequence Synthetic primer
gcccaagggccacaagagtatgac

SEQ ID NO: 45 - DNA, 24 bp, Artificial Sequence
  Description of Artificial Sequence Synthetic primer
cggttccacacctggaactggatt

SEQ ID NO: 46 - DNA, 24 bp, Artificial Sequence
  Description of Artificial Sequence Synthetic primer
ggtcctagagccgcttggcttcac

SEQ ID NO: 47 - DNA, 25 bp, Artificial Sequence
  Description of Artificial Sequence Synthetic primer
tgtcaaacctccatctgtggtcagg

SEQ ID NO: 48 - DNA, 24 bp, Artificial Sequence
  Description of Artificial Sequence Synthetic primer
tggggccgaagagatccttgaaca

SEQ ID NO: 49 - DNA, 22 bp, Artificial Sequence
  Description of Artificial Sequence Synthetic primer
gctgcagtttcgtcagcccttg

SEQ ID NO: 50 - DNA, 24 bp, Artificial Sequence
  Description of Artificial Sequence Synthetic primer
cagccctagggagacagcaggatg

SEQ ID NO: 51 - DNA, 24 bp, Artificial Sequence
  Description of Artificial Sequence Synthetic primer
ggctcaccttcaggaagcacctgt

SEQ ID NO: 52 - DNA, 24 bp, Artificial Sequence
  Description of Artificial Sequence Synthetic primer
gggccagttcctcctaccagcttc

SEQ ID NO: 53 - DNA, 24 bp, Artificial Sequence
  Description of Artificial Sequence Synthetic primer
ggtctttcccacacctctgcacct

SEQ ID NO: 54 - DNA, 24 bp, Artificial Sequence
  Description of Artificial Sequence Synthetic primer
gcctggccaccacagagaagaaga

SEQ ID NO: 55 - DNA, 24 bp, Artificial Sequence
  Description of Artificial Sequence Synthetic primer
tgctctctctccaaaggcgagttg

SEQ ID NO: 56 - DNA, 24 bp, Artificial Sequence
  Description of Artificial Sequence Synthetic primer
cctgggaaccagtgctggagaaag

SEQ ID NO: 57 - DNA, 25 bp, Artificial Sequence
  Description of Artificial Sequence Synthetic primer
gcccctgtaaaatggggatacagca

SEQ ID NO: 58 - DNA, 24 bp, Artificial Sequence
  Description of Artificial Sequence Synthetic primer
tgggcacttgtcatcatgggtgtt

SEQ ID NO: 59 - DNA, 24 bp, Artificial Sequence
  Description of Artificial Sequence Synthetic primer
acactttgaaaagcgtggcgttcc

SEQ ID NO: 60 - DNA, 24 bp, Artificial Sequence
  Description of Artificial Sequence Synthetic primer
aaaaggctccgacgatgctccaga

SEQ ID NO: 61 - DNA, 26 bp, Artificial Sequence
  Description of Artificial Sequence Synthetic primer
gcaccaagaactaacacatcctggag

SEQ ID NO: 62 - DNA, 25 bp, Artificial Sequence
  Description of Artificial Sequence Synthetic primer
gaagggaacgggctttcttttcagg

SEQ ID NO: 63 - DNA, 23 bp, Artificial Sequence
  Description of Artificial Sequence Synthetic primer
cgagaagggaggaggtgaaggag

SEQ ID NO: 64 - DNA, 24 bp, Artificial Sequence
  Description of Artificial Sequence Synthetic primer
tgagcggaggactgaggagaggaa

SEQ ID NO: 65 - DNA, 24 bp, Artificial Sequence
  Description of Artificial Sequence Synthetic primer
gctctgatgcctctccctccacac

SEQ ID NO: 66 - DNA, 24 bp, Artificial Sequence
  Description of Artificial Sequence Synthetic primer
gacatcctccttggcagcctttca

SEQ ID NO: 67 - DNA, 24 bp, Artificial Sequence
  Description of Artificial Sequence Synthetic primer
tgagcgcttaacgatccggaaaga

SEQ ID NO: 68 - DNA, 24 bp, Artificial Sequence
  Description of Artificial Sequence Synthetic primer
tgacctcaggtgatccacccgtct

SEQ ID NO: 69 - DNA, 29 bp, Artificial Sequence
  Description of Artificial Sequence Synthetic primer
ccccatagggaggacttgcgcacagttgg

SEQ ID NO: 70 - DNA, 24 bp, Artificial Sequence
  Description of Artificial Sequence Synthetic primer
cacagacaccctgagcttgcaaca

SEQ ID NO: 71 - DNA, 21 bp, Artificial Sequence
  Description of Artificial Sequence Synthetic primer
gcaaaggcaaggtggctgacg

SEQ ID NO: 72 - DNA, 24 bp, Artificial Sequence
  Description of Artificial Sequence Synthetic primer
tgccagagtgccctatcgaagcat

SEQ ID NO: 73 - DNA, 27 bp, Artificial Sequence
  Description of Artificial Sequence Synthetic primer
gtcatcagtgaatcgaccacaaagagc

SEQ ID NO: 74 - DNA, 24 bp, Artificial Sequence
  Description of Artificial Sequence Synthetic primer
ggagagcaccaagaggctcccaat

SEQ ID NO: 75 - DNA, 24 bp, Artificial Sequence
  Description of Artificial Sequence Synthetic primer
taaccacctgcacctctgctgcaa

SEQ ID NO: 76 - DNA, 22 bp, Artificial Sequence
  Description of Artificial Sequence Synthetic primer
acaccccgcggcaggacttcta

SEQ ID NO: 77 - DNA, 24 bp, Artificial Sequence
  Description of Artificial Sequence Synthetic primer
catctcctcctcgcaaaccccaag

SEQ ID NO: 78 - DNA, 23 bp, Artificial Sequence
  Description of Artificial Sequence Synthetic primer
ccacactcactcctcccggctct

SEQ ID NO: 79 - DNA, 24 bp, Artificial Sequence
  Description of Artificial Sequence Synthetic primer
tgggcggctcagatttatccttcg

SEQ ID NO: 80 - DNA, 24 bp, Artificial Sequence
  Description of Artificial Sequence Synthetic primer
tcggcagtgaaaagcgggagatta

SEQ ID NO: 81 - DNA, 22 bp, Artificial Sequence
  Description of Artificial Sequence Synthetic primer
ggctctcgggctctcgcttttt

SEQ ID NO: 82 - DNA, 24 bp, Artificial Sequence
  Description of Artificial Sequence Synthetic primer
ctggggagaagtgaccccattcaa

SEQ ID NO: 83 - DNA, 25 bp, Artificial Sequence
  Description of Artificial Sequence Synthetic primer
gcaccatcctcagagcttcagacca

SEQ ID NO: 84 - DNA, 24 bp, Artificial Sequence
  Description of Artificial Sequence Synthetic primer
gctgggaactggaggtgcagagaa

SEQ ID NO: 85 - DNA, 25 bp, Artificial Sequence
  Description of Artificial Sequence Synthetic primer
gcttcaaacgccgtatcatgttgct

SEQ ID NO: 86 - DNA, 24 bp, Artificial Sequence
  Description of Artificial Sequence Synthetic primer
tggtgccaggggttaccacaaaga

SEQ ID NO: 87 - DNA, 24 bp, Artificial Sequence
  Description of Artificial Sequence Synthetic primer
gagacaacagcccagacccccatc

SEQ ID NO: 88 - DNA, 34 bp, Artificial Sequence
  Description of Artificial Sequence Synthetic primer
tgtaagatgacacagctatattttctgggagagg

SEQ ID NO: 89 - DNA, 24 bp, Artificial Sequence
  Description of Artificial Sequence Synthetic primer
ggaggagctgaggtttcggctgag

SEQ ID NO: 90 - DNA, 24 bp, Artificial Sequence
  Description of Artificial Sequence Synthetic primer
aggaggagaccctgccccagaaat

SEQ ID NO: 91 - DNA, 24 bp, Artificial Sequence
  Description of Artificial Sequence Synthetic primer
tgagttctcttgagggcagcgaaa

SEQ ID NO: 92 - DNA, 24 bp, Artificial Sequence
  Description of Artificial Sequence Synthetic primer
ggctttgcttgtgcccttatcagc

SEQ ID NO: 93 - DNA, 23 bp, Artificial Sequence
  Description of Artificial Sequence Synthetic primer
ggtggtgggcgttaaggaagctc

SEQ ID NO: 94 - DNA, 24 bp, Artificial Sequence
  Description of Artificial Sequence Synthetic primer
gtacccttcacccacccagggttt

SEQ ID NO: 95 - DNA, 24 bp, Artificial Sequence
  Description of Artificial Sequence Synthetic primer
cccctatctccccctggctcttag

SEQ ID NO: 96 - DNA, 22 bp, Artificial Sequence
  Description of Artificial Sequence Synthetic primer
gaaccccagtggacccctcaga

SEQ ID NO: 97 - DNA, 23 bp, Artificial Sequence
  Description of Artificial Sequence Synthetic primer
aggggtggcgttgtctcggtaac

SEQ ID NO: 98 - DNA, 24 bp, Artificial Sequence
  Description of Artificial Sequence Synthetic primer
ggcctctgagtggacagacactgg

SEQ ID NO: 99 - DNA, 23 bp, Artificial Sequence
  Description of Artificial Sequence Synthetic primer
ctcgagtcctggaggagcctgtg

SEQ ID NO: 100 - DNA, 24 bp, Artificial Sequence
  Description of Artificial Sequence Synthetic primer
gataggtaccgggttcccggagag

SEQ ID NO: 101 - DNA, 22 bp, Artificial Sequence
  Description of Artificial Sequence Synthetic primer
cacaggcctggagctcctcaca

SEQ ID NO: 102 - DNA, 25 bp, Artificial Sequence
  Description of Artificial Sequence Synthetic primer
aaactcgtcgggcttttctgtttgg

SEQ ID NO: 103 - DNA, 24 bp, Artificial Sequence
  Description of Artificial Sequence Synthetic primer
gacagcctccctcccatgtacagc

SEQ ID NO: 104 - DNA, 22 bp, Artificial Sequence
  Description of Artificial Sequence Synthetic primer
cccgggaagccttaggaactgc

SEQ ID NO: 105 - DNA, 25 bp, Artificial Sequence
  Description of Artificial Sequence Synthetic primer
gggcactagaaaccacctcgagtcc

SEQ ID NO: 106 - DNA, 23 bp, Artificial Sequence
  Description of Artificial Sequence Synthetic primer
cccagccccgggtagttacacct

SEQ ID NO: 107 - DNA, 24 bp, Artificial Sequence
  Description of Artificial Sequence Synthetic primer
gcagacacacatgcgctcacataa

SEQ ID NO: 108 - DNA, 24 bp, Artificial Sequence
  Description of Artificial Sequence Synthetic primer
ccagttccaggtgtggaaccgaac

SEQ ID NO: 109 - DNA, 23 bp, Artificial Sequence
  Description of Artificial Sequence Synthetic primer
gtggtgagggctgttccatgctt

SEQ ID NO: 110 - DNA, 24 bp, Artificial Sequence
  Description of Artificial Sequence Synthetic primer
cgtgaagccaagcggctctaggac

SEQ ID NO: 111 - DNA, 20 bp, Artificial Sequence
  Description of Artificial Sequence Synthetic primer
gggctgcgggcaggcactac

SEQ ID NO: 112 - DNA, 23 bp, Artificial Sequence
  Description of Artificial Sequence Synthetic primer
atgatcctctggaagcgccgtct

SEQ ID NO: 113 - DNA, 22 bp, Artificial Sequence
  Description of Artificial Sequence Synthetic primer
catccagacccgcgtggacagc

SEQ ID NO: 114 - DNA, 24 bp, Artificial Sequence
  Description of Artificial Sequence Synthetic primer
aggcagccaagacaagcagagagg

SEQ ID NO: 115 - DNA, 24 bp, Artificial Sequence
  Description of Artificial Sequence Synthetic primer
acctcttgaccccagctctgatgc

SEQ ID NO: 116 - DNA, 24 bp, Artificial Sequence
  Description of Artificial Sequence Synthetic primer
ggaggcagggtttacgtgcagaag

SEQ ID NO: 117 - DNA, 24 bp, Artificial Sequence
  Description of Artificial Sequence Synthetic primer
ccctcctcattcccctggactctt

SEQ ID NO: 118 - DNA, 24 bp, Artificial Sequence
  Description of Artificial Sequence Synthetic primer
cagaggggcatgctgactgcctat

SEQ ID NO: 119 - DNA, 22 bp, Artificial Sequence
  Description of Artificial Sequence Synthetic primer
aaggaagggggcgaggcatcag

SEQ ID NO: 120 - DNA, 24 bp, Artificial Sequence
  Description of Artificial Sequence Synthetic primer
cggacagcaggagggatttctcag

SEQ ID NO: 121 - DNA, 24 bp, Artificial Sequence
  Description of Artificial Sequence Synthetic primer
tgactcctccagagcgaggttgtg

SEQ ID NO: 122 - DNA, 23 bp, Artificial Sequence
  Description of Artificial Sequence Synthetic primer
ccagaggggacaaagaggccaag

SEQ ID NO: 123 - DNA, 30 bp, Artificial Sequence
  Description of Artificial Sequence Synthetic primer
tgcatcacttgtatgtagaaggaacgatgg

SEQ ID NO: 124 - DNA, 22 bp, Artificial Sequence
  Description of Artificial Sequence Synthetic primer
ggccttcctgcagccgtctctc

SEQ ID NO: 125 - DNA, 24 bp, Artificial Sequence
  Description of Artificial Sequence Synthetic primer
tgggcaggcgcctctagatgaaat

SEQ ID NO: 126 - DNA, 24 bp, Artificial Sequence
  Description of Artificial Sequence Synthetic primer
gaacaagcagcgagacacactgag

SEQ ID NO: 127 - DNA, 24 bp, Artificial Sequence
  Description of Artificial Sequence Synthetic primer
aatccacggggctctcgctacagt

SEQ ID NO: 128 - DNA, 22 bp, Artificial Sequence
  Description of Artificial Sequence Synthetic primer
cggggcgttttcatctccctat

SEQ ID NO: 129 - DNA, 25 bp, Artificial Sequence
  Description of Artificial Sequence Synthetic primer
cctggctttggtaactgtgctgtgc

SEQ ID NO: 130 - DNA, 23 bp, Artificial Sequence
  Description of Artificial Sequence Synthetic primer
ggcacaggcaaatgccaaatcct

SEQ ID NO: 131 - DNA, 24 bp, Artificial Sequence
  Description of Artificial Sequence Synthetic primer
aaaggcccagagatcggagctgag

SEQ ID NO: 132 - DNA, 24 bp, Artificial Sequence
  Description of Artificial Sequence Synthetic primer
catgccgtcctcaaattccagagc

SEQ ID NO: 133 - DNA, 24 bp, Artificial Sequence
  Description of Artificial Sequence Synthetic primer
agacccagctcttgcccagctgta

SEQ ID NO: 134 - DNA, 28 bp, Artificial Sequence
  Description of Artificial Sequence Synthetic primer
ctttaaccctttcccctcgcccgcagca

SEQ ID NO: 135 - DNA, 24 bp, Artificial Sequence
  Description of Artificial Sequence Synthetic primer
gtccagacgagagtgacccggaga

SEQ ID NO: 136 - DNA, 24 bp, Artificial Sequence
  Description of Artificial Sequence Synthetic primer
gcccacaaatatggcattgactgg

SEQ ID NO: 137 - DNA, 26 bp, Artificial Sequence
  Description of Artificial Sequence Synthetic primer
caatggtcgctatgcagtgtctgagg

SEQ ID NO: 138 - DNA, 24 bp, Artificial Sequence
  Description of Artificial Sequence Synthetic primer
gtgcaggggacagcagacatcaga

SEQ ID NO: 139 - DNA, 24 bp, Artificial Sequence
  Description of Artificial Sequence Synthetic primer
ttgggatctggtagggggtggaag

SEQ ID NO: 140 - DNA, 24 bp, Artificial Sequence
  Description of Artificial Sequence Synthetic primer
ggagcccctctgcctccttattcc

SEQ ID NO: 141 - DNA, 24 bp, Artificial Sequence
  Description of Artificial Sequence Synthetic primer
gggaagagctggggtcagtgaagg

SEQ ID NO: 142 - DNA, 24 bp, Artificial Sequence
  Description of Artificial Sequence Synthetic primer
aaggagagccgggaggagtgagtg

SEQ ID NO: 143 - DNA, 22 bp, Artificial Sequence
  Description of Artificial Sequence Synthetic primer
cctgcggaggccgaaggataaa

SEQ ID NO: 144 - DNA, 24 bp, Artificial Sequence
  Description of Artificial Sequence Synthetic primer
aggtttgaactgtcgccgtgttcg

SEQ ID NO: 145 - DNA, 20 bp, Artificial Sequence
  Description of Artificial Sequence Synthetic primer
gcgcccagaccaccgaggac

SEQ ID NO: 146 - DNA, 25 bp, Artificial Sequence
  Description of Artificial Sequence Synthetic primer
gccaggcaggagaaagagcttgaaa

SEQ ID NO: 147 - DNA, 24 bp, Artificial Sequence
  Description of Artificial Sequence Synthetic primer
agaaagcagccccaagtgggaaga

SEQ ID NO: 148 - DNA, 23 bp, Artificial Sequence
  Description of Artificial Sequence Synthetic primer
atttgggagccaccagggaagga

SEQ ID NO: 149 - DNA, 24 bp, Artificial Sequence
  Description of Artificial Sequence Synthetic primer
aatagagccagccccagggcactc

SEQ ID NO: 150 - DNA, 23 bp, Artificial Sequence
  Description of Artificial Sequence Synthetic primer
gggtccacctatcagggcctgtg

SEQ ID NO: 151 - DNA, 24 bp, Artificial Sequence
  Description of Artificial Sequence Synthetic primer
caccagacaaccagcctgccaagt

SEQ ID NO: 152 - DNA, 24 bp, Artificial Sequence
  Description of Artificial Sequence Synthetic primer
cctgatgggggaaaaggcaccata

SEQ ID NO: 153 - DNA, 24 bp, Artificial Sequence
  Description of Artificial Sequence Synthetic primer
cacaggctggtcttccccacttca

SEQ ID NO: 154 - DNA, 25 bp, Artificial Sequence
  Description of Artificial Sequence Synthetic primer
tgtacagcgatggcctgataagcaa

SEQ ID NO: 155 - DNA, 25 bp, Artificial Sequence
  Description of Artificial Sequence Synthetic primer
tcacacggagggaatcaacaaaagg

SEQ ID NO: 156 - DNA, 108 bp, Artificial Sequence
  Description of Artificial Sequence Synthetic polynucleotide
cgatgcttggccaatgaaaagaggtctacccgagagtgcgacgcgcaatgggcgggacttccggcgtctcccctcggcggttgctttcgctgccctcaagagaactca

SEQ ID NO: 157 - DNA, 116 bp, Artificial Sequence
  Description of Artificial Sequence Synthetic polynucleotide
gcaaggtgcagatggtgaaggatgcacacgagggccgcatcaccacgctgcggaagaaaaagaaggggaaggatggagccggggccaaggaggctgataagggcacaagcaaagcc

SEQ ID NO: 158 - DNA, 115 bp, Artificial Sequence
  Description of Artificial Sequence Synthetic polynucleotide
ctcctctggctctcctgctccatcgcgctcctccgcgcccttgccacctccaacgcccgtgcccagcagcgcgcggctgcccaacagcgccggagcttccttaacgcccaccacc

SEQ ID NO: 159 - DNA, 124 bp, Artificial Sequence
  Description of Artificial Sequence Synthetic polynucleotide
gaggcaaaaaggacaatcggcaagtaaatagtaaatgaacaagaagaccccggttgtgagaaaatgttataaagcaaataaatcagagaaatgtgatcacaaaccctgggtgggtgaagggtac

SEQ ID NO: 160 - DNA, 81 bp, Artificial Sequence
  Description of Artificial Sequence Synthetic oligonucleotide
agctgagggaaagggggaagtcactgggctgggggccggggccgctcactctggcctcctctgaggggtccactggggttc

SEQ ID NO: 161 - DNA, 129 bp, Artificial Sequence
  Description of Artificial Sequence Synthetic polynucleotide
ccttccccagcctaagaaggtttcctctccgggagtcacccaaggtgtgctgaccctggcctgggaccctgggaccgtggcgctcccacgctagcagcgacacggccagtgtctgtccactcagaggcc

SEQ ID NO: 162 - DNA, 90 bp, Artificial Sequence
  Description of Artificial Sequence Synthetic oligonucleotide
gccagtgagtcagaggcagaggtgccagagaccccgcccgaagggaggagatctgagagcctgcagccacaggctcctccaggactcgag

SEQ ID NO: 163 - DNA, 109 bp, Artificial Sequence
  Description of Artificial Sequence Synthetic polynucleotide
gagctgggacaagaagggaacacggtaccagggtagcagaagacaggcaccccccgtcccccagtcctagggcttcctcaccgcgcctgtgaggagctccaggcctgtg

SEQ ID NO: 164 - DNA, 99 bp, Artificial Sequence
  Description of Artificial Sequence Synthetic oligonucleotide
gcgtctctgtggccgtgaagtgtatgcatgcgtgcccatgttgatgcggcgccgtgcgggaggcgggcatcccctgctgtacatgggagggaggctgtc

SEQ ID NO: 165 - DNA, 124 bp, Artificial Sequence
  Description of Artificial Sequence Synthetic polynucleotide
gcccaagggccacaagagtatgacggggctgtacgagctgctgtgacgggtgctgcatgcgctgctccgtctgcaccgcacgctcacctcctggctccgcgttcggttccacacctggaactgg

SEQ ID NO: 166 - DNA, 110 bp, Artificial Sequence
  Description of Artificial Sequence Synthetic polynucleotide
cggttccacacctggaactggatttggcggcgctgctgccgcgccgcctctgccgcggtcctagagccgcttggcttcacgctccgcaagcatggaacagccctcaccac

SEQ ID NO: 167 - DNA, 81 bp, Artificial Sequence
  Description of Artificial Sequence Synthetic oligonucleotide
cggttccacacctggaactggatttggcggcgctgctgccgcgccgcctctgccgcggtcctagagccgcttggcttcacg

SEQ ID NO: 168 - DNA, 91 bp, Artificial Sequence
  Description of Artificial Sequence Synthetic oligonucleotide
ggtcctagagccgcttggcttcacgctccgcaagcatggaacagccctcaccacacgcacccgcgcggggggtagtgcctgcccgcagccc

SEQ ID NO: 169 - DNA, 106 bp, Artificial Sequence
  Description of Artificial Sequence Synthetic polynucleotide
tggggccgaagagatccttgaacacgtcgtaggactcctcgtcggccgccacgcggcccacggccctgagtacgggtggcccgggctgtccacgcgggtctggatg

SEQ ID NO: 170 - DNA, 130 bp, Artificial Sequence
  Description of Artificial Sequence Synthetic polynucleotide
gctgcagtttcgtcagcccttggctccgggctctgcaggcggaatcccgagcctgcgtgagggccgccctggcctcggcgtgtgtcctgggaaggggcgttggaagcctctctgcttgtcttggctgcct

SEQ ID NO: 171 - DNA, 93 bp, Artificial Sequence
  Description of Artificial Sequence Synthetic oligonucleotide
ggctcaccttcaggaagcacctgtggcgggccgcgtcacccactcgggaccccggagaccaagtccgctcttctgcacgtaaaccctgcctcc

SEQ ID NO: 172 - DNA, 107 bp, Artificial Sequence
  Description of Artificial Sequence Synthetic polynucleotide
ggtctttcccacacctctgcaccttgttacctgactttcggcttcaggatccgcagcgtgcacccgcgttccgtgagtgccctataggcagtcagcatgcccctctg

SEQ ID NO: 173 - DNA, 116 bp, Artificial Sequence
  Description of Artificial Sequence Synthetic polynucleotide
gcctggccaccacagagaagaagacggagcagcagcggcggcgggagaaggctgtgcacaggctggtgagcgcctgggccagcggggcctgcctctgatgcctcgcccccttcctt

SEQ ID NO: 174 - DNA, 90 bp, Artificial Sequence
  Description of Artificial Sequence Synthetic oligonucleotide
tgctctctctccaaaggcgagttgatcacagacgctggcagtgagtcagcggcaccgccagggctgctgagaaatccctcctgctgtccg

SEQ ID NO: 175 - DNA, 88 bp, Artificial Sequence
  Description of Artificial Sequence Synthetic oligonucleotide
cctgggaaccagtgctggagaaagtatgtggaagctggcgatggagaaggcgcgcgcatgtgtgcacaacctcgctctggaggagtca

SEQ ID NO: 176 - DNA, 109 bp, Artificial Sequence
  Description of Artificial Sequence Synthetic polynucleotide
acactttgaaaagcgtggcgttccagcgcaaaccaacccgaacgggttggaagggggcagtcctttcttcccgcaagttcggggctcgagagacggctgcaggaaggcc

SEQ ID NO: 177 - DNA, 110 bp, Artificial Sequence
  Description of Artificial Sequence Synthetic polynucleotide
aaaaggctccgacgatgctccagacgcggacacggccatcatcaatgcagaaggcgggcagtcaggaggggacgacaagaaggaatatttcatctagaggcgcctgccca

SEQ ID NO: 178 - DNA, 123 bp, Artificial Sequence
  Description of Artificial Sequence Synthetic polynucleotide
cgagaagggaggaggtgaaggagggcgagctgagcacacgcgcttcatgccacaggagggtgggaatgagcggaggactgaggagaggaaggagggaaagaatagggagatgaaaacgccccg

SEQ ID NO: 179 - DNA, 119 bp, Artificial Sequence
  Description of Artificial Sequence Synthetic polynucleotide
gctctgatgcctctccctccacaccacacctgtgatctactgtgcataggatctcacaggcccaataacagagctggagttcctcttacgtgacacaggatttggcatttgcctgtgcc

SEQ ID NO: 180 - DNA, 116 bp, Artificial Sequence
  Description of Artificial Sequence Synthetic polynucleotide
gacatcctccttggcagcctttcaacacgtttctcaaatcctttcccagcttcctgtgcagcctttcctcctcagcctggctgccttactgtctcagctccgatctctgggccttt

SEQ ID NO: 181 - DNA, 106 bp, Artificial Sequence
  Description of Artificial Sequence Synthetic polynucleotide
tgagcgcttaacgatccggaaagaggaagatggagacgctggaaaggaagaggacgccaggacgcgcatcatcagacgcgcagctctggaatttgaggacggcatg

SEQ ID NO: 182 - DNA, 80 bp, Artificial Sequence
  Description of Artificial Sequence Synthetic oligonucleotide
ccccatagggaggacttgcgcacagttggcgctgggtaaatgctgggagaactgctgcgggcgaggggaaagggttaaag

SEQ ID NO: 183 - DNA, 78 bp, Artificial Sequence
  Description of Artificial Sequence Synthetic oligonucleotide
gtcatcagtgaatcgaccacaaagagcctttgcggaggtgatttacaggagagctctgatgtctgctgtcccctgcac

SEQ ID NO: 184 - DNA, 134 bp, Artificial Sequence
  Description of Artificial Sequence Synthetic polynucleotide
acaccccgcggcaggacttctagagaagcccaggatctgtcccgtgccgccgctgctcccctccccagacacctctccacgtctcctacccagggggtcgcatccctagcccttcactgaccccagctcttccc

SEQ ID NO: 185 - DNA, 131 bp, Artificial Sequence
  Description of Artificial Sequence Synthetic polynucleotide
tcggcagtgaaaagcgggagattagaaaatgtttcatgctaatttccatggagatttctttaatttagcgaagactgcttcccgggctccgcctggcccgcgccggcccgcgtcctcggtggtctgggcgc

SEQ ID NO: 186 - DNA, 125 bp, Artificial Sequence
  Description of Artificial Sequence Synthetic polynucleotide
ggctctcgggctctcgctttttttttttttttttctttccgcggcagtcttaggattcttgtcacatgatggcttcatcgggcccttctcctcctgatcctttcaagctctttctcctgcctggc

SEQ ID NO: 187 - DNA, 143 bp, Artificial Sequence
  Description of Artificial Sequence Synthetic polynucleotide
ctggggagaagtgaccccattcaatagtccttggtctccttctgccctgcggctgcgcttcctcggctctcacggcaccagcagaattccatgtgagagggagcttgtcgagcgtggcctcttcccacttggggctgctttct

SEQ ID NO: 188 - DNA, 121 bp, Artificial Sequence
  Description of Artificial Sequence Synthetic polynucleotide
gcaccatcctcagagcttcagaccatacattgacagtgagcaaagggggccccaggcaggcgggtctggggccaaggagggcggctcccctgcgcggatccttccctggtggctcccaaat

SEQ ID NO: 189 - DNA, 117 bp, Artificial Sequence
  Description of Artificial Sequence Synthetic polynucleotide
gcttcaaacgccgtatcatgttgctttaaaacctgcgggtaacagcataagctgagttttctatcttagaactcttaaccccaagaacactcttcacaggccctgataggtggaccc

SEQ ID NO: 190 - DNA, 74 bp, Artificial Sequence
  Description of Artificial Sequence Synthetic oligonucleotide
tggtgccaggggttaccacaaagaggcggcagagccatggcccaccagccacttggcaggctggttgtctggtg

SEQ ID NO: 191 - DNA, 112 bp, Artificial Sequence
  Description of Artificial Sequence Synthetic polynucleotide
gagacaacagcccagacccccatcacggagctgcacgtgaccctggaacttaacagcttccagttgttccctagacagtcattgtctttatggtgccttttcccccatcagg

SEQ ID NO: 192 - DNA, 116 bp, Artificial Sequence
  Description of Artificial Sequence Synthetic polynucleotide
tgtaagatgacacagctatattttctgggagagggcgggaggatgctcagcgagggtggcccggagtgtccttgtacagagtacagatgttatgaagtggggaagaccagcctgtg

SEQ ID NO: 193 - DNA, 108 bp, Artificial Sequence
  Description of Artificial Sequence Synthetic polynucleotide
ggaggagctgaggtttcggctgagcccccagcctcccccgaccgcacagcctcgggcatgaacccgcgaagccagacgcttagttgcttatcaggccatcgctgtaca

SEQ ID NO: 194 - DNA, 120 bp, Artificial Sequence
  Description of Artificial Sequence Synthetic polynucleotide
aggaggagaccctgccccagaaataggccagtgcttgttatgcaggccttggcggttccccgtttccttacgtaacctcagtgttcacgctgtttccttttgttgattccctccgtgtga

SEQ ID NO: 195 - DNA, 609 bp, Artificial Sequence
  Description of Artificial Sequence Synthetic polynucleotide
catctccatcttctccatcatctccatcttctccatcatctccatcttcatcatctccatttccatcatctccatctccatcatcatctctatctccatcatctccatctccatcatctccatcatctccatctccatctccatctccatcatctaccgtctccaatctccatctccgaagttatgcccacttcctcgaagtttggagccacgcgaactacactgcccagaaggcgccgcgccgtgagccgcgatgcttggccaatgaaaagaggtctacccgagagtgcgacgcgcaatgggcgggacttccggcgtctcccctcggcggttgctttcgctgccctcaagagaactcagcttgccggaagctggttgttcgctgcggcgaccagctccggaaagcgcggtggggacgcgctgtgttctcgcagctcagaggcgggtctgaggctcggtggcggcgcccagggtggcccgggccctttcctcggtcgttgtctcaccgccacaggctccgatggcggcggccacgctgagggaccccgctcaggtgagcgccgcgtcctcccggcctcccccgaatcctaaagccctgtgagggccgcc

SEQ ID NO: 196 - DNA, 617 bp, Artificial Sequence
  Description of Artificial Sequence Synthetic polynucleotide
cttcctcggactctcggccgacgagctcatcgccatcatctccagcgacggccttaacgtggagaaggaggaggcagtgttcgaggcggtgatgcggtgggcgggtagcggcgacgccgaggcgcaggctgagcgccagcgcgcgctgcccaccgtcttcgagagcgtgcgctgccgcttgctgccgcgcgcctttctggaaagccgcgtggagcgccaccctctcgtgcgtgcccagcccgagttgctgcgcaaggtgcagatggtgaaggatgcacacgagggccgcatcaccacgctgcggaagaaaaagaaggggaaggatggagccggggccaaggaggctgataagggcacaagcaaagccaaagcagaggaggatgaggaggccgaacgtatccttcctgggatcctcaatgacaccctgcgcttcggcatgttcctgcaggatctcatcttcatgatcagtgaggagggcgctgtggcctacgatccagcagccaacgagtgctactgtgcttccctctccaaccaggtccccaagaaccacgtcagcctggttaccaaggagaaccaggtcttcgtggctggaggcctcttctacaacgaagacaaca

SEQ ID NO: 197 - DNA, 616 bp, Artificial Sequence
  Description of Artificial Sequence Synthetic polynucleotide
ttccctttttctcctcacaaggaggtgaggctgggacctccgggccagcttctcacctcatagggtgtacctttcccggctccagcagccaatgtgcttcggagccactctctgcagagccagagggcaggccggcttctcggtgtgtgcctaagaggatggatcggaggtcccgggctcagcagtggcgccgagctcgccataattacaacgacctgtgcccgcccataggccgccgggcagccaccgcgctcctctggctctcctgctccatcgcgctcctccgcgcccttgccacctccaacgcccgtgcccagcagcgcgcggctgcccaacagcgccggagcttccttaacgcccaccaccgctccggcgcccaggtattccctgagtcccccgaatcggaatctgaccacgagcacgaggaggcagaccttgagctgtccctccccgagtgcctagagtacgaggaagagttcgactacgagaccgagagcgagaccgagtccgaaatcgagtccgagaccgacttcgagaccgagcctgagaccgcccccaccactgagcccgagaccgagcctgaagacgatcgcggcccggtggtgcccaagcactc

SEQ ID NO: 198 - DNA, 625 bp, Artificial Sequence
  Description of Artificial Sequence Synthetic polynucleotide
tggtcagggcctgaggcaaccctgtcccagcgctgaggacccaggaacatgccaccagcctgggatgggggaggccacggagggagggagcagtgagcccccagggaggaatctcgagctgagggaccaggagttcgggcttgttctgagaaacgcacagtgtcagagtcactcattcagaaagactgagagagcctgccgagagctgggtaccggagacgcgtccctgccctctcagagttgacagtccagaggcaaaaaggacaatcggcaagtaaatagtaaatgaacaagaagaccccggttgtgagaaaatgttataaagcaaataaatcagagaaatgtgatcacaaaccctgggtgggtgaagggtacaagtttaggaaacgggtcagggaaggcctctctgacatttgagctgagccttggatgaccagaaagaactattgaaagatctgggtggggccagaggaggggtgagtggcagatgccccaggagagaagaaagttgtccaggaggggcccgtgcactggaggcaggggacggggcaggcacaggggcagggggacgaagccagaggcactccctcccccagggtgctgagcaggggagccccctgactca

SEQ ID NO: 199 - DNA, 608 bp, Artificial Sequence
  Description of Artificial Sequence Synthetic polynucleotide
ccaaagaatgcagagaatgtgcacccgtctgtgacatagttggtaatttccagaggcggagaagatattattgacaataaggtgaacacgctgtgtgaccaccgtggatcgtcatatccagggggggagatgggggtgatatgactccccgcatcgcggggggcgcccgcccccctgcgatgtggatcatcatatccagggggggagaggggggtgatatgactcccctcatcgcggtgggcgcccgcccccctgcgacgtgaatcgtcatatccagaggggggtgatatgactccccgcatcgcgggggcctcaccccattgcgatgggggtcctaagagccagggggagataggggctggctcttactccccgtaccgccgggggggggggcctcaccgccctgcgacggggctccttagagccagggagggggaggggctggctcttactcccggtatcgcaggaggtgtgtacaacccctgcgatattgggagtaatatcatcctctccccctgaatataagaaacaatatcacaggagcatgtacaccccctgcgatattggaagtaacatcattttctccccctagggatattcggaacaat

SEQ ID NO: 200 - DNA, 582 bp, Artificial Sequence
  Description of Artificial Sequence Synthetic polynucleotide
ggggggcaaggacacggggccctccccaggctcgctggcagcccattgtgctgggctggaaggtctcccaacctgaggacacctaggggcaagggagccactggcctgagcctgagatctctgagcgggggcaggcagccctcgccatgccaagggcatccctaatccacccctacacaccagcggaagccactggcagtgagggcccagggccaccaagcagggctggggcaggaaagaccagcaggtgcagctgagggaaagggggaagtcactgggctgggggccggggccgctcactctggcctcctctgaggggtccactggggttccggctcctcagaccctggctctgcagcctcagggccaacttcccgcttggagaaagggcagcgcttgtccggggacccaccacatccatcctcgtagggggctgtctccacccagggtcccccccccaccccctcattcctcccagtggtgaaaggacagtgaaggaggagggcagcccaggagtggacatggagtgaccaggagcttcctggggggtccgggaggtggggcacaccctatcgcacacca

SEQ ID NO: 201 - DNA, 607 bp, Artificial Sequence
  Description of Artificial Sequence Synthetic polynucleotide
tggccgggtctaagctgtgctcctgctgcctggctggcttccgcccggtcagactgacagggtcttgcaggcaggaaccgtgcacacagtgtctagctccgagcctgagaatactcgtggcttcaaaagtttgctgagctaccgcagggaggacgaaggctataacactggtccagcctgagagaagcccaagtggggttcactgccctctgagccacagatttaagggggagggtgtggaaactgccggctgctgaggtccaaactcaccgaaggtactgaccgccgcggctcctctcttcacagcgtctgccggaggcctccgtttactccggttaccgagacaacgccacccctcttccagggaggcggaaccagggcgggccgtggggcgcatgcgcggccggcgtccagctctccgggaacccggtacctatccgccctttggtcgggccttctccgcctcatgacactggttcaaagccaaacagaaaagcccgacgagtttattatcccctaaaggacgtcatgtagataattaaatgacatgaataccgtcgaagatacctgcctgatattccaaaatggcccaacggagccctgatcg

SEQ ID NO: 202 - DNA, 630 bp, Artificial Sequence
  Description of Artificial Sequence Synthetic polynucleotide
tataaagtgcgcgcagtttgttttatttcctgagtttttgcaatctagataacagatgataccctgagtggctggcgctgcctctgtaatggcggcactgagcctttggagaagtattaataatagattgtgttgatgagtttggagaaagtagcaatcgaccccctgctgccaaggcattagcgcggctgttctgagcacagccagcactgtggctttgactgcaaatgcaggtcacccgccctgctgccccttccccagcctaagaaggtttcctctccgggagtcacccaaggtgtgctgaccctggcctgggaccctgggaccgtggcgctcccacgctagcagcgacacggccagtgtctgtccactcagaggccgcagaggtcaggctgcagaccttagtgtggccactaggtcaggtggagtgtggggaggggacagaggggcagtaggggttgggggaggaccaccctccatgtcagagcaccgggttctacaaacccaggctccttcctcagcccctcgggagagctggacagccagccagattcctagggcctctgcctaaagctgtcactgacagttgggtaggttgtgccctgaacaaggggattcagccagagggcc

SEQ ID NO: 203 - DNA, 591 bp, Artificial Sequence
  Description of Artificial Sequence Synthetic polynucleotide
agtgtgttcctcacttccacctgtggcggtcgcttctggctgtcaccctgagcacatccatgtggcctcttggagtggcctctccacgtggcctaggcttcctggcaacgcagccgcctcagggcagtgtgacttcctgatggtggtgactcaggacaacaaaagcgagaggccctgagagtcaggcgggcaccacagggccttgctgaggcagccggggactcccgctccctctgctgacaccattggtggccagtgagtcagaggcagaggtgccagagaccccgcccgaagggaggagatctgagagcctgcagccacaggctcctccaggactcgagcaccggggccgcacagagagccctttctctcctgggcaggccaggcggggatcccccagcgccctaacctgctctgtgaccacggcaatgtggccttggggatgtgccctgcctctctgggttccagtgcaggactcagggctggccacctgagaagcatctctaggacattccaaagcctggaacagggacagcattgtggccctgctctggaaggctgcgtggaagccaagaagttgtcctggcctgt

SEQ ID NO: 204 - DNA, 603 bp, Artificial Sequence
  Description of Artificial Sequence Synthetic polynucleotide
acagtgtctagctccgagcctgagaatactcgtggcttcaaaagtttgctgagctaccgcagggaggacgaaggctataacactggtccagcctgagagaagcccaagtggggttcactgccctctgagccacagatttaagggggagggtgtggaaactgccggctgctgaggtccaaactcaccgaaggtactgaccgccgcggctcctctcttcacagcgtctgccggaggcctccgtttactccggttaccgagacaacgccacccctcttccagggaggcggaaccagggcgggccgtggggcgcatgcgcggccggcgtccagctctccgggaacccggtacctatccgccctttggtcgggccttctccgcctcatgacactggttcaaagccaaacagaaaagcccgacgagtttattatcccctaaaggacgtcatgtagataattaaatgacatgaataccgtcgaagatacctgcctgatattccaaaatggcccaacggagccctgatcgcggcgttcctatgttgaggttttaacttcgattttaagaggggtcctgggagatagtaggcagcttgccggcaacatcaac

SEQ ID NO: 205 - DNA, 610 bp, Artificial Sequence
  Description of Artificial Sequence Synthetic polynucleotide
gatccagccgagtcagggactttccccacgccccacccgaccctcaggcctgacagccacaggggcagagcaggaggaggccaggcaggggcagtgagggaaacagggaggggccttggccacagcaatcttgggcctccagccccatgggaaccccagcacgatgagcatccagggtcattgagggggaggcggggagctggctgtggccacctggagtcacagcggggcaagggttgggggccccaggggagctgggacaagaagggaacacggtaccagggtagcagaagacaggcaccccccgtcccccagtcctagggcttcctcaccgcgcctgtgaggagctccaggcctgtgcagacgggggcagggcccggcagggcgggtgggaaggcgacctgagggcccatgatgaaggccaccagcagcagcagcaggagggcattgaaagccagcagggtcttgagaaagaggaagtaggagagcacgctggagccgaactggcccccgatgcgcttcagggcgtagcgccacggcatcagggcctgcagggcggagagcagcgccaggcccaggctgtgcaaggcctgcgggcacaggcagagag

SEQ ID NO: 206 - DNA, 596 bp, Artificial Sequence
  Description of Artificial Sequence Synthetic polynucleotide
taacactggtccagcctgagagaagcccaagtggggttcactgccctctgagccacagatttaagggggagggtgtggaaactgccggctgctgaggtccaaactcaccgaaggtactgaccgccgcggctcctctcttcacagcgtctgccggaggcctccgtttactccggttaccgagacaacgccacccctcttccagggaggcggaaccagggcgggccgtggggcgcatgcgcggccggcgtccagctctccgggaacccggtacctatccgccctttggtcgggccttctccgcctcatgacactggttcaaagccaaacagaaaagcccgacgagtttattatcccctaaaggacgtcatgtagataattaaatgacatgaataccgtcgaagatacctgcctgatattccaaaatggcccaacggagccctgatcgcggcgttcctatgttgaggttttaacttcgattttaagaggggtcctgggagatagtaggcagcttgccggcaacatcaacaacaaagatacatcgtgggatttttgttatttttaaaactatattatctctgttggcttttaagagtaaa

SEQ ID NO: 207 - DNA, 600 bp, Artificial Sequence
  Description of Artificial Sequence Synthetic polynucleotide
cacactcagcctggcctagaaaaaactcaaaattttgaattttcatcaaatgagagaataaatgattaaacaaatagaaatgcttcacccagcagcaagcgcttagattttaaggacccaagcaaagtgcatggaaaggtgcagctgtctggaaggacgattgggaggtgggatcttggggagaaagggaagaaaggggatggagcagggcttcccagtcgagggcggcggccgagcctgtgtccccaccagcgtctctgtggccgtgaagtgtatgcatgcgtgcccatgttgatgcggcgccgtgcgggaggcgggcatcccctgctgtacatgggagggaggctgtctgtgcagagcattgcccagttgccatagaaacgagcagaaggaggtgggtggctggagaaggaggcgggtcgggatcggggagtggggaggaggcagcggtggagggagctggctcctgcagttctggcgctgctgccttcctgagtgagcggtggagggaaccctagaggacagagcccccagcccggcagcaggccccctctccgcccgccaccacggaggagaaggaggacagccagcccctccagc

SEQ ID NO: 208 - DNA, 608 bp, Artificial Sequence
  Description of Artificial Sequence Synthetic polynucleotide
acaccacgtgggcccctcccgccctcccccagcacttgcacaaagcctggaggagggcctccctgtcccacacaacttcctgcttgtccccttcccacccctctcctccccaggagcggctcccaggcccacgaacagcggcttcaagaggtggaagccgaggtggcagccacaggcacctaccagcttagggagagcgagctggtgttcggggctaagcaggcctggcgcaacgctccccgctgcgtgggccggatccagtgggggaagctgcaggtgcggctggccagcgactgagagacccgggcgctaccaaaaggggagcggggtggcggggcagttcctaaggcttcccgggggctgggaggtcccaaactgtgggggagatccttgccttttcccttagagactggaaaggtagggggactgccccaccctcagcacccaggggaacctcagcccagtagtgaagacctggttatcaggccctatggtagtgccttggctggaggaggggaaagaagtctagacctgctgcaggggtgaggaagtctagacctgctgcaggggtgaggaagtctagacctgctgcaggggtgaggaagtct

SEQ ID NO: 209 - DNA, 602 bp, Artificial Sequence
  Description of Artificial Sequence Synthetic polynucleotide
tgaaatatatgtccacacacggagaatttaagagtatttttatatttctctctagatctaaatattcagatgtgttaattacatgccctagaagctggaagcgatcagtggtgttcacactggacgtggagctgtttgtataattttcatctccctgcacttaaacatgactctcagtctaataaattcaaccttgtcatttttagaatcgacgggatttctctggctgtcgtttgcgctgcatttatccgaatacatccagctcgcaggcatcctgcaagaaacggctcccggctcgcgtgtacgccgacacctcggcccaacgcaggactcgaggtggtttctagtgcccgggtggctgcaagtctgccctccgagggaggctggacaagcggcgcccccaggtcgagcggcctctcgctgcctggcagtgcctggcagcccccacctctgccagtgcttcggaaacccgcctggccaggttcgcccgcggtgaaaaatgaaagcaaattccccaacagaggtagccggaactttcctcgacgaaggctccctcctgcgcctgtgtctggagaacccccagagcgctgcaagttagcaag

SEQ ID NO: 210 - DNA, 608 bp, Artificial Sequence
  Description of Artificial Sequence Synthetic polynucleotide
agtccaagtttctgccacagttccagggccgaggctgtttccaaagagccctgtaattgttttccacctgtgtctcacccaaacaccaaggctggcgcaggtggacaccttcccacttttctccctccaggctgggccccagaaatcagtagaggagggaggaatcagtcagcgtggccatgcctgggaggagaggcccgtgtgggtctgtggggctaagaggcaaaggcgggtggcggatgtgggccagcgggagcctggaggggttgacaccgcctgctccaccgcaagcccctggaggaagagccccgctgtgcccgagagcgagcgcgggcaggtgtaactacccggggctggggctccgggggctccgcgcagcctccttccctcccagggacaccgcccagctgcgccccgcgccccgccgactgcgcgggccttgagacgctggtggctgcctcggggttggcctgctcctcgcgcacatgttcagggtcatccgcgctgcgcctctgcttcaggtgcttggctagagaaagggcggcaagacggggcagtgcgtgtgcgcgcgcgggcaagtgcatgtgagtgcacacttatgtgagcgc

SEQ ID NO: 211 - DNA, 612 bp, Artificial Sequence
  Description of Artificial Sequence Synthetic polynucleotide
tggaggggttgacaccgcctgctccaccgcaagcccctggaggaagagccccgctgtgcccgagagcgagcgcgggcaggtgtaactacccggggctggggctccgggggctccgcgcagcctccttccctcccagggacaccgcccagctgcgccccgcgccccgccgactgcgcgggccttgagacgctggtggctgcctcggggttggcctgctcctcgcgcacatgttcagggtcatccgcgctgcgcctctgcttcaggtgcttggctagagaaagggcggcaagacggggcagtgcgtgtgcgcgcgcgggcaagtgcatgtgagtgcacacttatgtgagcgcatgtgtgtctgcgcttgtgcgtgtccaggggaaccacagggagcaccctcattctaagcctccagaggactgcctgaagccgctagatagaaactcccctagaatgtaagctccgggggggagggagctttgtttgatggctgctgtattcccagtgcccattgaagtactggggacacattagatgcttaataaacagctgttgagttaatcaacggactctaggaatggaggcagaccggcccttctggaactggagaaa

SEQ ID NO: 212 - DNA, 625 bp, Artificial Sequence
  Description of Artificial Sequence Synthetic polynucleotide
ttgaaaccccgtctctactaaaaatacagaaaaaaaaaaaatagccgggcgtggtggcgggagcctgtagtctcagctactcgggaggctgaggcaggagaatgtcgtgaacctgggaggcggagattgcagtgagcccagatcgcaccactgcactccagcctgggtgacagagcgagactccgtctcaaaaaaaaaaaaaaaaaaaaaaagccgtcgcgcctcgggagtgggctggggggagagggggtgcccaagggccacaagagtatgacggggctgtacgagctgctgtgacgggtgctgcatgcgctgctccgtctgcaccgcacgctcacctcctggctccgcgttcggttccacacctggaactggatttggcggcgctgctgccgcgccgcctctgccgcggtcctagagccgcttggcttcacgctccgcaagcatggaacagccctcaccacacgcacccgcgcggggggtagtgcctgcccgcagcccaccaccgaatgcgctggcgcgcggacggcccttccctggagaagctgcctgtgcgcatgggcctggtgatcaccgaggtggagcaggaacccagcttctcggacatcgcgagcctcgtggtgtg

SEQ ID NO: 213 - DNA, 611 bp, Artificial Sequence
  Description of Artificial Sequence Synthetic polynucleotide
gtcgtgaacctgggaggcggagattgcagtgagcccagatcgcaccactgcactccagcctgggtgacagagcgagactccgtctcaaaaaaaaaaaaaaaaaaaaaaagccgtcgcgcctcgggagtgggctggggggagagggggtgcccaagggccacaagagtatgacggggctgtacgagctgctgtgacgggtgctgcatgcgctgctccgtctgcaccgcacgctcacctcctggctccgcgttcggttccacacctggaactggatttggcggcgctgctgccgcgccgcctctgccgcggtcctagagccgcttggcttcacgctccgcaagcatggaacagccctcaccacacgcacccgcgcggggggtagtgcctgcccgcagcccaccaccgaatgcgctggcgcgcggacggcccttccctggagaagctgcctgtgcgcatgggcctggtgatcaccgaggtggagcaggaacccagcttctcggacatcgcgagcctcgtggtgtggtgtatggccgtgggcatctcctacattagcatctacgaccaccaaggtattttcaaaagaaataattccagattgatggatggaattt

SEQ ID NO: 214 - DNA, 582 bp, Artificial Sequence
  Description of Artificial Sequence Synthetic polynucleotide
gtcgtgaacctgggaggcggagattgcagtgagcccagatcgcaccactgcactccagcctgggtgacagagcgagactccgtctcaaaaaaaaaaaaaaaaaaaaaaagccgtcgcgcctcgggagtgggctggggggagagggggtgcccaagggccacaagagtatgacggggctgtacgagctgctgtgacgggtgctgcatgcgctgctccgtctgcaccgcacgctcacctcctggctccgcgttcggttccacacctggaactggatttggcggcgctgctgccgcgccgcctctgccgcggtcctagagccgcttggcttcacgctccgcaagcatggaacagccctcaccacacgcacccgcgcggggggtagtgcctgcccgcagcccaccaccgaatgcgctggcgcgcggacggcccttccctggagaagctgcctgtgcgcatgggcctggtgatcaccgaggtggagcaggaacccagcttctcggacatcgcgagcctcgtggtgtggtgtatggccgtgggcatctcctacattagcatctacgaccaccaaggtattttcaaaag

SEQ ID NO: 215 - DNA, 592 bp, Artificial Sequence
  Description of Artificial Sequence Synthetic polynucleotide
agcctgggtgacagagcgagactccgtctcaaaaaaaaaaaaaaaaaaaaaaagccgtcgcgcctcgggagtgggctggggggagagggggtgcccaagggccacaagagtatgacggggctgtacgagctgctgtgacgggtgctgcatgcgctgctccgtctgcaccgcacgctcacctcctggctccgcgttcggttccacacctggaactggatttggcggcgctgctgccgcgccgcctctgccgcggtcctagagccgcttggcttcacgctccgcaagcatggaacagccctcaccacacgcacccgcgcggggggtagtgcctgcccgcagcccaccaccgaatgcgctggcgcgcggacggcccttccctggagaagctgcctgtgcgcatgggcctggtgatcaccgaggtggagcaggaacccagcttctcggacatcgcgagcctcgtggtgtggtgtatggccgtgggcatctcctacattagcatctacgaccaccaaggtattttcaaaagaaataattccagattgatggatggaattttaaaacaacagcaagaacttctgggcctagattgttc

SEQ ID NO: 216 - DNA, 581 bp, Artificial Sequence
  Description of Artificial Sequence Synthetic polynucleotide
acgggctgagcctcaaacgagctgcaggccgagttctaacgggctgagcctcaaacgagctgcaggccgagttctaacgggctgagcctctaaggagggaaacgtcacttcctgcctcacacagagcccagcgtctccatgtccactgatagccttggtatttgcaactatgtccatgaccatctctgtttctccaaaacagcctctagctacataaactgtttagaaaacctcatgcgtaaagcagagtatgtcaaacctccatctgtggtcaggagttaggacatccccagctgcaatttgagcaaagacggcgcttccagaggatcatcggatcctgtgtcttggttggggttggggcccatcaacttaaaatagcttctgtttatgctggtgaaggaggcacagacttcaccctatctaattccaaggaacaggcgagggtgggagctgtagcggaagagacaaaagcaaaaggcagattcgcccctttgtgtggtcccgtaagtgacactgtccctccctctccctggaaacagcagcccccaggcaccccccccagcaactgggacaagggcaca

SEQ ID NO: 217 - DNA, 607 bp, Artificial Sequence
  Description of Artificial Sequence Synthetic polynucleotide
tggctgtcgaggagctgctgttgctgttccgcgtcggtcctgctcctgcgcgcgtcgtccaggcccgccaggtcgccgaccagtctctagggcgtccatcgcgggacccacgggaggcagaagtggaggccgtgcgcaccgcgagctcaacacagttgggggccaggtggccgcctcccagcaggttgtcggggttgagctgggtcttgtgctcatcgctgggcttgtagtgcggtgccggtcctcaaggatggggccgaagagatccttgaacacgtcgtaggactcctcgtcggccgccacgcggcccacggccctgagtacgggtggcccgggctgtccacgcgggtctggatggcgcctccagcgcgaagccacccctggcgcgcagctccgcgttcagctggggcagcgcctcggccactgggtcttggtggccgctcaggtcgggaaactcgtcctgcgccgggaggcgagcttcagcgccccgcggctgtcggagaagggcatgtgcgggcgctcggtgggtccgcagctctgagcgtggccactttttaactgttataaataattctgctatcaacattcatatgtacacttttcttat

SEQ ID NO: 218 - DNA, 631 bp, Artificial Sequence
  Description of Artificial Sequence Synthetic polynucleotide
ctgctattgatgttttctctgcccaggttctccccacacacggggttagggagggtgtgccagcctgccctcacatccccagacagagtccccctccagcatctgctgcctacctccttctccctcagtgcctgtttgtttttcttccagaaccatcgcctctcaccaaggcagccatccaaggggggcggtgttccggagacatcctctgccccccgcacccctgcagcggtagcctggtgggggctggtgctgcagtttcgtcagcccttggctccgggctctgcaggcggaatcccgagcctgcgtgagggccgccctggcctcggcgtgtgtcctgggaaggggcgttggaagcctctctgcttgtcttggctgcctctgctcgctcagctctgcccccactggggccgccagcctctgcactcccccttggaggagccaggcagggtttgggtcggagctggggtagaggaaggctccaggcggcttgccgcaggatctccctgctgtagccagcccttggggcgctcagcagggtgggggaccatcagtcagggtgggggaccctcagtcaggataggggggctcctgttctttccactgccaccaagctacccttcccctaact

SEQ ID NO: 219 - DNA, 618 bp, Artificial Sequence
  Description of Artificial Sequence Synthetic polynucleotide
taccccagctgcctgaccgggagagcatcctgttcttcccctctggaattccgggtccacagctgtcttcctactcacatctggcctcggcattcccgccaagccctccccttgaagcacaaggatgttttgtccaggatcctgagcccagggccttccaggtggcagagagagatccggatgtccagccagctctgggggttcccccatcctgccagtgtggggacctccttgctgtagccaggtcaggccagccctagggagacagcaggatggttccaggaagcctgggccgctccccagatcaatgcagggacggacagcagccagcaggctgggccacggcatcagagctggggtcaagaggtttctagccctcttgtggctctcagccccgggtcctggctgcttcctgctgggcagtgacctccccagtccatttccctccctccttcctcccctggcctgagctcagctcatggaaggaggccctgtgtgcaggaaccttgatctgcacctctgaaggatgtcagggcagctttttctctgggcctgtatgactcagcgcaggatttagggcaggtggctccaccgtggagcctcagtttcctcatctgg

SEQ ID NO: 220 - DNA, 594 bp, Artificial Sequence
  Description of Artificial Sequence Synthetic polynucleotide
cccgtctcggctctggctccgtcccctggcctacccactagcgggtcggactccgcccctgcttctgaccacgcccccgcgcccaccctcttcccaccctcctcccacccagggctctccagacgcgcatgcgcacccgttgtgcatctgccgcgtggtgaccgacacgccgtcggcgccgtccccgctgggccgcagcagcaggttcccgcactcggggtagcgctccaggagcagttgtgcctccagccggctcaccttcaggaagcacctgtggcgggccgcgtcacccactcgggaccccggagaccaagtccgctcttctgcacgtaaaccctgcctcctctgagacccagccccatccccatcccctaggcccaggagaccctgccctgctctccagacccaggcccctcccacggagacccagtccggccttccaggctcctagtttttgtggggttttttgttttttttttgagacagggtttcgctcttgttgcccaggctggagtgcaatggcgctatctcggctcaccgcaacctccgcctcccgggttcaagcgattccccttcctcacaggcccggctaat

SEQ ID NO: 221 - DNA, 614 bp, Artificial Sequence
  Description of Artificial Sequence Synthetic polynucleotide
gattttcaggaaccatgcatggctatcgcctcctcccgcctggagggctgctcctgcgcctctgaccggcgctggttccagccgcggcccagctgagcacagcaggaaccgcagtagcagccggagcgcccacgcccggggtcgcctagcccaggaacgccttagttgcaaccctgcgtcgaggcccagctccgtgcgcagaaagccgaggccaaccagagcatttcctggacgagtcctctcggcctgcggggccagttcctcctaccagcttcctgctgccacctcggcttccatcagagggacgcttaggatggcgcaggggcccggagacactgtgaagagtccaggggaatgaggaggggctgggccgggcagcctcaggcccagcgcaggttagcgcttctcacgcctgagcagagatcagctactgccactgcggggaggacagaaggacccaggctccccagcctccctctgcaccgggagtgtaggaaactatttaaaaataataataataataataataataagtatggaatagaacttgcagatctaacccaaccaagttttcattctttttccttttccttttcttttttttaatgtatttt

SEQ ID NO: 222 - DNA, 608 bp, Artificial Sequence
  Description of Artificial Sequence Synthetic polynucleotide
tcacttgggcatcttaagagtgggttcgtaaacttggttgtgtgcgctgtgcagatgtcagtcaccctgtgtggtgggcaaagccgacttctccgcctctgtagctccgaaactacaatccccagaggcctctgcggtcacttccgctcccctccctacccttcagtgtgtagcgttgacgtcagaaacacttccggtcggtggcccaggcgcgttaagctggttgggacccgggaaggcctccctcttaaggtctttcccacacctctgcaccttgttacctgactttcggcttcaggatccgcagcgtgcacccgcgttccgtgagtgccctataggcagtcagcatgcccctctgcgtgtccctgtgttacggggacgccggctgggagccgcagagctatctcagaactagggcgctctcctttgggcacctccaggccattttcctttcattcgagcccacagggttagagataaaccctcactccgttgcttggggacaagggcttcactccctgtcccgagcttgcggctgagcttgagggtggctgggtcatcctggccccccactggatgggaattggctgctctggtgatttctgtga

SEQ ID NO: 223 - DNA, 617 bp, Artificial Sequence
  Description of Artificial Sequence Synthetic polynucleotide
cggagaagctggagcggcagctggccctgcccgccacggagcaggccgccacccaggtgagccccgcacctgcccactccctcccctccccgggcctcctacccacccctgacactgcaccccgcctccccaggagtccacattccaggagctgtgcgaggggctgctggaggagtcggatggtgagggggagccaggccagggcgaggggccggaggctggggatgccgaggtctgtcccacgcccgcccgcctggccaccacagagaagaagacggagcagcagcggcggcgggagaaggctgtgcacaggctggtgagcgcctgggccagcggggcctgcctctgatgcctcgcccccttccttccttcctcccaccatgggctgccctgggtgctgcgggcagcctgcacaccccaagccccgcatgtggcctgtggtttgggctgtttgggatcctcacagctgagactcatttcccagcctcttccaggcagggctcgggctggggtgggacagggtccctggcgcttctgtttgaggggcggggtggggggaggtttctgcaccgcagaccaggggagatggatgacaaaaggggcttcagcaaacagct

SEQ ID NO: 224 - DNA, 591 bp, Artificial Sequence
  Description of Artificial Sequence Synthetic polynucleotide
gatctccctaagaggttatgccagtcacactcctgccaagagagtatctctgcgccacggccaagggtgagtcatcctgctgagaggttgagctggggacgcctgcccagatgggctccaagtgagggagagcctggcggggagaacagcccggacagaggcagggcagggcgccgggacactgcttggcgcgtcctgggagtgaagcgcattgaacccagctcaggctggtggtgggggagtcttggcaatgctctctctccaaaggcgagttgatcacagacgctggcagtgagtcagcggcaccgccagggctgctgagaaatccctcctgctgtccgatcgcattcctggaagggtgggccgctcagggccccccagctccagtcccactcaggccccagaatcccagcagcccaccactcacttctttgcgctcactcttccttctggtccccacacaccgctccctctctcgctaccttcagtctttgctcagatgtcgagttcccagaggggcctccctgacgccaccgttctagcagcatttagcatttagataaatgacaaattttagattaaatgttagat

SEQ ID NO: 225 - DNA, 589 bp, Artificial Sequence
  Description of Artificial Sequence Synthetic polynucleotide
agagcctgcactggggaagatacacaccacagaagccggcgctgcagaagcacatatgccaaggacctagcgctggagacgtgcacacgcctgggaacaggtgctggggcggcacccaagcacgggagccagcgttggggaagcggcacaccccagggagctagcgctggcaaagcacacccaccaagagtgagcgctagaaagccgcacacactatgggagctccgccctggagaagcgtcacgtgtgtgcctgggaaccagtgctggagaaagtatgtggaagctggcgatggagaaggcgcgcgcatgtgtgcacaacctcgctctggaggagtcacggccaggtgcgcgcacgacaaccttcaccggagaagtcacacgcatgcgtgcgctggagaacctgaatttgtaatttcaaatttccctataaagaaatatccacgaactgatgactttgtgagtgaattctatcaaatatttgaagaaaaaaaaataccaatccttcacaaactctgaaaaaataggagggaacacttcccaactcattctaagatgccactattacggtaataccaaagccagacaga

SEQ ID NO: 226 - DNA, 601 bp, Artificial Sequence
  Description of Artificial Sequence Synthetic polynucleotide
aagtgttgggattacagacataagccaggtcgcctggcccaagctagatattgaggactgccagatggcaacagtagacaagacaccctagaatggcccatctagaaggaagtagataccttctctgtagggattcaacaagggggcaaagtgatgggcatcttgggtgaggaacccagcccgcggaatgggaaagggctgggacactggctttacagctgggttgggaaagggatctgatccttgagtcagcccctgtaaaatggggatacagcagggcacgacgtctgttggtcgcctggcactgggtcggccaccgaggccgcgccttggcctctttgtcccctctggccaccggccccagggagcccgctcgggaagcagcgcggccccaggaggaaggcggcgcggccgaggccagagccggcggctactgcgaccttccggctggcgggcgcgtttcatgttcctgcctcaccctgggctgcacggactcagatcgggaagggggaggatccatggtaaaggccacgccccctctgggacctcgattccccttctgggcggccgagggatgggctgcaaggagtcaaatcctctt

SEQ ID NO: 227 - DNA, 586 bp, Artificial Sequence
  Description of Artificial Sequence Synthetic polynucleotide
gccagcagacaataacctctcctcttgaattggaaaaacaaatctgtggaggccttctctgccccttgttataggggccccatatgccgttcccaggattaaaccaaaaagcacacattcctctctggcaggtgcaggtcccacccactctgggcagccaactgatgtgcacattttaatttcctaaaacaccaggacagaaccttcctcaggggtcaggtggctcacccttggccctcaggctttggagatgggcacttgtcatcatgggtgtttggaaagcaactctacgttctagcctgtgctccatcgttccttctacatacaagtgatgcaaacatcaaaatatgttttttctttcttccttcctttaaaaaaaattgaatcctggatgaagttttagctctgtcacttgacaactgcattatataacctagggtacttgaatctcagccccaaatctctaaaatgggggcagtaacatgcttctgccaggtcccagacctgtgggtctccaaatctgcacattcttcaagccttacaggtccttccctggtctctctaaggatgtcatgggcacagagcc

SEQ ID NO: 228 - DNA, 610 bp, Artificial Sequence
  Description of Artificial Sequence Synthetic polynucleotide
gaggcagagggtagggggtgaggaggtgtttcttgtcttcttcttccaatctcagaagtaaacattggaaagtggggcccccagcagtgtacagcccgtttccaaaccaggcctgtaaggaggagctgaggtttcggctgagcccccagcctcccccgaccgcacagcctcgggcatgaacccgcgaagccagacgcttagttgcttatcaggccatcgctgtacatatttagaaagtacctatcactcagacactttgaaaagcgtggcgttccagcgcaaaccaacccgaacgggttggaagggggcagtcctttcttcccgcaagttcggggctcgagagacggctgcaggaaggccatcacccctggcttcctgcagccacagcttccagccccacacgatgcccaacttcattttagcagtggcccccaggggaaatcacaccattcttggttttgtccctccctcctgaggttgggacattgttcaaacaaaagtaagccttcagctgacagagaagctgccccgcctcttccctgcccttgtcttgctggcattcattgggactaccaggtagctttccttcccagctcaggtgtttacctgc

SEQ ID NO: 229 - DNA, 611 bp, Artificial Sequence
  Description of Artificial Sequence Synthetic polynucleotide
ttatagatgagattctacttaggggtaggattcattattcatgaagggtgtggtcaggtgaggcatgttggaagcaaaatgcgaattaggtaaggtggagtagaagagagctattggcaagagaaaaattacttgagcagtgtgtgagtgggtgggtgagaaagtgggcagggtggactcagaggttgggaagctgctcctgagaggagaagcctctgtctctacacaggaacctacctgacacatgaggcaaaaggctccgacgatgctccagacgcggacacggccatcatcaatgcagaaggcgggcagtcaggaggggacgacaagaaggaatatttcatctagaggcgcctgcccacttcctgcgccccccaggggccctgtggggactgctggggccgtcaccaacccggacttgtacagagcaaccgcagggccgcccctcccgcttgctccccagcccacccacccccctgtacagaatgtctgctttgggtgcggttttgtactcggtttggaatggggagggaggagggcggggggaggggagggttgccctcagccctttccgtggcttctctgcatttgggttattattatttttgtaa

SEQ ID NO: 230 - DNA, 651 bp, Artificial Sequence
  Description of Artificial Sequence Synthetic polynucleotide
aatcaaattacaagaaagggaaagagaaaggaagagggagtgggacccagagagctggcgggaggcagcgaaggggaaagcttcagtgcacgcatagctcctgcacagcggctcctgcagccccccaggatgcgcctgagctgaggctgcttgtgggcaggccctagagagaggcaaactttgactccaggcacgcagcaggtttaactcctcactggctgggttctgggagctctgggcacacaggataggcaccaagaactaacacatcctggagctgcccggagttccgctcctgcgggcttagcaggaaagggtgcctaaggtgagtgcccacttgcgtccgatcctctgggggcgatgcagggtcggggcgcctcagtgtgtctcgctgcttgttctggttgcagtcgggaaatgtgggactttggggtcttctcctttctccggctttcttttttctccttctttcctctctgttttcttgtaaattacacttcgactttcaaaaaaaaaaatgtaggggaccggtggggtcgctggggttgggggagagactgaagaaagtgcgcctgggcggaggcggcgaagggaatctctgggcccgaggaatataccttgtccctgcactagtgtgtgttctcttgtggc

SEQ ID NO: 231 - DNA, 642 bp, Artificial Sequence
  Description of Artificial Sequence Synthetic polynucleotide
tggttctttctgttgccctcatagaccgtatgtagcagttcgcgtgggcacagaacccacggtttcccgctagttcttcaaaggtgagggcaggtgccccgagttattttcctggggactgagcccagagcggggcgatgttgtgctactgcacctccccgccgcagccctccgctgttttcttttgggtagtggtccaggaacttaagacagttcctcctggcgatgtgatggaatttaatgggacaggagaagggaacgggctttcttttcaggccagcgtggcagcgggcggtagggcgaaagggagaaggaaacgagggtttattccgttgcccactccgcggtaagcgacgttgtagggctccactgtagcgagagccccgtggattcctttttttttagccatttagtttgtaaacatcactttaaagaatacatagtgtattcatgacactcggtgaaaaaaaactttccttcccctcccgcccccccggggcagtagatatttacaaccgtaacagagaaaatggaaaagcaaaagccctttgcattgttcgtaccaccgagatcaagcagcagtcaggtgtctgcggtgaaacctcagaccctgggaggcgattccactttcttcaaggtaaa

SEQ ID NO: 232 - DNA, 624 bp, Artificial Sequence
  Description of Artificial Sequence Synthetic polynucleotide
ccagttcgggatcgtgtagccggcggggcgggggccgtggggggcctggaggagggcaggggccgcgggaggccgggaggagggtggggaccttgcagcccccatcctctccgtgcgcttggagcctctttttgcaaataaagttggtgcagcttcgcggagaggagaggcgctgcagtctgtgctgtgtccgcggggcggggaggaggtcccaggagccggttcgaaagctccctccgtgatgaagtaggcgagaagggaggaggtgaaggagggcgagctgagcacacgcgcttcatgccacaggagggtgggaatgagcggaggactgaggagaggaaggagggaaagaatagggagatgaaaacgccccggtctgctgctaagcacagcacagttaccaaagccaggaaactaacactgacacgatattttatttacgttacagctctattcaaagctccaggcttctttttgtagaatcgtttccatctgctggaatccagcatcgcccccaccccccgccccatttctagggggatgcccccactgctgacctctcctgctgtagatctatttctgggaggcactgacatgctgactcttgctatggggtcggcgggg

SEQ ID NO: 233 - DNA, 595 bp, Artificial Sequence
  Description of Artificial Sequence Synthetic polynucleotide
gggaggccgggaggagggtggggaccttgcagcccccatcctctccgtgcgcttggagcctctttttgcaaataaagttggtgcagcttcgcggagaggagaggcgctgcagtctgtgctgtgtccgcggggcggggaggaggtcccaggagccggttcgaaagctccctccgtgatgaagtaggcgagaagggaggaggtgaaggagggcgagctgagcacacgcgcttcatgccacaggagggtgggaatgagcggaggactgaggagaggaaggagggaaagaatagggagatgaaaacgccccggtctgctgctaagcacagcacagttaccaaagccaggaaactaacactgacacgatattttatttacgttacagctctattcaaagctccaggcttctttttgtagaatcgtttccatctgctggaatccagcatcgcccccaccccccgccccatttctagggggatgcccccactgctgacctctcctgctgtagatctatttctgggaggcactgacatgctgactcttgctatggggtcggcggggagtggggagctgggcattccccttcttcctcaggaca

SEQ ID NO: 234 - DNA, 620 bp, Artificial Sequence
  Description of Artificial Sequence Synthetic polynucleotide
acacacactccctcagaccagatgcccaaccacttccagatgctacagtctcggatatccttggttaaggaagaggaagaaaaagctcgcccttcacgtccagatacttgggttcgggttacatgaaacaggattagttcagaaaatcgtgccacttcacagccaagacaaaaacccaagaatgaaaaccatgtatacagccaacacaatagcaagactgaagacagtgacaaagagagttttctggttctgctctgatgcctctccctccacaccacacctgtgatctactgtgcataggatctcacaggcccaataacagagctggagttcctcttacgtgacacaggatttggcatttgcctgtgccgggctatcactcctgccctgcaacacgctggtcagctggagaagcctgctgctcacacactcaccagcaacttctctaccctggatggtcaccaaaaaggaagagcaatgtctgtgcccccagcattggtgcaaaggaagtggcagagaagcaacaaggagggtggtgccttgccccaactgcccgccagcacccacagccaaggcaactgttctctggtgaaggcagagctggaaatgcatgcctgagc

SEQ ID NO: 235 - DNA, 617 bp, Artificial Sequence
  Description of Artificial Sequence Synthetic polynucleotide
caagggattctcctgcctcagcctcccgagtagctgggataacaggcatgcaccaccacgcctagctaatttttttttttaatgtagtagagatggggtttcaccatgttagccaggatggtctcgatctcctgatcttgtgatccacccacctcggcctcccaaagtgcagggattacaggcgtgagcaccgtgcccgaccaagattgaccttcttaaacaactttgtcatcatgtgcttctcctgctcagacatcctccttggcagcctttcaacacgtttctcaaatcctttcccagcttcctgtgcagcctttcctcctcagcctggctgccttactgtctcagctccgatctctgggccttttcccatatggctgcttccctctacagtgttcctcctagcccataccccaacccaccccacctttccctcctctccaggttgtaccagttccaggcccctgcccttgacaatactccttcccacgaggagcacttcctcggctacctccttagcgtgtattggaattcccactcacttggcagttgcactttgtgacacttaattctgccattttattttcctaactgctatttagtctccctgtttattt

SEQ ID NO: 236 - DNA, 607 bp, Artificial Sequence
  Description of Artificial Sequence Synthetic polynucleotide
gagggagttcaacggcgaccacttccttttggagcgcgccatccgggcagacttcgccctggtgaaagggtggaaggccgaccgggcaggaaacgtggtcttcaggagaagcgcccgcaatttcaacgtgcccatgtgcaaagctgcagacgtcacggcggtggaggtgggggcttccccccagaagacatccacgttcctaacatttatgtaggtcgcgtgataaaggggcagaaatacgagaaacgaattgagcgcttaacgatccggaaagaggaagatggagacgctggaaaggaagaggacgccaggacgcgcatcatcagacgcgcagctctggaatttgaggacggcatgtacgccaatctgggcataggcatccccctgctggccagcaacttcatcagtcccagcatgactgtccatcttcacagtgagaacgggatcctgggcctgggcccgtttcccacggaagatgaggtggatgccgacctcatcaatgcaggcaagcagacggtcacggtgcttcccgggggctgcttcttcgccagcgacgactccttcgccatgatccgagggggacacatccaactaaccatgcttggag

SEQ ID NO: 237 - DNA, 631 bp, Artificial Sequence
  Description of Artificial Sequence Synthetic polynucleotide
agaactggccctcccctcttcactcttttttttttttttcttgagacagagtctcgctctgttgcccaggctggaatgcagtggtgcgatcttggctcactgcaacctctgcctcctgggttcaagcaattctcctgcctcagcctcttgagtagctgggattacaagtgtgtgctaccacacctggctaatttttgtatttttagtagagacagggtttcaccatgttggctgggctcgtcacaaactcctgacctcaggtgatccacccgtctcggcctcccaaagtgttgggattacaggcgtgagccgccgcgcccagccccctcctcactctctttctcttcctgtaacttctacagctgggcaagagctgggtctccagcggttgcacggagaagtgtgtctgcacgggaggagccattcagtgcggggacttccgatgcccctctgggtcccactgccagctcacttccgacaacagcaacagcaattgtgtctcagacagtaaggggagcgaccggggaggttggagaggggagcacctgtggccagggcggaggtggaggaagaggcagggtgggaaggggcttagcctgaaccccagcacagtcaggggttggggcgggcg

SEQ ID NO: 238 - DNA, 581 bp, Artificial Sequence
  Description of Artificial Sequence Synthetic polynucleotide
gaaggatgagaagcattttgccagactcacagcgggacagtattccaagtagagcatggacaaggtacagagaccaggtggctggctttctctagcaacctgtctgctaccccagttcttccatgaccaaactgggtgtattgaacaagttacctcccctctctgagcctcagtttgctcatcagcaaaatgggggtgttggcagagacctttcagacctttcggagttaccaggggtggggtccgcagatccccatagggaggacttgcgcacagttggcgctgggtaaatgctgggagaactgctgcgggcgaggggaaagggttaaagctaggcgctttttaattgtcaaatgactgcgggcgattagcactggcagcttcctcaataatcgctcttctctgtacctgctgggagcttaattaaaaaacaaagaggctcaatttaaaggccattactatgctaatgcggccgggcgggcggtgattaagcggctcaggcaggcagcgggcggctggggcggggcatggggcgatcagttacccactaaatgggcgggctgcgtgccctgcctgtcccg

SEQ ID NO: 239 - DNA, 628 bp, Artificial Sequence
  Description of Artificial Sequence Synthetic polynucleotide
caggacagtgtgggggcggtccctagttcacagcagggaggccctaagcagtgaggtggcctgcccgccatgtcgcaggagcccctagccctggcagactgggcagtagcgctggctggccgctccgggttgtgctgcaggaagcccttctccagccgccagcctcgtcctgcccagccctgggccccacatggcaggaaacaaggccaaagaggcaccgcttagcaagcggcaggacgtgccagggctcacacagacaccctgagcttgcaacactccgggcctctgccgcgtgtttatttcaggatgccgtggcatttgggtgaccttttgtgctcaccatggcttgcgtcgtctccgggtcactctcgtctggactgaagtcccgtctccctcagctgagcctgtgccatggccagcctcagcgggaactggcaaagggaaaggggttccttggggaggcagcaggggtttctgaaggcttcatcttcaagcaaggggtttagagctcaggagtgtatttgtgtttttttgttttttgtttattttgagacagggtctggctgtgtcactcaggctggaatgcaatggcacagtcctggctcactgcagcctcaacctactgggc

SEQ ID NO: 240 - DNA, 651 bp, Artificial Sequence
  Description of Artificial Sequence Synthetic polynucleotide
cttgaatctcctccagtccttccactatatgcccctctggccttgtcttgcacacctccagagacagaagcctcactactttagaggctgtcctttccatcttcctgactggaaggaaattcttccctgaatggagctgaaacttgtgtccctgctctgccctctagggttttttcccagtaactggaagagtcttgaaagggctaaaatgattttatttttaaatgtggacaggcaagcagaggtggttggcaaaggcaaggtggctgacgatccggaagctgtacaggagagataagggcactggctgccagagtgccctatcgaagcatcatccgaaccctgcggtaggggtggcccacaccacggcctgaggcccagtcaatgccatatttgtgggcggcagcctcagacactgcatagcgaccattgagatttgatcggtaacaggatgcataccaccaggcaccgtggacaatcactgcacagttgctgttgcttgaatcgtggtcagcgtcataggtggtaaagggcctcccactgtggaggctcagggaatcccctagcagggaagggatggaaagcaccttggtgcccagcaccacgcctggcacctttggagatataatgccatgggagtctcagagcaac

SEQ ID NO: 241 - DNA, 625 bp, Artificial Sequence
  Description of Artificial Sequence Synthetic polynucleotide
ccagagacagaagcctcactactttagaggctgtcctttccatcttcctgactggaaggaaattcttccctgaatggagctgaaacttgtgtccctgctctgccctctagggttttttcccagtaactggaagagtcttgaaagggctaaaatgattttatttttaaatgtggacaggcaagcagaggtggttggcaaaggcaaggtggctgacgatccggaagctgtacaggagagataagggcactggctgccagagtgccctatcgaagcatcatccgaaccctgcggtaggggtggcccacaccacggcctgaggcccagtcaatgccatatttgtgggcggcagcctcagacactgcatagcgaccattgagatttgatcggtaacaggatgcataccaccaggcaccgtggacaatcactgcacagttgctgttgcttgaatcgtggtcagcgtcataggtggtaaagggcctcccactgtggaggctcagggaatcccctagcagggaagggatggaaagcaccttggtgcccagcaccacgcctggcacctttggagatataatgccatgggagtctcagagcaactaagagttgaattttatcaggccccacgagc

SEQ ID NO: 242 - DNA, 579 bp, Artificial Sequence
  Description of Artificial Sequence Synthetic polynucleotide
ttccatggcccagaagtctgcaggacccacagcaggtattcgggactatttgttcaatccacacctgagtcgttgcacgattatgctcaagtccctcggaacacctcgcctgccatctgacagcttcccatccagaaaccacacagtacagtaaaaaacagaaaaaagaaagccgttagaccccagtgaatgttatttttaatgaaagtggtgcattttgactcacaatgttgaaaccagattataaatgagtcatcagtgaatcgaccacaaagagcctttgcggaggtgatttacaggagagctctgatgtctgctgtcccctgcacacgcttcacagagatgctgtcagacgcagagctggtctggggcatctgttgccgcgtcagctcaaaaggatgctgtgttgtcaccaatgggattccccagcccaggcggtgttgcggtcccacccacacaaggaaggcggccatcactgaataatgcttgtggttacatcatcattgctggtttccaggtagtgactagcagatactggagagagacaggccatctgctcttcctgtgcgcctcagctcc

SEQ ID NO: 243 - DNA, 650 bp, Artificial Sequence
  Description of Artificial Sequence Synthetic polynucleotide
tagcttgtcagcatgaacctacatgcaagccagagatctatgattttgtttcccagggagggagtgactaatgcgcgcaccctgaccatcaccgtaaagaggtaaagagagagtgaaatggctcaacgtacacacaccccgctccataccggggacgagtctccgagctgcggcttgtgctctcggagggccaggctgaagctgaccgcccccacggccacgctggacacccccagccctatctccagcacggagagcaccaagaggctcccaataatctgaccgctggtgcacatccttcctcggtcatcttccttccagatcagagagggaaatcaaccatctacctttttttcttccactatcctccttaccccttccaccccctaccagatcccaaaacttttctttcttcaagagcgaggcattatccacaagggctggatttccagaaacgaagaccttccctggctgggccagaggcaaaggagctgctccaccccctggcacgttcagatagggatcgtagaaggatcttcctgggttcggtggtgcgaagattgcacaccggtaccggggcttttaagcagcggaaaacctggaggagcccagggagctccgagccttgctccccaggcgctgtccagagt

SEQ ID NO: 244 - DNA, 613 bp, Artificial Sequence
  Description of Artificial Sequence Synthetic polynucleotide
tgtcttagtcaggagggttggatgtaagaaacaagcccttaacattcgcttctttgtggacgagatgcagtagaatcatttagtccttgcactctgagcctctccacagaatttgctgttggaagtcatctcagtaagaaatacacagagaaatctggtctttgttcctatgatgacaaagcagtttcataatctgccctcttgcagcttgctctgttttgggtgcagataaaacaagcatggttctctaataaccacctgcacctctgctgcaatgtaaacagcagatgtgggcgcagggtgagaagggagaggaagctacgtgcaatggcaggttggggaataaggaggcagaggggctccttcatcttttacagggtaaaatgggatcaggacagttgcaggacagacttgtttctcaaccacgctgttaagagaatttcatactgcaagtcacaagggcccagggctccacggcctttagcccaccctggctttctaacaacccaaagtgggtatggagaaattgtcctttaaaaacctaaaaactatgttaattttcattttcaaaataaagaataaatcagcacttttggaaaggaggtgggaaggg

SEQ ID NO: 245 - DNA, 635 bp, Artificial Sequence
  Description of Artificial Sequence Synthetic polynucleotide
acgcttgtgataacgataagacagaaactattgaaaagggtgcagtggtggtgtgaaggattaatcctttgcttgcttcacatctgaacaggaatctccacacaaatgtcccacatgtggaagaacctttaatcagagaagtaatctgaaaactcaccttctcacccatacagacatcaagccctacagctgcgagcagtgcggcaaagtgttcaggcgaaactgtgatctgcggcggcacagcctgactcacaccccgcggcaggacttctagagaagcccaggatctgtcccgtgccgccgctgctcccctccccagacacctctccacgtctcctacccagggggtcgcatccctagcccttcactgaccccagctcttcccttgctgcagccgcacctgcagctccagggagttaactcttcttctgggggactgagaactgtagaaagccacacactactacatcccttcacaaagagtatatgctagtttcttgtagatattcacagctcattttagagctctgtacataatgttgtgggtctttgttttgttgttttgtttgctttgggatcttgttggatgcacttagatatggaaaatggaagccaaattttatctttaaagactg

SEQ ID NO: 246 - DNA, 645 bp, Artificial Sequence
  Description of Artificial Sequence Synthetic polynucleotide
ggggaaaactccaggtcagatggggtgaaccagagggaacaatgcacttcttcacaaaccaacatataaacacttgcgaatgaaatcacgcagagacattcatcagcttcaaaaggagagcggaactgggaaaggagtcggcagaattgagagaggagaatttgggaaagcttctccatgagagcggtgcctggagaggtgggttgggaaccgtcgctgagaataaggcacaggtcagccacctttcccagcatctcctcctcgcaaaccccaagccaaggcaagctggatgaagcgctccctgggcaggcccggctctccgtgtccctccatcacctgaccccgctggctctcgcagaccccttcctccacactcactcctcccggctctccttctataatctcctgacatctcttcaaatccaattattgaattaattgacgtacgaacccagaggcaaacagaaaggggcggcaaacactgggcggctcagatttatccttcggcctccgcagggcccggccggacgagatttactgggcctcgaacacggcgacagttcaaacctttgattaatcatgtttttctgcctaccccataatttagttgctctttttccctccctgccttttttttttttttta

SEQ ID NO: 247 - DNA, 650 bp, Artificial Sequence
  Description of Artificial Sequence Synthetic polynucleotide
gagcggaactgggaaaggagtcggcagaattgagagaggagaatttgggaaagcttctccatgagagcggtgcctggagaggtgggttgggaaccgtcgctgagaataaggcacaggtcagccacctttcccagcatctcctcctcgcaaaccccaagccaaggcaagctggatgaagcgctccctgggcaggcccggctctccgtgtccctccatcacctgaccccgctggctctcgcagaccccttcctccacactcactcctcccggctctccttctataatctcctgacatctcttcaaatccaattattgaattaattgacgtacgaacccagaggcaaacagaaaggggcggcaaacactgggcggctcagatttatccttcggcctccgcagggcccggccggacgagatttactgggcctcgaacacggcgacagttcaaacctttgattaatcatgtttttctgcctaccccataatttagttgctctttttccctccctgcctttttttttttttttatcagcggaaacagagacggagtcctcatcagcttcaattacaaatattaaggtcccggacagcactttgacagagaggcggccagccccccacttcgtaccacccccctaaatcatctccga

SEQ ID NO: 248 - DNA, 588 bp, Artificial Sequence
  Description of Artificial Sequence Synthetic polynucleotide
aggtcagccacctttcccagcatctcctcctcgcaaaccccaagccaaggcaagctggatgaagcgctccctgggcaggcccggctctccgtgtccctccatcacctgaccccgctggctctcgcagaccccttcctccacactcactcctcccggctctccttctataatctcctgacatctcttcaaatccaattattgaattaattgacgtacgaacccagaggcaaacagaaaggggcggcaaacactgggcggctcagatttatccttcggcctccgcagggcccggccggacgagatttactgggcctcgaacacggcgacagttcaaacctttgattaatcatgtttttctgcctaccccataatttagttgctctttttccctccctgcctttttttttttttttatcagcggaaacagagacggagtcctcatcagcttcaattacaaatattaaggtcccggacagcactttgacagagaggcggccagccccccacttcgtaccacccccctaaatcatctccgaattaacatcacatcggcggctggcgcgtgttcagatttaaatggtggcatat

SEQ ID NO: 249 - DNA, 632 bp, Artificial Sequence
  Description of Artificial Sequence Synthetic polynucleotide
agctagcgtgttcatgctggatgtggtgataataacagtaacagcagcaacagcaataataatactgtcctatcttttttttttttttttttttttttttttcagaaaagatagcctaaaagggttaagaatcccagcaagacacaacatagatgggctgaaaactcgtggcaggatggaagggtataaagacgccggggaagtggctggggaataataaaataagagggaagctaaaccagtgacccttgtcggcagtgaaaagcgggagattagaaaatgtttcatgctaatttccatggagatttctttaatttagcgaagactgcttcccgggctccgcctggcccgcgccggcccgcgtcctcggtggtctgggcgccccggctgagccgctagcgggtcactcgggcggctccgacgtctctatcagccgcgcccgcgccgcccgcctccccgcgctgctgcccggctctcgggctctcgctttttttttttttttttctttccgcggcagtcttaggattcttgtcacatgatggcttcatcgggcccttctcctcctgatcctttcaagctctttctcctgcctggcatatcaaaggagatttgtgggtcaccgagccgggacg

SEQ ID NO: 250 - DNA, 626 bp, Artificial Sequence
  Description of Artificial Sequence Synthetic polynucleotide
aaataagagggaagctaaaccagtgacccttgtcggcagtgaaaagcgggagattagaaaatgtttcatgctaatttccatggagatttctttaatttagcgaagactgcttcccgggctccgcctggcccgcgccggcccgcgtcctcggtggtctgggcgccccggctgagccgctagcgggtcactcgggcggctccgacgtctctatcagccgcgcccgcgccgcccgcctccccgcgctgctgcccggctctcgggctctcgctttttttttttttttttctttccgcggcagtcttaggattcttgtcacatgatggcttcatcgggcccttctcctcctgatcctttcaagctctttctcctgcctggcatatcaaaggagatttgtgggtcaccgagccgggacgcagcatataaagtcatcagcctggccggcaccacctcgatcatttgccgcattgttcttgcaaggagcccaggatggctgtggctttttaataactagcttagtagttagccgaaaaatcttagtttttaaaaatacaaaaaaaaaaaaaaaaaaaaaagagacagtctgatagtttatttgtttttccatacactcttaattgaaactcagt

SEQ ID NO: 251 - DNA, 644 bp, Artificial Sequence
  Description of Artificial Sequence Synthetic polynucleotide
ctgggggcaaactggagttgtcaggaagatctgggctttggaagaatgcgaagtgtcggtagaaggagaaggggcaggtgatttcagactgggaggaccttgtgggcaaaggcacaaaggcgagactgacctggagatgataaggccagttgaagagacactggagaagagaagacagtttgttttacacattgcaggaaatcagattagacagttagggtgtggacacaaaagcgaggaccttgcaggcactggggagaagtgaccccattcaatagtccttggtctccttctgccctgcggctgcgcttcctcggctctcacggcaccagcagaattccatgtgagagggagcttgtcgagcgtggcctcttcccacttggggctgctttctgcatccctgtgcctggctgtgggcctccatttgccctctactgtcttcccttaggacatcatttatgcagagaaaggttcgtgtggctcggggtaccagtaagacctccacctctggtttcttcattttaaggaggcccttcaattatccaggaattaaagtggccttcctcttgggagaacgagttggttgatgaatgataagcaagtctctattcctcaaaagccagtccccaaattccatgaaatat

SEQ ID NO: 252 - DNA, 622 bp, Artificial Sequence
  Description of Artificial Sequence Synthetic polynucleotide
acacacacacacttccctgagcattcccactttggtaaggaaggagtataatttgctgaatggtgcaagcaagccaggaggacagaagatgttacactttactcagggaacagaggcgggcaactggccctgtgactgcagccaacagctttaagaacacagtcctttctgcttcaaggttagggagacgttctcgcctctttcttctttgcagttattattcaagaggcttcccccgaccccagtccccagcaccatcctcagagcttcagaccatacattgacagtgagcaaagggggccccaggcaggcgggtctggggccaaggagggcggctcccctgcgcggatccttccctggtggctcccaaatccggcgttttctctgccgcctctccctcgggggagactcggaaaggctgcaaaaatctgggcgcccgttcgctcgcttgtcaagaagcaaactgtcttcacattctccaagagcaacatccctgcctaggaagaggaaggaagaggcaaaataaataaaaccagttaatgttgtagttaacttgcaaatcaagtaaatctgttggtgccgtatttgagaaataaaccatcacagcgtcacagcaaacaca

SEQ ID NO: 253 - DNA, 645 bp, Artificial Sequence
  Description of Artificial Sequence Synthetic polynucleotide
ccctgccccgggaggtgctcaggaaagggttgtgaccccgagtgacagtagaggctcagagaggtcaggatgtgtagtgcatggtggagctggccactaactcgggccgcttcttgtcttgtttgagtagcaattgaggggctcctgggtgccccgggctgggctgggcctggagtcagcaagccccaagtcttgccctcccttgccagggaggaaggaaaggtaaccggctgtgacactgagggaggtgagctgggaactggaggtgcagagaaggccccgacgctgtttgtaggttgtgggggtgcagcaagacctagatcttaagaatttcgaaggactgtgacgatcaccggctgcgccctgccggcgagtgccctggggctggctctatttgttgcgcgatccagccctggtggggagatttgtgaggggagacctggctcaggctgtgtcttcctgttcaaacgggggttagtagagagggggttggggaggtccagggagaactgggcatgcagcctgcaggggagagggaccccttggagggctgcgggagaggctccttgtaaatgtcaacaaagacccagccaggcaggtccatgggttaccctaagagcttagagtttatcggagaggaaatgg

SEQ ID NO: 254 - DNA, 618 bp, Artificial Sequence
  Description of Artificial Sequence Synthetic polynucleotide
aggttggggagttgagaaggatggagatgggtgcatctggaagggagtccgtcctgaggagtcccccatcagctgtcagccagccagcagcaaagcaaattaagactacacagctccgaagaagccagttcccaaccaagccagtggagaaaagtcagcccggtccccaggagtgcttgaggctctgtcactcttggacgtcaaaaagggtcatttgatgactggacgcttacctcaccggtgtgaggtaagcttcaaacgccgtatcatgttgctttaaaacctgcgggtaacagcataagctgagttttctatcttagaactcttaaccccaagaacactcttcacaggccctgataggtggacccacaaaaaaaccactcaggctatatttgactcggatttgaaacgctgccgaaacggtattaagtgtcctcctcaactggaaaagacaaataacaaatgatgcctgaatgagaaaaagactagacgtgcacacagtaatgtgtgagcagggaaacttcagcgaaggtttttatcatgctttaccccttttacatgctttaccccatgtgcaaacatttttcatgggtttttttctattttttatttattttt

SEQ ID NO: 255 - DNA, 575 bp, Artificial Sequence
  Description of Artificial Sequence Synthetic polynucleotide
gtcccagcgtcccagcccagctacccaagtagaaggtggggcggcatcttctatggctgagtcttgggcagtgggtgctctgtcatattgtcaggtttcttcccccagctccacaatgtgaagactgaggtggtccctccaagccccactcagcaaggaggacagggctggtggatcctccagggtcagatggggaaataaaagtgtttcattcttgaaggggaagctgcacttctccacggcacgcctggtggtgccaggggttaccacaaagaggcggcagagccatggcccaccagccacttggcaggctggttgtctggtgaagcttttcagggtttggcacagggcccagtcctcagtcccccccatgtccaccacctccactggtgcccccaggcctgggaggtgtaggaggtgccgggggggcatgtaggtgtgagtgaagaggagtgtgtagtgggtgggtgtgcttgggagcacaggggcatggaccacctgctccaggtactccaggccaggcaccaggcccccctgatgcaggcagccgaagaggagggcaccgagggcgttga

SEQ ID NO: 256 - DNA, 613 bp, Artificial Sequence
  Description of Artificial Sequence Synthetic polynucleotide
gaggaatcatgattgtctaaactagtcatggccatcccagtcttctttgctagatacttgagtaatctctttcccagtttctctgttctggccagtgagatgtaagagagagtctgctgagttcttgtgagaactgctttatttcataaagagaacaactaagagagtacttccttcccttcctgtttgggtaaggacttgatgctgcaggagccaccctgcatccataaaggagaagtcaaaaggaatcagagacaacagcccagacccccatcacggagctgcacgtgaccctggaacttaacagcttccagttgttccctagacagtcattgtctttatggtgccttttcccccatcagggaggaaggtgccttgcttcaagtcttttcaattaaactgcagtttaacagagaaaattgccttatgaacagtcaaaagtcagtacaacttaaatatggcagtttatctcaggacatctcccagcacacgtcttcctgagagcaagctgctctcaaaaccaaggcaatgggaaaatggtcagaaacatgacttctgtattttccagttcatacactgaagacagcattcattcattcatttggaaagtcag

SEQ ID NO: 257 - DNA, 617 bp, Artificial Sequence
  Description of Artificial Sequence Synthetic polynucleotide
tgactgtataacactagtagatatttttaaaatgcaagagcatcttcttagatcattacttttccttggaatgcttggcccctgtaatataatacaccggtattttgcatgatgaaattgatgtcctgtgtgttgcttcatgttgctatcctagctgccgattaaaacgttttttttttttcatgccagagcagaacaaaattgtctgcttctcaatctgcacatcataagcagatgacattaaaaatgtctgtaagatgacacagctatattttctgggagagggcgggaggatgctcagcgagggtggcccggagtgtccttgtacagagtacagatgttatgaagtggggaagaccagcctgtgttcattgattcacctattgattccaggagcaagctcaccctgtttcatacactgctcaggaggtaaacaggaggaagggagccagcctggcttttttgccacatgctctgctgtttggtagaactgtattatagtcagaaaccttccgcttttctgcagttgtttgcatgctgtttccaaggctagccctctgagtctgttttctagagttgttttgaaattcaacctaaagataacagaggaaatgtga

SEQ ID NO: 258 - DNA, 609 bp, Artificial Sequence
  Description of Artificial Sequence Synthetic polynucleotide
gcgcggcacaacggcgcattgtggggccaagcgaggggcgaagggggctgggggtggccggcgcgatggggacgctccggttgcgccaagttgactctgccgtttgggtcacctgggctgagtcgcgggcgtggaggcagagggtagggggtgaggaggtgtttcttgtcttcttcttccaatctcagaagtaaacattggaaagtggggcccccagcagtgtacagcccgtttccaaaccaggcctgtaaggaggagctgaggtttcggctgagcccccagcctcccccgaccgcacagcctcgggcatgaacccgcgaagccagacgcttagttgcttatcaggccatcgctgtacatatttagaaagtacctatcactcagacactttgaaaagcgtggcgttccagcgcaaaccaacccgaacgggttggaagggggcagtcctttcttcccgcaagttcggggctcgagagacggctgcaggaaggccatcacccctggcttcctgcagccacagcttccagccccacacgatgcccaacttcattttagcagtggcccccaggggaaatcacaccattcttggttttgtccctccctcctgag

SEQ ID NO: 259 - DNA, 621 bp, Artificial Sequence
  Description of Artificial Sequence Synthetic polynucleotide
cttagcaatctgcatttctaaagtgctttgtacacattccgtctatgttaaaagcctcagcagcaggttggaggcgggttctggggctagtgtttccgatgggaagctcaggctccatccagcctgtggctggactggccaggctcaatgtcactccccaggtagctgcccttgatctatactaggagcaccttgagagctgggaattgatttctaagcctggtttgagctgagggccacagagccagtgcaggaggagaccctgccccagaaataggccagtgcttgttatgcaggccttggcggttccccgtttccttacgtaacctcagtgttcacgctgtttccttttgttgattccctccgtgtgactgtttttctgtcaatctccttagctaatgagctccttataaggagaatggatggatcagagcacagctccgtacacagtggtggggcatagccatttcccagagtgtggactttcccagaactcccctgttgtgtgggcctgcaaaggctgggattgtttctgccttgtttggaataataaagctgcctgtgtttcctgtgttcacttttcagtcgcctgttattcactctcctacatttggggcggtt